(19) United States
(12) Patent Application Publication
(10) Pub. No.: US 2024/0366722 A1
(43) Pub. Date: Nov. 7, 2024

(54) COMPOSITIONS AND METHODS FOR TREATING RENAL DISEASES OR CONDITIONS

(71) Applicant: ACCELERON PHARMA INC., Cambridge, MA (US)

(72) Inventor: Ravindra Kumar, Cambridge, MA (US)

(21) Appl. No.: 18/683,894

(22) PCT Filed: Aug. 15, 2022
(86) PCT No.: PCT/US2022/040308

§ 371 Date: Feb. 15, 2024

Related U.S. Application Data
(60) Provisional application No. 63/233,470, filed on Aug. 16, 2021.

(51) Int. Cl. A61K 38/17

(52) U.S. Cl. CPC A61K 38/179; A61K 38/1774

(57) ABSTRACT

In some aspects, the disclosure relates to compositions and methods comprising ActRII antagonists to treat, prevent, or reduce the progression rate and/or severity of a renal disease or condition, particularly treating, preventing or reducing the progression rate and/or severity of one or more renal diseases or conditions, particularly treating, preventing or reducing the progression rate and/or severity of one or more renal-associated complications. The disclosure also provides methods of using an ActRII antagonist to treat, prevent, or reduce the progression rate and/or severity of a variety of conditions including, but not limited to, Alport syndrome, focal segmental glomerulosclerosis (FSGS), polycystic kidney disease, and/or chronic kidney disease.

### CROSS-REFERENCE TO RELATED APPLICATIONS

This application claims the benefit of and priority to U.S. Provisional Application No. 63/233,470, filed Aug. 16, 2021. The foregoing application is incorporated herein by reference in its entirety.

### FIELD OF INVENTION

This application relates to ActRII antagonists, compositions, and methods comprising ActRII antagonists to treat, prevent, or reduce the progression rate and/or severity of a renal disease or condition, particularly treating, preventing or reducing the progression rate and/or severity of one or more renal-associated complications.

### BACKGROUND OF THE INVENTION

Renal diseases include a range of conditions that can lead to loss of kidney function, and, in some cases, can be fatal. Normally-functioning kidneys filter wastes and excess fluids from the blood, which are then excreted in urine. For example, when chronic kidney disease reaches an advanced stage, dangerous levels of fluid, electrolytes and wastes can build up in the bloodstream. If left untreated, renal disease can progress to end-stage renal disease (e.g., end-stage kidney failure), which is fatal without artificial filtering (dialysis) or a kidney transplant. Thus, there is a high, unmet need for effective therapies for treating renal diseases or conditions (e.g., Alport syndrome, focal segmental glomerulosclerosis (FSGS), polycystic kidney disease, chronic kidney disease).

### SUMMARY OF THE INVENTION

In part, the disclosure provides ActRII antagonists (e.g., ActRII polypeptides) that can be used to treat renal diseases or conditions (e.g., Alport syndrome, focal segmental glomerulosclerosis (FSGS), polycystic kidney disease, chronic kidney disease). Therefore, in some embodiments, the disclosure provides methods for using various ActRII signaling antagonists for treating renal diseases or conditions (e.g., Alport syndrome, focal segmental glomerulosclerosis (FSGS), polycystic kidney disease, chronic kidney disease), including, for example, antagonists that inhibit one or more TGF-beta family ligands (e.g., activin A, activin B, activin AB, activin AC, BMP6, BMP7, BMP9, BMP10, GDF3, GDF8, and GDF11); antagonists that inhibit ActRIIA or ActRIIB; and antagonists that inhibit one or more downstream signaling components (e.g., SMAD proteins). As used herein, such signaling antagonists are collectively referred to as “ActRII antagonists” or “ActRII inhibitors”. Accordingly, the disclosure provides, in part, ActRII antagonist compositions and methods for renal diseases or conditions (e.g., Alport syndrome, focal segmental glomerulosclerosis (FSGS), polycystic kidney disease, chronic kidney disease), particularly treating one or more complications of renal diseases or conditions. ActRII antagonists to be used in accordance with the methods and uses of the disclosure include, for example, ligand traps (e.g., soluble ActRIIA polypeptides or ActRIIB polypeptides). Optionally, ActRII antagonists may be used in combination with one or more supportive therapies and/or additional active agents for treating renal diseases or conditions. In some embodiments, the present disclosure provides methods of treating renal diseases or conditions, comprising administering to a patient in need thereof an effective amount of an ActRIIA variant polypeptide. In some embodiments, the present disclosure provides methods of treating, preventing, or reducing the progression rate and/or severity of one or more complications of a renal disease or condition, comprising administering to a patient in need thereof an effective amount of an ActRIIA variant polypeptide. In some embodiments, the present disclosure provides methods of treating a renal disease or condition, comprising administering to a patient in need thereof an effective amount of an ActRIIB variant polypeptide. In some embodiments, the present disclosure provides methods of treating, preventing, or reducing the progression rate and/or severity of one or more complications of a renal disease or condition, comprising administering to a patient in need thereof an effective amount of an ActRIIB variant polypeptide.

In some embodiments of the present disclosure, the renal disease or condition is Alport syndrome. In some embodiments, the renal disease or condition is focal segmental glomerulosclerosis (FSGS). In some embodiments, the FSGS is primary FSGS. In some embodiments, the FSGS is secondary FSGS. In some embodiments, the FSGS is genetic FSGS. In some embodiments the renal disease or condition is autosomal dominant polycystic kidney disease (ADPKD). In some embodiments, the renal disease or condition is autosomal recessive polycystic kidney disease (ARPKD). In some embodiments, the renal disease or condition is chronic kidney disease (CKD). In some embodiments, the patient has a decline in kidney function. In some embodiments, methods of the present disclosure slow kidney function decline.

In some embodiments, methods of the present disclosure comprise further administering to the patient an additional active agent and/or supportive therapy for treating a renal disease or condition. In some embodiments, the additional active agent and/or supportive therapy for treating a renal disease or condition is selected from the group consisting of: an angiotensin receptor blocker (ARB) (e.g., losartan, irbesartan, olmesartan, candesartan, valsartan, fimasartan, azilsartan, salprisartan, and telmisartan), an angiotensin-converting enzyme (ACE) inhibitor (e.g., benazepril, captopril, enalapril, lisinopril, perindopril, ramipril, trandolapril, and zofenopril), a glucocorticoid (e.g., beclomethasone, betamethasone, budesonide, cortisone, dexamethasone, hydrocortisone, methylprednisolone, prednisolone, methylprednisone, prednisone, and triamcinolone), a calcineurin inhibitor (e.g., cyclosporine, tacrolimus), cyclophosphamide, chlorambucil, a janus kinase inhibitor (e.g., tofacitinib), an mTOR inhibitor (e.g., sirolimus, everolimus), an IMDH inhibitor (e.g., azathioprinc, leflunomide, mycophenolate), a biologic (e.g., abatacept, adalimumab, anakinra, basiliximab, certolizumab, daclizumab, etanercept, fresolimumab, golimumab, infliximab, ixckizumab, natalizumab, rituximab, secukinumab, tocilizumab, ustekinumab, vedolizumab), a statin (e.g., benazepril, valsartan, fluvastatin, pravastatin), lademirsen (anti-miRNA-21), bardoxolone methyl, Achtar gel, tolvaptan, abatacept in combination with sparsentan, aliskiren, allopurinol, ANG-3070, atorvastatin, bleselumab, bosutinib, CCX140-B, CXA-10, D6-25-hydroxyvitamin D3, dapagliflozin, dexamethasone in combination with MMF, emodin, FG-3019, FK506, FK-506 and MMF, FT-011, galactose, GC1008, GFB-887, isotretinoin, lanreotide, levamisole, lixivaptan, losmapimod, metformin, mizorbine, N-acetylmannosamine, octreotide, paricalcitol, PF-06730512, pioglitazone, propagermanium, propagermanium and irbesartan, rapamune, rapamycin, RE-021 (e.g., sparsentan), RG012, rosiglitazone (e.g., Avandia), saquinivir, SAR339375, somatostatin, spironolactone, tesevatinib (KD019), tetracosactin, tripterygium wilfordii (TW), valproic acid, VAR-200, venglustat (GZ402671), verinurad, voclosporin, VX-147, kidney dialysis, kidney transplant, mesenchymal stem cell therapy, bone marrow stem cells, lipoprotein removal, a Liposorber LA-15 device, plasmapheresis, plasma exchange, and a change in diet (e.g., dietary sodium intake). In some embodiments, an additional active agent and/or supportive therapy for treating a renal disease or condition is an angiotensin receptor blocker (ARB) selected from the group consisting of losartan, irbesartan, olmesartan, candesartan, valsartan, fimasartan, azilsartan, salprisartan, and telmisartan. In some embodiments, an additional active agent and/or supportive therapy for treating a renal disease or condition is an angiotensin-converting enzyme (ACE) inhibitor selected from the group consisting of benazepril, captopril, enalapril, lisinopril, perindopril, ramipril, trandolapril, and zofenopril. In some embodiments, an additional active agent and/or supportive therapy for treating a renal disease or condition is a combination of an ARB and an ACE inhibitor.

In some embodiments, methods of the present disclosure reduce severity, occurrence and/or duration of one or more of albuminuria, proteinuria, microalbuminuria, and macroalbuminuria in a patient in need thereof. In some embodiments of the present disclosure, the patient has proteinuria prior to treatment via a method of the invention. In some embodiments, the patient has albuminuria prior to treatment. In some embodiments, the patient has moderate albuminuria prior to treatment. In some embodiments, the patient has severe albuminuria prior to treatment. In some embodiments, the patient has an albumin-creatinine ratio (ACR) of between about 30 and about 300 mg albumin per 24 hours of urine collection prior to treatment. In some embodiments, the patient has an ACR of between about 30 and about 300 mg albumin/g of creatinine prior to treatment. In some embodiments, the patient has an ACR of above about 300 mg albumin/24 hours prior to treatment. In some embodiments, the patient has an ACR of above about 300 mg albumin/g of creatinine prior to treatment. In some embodiments, the patient has Stage A1 albuminuria. In some embodiments, the patient has Stage A2 albuminuria. In some embodiments, the patient has Stage A3 albuminuria. In some embodiments, the present disclosure provides methods of reducing severity, occurrence and/or duration of Stage A1 albuminuria. In some embodiments, the present disclosure provides methods of reducing severity, occurrence and/or duration of Stage A2 albuminuria. In some embodiments, the present disclosure provides methods of reducing severity, occurrence and/or duration of Stage A3 albuminuria. In some embodiments, methods of the present disclosure delay or prevent a patient with Stage A1 albuminuria from progressing to Stage A2 albuminuria. In some embodiments, methods of the present disclosure delay or prevent a patient with Stage A2 from progressing to Stage A3 albuminuria. In some embodiments, methods of the present disclosure delay or prevent worsening of albuminuria stage progression in a patient in need thereof. In some embodiments, methods of the present disclosure improve albuminuria classification in a patient by one or more stages.

In some embodiments, methods of the present disclosure reduce an ACR of the patient. In some embodiments, the method reduces the patient's ACR by between about 0.1 and about 100.0 mg albumin/g creatinine (e.g., by between about 0.1 and about 2.5 mg albumin/gg, between about 2.5 and about 3.5 mg albumin/g creatinine, between about 3.5 and about 5.0 mg albumin/g creatinine, between about 5.0 and about 7.5 mg albumin/g creatinine, between about 7.5 and about 10.0 mg albumin/g creatinine, between about 10.0 and about 15.0 mg albumin/g creatinine, between about 15.0 and about 20.0 mg albumin/g creatinine, between about 20.0 and about 25.0 mg albumin/g creatinine, between about 30.0 and about 35.0 mg albumin/g creatinine, between about 40.0 and about 45.0 mg albumin/g creatinine, between about 45.0 and about 50.0 mg albumin/g creatinine, between about 50.0 and about 60.0 mg albumin/g creatinine, between about 60.0 and about 70.0 mg albumin/g creatinine, between about 70.0 and about 80.0 mg albumin/g creatinine, between about 80.0 and about 90.0 mg albumin/g creatinine, between about 90.0 and about 100.0 mg albumin/g creatinine). In some embodiments, the method reduces the patient's ACR by at least 2.5% (e.g., 5%, 10%, 15%, 20%, 25%, 30%, 40%, 50%, 60%, 70%, 80%, 90%, 95%, or 99%) compared to a baseline measurement.

In some embodiments, methods of the present disclosure reduce a urinary protein-creatinine ratio (UPCR) of the patient. In some embodiments, the method reduces the patient's UPCR by between about 0.1 and about 100.0 mg urinary protein/mg creatinine (e.g., by between about 0.1 and about 2.5 mg urinary protein/mg creatinine, between about 2.5 and about 3.5 mg urinary protein/mg creatinine, between about 3.5 and about 5.0 mg urinary protein/mg creatinine, between about 5.0 and about 7.5 mg urinary protein/mg creatinine, between about 7.5 and about 10.0 mg urinary protein/mg creatinine, between about 10.0 and about 15.0 mg urinary protein/mg creatinine, between about 15.0 and about 20.0 mg urinary protein/mg creatinine, between about 20.0 and about 25.0 mg urinary protein/mg creatinine, between about 30.0 and about 35.0 mg urinary protein/mg creatinine, between about 40.0 and about 45.0 mg urinary protein/mg creatinine, between about 45.0 and about 50.0 mg urinary protein/mg creatinine, between about 50.0 and about 60.0 mg urinary protein/mg creatinine, between about 60.0 and about 70.0 mg urinary protein/mg creatinine, between about 70.0 and about 80.0 mg urinary protein/mg creatinine, between about 80.0 and about 90.0 mg urinary protein/mg creatinine, between about 90.0 and about 100.0 mg urinary protein/mg creatinine).

In some embodiments, methods of the present disclosure reduce a urinary protein-creatinine ratio (UPCR) of the patient. In some embodiments, the method reduces the patient's UPCR by between about 0.1 and about 100.0 g urinary protein/g creatinine (e.g., by between about 0.1 and about 2.5 g urinary protein/g creatinine, between about 2.5 and about 3.5 g urinary protein/g creatinine, between about 3.5 and about 5.0 g urinary protein/g creatinine, between about 5.0 and about 7.5 g urinary protein/g creatinine, between about 7.5 and about 10.0 g urinary protein/g creatinine, between about 10.0 and about 15.0 g urinary protein/g creatinine, between about 15.0 and about 20.0 g urinary protein/g creatinine, between about 20.0 and about 25.0 g urinary protein/g creatinine, between about 30.0 and about 35.0 g urinary protein/g creatinine, between about 40.0 and about 45.0 g urinary protein/g creatinine, between about 45.0 and about 50.0 g urinary protein/g creatinine, between about 50.0 and about 60.0 g urinary protein/g creatinine, between about 60.0 and about 70.0 g urinary protein/g creatinine, between about 70.0 and about 80.0 g urinary protein/g creatinine, between about 80.0 and about 90.0 g urinary protein/g creatinine, between about 90.0 and about 100.0 g urinary protein/g creatinine). In some embodiments, the method reduces the patient's absolute UPCR by greater than or equal to 0.5 g urinary protein/g creatinine compared to a baseline measurement. In some embodiments, the method reduces the patient's UPCR to less than 0.5 g urinary protein/g creatinine compared to a baseline measurement. In some embodiments, the method reduces the patient's UPCR to less than 0.3 g urinary protein/g creatinine compared to a baseline measurement (e.g. a measurement taken before treatment with a method of the invention). In some embodiments, the method reduces the patient's UPCR by at least 2.5% (e.g., 5%, 10%, 15%, 20%, 25%, 30%, 40%, 50%, 60%, 70%, 80%, 90%, 95%, or 99%) compared to a baseline measurement. In some embodiments, the method reduces the patient's UPCR by greater than or equal to 30% compared to a baseline measurement. In some embodiments, the method reduces the patient's UPCR by greater than or equal to 40% compared to a baseline measurement. In some embodiments, the method reduces the patient's UPCR by greater than or equal to 50% compared to a baseline measurement.

In some embodiments, methods of the present disclosure increase the patient's estimated glomerular filtration rate (eGFR) and/or glomerular filtration rate (GFR). In some embodiments, the eGFR is measured using serum creatinine, age, ethnicity, and gender variables. In some embodiments, the eGFR is measured using one or more of Cockcroft-Gault formula, Modification of Diet in Renal Disease (MDRD) formula, CKD-EPI formula, Mayo quadratic formula, and Schwartz formula. In some embodiments, the eGFR and/or GFR is increased by at least 2.5% (e.g., 5%, 10%, 15%, 20%, 25%, 30%, 40%, 50%, 60%, 70%, 80%, 90%, 95%, or 99%) compared to a baseline measurement. In some embodiments, the eGFR and/or GFR is increased by greater than or equal to 30% compared to a baseline measurement. In some embodiments, the eGFR and/or GFR is increased by greater than or equal to 40% compared to a baseline measurement.

In some embodiments, the eGFR and/or GFR is increased by at least 1 ml/min/1.73 m² (e.g., 3, 5, 7, 9, 10, 15, 20, 25, 30, 35, 40, 45, 50, 55, 60, 65, 70, 75, 80, 85, 90, 95, or 100 mL/min/1.73 m²) compared to a baseline measurement. In some embodiments, the eGFR and/or GFR is increased by at least 1 mL/min/year (e.g., 2, 3, 5, 7, 9, 10, 15, 20, 25, 30, 35, 40, 45, 50, 55, 60, 65, 70, 75, 80, 85, 90, 95, or 100 mL/min/year) compared to a baseline measurement. In some embodiments, the eGFR and/or GFR is increased by greater than or equal to 1 mL/min/year compared to a baseline measurement. In some embodiments, the eGFR and/or GFR is increased by greater than or equal to 3 mL/min/year compared to a baseline measurement.

In some embodiments of the present disclosure, the renal disease or condition of a patient is evaluated in stages of chronic kidney disease (CKD). In some embodiments, the patient has stage one CKD. In some embodiments, the patient has stage two CKD. In some embodiments, the patient has stage three CKD. In some embodiments, the patient has stage four CKD. In some embodiments, the patient has stage five CKD. In some embodiments, methods of the present disclosure reduce severity, occurrence and/or duration of Stage 1 CKD. In some embodiments, methods of the present disclosure reduce severity, occurrence and/or duration of Stage 2 CKD. In some embodiments, methods of the present disclosure reduce severity, occurrence and/or duration of Stage 3 CKD. In some embodiments, methods of the present disclosure reduce severity, occurrence and/or duration of Stage 3a CKD. In some embodiments, methods of the present disclosure reduce severity, occurrence and/or duration of Stage 3b CKD. In some embodiments, methods of the present disclosure reduce severity, occurrence and/or duration of Stage 4 CKD. In some embodiments, methods of the present disclosure reduce severity, occurrence and/or duration of Stage 5 CKD. In some embodiments, methods of the present disclosure prevent or delay a patient with Stage 1 CKD from progressing to Stage 2 CKD. In some embodiments, methods of the present disclosure prevent or delay a patient with Stage 2 CKD from progressing to Stage 3 CKD. In some embodiments, methods of the present disclosure prevent or delay a patient with Stage 2 CKD from progressing to Stage 3a CKD. In some embodiments, methods of the present disclosure prevent or delay a patient with Stage 3a CKD from progressing to Stage 3b CKD. In some embodiments, methods of the present disclosure prevent or delay a patient with Stage 3 CKD from progressing to Stage 4 CKD. In some embodiments, methods of the present disclosure prevent or delay a patient with Stage 3b CKD from progressing to Stage 4 CKD. In some embodiments, methods of the present disclosure prevent or delay a patient with Stage 4 CKD rom progressing to Stage 5 CKD. In some embodiments, methods of the present disclosure prevent or delay worsening of CKD stage progression in a patient in need thereof. In some embodiments, methods of the present disclosure improve renal damage CKD classification in a patient by one or more stages.

In some embodiments, methods of the present disclosure reduce total kidney volume in a patient. In some embodiments, the total kidney volume is reduced by at least 2.5% (e.g., 5%, 10%, 15%, 20%, 25%, 30%, 40%, 50%, 60%, 70%, 80%, 90%, 95%, or 99%) compared to a baseline measurement.

In some embodiments, methods of the present disclosure reduce the patient's blood urea nitrogen (BUN). In some embodiments, the BUN is reduced by at least 2.5% (e.g., 5%, 10%, 15%, 20%, 25%, 30%, 40%, 50%, 60%, 70%, 80%, 90%, 95%, or 99%) compared to a baseline measurement.

In some embodiments, methods of the present disclosure reduce urine Neutrophil Gelatinase-Associated Lipocalin (uNGAL) concentration in a patient. In some embodiments, the uNGAL is reduced by at least 2.5% (e.g., 5%, 10%, 15%, 20%, 25%, 30%, 40%, 50%, 60%, 70%, 80%, 90%, 95%, or 99%) compared to a baseline measurement. In some embodiments, the patient has a uNGAL measurement of <50 ng/ml, an indication of low risk of acute kidney injury. In some embodiments, the patient has a uNGAL measurement of between about 50 and about 149 ng/mL, an indication of equivocal risk of acute kidney injury. In some embodiments, the patient has a uNGAL measurement of between about 150 and about 300 ng/mL, an indication of moderate risk of acute kidney injury. In some embodiments, the patient has a uNGAL measurement of >300 ng/ml, an indication of high risk of acute kidney injury. In some embodiments of the present disclosure, the method reduces the patient's uNGAL by between about 0.1 and about 300.0 ng/ml (e.g., by between about 0.1 and about 50 ng/mL, by between about 0.1 and about 100.0 ng/ml, by between about 0.1 and about 150.0 ng/ml, by between about 0.1 and about 200.0 ng/ml, by between about 0.1 and about 250.0 ng/ml, by between about 0.1 and about 300.0 ng/mL, by between about 0.1 and about 25 ng/ml, by between about 25 and about 50 ng/ml, by between about 50 and about 100 ng/ml, by between about 100 and about 150 ng/ml, by between about 150 and about 200 ng/mL, by between about 200 and about 250 ng/ml, by between about 250 and about 300 ng/ml, by more than 300 ng/ml).

In some embodiments, methods of the present disclosure prevent or delay clinical worsening of a renal disease or condition (e.g., Alport syndrome, focal segmental glomerulosclerosis (FSGS), polycystic kidney disease, chronic kidney disease). In some embodiments, methods of the present disclosure reduce risk of hospitalization for one or more complications associated with a renal disease or condition (e.g., Alport syndrome, focal segmental glomerulosclerosis (FSGS), polycystic kidney disease, chronic kidney disease)

In certain aspects, an ActRIIA variant polypeptide, to be used in accordance with methods and uses described herein, is a ActRIIA variant polypeptide that comprises the sequence of GAILGRSETQECLXIX₂NANWX₃X₄X₅X₆TNQTGVEX-CX&GX₉X₁₀X₁₁X₁₂X₁₃X₁₄HCX₁₅A TWX₁₆NISGSIEIVX₁₇X₁₈GCX₁₉X₂₀X₂₁DX₂₂NCYDRTDCVEX₂₃X₂₄X₂₅X₂₆PX₂₇VYFCCCE GNMCNEKFSYFPEMEVTQPTS (SEQ ID NO: 139), wherein X₁ is F or Y; X₂ is F or Y; X₃ is E or A; X₄ is K or L; X₅ is D or E; X₆ is R or A; X₇ is P or R; X₈ is Y or E; X₉ is D or E; X₁₀ is K or Q; X₁₁ is D or A; X₁₂ is K or A; X₁₃ is R or A; X₁₄ is R or L; X₁₅ is F or Y; X₁₆ is K, R, or A; X₁₇ is K, A, Y, F, or I; X₁₈ is Q or K; X₁₉ is W or A; X₂₀ is L or A; X₂₁ is D, K, R, A, F, G, M, N, or I; X₂₂ is I, F, or A; X₂₃ is K or T; X₂₄ is K or E; X₂₅ is D or E; X₂₆ is S or N; and X₂₇ is E or Q, and wherein the ActRIIA variant polypeptide has at least one amino acid substitution relative to a wild-type extracellular ActRIIA having the sequence of SEQ ID NO: 211 or an extracellular ActRIIA having any one of the sequences of SEQ ID NOs: 212-232. In some embodiments, the ActRIIA variant polypeptide has a sequence of GAILGRSETQECLFX₂NANWX₃X₄X₅X₆TNQTGVEX-CX&GX₉KX₁₁X₁₂X₁₃X₁₄HCX₁₅AT WX₁₆NISGSIEIVX₁₇X₁₈GCX₁₉X₂₀X₂₁DX₂₂NCYDRTDCVEX₂₃X₂₄X₂₅X₂₆PX₂₇VYFCCCEG NMCNEKFSYFPEMEVTQPTS (SEQ ID NO: 140). In some embodiments, the ActRIIA variant polypeptide has a sequence of GAILGRSETQECLFX₂NANWEX₄X₅RTNQTGVEX-CX₇GX₈KDKRX₁₄HCX₁₅ATWX₁₆NI SGSIEIVKX₁₈GCWLDDX₂₂NCYDRTDCVEX₂₃X₂₄X₂₅X₂₆PX₂₇VYFCCCEGNMCNEKFSY FPEMEVTQPTS (SEQ ID NO: 141). In some embodiments, the ActRIIA variant polypeptide has a sequence of GAILGRSETQECLFX₂NANWEX₄DRTNQTGVEX₇CX₈GX₉KDKRX₁₄HCX₁₅ATWX₁₆NIS GSIEIVKX₁₈GCWLDDX₂₂NCYDRTDCVEX₂₃KX₂₅X₂₆PX₂₇VYFCCCEGNMCNEKFSYFP EMEVTQPTS (SEQ ID NO: 142). In some embodiments, the ActRIIA variant polypeptide has a sequence of GAILGRSETQECLFX₂NANWEX₄DRTNQTGVEPCX₈GX₉KDKRX₁₄HCFATWKNISGSI EIVKX₁₈GCWLDDINCYDRTDCVEX₂₃KX₂₅X₂₆PX₂₇VYFCCCEGNMCNEKFSYFPEMEV TQPTS (SEQ ID NO: 143). In some embodiments, X₁ is F. In some embodiments, X₁ is Y. In some embodiments, X₁₀ is K. In some embodiments, X₁₀ is Q. In some embodiments, X₂ is F. In some embodiments, X₂ is Y. In some embodiments, X₃ is E. In some embodiments, X₃ is A. In some embodiments, X₄ is K. In some embodiments, X₄ is L. In some embodiments, X₅ is D. In some embodiments, X₅ is E. In some embodiments, X₆ is R. In some embodiments, X₆ is A. In some embodiments, X₇ is P. In some embodiments, X₇ is R. In some embodiments, X₈ is Y. In some embodiments, X₈ is E. In some embodiments, X₉ is D. In some embodiments, X₉ is E. In some embodiments, X₁₁ is D. In some embodiments, X₁₁ is A. In some embodiments, X₁₂ is K. In some embodiments, X₁₂ is A. In some embodiments, X₁₃ is R. In some embodiments, X₁₃ is A. In some embodiments, X₁₄ is R. In some embodiments, X₁₄ is L. In some embodiments, X₁₅ is F. In some embodiments, X₁₅ is Y. In some embodiments, X₁₆ is K. In some embodiments, X₁₆ is R. In some embodiments, X₁₆ is A. In some embodiments, X₁₇ is K. In some embodiments, X₁₇ is A. In some embodiments, X₁₇ is Y. In some embodiments, X₁₇ is F. In some embodiments, X₁₇ is I. In some embodiments, X₁₈ is Q. In some embodiments, X₁₈ is K. In some embodiments, X₁₉ is W. In some embodiments, X₁₉ is A. In some embodiments, X₂₀ is L. In some embodiments, X₂₀ is A. In some embodiments, X₂₁ is D. In some embodiments, X₂₁ is K. In some embodiments, X₂₁ is R. In some embodiments, X₂₁ is A. In some embodiments, X₂₁ is F. In some embodiments, X₂₁ is G. In some embodiments, X₂₁ is M. In some embodiments, X₂₁ is N. In some embodiments, X₂₁ is I. In some embodiments, X₂₂ is I. In some embodiments, X₂₂ is F. In some embodiments, X₂₂ is A. In some embodiments, X₂₃ is K. In some embodiments, X₂₃ is T. In some embodiments, X₂₄ is K. In some embodiments, X₂₄ is E. In some embodiments, X₂₅ is D. In some embodiments, X₂₅ is E. In some embodiments, X₂₆ is S. In some embodiments, X₂₆ is N. In some embodiments, X₂₇ is E. In some embodiments, X₂₇ is Q. In some embodiments, X₂₃ is T, X₂₄ is E, X₂₅ is E, and X₂₆ is N. In some embodiments, X₂₃ is T, X₂₄ is E, X₂₅ is E, and X₂₆ is N. In some embodiments, X₁₇ is K. In some embodiments, the ActRIIA variant polypeptide has the sequence of any one of SEQ ID NOs: 145-210. In some embodiments, the amino acid at position X₂₄ is replaced with the amino acid K. In some embodiments, the amino acid at position X₂₄ is replaced with the amino acid E. In some embodiments, the ActRIIA variant polypeptide further comprises a C-terminal extension of one or more amino acids. In some embodiments, the C-terminal extension is NP. In some embodiments, the C-terminal extension is NPVTPK (SEQ ID NO: 410). In some embodiments, the ActRIIA variant polypeptide binds to one or more ligands selected from the group consisting of: activin A, activin B, GDF11, GDF8, and BMP6. In some embodiments, the ActRIIA variant polypeptide does not bind or does not substantially bind to one or more ligands selected from the group consisting of: BMP10, BMP9, and GDF3. In some embodiments, the ActRIIA variant polypeptide is lyophilized. In some embodiments, the ActRIIB variant polypeptide is reconstituted following lyophilization. In some embodiments, the ActRIIA variant polypeptide is soluble. In some embodiments, the ActRIIA variant polypeptide is administered using subcutaneous injection. In some embodiments, ActRIIA variant polypeptide is administered about every 4 weeks. In some embodiments, ActRIIA variant polypeptide is administered every 4 weeks.

In some embodiments, the ActRIIA variant polypeptide if part of a homodimer protein complex. In some embodiments, the ActRIIA variant polypeptide comprises an amino acid sequence selected from the group consisting of SEQ ID NO: 196 and SEQ ID NO: 207. In some embodiments, the ActRIIA polypeptide is a fusion protein further comprising a heterologous domain. In some embodiments, the heterologous domain is an Fc immunoglobulin domain. In some embodiments, the ActRIIA variant polypeptide and/or fusion protein further comprises a linker domain positioned between the ActRIIA variant polypeptide and the heterologous domain. In some embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In some embodiments, the ActRIIA variant polypeptide or fusion protein comprises one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety. In some embodiments, the ActRIIA variant polypeptide or fusion protein is glycosylated and has a mammalian glycosylation pattern. In some embodiments, the ActRIIA variant polypeptide or fusion protein has a glycosylation pattern obtainable from a Chinese hamster ovary cell line. In some embodiments, the ActRIIA variant polypeptide or fusion protein binds to one or more ligands selected from the group consisting of: activin A, activin B, GDF11, GDF8, and BMP6. In some embodiments, the ActRIIA variant polypeptide or fusion protein binds to activin A. In some embodiments, the ActRIIA variant polypeptide or fusion protein inhibits one or more TGFβ superfamily ligands selected from the group consisting of: activin A, activin B, GDF11, GDF8, and BMP6. In some embodiments, the ActRIIA variant polypeptide or fusion protein inhibits activin A. In some embodiments, the ActRIIA variant polypeptide or fusion protein does not bind or does not substantially bind to one or more ligands selected from the group consisting of: BMP10, BMP9, and GDF3. In some embodiments, the ActRIIA variant polypeptide or fusion protein binds to one or more of BMP10, BMP9, or GDF3 with lower affinity compared to a corresponding wild-type ActRIIA polypeptide. In some embodiments, the ActRIIA variant polypeptide is in a pharmaceutical preparation.

In certain aspects, a ActRIIB variant polypeptide, to be used in accordance with methods and uses described herein, is a ActRIIB variant polypeptide that comprises the amino acid sequence of SEQ ID NO: 303, wherein at least one of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 303 is substituted with another amino acid, and wherein said ActRIIB variant polypeptide is capable of binding myostatin and activin A, but demonstrates a decreased binding affinity for BMP9 relative to a wild-type ActRIIB polypeptide. In some embodiments, at least one of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 of SEQ ID NO: 303 is substituted with the amino acid at the corresponding position of SEQ ID NO: 304, and wherein the ActRIIB variant polypeptide is capable of binding myostatin and activin A, but demonstrates a decreased binding affinity for BMP9 relative to a wild-type ActRIIB polypeptide. In some embodiments, the ActRIIB variant polypeptide comprises the amino acid sequence selected from the group consisting of: SEQ ID NO: 305, SEQ ID NO: 306, SEQ ID NO: 307, SEQ ID NO: 308, SEQ ID NO: 309, SEQ ID NO: 310, SEQ ID NO: 311, SEQ ID NO: 312, SEQ ID NO: 313, SEQ ID NO: 314, SEQ ID NO: 315, SEQ ID NO: 316, SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338 and SEQ ID NO: 339. In some embodiments, the ActRIIB variant polypeptide comprises the amino acid sequence selected from the group consisting of: SEQ ID NO: 340, SEQ ID NO: 341, SEQ ID NO: 342, SEQ ID NO: 343, SEQ ID NO: 344, SEQ ID NO: 345, SEQ ID NO: 346, SEQ ID NO: 347, SEQ ID NO: 348, SEQ ID NO: 349, SEQ ID NO: 350, SEQ ID NO: 351, SEQ ID NO: 352, SEQ ID NO: 353, SEQ ID NO: 354, SEQ ID NO: 355, SEQ ID NO: 356, SEQ ID NO: 357, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 366, SEQ ID NO: 367, SEQ ID NO: 368, SEQ ID NO: 369, SEQ ID NO: 370, SEQ ID NO: 371, SEQ ID NO: 372, SEQ ID NO: 373, SEQ ID NO: 374, SEQ ID NO: 375, SEQ ID NO: 376, SEQ ID NO: 377, SEQ ID NO: 378, SEQ ID NO: 379, SEQ ID NO: 380, SEQ ID NO: 381, SEQ ID NO: 382, SEQ ID NO: 383, SEQ ID NO: 384, SEQ ID NO: 385, SEQ ID NO: 386, SEQ ID NO: 387, SEQ ID NO: 388, SEQ ID NO: 389, SEQ ID NO: 390, SEQ ID NO: 391, SEQ ID NO: 392, SEQ ID NO: 393, SEQ ID NO: 394, SEQ ID NO: 395, SEQ ID NO: 396, SEQ ID NO: 397, SEQ ID NO: 398, SEQ ID NO: 399, SEQ ID NO: 400, SEQ ID NO: 401, SEQ ID NO: 402, SEQ ID NO: 403, SEQ ID NO: 404, SEQ ID NO: 405, SEQ ID NO: 406, and SEQ ID NO: 407. In some embodiments, the ActRIIB variant polypeptide comprises an amino acid sequence selected from the group consisting of SEQ ID NO: 318 and SEQ ID NO: 331. In some embodiments, the ActRIIB variant polypeptide is selected from the group consisting of: a polypeptide comprising an amino acid sequence that is at least 90% identical to SEQ ID NO: 318; a polypeptide comprising an amino acid sequence that is at least 95% identical to SEQ ID NO: 318; a polypeptide comprising an amino acid sequence that is at least 97% identical to SEQ ID NO: 318; a polypeptide comprising an amino acid sequence that is at least 99% identical to SEQ ID NO: 318; and a polypeptide comprising the amino acid sequence of SEQ ID NO: 318. In some embodiments, the ActRIIB variant polypeptide is selected from the group consisting of: a polypeptide comprising an amino acid sequence that is at least 90% identical to SEQ ID NO: 331; a polypeptide comprising an amino acid sequence that is at least 95% identical to SEQ ID NO: 331; a polypeptide comprising an amino acid sequence that is at least 97% identical to SEQ ID NO: 331; a polypeptide comprising an amino acid sequence that is at least 99% identical to SEQ ID NO: 331; and a polypeptide comprising the amino acid sequence of SEQ ID NO: 331. In some embodiments, the ActRIIB variant polypeptide binds to one or more ligands selected from the group consisting of: activin A, activin B, GDF11, GDF8, and BMP6. In some embodiments, the ActRIIB variant polypeptide does not bind or does not substantially bind to one or more ligands selected from the group consisting of: BMP10, BMP9, and GDF3. In some embodiments, the ActRIIB variant polypeptide is lyophilized. In some embodiments, the ActRIIB variant polypeptide is reconstituted following lyophilization. In some embodiments, the ActRIIB variant polypeptide is soluble. In some embodiments, the ActRIIB variant polypeptide is administered using subcutaneous injection. In some embodiments, the ActRIIB variant polypeptide is administered about every 4 weeks. In some embodiments, the ActRIIB variant polypeptide is administered every 4 weeks.

As described herein, in certain aspects, ActRIIB variant polypeptides may be homomultimers. In some embodiments, the ActRIIB variant polypeptide is part of a homodimer protein complex. In some embodiments, the ActRIIB variant polypeptide does not comprise an acidic amino acid at the position corresponding to L79 of SEQ ID NO: 1. In some embodiments, the ActRIIB variant polypeptide is a fusion protein further comprises a heterologous domain. In some embodiments, the heterologous domain is an Fc immunoglobulin domain. In some embodiments, the fusion protein further comprises a linker domain positioned between the ActRIIB variant polypeptide domain and the heterologous domain. In some embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In some embodiments, the ActRIIB variant polypeptide or fusion protein comprises one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety. In some embodiments, the ActRIIB variant polypeptide or fusion protein is glycosylated and has a mammalian glycosylation pattern. In some embodiments, the ActRIIB variant polypeptide or fusion protein has a glycosylation pattern obtainable from a Chinese hamster ovary cell line. In some embodiments, the ActRIIB variant polypeptide or fusion protein binds to one or more ligands selected from the group consisting of: activin A, activin B, GDF11, GDF8, and BMP6. In some embodiments, the ActRIIB variant polypeptide or fusion protein binds to activin A. In some embodiments, the ActRIIB variant polypeptide or fusion protein inhibits one or more TGFβ superfamily ligands selected from the group consisting of: activin A, activin B, GDF11, GDF8, and BMP6. In some embodiments, the ActRIIB variant polypeptide or fusion protein inhibits activin A. In some embodiments, the ActRIIB variant polypeptide or fusion protein does not bind or does not substantially bind to one or more ligands selected from the group consisting of: BMP10, BMP9, and GDF3. In some embodiments, the ActRIIB variant polypeptide or fusion protein binds to one or more of BMP10, BMP9, or GDF3 with lower affinity compared to a corresponding wild-type ActRIIB polypeptide. In some embodiments, the ActRIIB variant polypeptide and/or fusion protein is in a pharmaceutical preparation. In some embodiments, the pharmaceutical preparation is administered using subcutaneous injection. In some embodiments, the pharmaceutical preparation is administered about every 4 weeks. In some embodiments, methods of the present disclosure further comprise administering to the patient an additional active agent and/or supportive therapy.

In certain aspects, the disclosure provides a kit comprising a lyophilized polypeptide and an injection device. In certain aspects the disclosure provides a kit comprising a lyophilized polypeptide and an injection device, wherein the polypeptide is an ActRIIA polypeptide comprising an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 9, 10, 11, 36, 39, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 283, 304, 408, and 409. In certain embodiments, the disclosure provides a kit comprising a lyophilized polypeptide and an injection device, wherein the polypeptide is an ActRIIB polypeptide comprising an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 40, 42, 45, 46, 47, 48, 69, 74, 77, 78, 79, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, and 407. In some embodiments, the polypeptide is glycosylated. In some embodiments, the polypeptide binds to one or more ligands selected from the group consisting of: activin A, activin B, and GDF11. In some embodiments, the polypeptide further binds to one or more ligands selected from the group consisting of: BMP10, GDF8, and BMP6. In some embodiments, the polypeptide binds to activin and/or GDF11. In some embodiments, the kit comprises one or more vials containing the lyophilized polypeptide. In some embodiments, the injection device comprises a pre-filled syringe. In some embodiments, the injection device comprises a pump apparatus. In some embodiments, the pump apparatus comprises an electromechanical pumping assembly. In some embodiments, the pump apparatus is a wearable pump apparatus. In some embodiments, the pre-filled syringe comprises a reconstitution solution. In some embodiments, the reconstitution solution comprises a pharmaceutically acceptable carrier and/or excipient. In some embodiments, the pharmaceutically acceptable carrier is selected from saline solution, purified water, and sterile water for injection. In some embodiments, the pharmaceutically acceptable excipient is selected from a buffering agent (e.g., citric acid (monohydrate) and/or trisodium citrate (dehydrate)), a surfactant (e.g., polysorbate 80), a stabilizer (e.g., sucrose), and a lyoprotectant (e.g., sucrose). In some embodiments, the injection device comprises a vial adapter. In some embodiments, the vial adapter is capable of attaching to a vial. In some embodiments, the vial adapter is capable of attaching to a pre-filled syringe. In some embodiments, the pre-filled syringe and the vial are attached to opposite ends of the vial adapter. In some embodiments, the reconstitution solution is transferred from the pre-filled syringe to the vial. In some embodiments, the lyophilized polypeptide is reconstituted into a sterile injectable solution. In some embodiments, the lyophilized polypeptide is reconstituted into a sterile injectable solution prior to use. In some embodiments, the sterile injectable solution is sterile water for injection. In some embodiments, the sterile injectable solution is administered parenterally. In some embodiments, the injection device is used to administer the sterile injectable solution parenterally. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose. In some embodiments, the therapeutically effective dose comprises a weight based dose. In some embodiments, the lyophilized polypeptide is administered every 4 weeks. In some embodiments, the kit is used to treat renal diseases or conditions (e.g., Alport syndrome, focal segmental glomerulosclerosis (FSGS), polycystic kidney disease, chronic kidney disease). In some embodiments, the shelf life of the lyophilized polypeptide is at least 1, 1.5, 2, 2.5, or 3 years. In some embodiments, the lyophilized polypeptide is reconstituted. In some embodiments, the reconstituted polypeptide has a shelf life of at least 2 hrs, 3 hrs, or 4 hrs.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fec.

FIG. 1 shows an alignment of extracellular domains of human ActRIIB (SEQ ID NO: 2) and human ActRIIA (SEQ ID NO: 10) with the residues that are deduced herein, based on composite analysis of multiple ActRIIB and ActRIIA crystal structures, to directly contact ligand indicated with boxes.

FIG. 2 shows a multiple sequence alignment of various vertebrate ActRIIB proteins (SEQ ID NOs: 53-58) and human ActRIIA (SEQ ID NO: 59) as well as a consensus ActRII sequence derived from the alignment (SEQ ID NO: 60).

FIG. 3 shows a multiple sequence alignment of various vertebrate ActRIIA proteins and human ActRIIA (SEQ ID NOs: 10 and 62-68).

FIG. 4 shows multiple sequence alignment of Fc domains from human IgG isotypes using Clustal 2.1. Hinge regions are indicated by dotted underline. Double underline indicates examples of positions engineered in IgG1 (SEQ ID NO: 133) Fc to promote asymmetric chain pairing and the corresponding positions with respect to other isotypes IgG2 (SEQ ID NO: 135), IgG3 (SEQ ID NO: 136) and IgG4 (SEQ ID NO: 134).

FIG. 5A and FIG. 5B show the purification of ActRIIA-hFc expressed in CHO cells. The protein purifies as a single, well-defined peak as visualized by sizing column (FIG. 5A) and Coomassie stained SDS-PAGE (FIG. 5B) (left lane: molecular weight standards; right lane: ActRIIA-hFc).

FIG. 6A and FIG. 6B show the binding of ActRIIA-hFc to activin (FIG. 6A) and GDF-11 (FIG. 6B), as measured by Biacore™ assay.

FIG. 7 shows the full, unprocessed amino acid sequence for ActRIIB (25-131)-hFc (SEQ ID NO: 69). The TPA leader (residues 1-22) and double-truncated ActRIIB extracellular domain (residues 24-131, using numbering based on the native sequence in SEQ ID NO: 1) are each underlined. Boxed is the glutamate revealed by sequencing to be the N-terminal amino acid of the mature fusion protein, which is at position 25 relative to SEQ ID NO: 1.

FIGS. 8A and 8B show a nucleotide sequence encoding ActRIIB (25-131)-hFc (the coding strand is shown at top, SEQ ID NO: 70, and the complement shown at bottom 3′-5′, SEQ ID NO: 71). Sequences encoding the TPA leader (nucleotides 1-66) and ActRIIB extracellular domain (nucleotides 73-396) are underlined. The corresponding amino acid sequence for ActRIIB (25-131) (SEQ ID NO: 138) is also shown.

FIGS. 9A and 9B show an alternative nucleotide sequence encoding ActRIIB (25-131)-hFc (the coding strand is shown at top, SEQ ID NO: 72, and the complement shown at bottom 3′-5′, SEQ ID NO: 73). This sequence confers a greater level of protein expression in initial transformants, making cell line development a more rapid process. Sequences encoding the TPA leader (nucleotides 1-66) and ActRIIB extracellular domain (nucleotides 73-396) are underlined, and substitutions in the wild type nucleotide sequence of the ECD (see FIG. 8) are boxed. The corresponding amino acid sequence for ActRIIB (25-131) (SEQ ID NO: 138) is also shown.

FIG. 10 shows the full amino acid sequence for the ActRIIB (L79D 20-134)-hFc (SEQ ID NO: 74), including the TPA leader sequence (double underline), ActRIIB extracellular domain (residues 20-134 in SEQ ID NO: 1; single underline), and hFc domain. The aspartate substituted at position 79 in the native sequence is double underlined and boxed, as is the glycine revealed by sequencing to be the N-terminal residue in the mature fusion protein.

FIGS. 11A and 11B show a nucleotide sequence encoding ActRIIB (L79D 20-134)-hFc. SEQ ID NO: 75 corresponds to the sense strand, and SEQ ID NO: 76 corresponds to the antisense strand. The TPA leader (nucleotides 1-66) is double underlined, and the ActRIIB extracellular domain (nucleotides 76-420) is single underlined.

FIG. 12 shows the full amino acid sequence for the truncated ActRIIB (L79D 25-131)-hFc (SEQ ID NO: 77), including the TPA leader (double underline), truncated ActRIIB extracellular domain (residues 25-131 in SEQ ID NO: 1; single underline), and hFc domain. The aspartate substituted at position 79 in the native sequence is double underlined and boxed, as is the glutamate revealed by sequencing to be the N-terminal residue in the mature fusion protein.

FIG. 13 shows the amino acid sequence for the truncated ActRIIB (L79D 25-131)-hFc without a leader (SEQ ID NO: 78). The truncated ActRIIB extracellular domain (residues 25-131 in SEQ ID NO: 1) is underlined. The aspartate substituted at position 79 in the native sequence is double underlined and boxed, as is the glutamate revealed by sequencing to be the N-terminal residue in the mature fusion protein.

FIG. 14 shows the amino acid sequence for the truncated ActRIIB (L79D 25-131) without the leader, hFc domain, and linker (SEQ ID NO: 79). The aspartate substituted at position 79 in the native sequence is underlined and boxed, as is the glutamate revealed by sequencing to be the N-terminal residue in the mature fusion protein.

FIGS. 15A and 15B show a nucleotide sequence encoding ActRIIB (L79D 25-131)-hFc. SEQ ID NO: 80 corresponds to the sense strand, and SEQ ID NO: 81 corresponds to the antisense strand. The TPA leader (nucleotides 1-66) is double underlined, and the truncated ActRIIB extracellular domain (nucleotides 76-396) is single underlined. The amino acid sequence for the ActRIIB extracellular domain (SEQ ID NO: 79) is also shown.

FIGS. 16A and 16B show an alternative nucleotide sequence encoding ActRIIB (L79D 25-131)-hFc. SEQ ID NO: 82 corresponds to the sense strand, and SEQ ID NO: 83 corresponds to the antisense strand. The TPA leader (nucleotides 1-66) is double underlined, the truncated ActRIIB extracellular domain (nucleotides 76-396) is underlined, and substitutions in the wild-type nucleotide sequence of the extracellular domain are double underlined and boxed (compare with SEQ ID NO: 81, FIG. 15). The amino acid sequence for the ActRIIB extracellular domain (SEQ ID NO: 79) is also shown.

FIG. 17 shows nucleotides 76-396 (SEQ ID NO: 84) of the alternative nucleotide sequence shown in FIG. 16 (SEQ ID NO: 82). The same nucleotide substitutions indicated in FIG. 16 are also underlined and boxed here. SEQ ID NO: 84 encodes only the truncated ActRIIB extracellular domain (corresponding to residues 25-131 in SEQ ID NO: 1) with a L79D substitution, e.g., ActRIIB (L79D 25-131).

FIG. 18 shows components of a kit comprising a lyophilized polypeptide and an injection device. A vial (1) holds lyophilized polypeptide, reconstituted sterile injectable solution, or sterile injectable solution. A prefilled syringe (2) containing a reconstitution solution is used to reconstitute lyophilized polypeptide from (1) into a sterile injectable solution. A vial adapter (3) couples the vial (1) to the pre-filled syringe (2) via attachment to the vial at one end, and attachment to the pre-filled syringe at an opposite end. A syringe (4) and needle (5) are provided for administration of sterile injectable solution. Swab wipes (6) are provided for sterilization of individual kit components.

### DETAILED DESCRIPTION

### 1. Overview

The present disclosure relates to compositions and methods of treating renal diseases or conditions (e.g., Alport syndrome, focal segmental glomerulosclerosis (FSGS), polycystic kidney disease, chronic kidney disease) comprising administering to a patient in need thereof an effective amount of an ActRII polypeptide as described herein. In certain embodiments, the present disclosure provides methods of treating or preventing a renal disease or condition (e.g., Alport syndrome, focal segmental glomerulosclerosis (FSGS), polycystic kidney disease, chronic kidney disease) in an individual in need thereof through administering to the individual a therapeutically effective amount of an ActRII polypeptide as described herein. In certain embodiments, the present disclosure provides methods of treating or preventing Alport Syndrome in an individual in need thereof through administering to the individual a therapeutically effective amount of an ActRII polypeptide as described herein. In certain embodiments, the present disclosure provides methods of treating or preventing focal segmental glomerulosclerosis (FSGS) in an individual in need thereof through administering to the individual a therapeutically effective amount of an ActRII polypeptide as described herein. In certain embodiments, the present disclosure provides methods of treating or preventing polycystic kidney disease in an individual in need thereof through administering to the individual a therapeutically effective amount of an ActRII polypeptide as described herein. In certain embodiments, the present disclosure provides methods of treating or preventing chronic kidney disease in an individual in need thereof through administering to the individual a therapeutically effective amount of an ActRII polypeptide as described herein.

The TGF-β superfamily is comprised of over 30 secreted factors including TGF-betas, activins, nodals, bone morphogenetic proteins (BMPs), growth and differentiation factors (GDFs), and anti-Mullerian hormone (AMH) [Weiss et al. (2013) Developmental Biology, 2 (1): 47-63]. Members of the superfamily, which are found in both vertebrates and invertebrates, are ubiquitously expressed in diverse tissues and function during the earliest stages of development throughout the lifetime of an animal. Indeed, TGF-β superfamily proteins are key mediators of stem cell self-renewal, gastrulation, differentiation, organ morphogenesis, and adult tissue homeostasis. Consistent with this ubiquitous activity, aberrant TGF-beta superfamily signaling is associated with a wide range of human pathologies including, for example, autoimmune disease, cardiovascular disease, fibrotic disease, and cancer.

Ligands of the TGF-β superfamily (e.g., ligands binding to ActRIIA or ActRIIB) share the same dimeric structure in which the central 3½ turn helix of one monomer packs against the concave surface formed by the beta-strands of the other monomer. The majority of TGF-β family members are further stabilized by an intermolecular disulfide bond. This disulfide bonds traverses through a ring formed by two other disulfide bonds generating what has been termed a ‘cysteine knot’ motif [Lin et al. (2006) Reproduction 132:179-190; and Hinck et al. (2012) FEBS Letters 586:1860-1870].

TGF-β superfamily (e.g., ActRIIA or ActRIIB) signaling is mediated by heteromeric complexes of type I and type II serine/threonine kinase receptors, which phosphorylate and activate downstream SMAD proteins (e.g., SMAD proteins 1, 2, 3, 5, and 8) upon ligand stimulation [Massaguć (2000) Nat. Rev. Mol. Cell Biol. 1:169-178]. These type I and type II receptors are transmembrane proteins, composed of a ligand-binding extracellular domain with cysteine-rich region, a transmembrane domain, and a cytoplasmic domain with predicted serine/threonine kinase specificity. In general, type I receptors mediate intracellular signaling while the type II receptors are required for binding TGF-beta superfamily ligands. Type I and II receptors form a stable complex after ligand binding, resulting in phosphorylation of type I receptors by type II receptors.

The TGF-β family can be divided into two phylogenetic branches based on the type I receptors they bind and the SMAD proteins they activate. One is the more recently evolved branch, which includes, e.g., the TGF-betas, activins, GDF8, GDF9, GDF11, BMP3 and nodal, which signal through type I receptors that activate SMADs 2 and 3 [Hinck (2012) FEBS Letters 586:1860-1870]. The other branch comprises the more distantly related proteins of the superfamily and includes, e.g., BMP2, BMP4, BMP5, BMP6, BMP7, BMP8a, BMP8b, BMP9, BMP10, GDF1, GDF5, GDF6, and GDF7, which signal through SMADs 1, 5, and 8.

Activins are members of the TGF-β superfamily and were initially discovered as regulators of secretion of follicle-stimulating hormone, but subsequently various reproductive and non-reproductive roles have been characterized. There are three principal activin forms (A, B, and AB) that are homo/heterodimers of two closely related β subunits (β_{A}β_{A}, β_{B}β_{B}, and β_{A}β_{B}, respectively). The human genome also encodes an activin C and an activin E, which are primarily expressed in the liver, and heterodimeric forms containing Be or BE are also known. In the TGF-beta superfamily, activins are unique and multifunctional factors that can stimulate hormone production in ovarian and placental cells, support neuronal cell survival, influence cell-cycle progress positively or negatively depending on cell type, and induce mesodermal differentiation at least in amphibian embryos [DePaolo et al. (1991) Proc Soc Ep Biol Med. 198:500-512; Dyson et al. (1997) Curr Biol. 7:81-84; and Woodruff (1998) Biochem Pharmacol. 55:953-963]. In several tissues, activin signaling is antagonized by its related heterodimer, inhibin. For example, in the regulation of follicle-stimulating hormone (FSH) secretion from the pituitary, activin promotes FSH synthesis and secretion, while inhibin reduces FSH synthesis and secretion. Other proteins that may regulate activin bioactivity and/or bind to activin include follistatin (FS), follistatin-related protein (FSRP, also known as FLRG or FSTL3), and α₂-macroglobulin.

As described herein, agents that bind to “activin A” are agents that specifically bind to the β_{A} subunit, whether in the context of an isolated β_{A} subunit or as a dimeric complex (e.g., a BABA homodimer or a β_{A}β_{B} heterodimer). In the case of a heterodimer complex (e.g., a β_{A}β_{B} heterodimer), agents that bind to “activin A” are specific for epitopes present within the β_{A} subunit, but do not bind to epitopes present within the non-β_{A} subunit of the complex (e.g., the β_{B} subunit of the complex). Similarly, agents disclosed herein that antagonize (inhibit) “activin A” are agents that inhibit one or more activities as mediated by a β_{A} subunit, whether in the context of an isolated β_{A} subunit or as a dimeric complex (e.g., a BABA homodimer or a β_{A}β_{B} heterodimer). In the case of β_{A}β_{B} heterodimers, agents that inhibit “activin A” are agents that specifically inhibit one or more activities of the β_{A} subunit, but do not inhibit the activity of the non-β_{A} subunit of the complex (e.g., the β_{B} subunit of the complex). This principle applies also to agents that bind to and/or inhibit “activin B”, “activin C”, and “activin E”. Agents disclosed herein that antagonize “activin AB” are agents that inhibit one or more activities as mediated by the β_{A} subunit and one or more activities as mediated by the β_{B} subunit.

The BMPs and GDFs together form a family of cysteine-knot cytokines sharing the characteristic fold of the TGF-beta superfamily [Rider et al. (2010) Biochem J., 429 (1): 1-12]. This family includes, for example, BMP2, BMP4, BMP6, BMP7, BMP2a, BMP3, BMP3b (also known as GDF10), BMP4, BMP5, BMP6, BMP7, BMP8, BMP8a, BMP8b, BMP9 (also known as GDF2), BMP10, BMP11 (also known as GDF11), BMP12 (also known as GDF7), BMP13 (also known as GDF6), BMP14 (also known as GDF5), BMP15, GDF1, GDF3 (also known as VGR2), GDF8 (also known as myostatin), GDF9, GDF15, and decapentaplegic. Besides the ability to induce bone formation, which gave the BMPs their name, the BMP/GDFs display morphogenetic activities in the development of a wide range of tissues. BMP/GDF homo- and hetero-dimers interact with combinations of type I and type II receptor dimers to produce multiple possible signaling complexes, leading to the activation of one of two competing sets of SMAD transcription factors. BMP/GDFs have highly specific and localized functions. These are regulated in a number of ways, including the developmental restriction of BMP/GDF expression and through the secretion of several specific BMP antagonist proteins that bind with high affinity to the cytokines. Curiously, a number of these antagonists resemble TGF-beta superfamily ligands.

Growth and differentiation factor-8 (GDF8) is also known as myostatin. GDF8 is a negative regulator of skeletal muscle mass and is highly expressed in developing and adult skeletal muscle. The GDF8 null mutation in transgenic mice is characterized by a marked hypertrophy and hyperplasia of skeletal muscle [McPherron et al. Nature (1997) 387:83-90]. Similar increases in skeletal muscle mass are evident in naturally occurring mutations of GDF8 in cattle and, strikingly, in humans [Ashmore et al. (1974) Growth, 38:501-507; Swatland and Kieffer, J. Anim. Sci. (1994) 38:752-757; McPherron and Lee, Proc. Natl. Acad. Sci. USA (1997) 94:12457-12461; Kambadur et al. Genome Res. (1997) 7:910-915; and Schuelke et al. (2004) N Engl J Med, 350:2682-8]. Studies have also shown that muscle wasting associated with HIV-infection in humans is accompanied by increases in GDF8 protein expression [Gonzalez-Cadavid et al., PNAS (1998) 95:14938-43]. In addition, GDF8 can modulate the production of muscle-specific enzymes (e.g., creatine kinase) and modulate myoblast cell proliferation [International Patent Application Publication No. WO 00/43781]. The GDF8 propeptide can noncovalently bind to the mature GDF8 domain dimer, inactivating its biological activity [Miyazono et al. (1988) J. Biol. Chem., 263:6407-6415; Wakefield et al. (1988) J. Biol. Chem., 263; 7646-7654; and Brown et al. (1990) Growth Factors, 3:35-43]. Other proteins which bind to GDF8 or structurally related proteins and inhibit their biological activity include follistatin, and potentially, follistatin-related proteins [Gamer et al. (1999) Dev. Biol., 208:222-232].

GDF11, also known as BMP11, is a secreted protein that is expressed in the tail bud, limb bud, maxillary and mandibular arches, and dorsal root ganglia during mouse development [McPherron et al. (1999) Nat. Genet., 22:260-264; and Nakashima et al. (1999) Mech. Dev., 80:185-189]. GDF11 plays a unique role in patterning both mesodermal and neural tissues [Gamer et al. (1999) Dev Biol., 208:222-32]. GDF11 was shown to be a negative regulator of chondrogenesis and myogenesis in developing chick limb [Gamer et al. (2001) Dev Biol., 229:407-20]. The expression of GDF11 in muscle also suggests its role in regulating muscle growth in a similar way to GDF8. In addition, the expression of GDF11 in brain suggests that GDF11 may also possess activities that relate to the function of the nervous system. Interestingly, GDF11 was found to inhibit neurogenesis in the olfactory epithelium [Wu et al. (2003) Neuron., 37:197-207]. Hence, GDF11 may have in vitro and in vivo applications in the treatment of diseases such as muscle diseases and neurodegenerative diseases (e.g., amyotrophic lateral sclerosis).

As used herein, ActRII refers to the family of type II activin receptors. This family includes both the activin receptor type IIA (ActRIIA), encoded by the ACVR2A gene, and the activin receptor type IIB (ActRIIB), encoded by the ACVR2B gene. ActRII receptors are TGF-beta superfamily type II receptors that bind a variety of TGF-beta superfamily ligands including activins, GDF8 (myostatin), GDF11, and a subset of BMPs, notably BMP6 and BMP7. ActRII receptors are implicated in a variety of biological disorders including muscle and neuromuscular disorders (e.g., muscular dystrophy, amyotrophic lateral sclerosis (ALS), and muscle atrophy), undesired bone/cartilage growth, adipose tissue disorders (e.g., obesity), metabolic disorders (e.g., type 2 diabetes), and neurodegenerative disorders. Sec, e.g., Tsuchida et al., (2008) Endocrine Journal 55 (1): 11-21, Knopf et al., U.S. Pat. No. 8,252,900, and OMIM entries 102581 and 602730.

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is +10%. Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. ActRII Antagonists

In certain aspects, the disclosure relates to ActRII antagonists (e.g., ActRII polypeptides) and uses thereof (e.g., of treating, preventing, or reducing the progression rate and/or severity of renal diseases or conditions, or one or more complications of renal diseases or conditions). As used herein, the term “ActRII” refers to the family of type II activin receptors. This family includes activin receptor type IIA (ActRIIA) and activin receptor type IIB (ActRIIB).

As used herein, the term “ActRIIB” refers to a family of activin receptor type IIB (ActRIIB) proteins from any species and variants derived from such ActRIIB proteins by mutagenesis or other modification. Reference to ActRIIB herein is understood to be a reference to any one of the currently identified forms. Members of the ActRIIB family are generally transmembrane proteins, composed of a ligand-binding extracellular domain comprising a cysteine-rich region, a transmembrane domain, and a cytoplasmic domain with predicted serine/threonine kinase activity.

The term “ActRIIB polypeptide” includes polypeptides comprising any naturally occurring polypeptide of an ActRIIB family member as well as any variants thereof (including mutants, fragments, fusions, and peptidomimetic forms) that retain a useful activity. Examples of such variant ActRIIB polypeptides are provided throughout the present disclosure as well as in International Patent Application Publication Nos. WO 2006/012627, WO 2008/097541, WO 2010/151426, WO 2011/020045, WO2019140283, WO2018/089706, WO2018/089715, WO2019/094751, WO2016/171948, and WO2018/075747 which are incorporated herein by reference in their entirety. Numbering of amino acids for all ActRIIB-related polypeptides described herein is based on the numbering of the human ActRIIB precursor protein sequence provided below (SEQ ID NO: 1), unless specifically designated otherwise.

The human ActRIIB precursor protein sequence is as follows:

```
(SEQ ID NO: 1)
    1 MTAPWVALAL LWGSLCAGSG RGEAETRECI YYNANWELER TNQSGLERCE
  51 GEQDKRLHCY ASWRNSSGTI ELVKKGCWLD DENCYDRQEC VATEENPQVY
101 FCCCEGNFCN ERFTHLPEAG GPEVTYEPPP TAPTLLTVLA YSLLPIGGLS
151 LIVLLAFWMY RHRKPPYGHV DIHEDPGPPP PSPLVGLKPL QLLEIKARGR
201 FGCVWKAQLM NDFVAVKIFP LQDKQSWOSE REIFSTPGMK HENLLQFIAA
251 EKRGSNLEVE LWLITAFHDK GSLTDYLKGN IITWNELCHV AETMSRGLSY
301 LHEDVPWCRG EGHKPSIAHR DFKSKNVLLK SDLTAVLADF GLAVRFEPGK
351 PPGDTHGQVG TRRYMAPEVL EGAINFORDA FLRIDMYAMG LVLWELVSRC
401 KAADGPVDEY MLPFEEEIGQ HPSLEELQEV VVHKKMRPTI KDHWLKHPGL
451 AQLCVTIEEC WDHDAEARLS AGCVEERVSL IRRSVNGTTS DCLVSLVTSV
501 TNVDLPPKES SI
```

The signal peptide is indicated with a single underline; the extracellular domain is indicated in bold font; and the potential, endogenous N-linked glycosylation sites are indicated with a double underline.

The processed (mature) extracellular ActRIIB polypeptide sequence is as follows:

(SEQ ID NO: 2)
GRGEAETRECIYYNANWELERTNQSGLERCEGEQDKRLHCYASWRNSSG
TIELVKKGCWLDDFNCYDRQECVATEENPQVYFCCCEGNFCNERFTHLP
EAGGPEVTYEPPPTAPT.

In some embodiments, the protein may be produced with an “SGR . . . ” sequence at the N-terminus. The C-terminal “tail” of the extracellular domain is indicated by a single underline. The sequence with the “tail” deleted (a 415 sequence) is as follows:

(SEQ ID NO: 3)
GRGEAETRECIYYNANWELERTNOSGLERCEGEQDKRLHCYASWRNSSG
TIELVKKGCWLDDFNCYDRQECVATEENPQVYFCCCEGNFCNERFTHLP
EA.

A form of ActRIIB with an alanine at position 64 of SEQ ID NO: 1 (A64) is also reported in the literature. See, e.g., Hilden et al. (1994) Blood, 83 (8): 2163-2170. Applicants have ascertained that an ActRIIB-Fc fusion protein comprising an extracellular domain of ActRIIB with the A64 substitution has a relatively low affinity for activin and GDF11. By contrast, the same ActRIIB-Fc fusion protein with an arginine at position 64 (R64) has an affinity for activin and GDF11 in the low nanomolar to high picomolar range. Therefore, sequences with an R64 are used as the “wild-type” reference sequence for human ActRIIB in this disclosure.

The form of ActRIIB with an alanine at position 64 is as follows:

```
(SEQ ID NO: 4)
    1 MTAPWVALAL LWGSLCAGSG RGEAETRECI YYNANWELER TNQSGLERCE
  51 GEQDKRLHCY ASWANSSGTI ELVKKGCWLD DENCYDRQEC VATEENPQVY
101 FCCCEGNFCN ERFTHLPEAG GPEVTYEPPP TAPTLLTVLA YSLLPIGGLS
151 LIVLLAFWMY RHRKPPYGHV DIHEDPGPPP PSPLVGLKPL QLLEIKARGR
201 FGCVWKAQLM NDEVAVKIFP LQDKQSWQSE REIFSTPGMK HENLLOFIAA
251 EKRGSNLEVE LWLITAFHDK GSLTDYLKGN IITWNELCHV AETMSRGLSY
301 LHEDVPWCRG EGHKPSIAHR DFKSKNVLLK SDLTAVLADF GLAVRFEPGK
351 PPGDTHGQVG TRRYMAPEVL EGAINFQRDA FLRIDMYAMG LVLWELVSRC
401 KAADGPVDEY MLPFEEEIGQ HPSLEELQEV VVHKKMRPTI KDHWLKHPGL
451 AQLCVTIEEC WDHDAEARLS AGCVEERVSL IRRSVNGTTS DCLVSLVTSV
501 TNVDLPPKES SI
```

The signal peptide is indicated by single underline and the extracellular domain is indicated by bold font.

The processed (mature) extracellular ActRIIB polypeptide sequence of the alternative A64 form is as follows:

(SEQ ID NO: 5)
GRGEAETRECIYYNANWELERINQSGLERCEGEQDKRLHCYASWANSSG
TIELVKKGCWLDDFNCYDRQECVATEENPQVYFCCCEGNFCNERFTHLP
EAGGPEVTYEPPPTAPT

In some embodiments, the protein may be produced with an “SGR . . . ” sequence at the N-terminus. The C-terminal “tail” of the extracellular domain is indicated by single underline. The sequence with the “tail” deleted (a 415 sequence) is as follows:

(SEQ ID NO: 6)
GRGEAETRECIYYNANWELERTNOSGLERCEGEQDKRLHCYASWANSSG
TIELVKKGCWLDDFNCYDRQECVATEENPQVYFCCCEGNFCNERFTHLP
EA

A nucleic acid sequence encoding the human ActRIIB precursor protein is shown below (SEQ ID NO: 7), representing nucleotides 25-1560 of Genbank Reference Sequence NM_001106.3, which encode amino acids 1-513 of the ActRIIB precursor. The sequence as shown provides an arginine at position 64 and may be modified to provide an alanine instead. The signal sequence is underlined.

```
(SEQ ID NO: 7)
      1 ATGACGGCGC CCTGGGTGGC CCTCGCCCTC CTCTGGGGAT CGCTGTGCGC
    51 CGGCTCTGGG CGTGGGGAGG CTGAGACACG GGAGTGCATC TACTACAACG
  101 CCAACTGGGA GCTGGAGCGC ACCAACCAGA GCGGCCTGGA GCGCTGCGAA
  151 GGCGAGCAGG ACAAGCGGCT GCACTGCTAC GCCTCCTGGC GCAACAGCTC
  201 TGGCACCATC GAGCTCGTGA AGAAGGGCTG CTGGCTAGAT GACTTCAACT
  251 GCTACGATAG GCAGGAGTGT GTGGCCACTG AGGAGAACCC CCAGGTGTAC
  301 TTCTGCTGCT GTGAAGGCAA CTTCTGCAAC GAACGCTTCA CTCATTTGCC
  351 AGAGGCTGGG GGCCCGGAAG TCACGTACGA GCCACCCCCG ACAGCCCCCA
  401 CCCTGCTCAC GGTGCTGGCC TACTCACTGC TGCCCATCGG GGGCCTTTCC
  451 CTCATCGTCC TGCTGGCCTT TTGGATGTAC CGGCATCGCA AGCCCCCCTA
  501 CGGTCATGTG GACATCCATG AGGACCCTGG GCCTCCACCA CCATCCCCTC
  551 TGGTGGGCCT GAAGCCACTG CAGCTGCTGG AGATCAAGGC TCGGGGGCGC
  601 TTTGGCTGTG TCTGGAAGGC CCAGCTCATG AATGACTTTG TAGCTGTCAA
  651 GATCTTCCCA CTCCAGGACA AGCAGTCGTG GCAGAGTGAA CGGGAGATCT
  701 TCAGCACACC TGGCATGAAG CACGAGAACC TGCTACAGTT CATTGCTGCC
  751 GAGAAGCGAG GCTCCAACCT CGAAGTAGAG CTGTGGCTCA TCACGGCCTT
  801 CCATGACAAG GGCTCCCTCA CGGATTACCT CAAGGGGAAC ATCATCACAT
  851 GGAACGAACT GTGTCATGTA GCAGAGACGA TGTCACGAGG CCTCTCATAC
  901 CTGCATGAGG ATGTGCCCTG GTGCCGTGGC GAGGGCCACA AGCCGTCTAT
  951 TGCCCACAGG GACTTTAAAA GTAAGAATGT ATTGCTGAAG AGCGACCTCA
1001 CAGCCGTGCT GGCTGACTTT GGCTTGGCTG TTCGATTTGA GCCAGGGAAA
1051 CCTCCAGGGG ACACCCACGG ACAGGTAGGC ACGAGACGGT ACATGGCTCC
1101 TGAGGTGCTC GAGGGAGCCA TCAACTTCCA GAGAGATGCC TTCCTGCGCA
1151 TTGACATGTA TGCCATGGGG TTGGTGCTGT GGGAGCTTGT GTCTCGCTGC
1201 AAGGCTGCAG ACGGACCCGT GGATGAGTAC ATGCTGCCCT TTGAGGAAGA
1251 GATTGGCCAG CACCCTTCGT TGGAGGAGCT GCAGGAGGTG GTGGTGCACA
1301 AGAAGATGAG GCCCACCATT AAAGATCACT GGTTGAAACA CCCGGGCCTG
1351 GCCCAGCTTT GTGTGACCAT CGAGGAGTGC TGGGACCATG ATGCAGAGGC
1401 TCGCTTGTCC GCGGGCTGTG TGGAGGAGCG GGTGTCCCTG ATTCGGAGGT
1451 CGGTCAACGG CACTACCTCG GACTGTCTCG TTTCCCTGGT GACCTCTGTC
1501 ACCAATGTGG ACCTGCCCCC TAAAGAGTCA AGCATC
```

A nucleic acid sequence encoding processed extracellular human ActRIIB polypeptide is as follows (SEQ ID NO: 8). The sequence as shown provides an arginine at position 64, and may be modified to provide an alanine instead.

```
(SEQ ID NO: 8)
    1 GGGCGTGGGG AGGCTGAGAC ACGGGAGTGC ATCTACTACA ACGCCAACTG
  51 GGAGCTGGAG CGCACCAACC AGAGCGGCCT GGAGCGCTGC GAAGGCGAGC
101 AGGACAAGCG GCTGCACTGC TACGCCTCCT GGCGCAACAG CTCTGGCACC
151 ATCGAGCTCG TGAAGAAGGG CTGCTGGCTA GATGACTTCA ACTGCTACGA
201 TAGGCAGGAG TGTGTGGCCA CTGAGGAGAA CCCCCAGGTG TACTTCTGCT
251 GCTGTGAAGG CAACTTCTGC AACGAACGCT TCACTCATTT GCCAGAGGCT
301 GGGGGCCCGG AAGTCACGTA CGAGCCACCC CCGACAGCCC CCACC
```

In some embodiments the ActRIIB polypeptide comprises the accession number NP_001097.2 (SEQ ID NO: 1 herein), and variants thereof. In some embodiments, the term “wild-type ActRIIB” refers to the extracellular domain of ActRIIB, amino acids 1 to 134 (with signal sequence), or amino acids 19 through 134 of SEQ ID NO: 1 (without signal sequence) (referred to herein as SEQ ID NO: 407).

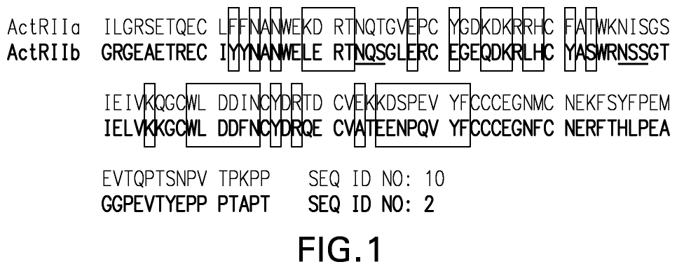

An alignment of the amino acid sequences of human ActRIIB extracellular domain and human ActRIIA extracellular domain are illustrated in FIG. 1. This alignment indicates amino acid residues within both receptors that are believed to directly contact ActRII ligands. For example, the composite ActRII structures indicated that the ActRIIB-ligand binding pocket is defined, in part, by residues Y31, N33, N35, L38 through T41, E47, E50, Q53 through K55, L57, H58, Y60, S62, K74, W78 through N83, Y85, R87, A92, and E94 through F101 (based on the numbering of SEQ ID NO: 1). At these positions, it is expected that conservative mutations will be tolerated.

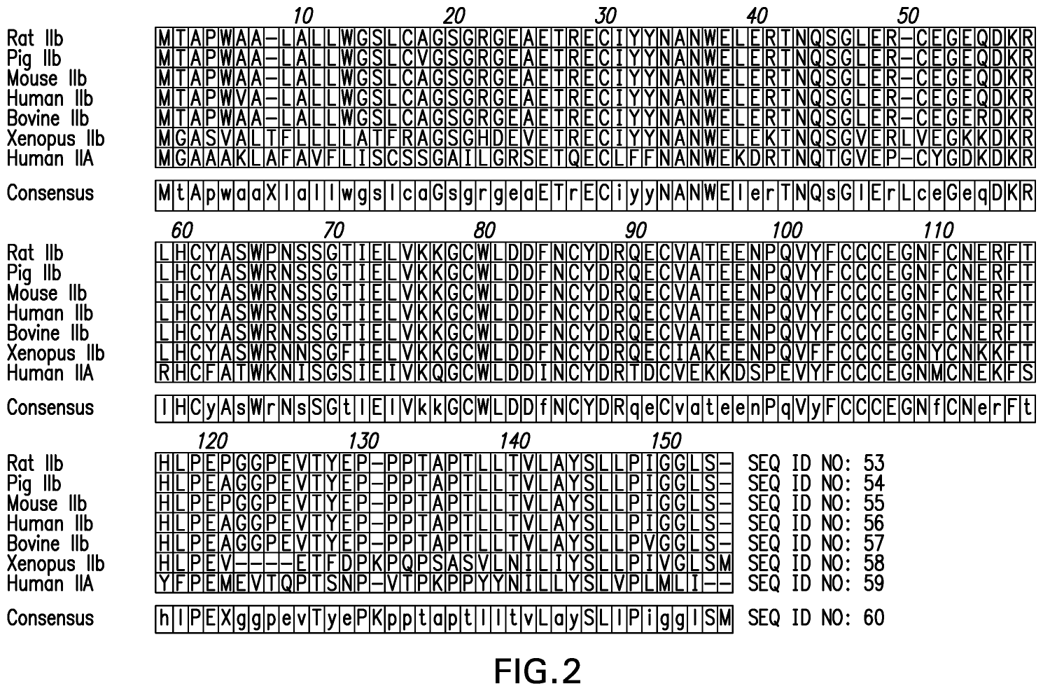

In addition, ActRIIB is well-conserved among vertebrates, with large stretches of the extracellular domain completely conserved. For example, FIG. 2 depicts a multi-sequence alignment of a human ActRIIB extracellular domain compared to various ActRIIB orthologs. Many of the ligands that bind to ActRIIB are also highly conserved. Accordingly, from these alignments, it is possible to predict key amino acid positions within the ligand-binding domain that are important for normal ActRIIB-ligand binding activities as well as to predict amino acid positions that are likely to be tolerant to substitution without significantly altering normal ActRIIB-ligand binding activities. Therefore, an active, human ActRIIB variant polypeptide useful in accordance with the presently disclosed methods may include one or more amino acids at corresponding positions from the sequence of another vertebrate ActRIIB, or may include a residue that is similar to that in the human or other vertebrate sequences. Without meaning to be limiting, the following examples illustrate this approach to defining an active ActRIIB variant. L46 in the human extracellular domain (SEQ ID NO: 2) is a valine in Xenopus ActRIIB (SEQ ID NO: 58), and so this position may be altered, and optionally may be altered to another hydrophobic residue, such as V, I or F, or a non-polar residue such as A. E52 in the human extracellular domain is a K in Xenopus, indicating that this site may be tolerant of a wide variety of changes, including polar residues, such as E, D, K, R, H, S, T, P, G, Y and probably A. T93 in the human extracellular domain is a K in Xenopus, indicating that a wide structural variation is tolerated at this position, with polar residues favored, such as S, K, R, E, D, H, G, P, G and Y. F108 in the human extracellular domain is a Y in Xenopus, and therefore Y or other hydrophobic group, such as I, V or L should be tolerated. E111 in the human extracellular domain is K in Xenopus, indicating that charged residues will be tolerated at this position, including D, R, K and H, as well as Q and N. R112 in the human extracellular domain is K in Xenopus, indicating that basic residues are tolerated at this position, including R and H. A at position 119 in the human extracellular domain is relatively poorly conserved, and appears as P in rodents and V in Xenopus, thus essentially any amino acid should be tolerated at this position.

Moreover, ActRII proteins have been characterized in the art in terms of structural and functional characteristics, particularly with respect to ligand binding [Attisano et al. (1992) Cell 68 (1): 97-108; Greenwald et al. (1999) Nature Structural Biology 6 (1): 18-22; Allendorph et al. (2006) PNAS 103 (20:7643-7648; Thompson et al. (2003) The EMBO Journal 22 (7): 1555-1566; as well as U.S. Pat. Nos. 7,709,605, 7,612,041, and 7,842,663]. In addition to the teachings herein, these references provide ample guidance for how to generate ActRIIB variants that retain one or more normal activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIB, as demarcated by the outermost of these conserved cysteines, corresponds to positions 29-109 of SEQ ID NO: 1 (ActRIIB precursor). The structurally less-ordered amino acids flanking these cysteine-demarcated core sequences can be truncated by 1, 2, 3, 4, 5, 6, 7, 8, 9, 10, 11, 12, 13, 14, 15, 16, 17, 18, 19, 20, 21, 22, 23, 24, 25, 26, 27, or 28 residues at the N-terminus and/or 1, 2, 3, 4, 5, 6, 7, 8, 9, 10, 11, 12, 13, 14, 15, 16, 17, 18, 19, 20, 21, or 22 residues a the C-terminus without necessarily altering ligand binding. Exemplary ActRIIB extracellular domains for N-terminal and/or C-terminal truncation include SEQ ID NOs: 2, 3, 5, 6, 318, and 331.

Attisano et al. showed that a deletion of the proline knot at the C-terminus of the extracellular domain of ActRIIB reduced the affinity of the receptor for activin. An ActRIIB-Fc fusion protein containing amino acids 20-119 of present SEQ ID NO: 1, “ActRIIB (20-119)-Fc”, has reduced binding to GDF11 and activin relative to an ActRIIB (20-134)-Fc, which includes the proline knot region and the complete juxtamembrane domain (see, e.g., U.S. Pat. No. 7,842,663). However, an ActRIIB (20-129)-Fc protein retains similar, but somewhat reduced activity, relative to the wild-type, even though the proline knot region is disrupted.

Thus, ActRIIB extracellular domains that stop at amino acid 134, 133, 132, 131, 130 and 129 (with respect to SEQ ID NO: 1) are all expected to be active, but constructs stopping at 134 or 133 may be most active. Similarly, mutations at any of residues 129-134 (with respect to SEQ ID NO: 1) are not expected to alter ligand-binding affinity by large margins. In support of this, it is known in the art that mutations of P129 and P130 (with respect to SEQ ID NO: 1) do not substantially decrease ligand binding. Therefore, an ActRIIB polypeptide of the present disclosure may end as early as amino acid 109 (the final cysteine), however, forms ending at or between 109 and 119 (e.g., 109, 110, 111, 112, 113, 114, 115, 116, 117, 118, or 119) are expected to have reduced ligand binding. Amino acid 119 (with respect to present SEQ ID NO:1) is poorly conserved and so is readily altered or truncated. ActRIIB polypeptides ending at 128 (with respect to SEQ ID NO: 1) or later should retain ligand-binding activity. ActRIIB polypeptides ending at or between 119 and 127 (e.g., 119, 120, 121, 122, 123, 124, 125, 126, or 127), with respect to SEQ ID NO: 1, will have an intermediate binding ability. Any of these forms may be desirable to use, depending on the clinical or experimental setting.

At the N-terminus of ActRIIB, it is expected that a protein beginning at amino acid 29 or before (with respect to SEQ ID NO: 1) will retain ligand-binding activity. Amino acid 29 represents the initial cysteine. An alanine-to-asparagine mutation at position 24 (with respect to SEQ ID NO: 1) introduces an N-linked glycosylation sequence without substantially affecting ligand binding [U.S. Pat. No. 7,842,663]. This confirms that mutations in the region between the signal cleavage peptide and the cysteine cross-linked region, corresponding to amino acids 20-29, are well tolerated. In particular, ActRIIB polypeptides beginning at position 20, 21, 22, 23, and 24 (with respect to SEQ ID NO: 1) should retain general ligand-biding activity, and ActRIIB polypeptides beginning at positions 25, 26, 27, 28, and 29 (with respect to SEQ ID NO: 1) are also expected to retain ligand-biding activity. It has been demonstrated, e.g., U.S. Pat. No. 7,842,663, that, surprisingly, an ActRIIB construct beginning at 22, 23, 24, or 25 will have the most activity.

Taken together, a general formula for an active portion (e.g., ligand-binding portion) of ActRIIB comprises amino acids 29-109 of SEQ ID NO: 1. Therefore ActRIIB polypeptides may, for example, comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to any one of amino acids 20-29 (e.g., beginning at any one of amino acids 20, 21, 22, 23, 24, 25, 26, 27, 28, or 29) of SEQ ID NO: 1 and ending at a position corresponding to any one amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. Other examples include polypeptides that begin at a position from 20-29 (e.g., any one of positions 20, 21, 22, 23, 24, 25, 26, 27, 28, or 29) or 21-29 (e.g., any one of positions 21, 22, 23, 24, 25, 26, 27, 28, or 29) of SEQ ID NO: 1 and end at a position from 119-134 (e.g., any one of positions 119, 120, 121, 122, 123, 124, 125, 126, 127, 128, 129, 130, 131, 132, 133, or 134), 119-133 (e.g., any one of positions 119, 120, 121, 122, 123, 124, 125, 126, 127, 128, 129, 130, 131, 132, or 133), 129-134 (e.g., any one of positions 129, 130, 131, 132, 133, or 134), or 129-133 (e.g., any one of positions 129, 130, 131, 132, or 133) of SEQ ID NO: 1. Other examples include constructs that begin at a position from 20-24 (e.g., any one of positions 20, 21, 22, 23, or 24), 21-24 (e.g., any one of positions 21, 22, 23, or 24), or 22-25 (e.g., any one of positions 22, 22, 23, or 25) of SEQ ID NO: 1 and end at a position from 109-134 (e.g., any one of positions 109, 110, 111, 112, 113, 114, 115, 116, 117, 118, 119, 120, 121, 122, 123, 124, 125, 126, 127, 128, 129, 130, 131, 132, 133, or 134), 119-134 (e.g., any one of positions 119, 120, 121, 122, 123, 124, 125, 126, 127, 128, 129, 130, 131, 132, 133, or 134) or 129-134 (e.g., any one of positions 129, 130, 131, 132, 133, or 134) of SEQ ID NO: 1. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 1.

The variations described herein may be combined in various ways. In some embodiments, ActRIIB variants comprise no more than 1, 2, 5, 6, 7, 8, 9, 10 or 15 conservative amino acid changes in the ligand-binding pocket, optionally zero, one or more non-conservative alterations at positions 40, 53, 55, 74, 79 and/or 82 in the ligand-binding pocket. Sites outside the binding pocket, at which variability may be particularly well tolerated, include the amino and carboxy termini of the extracellular domain (as noted above), and positions 42-46 and 65-73 (with respect to SEQ ID NO: 1). An asparagine-to-alanine alteration at position 65 (N65A) does not appear to decrease ligand binding in the R64 background [U.S. Pat. No. 7,842,663]. This change probably eliminates glycosylation at N65 in the A64 background, thus demonstrating that a significant change in this region is likely to be tolerated. While an R64A change is poorly tolerated, R64K is well-tolerated, and thus another basic residue, such as H may be tolerated at position 64 [U.S. Pat. No. 7,842,663]. Additionally, the results of the mutagenesis program described in the art indicate that there are amino acid positions in ActRIIB that are often beneficial to conserve. With respect to SEQ ID NO: 1, these include position 80 (acidic or hydrophobic amino acid), position 78 (hydrophobic, and particularly tryptophan), position 37 (acidic, and particularly aspartic or glutamic acid), position 56 (basic amino acid), position 60 (hydrophobic amino acid, particularly phenylalanine or tyrosine). Thus, the disclosure provides a framework of amino acids that may be conserved in ActRIIB polypeptides. Other positions that may be desirable to conserve are as follows: position 52 (acidic amino acid), position 55 (basic amino acid), position 81 (acidic), 98 (polar or charged, particularly E, D, R or K), all with respect to SEQ ID NO: 1.

It has been previously demonstrated that the addition of a further N-linked glycosylation site (N-X-S/T) into the ActRIIB extracellular domain is well-tolerated (see, e.g., U.S. Pat. No. 7,842,663). Therefore, N-X-S/T sequences may be generally introduced at positions outside the ligand binding pocket defined in FIG. 1 in ActRIIB polypeptide of the present disclosure. Particularly suitable sites for the introduction of non-endogenous N-X-S/T sequences include amino acids 20-29, 20-24, 22-25, 109-134, 120-134 or 129-134 (with respect to SEQ ID NO: 1). N-X-S/T sequences may also be introduced into the linker between the ActRIIB sequence and an Fc domain or other fusion component as well as optionally into the fusion component itself. Such a site may be introduced with minimal effort by introducing an N in the correct position with respect to a pre-existing S or T, or by introducing an S or T at a position corresponding to a pre-existing N. Thus, desirable alterations that would create an N-linked glycosylation site are: A24N, R64N, S67N (possibly combined with an N65A alteration), E105N, R112N, G120N, E123N, P129N, A132N, R112S and R112T (with respect to SEQ ID NO: 1). Any S that is predicted to be glycosylated may be altered to a T without creating an immunogenic site, because of the protection afforded by the glycosylation. Likewise, any T that is predicted to be glycosylated may be altered to an S. Thus, the alterations S67T and S44T (with respect to SEQ ID NO: 1) are contemplated. Likewise, in an A24N variant, an S26T alteration may be used. Accordingly, an ActRIIB polypeptide of the present disclosure may be a variant having one or more additional, non-endogenous N-linked glycosylation consensus sequences as described above.

In certain embodiments, the disclosure relates to ActRII antagonists (inhibitors) that comprise a ActRIIB polypeptide, which includes fragments, functional variants, and modified forms thereof as well as uses thereof (e.g., treating or preventing a renal disease or condition, or one or more renal-associated complications). Preferably, ActRIIB polypeptides are soluble (e.g., comprise an extracellular domain of ActRIIB). In some embodiments, ActRIIB polypeptides antagonize activity (e.g., SMAD signaling) of one or more TGF-β family ligands (e.g., activin A, activin B, BMP6, BMP9, BMP10, GDF3, GDF8, and/or GDF11). Therefore, in some embodiments, ActRIIB polypeptides bind to one or more TGF-beta family ligands (e.g., activin A, activin B, BMP6, BMP9, BMP10, GDF3, GDF8, and/or GDF11). In some embodiments, ActRIIB polypeptides of the disclosure demonstrate a decreased binding affinity for BMP9. In some embodiments, ActRIIB polypeptides of the disclosure do not bind BMP9. In some embodiments, ActRIIB polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIB beginning at a residue corresponding to amino acids 20-29 (e.g., beginning at any one of amino acids 20, 21, 22, 23, 24, 25, 26, 27, 28, or 29) of SEQ ID NO: 1 and ending at a position corresponding to amino acids 109-134 (e.g., ending at any one of amino acids 109, 110, 111, 112, 113, 114, 115, 116, 117, 118, 119, 120, 121, 122, 123, 124, 125, 126, 127, 128, 129, 130, 131, 132, 133, or 134) of SEQ ID NO: 1. In some embodiments, ActRIIB polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 29-109 of SEQ ID NO: 1. In some embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 29-109 of SEQ ID NO: 1, wherein the position corresponding to L79 of SEQ ID NO: 1 is an acidic amino acid (naturally occurring acidic amino acids D and E or an artificial acidic amino acid). In certain embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 25-131 of SEQ ID NO: 1. In certain embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 25-131 of SEQ ID NO: 1, wherein the position corresponding to L79 of SEQ ID NO: 1 is an acidic amino acid. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 40, 42, 45, 46, 47, 48, 69, 74, 77, 78, 79, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, and 407. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 1. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 2. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 3. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 4. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 5. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 6. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 40. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 42. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 45. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 46. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 47. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 48. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 69. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 74. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 77. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 78. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 79. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 138. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 282. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 289. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 290. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 291. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 292. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 293. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 294. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 295. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 296. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 297. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 298. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 299. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 300. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 301. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 302. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 303. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 305. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 306. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 307. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 308. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 309. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 310. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 311. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 312. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 313. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 314. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 315. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 316. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 317. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 318. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 319. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 320. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 321. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 322. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 323. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 324. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 325. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 326. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 327. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 328. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 329. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 330. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 331. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 332. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 333. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 334. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 335. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 336. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 337. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 338. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 339. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 340. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 341. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 342. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 343. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 344. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 345. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 346. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 347. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 348. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 349. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 350. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 351. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 352. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 353. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 354. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 355. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 356. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 357. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 358. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 359. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 360. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 361. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 362. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 363. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 364. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 365. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 366. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 367. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 368. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 369. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 370. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 371. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 372. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 373. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 374. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 375. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 376. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 377. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 378. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 379. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 380. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 381. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 382. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 383. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 384. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 385. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 386. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 387. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 388. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 389. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 390. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 391. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 392. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 393. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 394. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 395. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 396. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 397. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 398. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 399. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 400. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 401. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 402. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 403. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 404. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 405. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 406. In some embodiments, ActRIIB polypeptides of disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to SEQ ID NO: 407. In some embodiments, ActRIIB polypeptides of the disclosure comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 40, 42, 45, 46, 47, 48, 69, 74, 77, 78, 79, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, and 407, wherein the position corresponding to L79 of SEQ ID NO: 1 is an acidic amino acid. In some embodiments, ActRIIB polypeptides of the disclosure comprise, consist, or consist essentially of, at least one ActRIIB polypeptide wherein the position corresponding to L79 of SEQ ID NO: 1 is not an acidic amino acid (i.e., is not naturally occurring acid amino acids D or E or an artificial acidic amino acid residue).

In some embodiments, the ActRIIB polypeptide of the disclosure comprises an alternate, soluble form of ActRIIB (designated ActRIIB5), in which exon 4, including the ActRIIB transmembrane domain, has been replaced by a different C-terminal sequence (see, e.g., WO 2007/053775). In some embodiments, ActRIIB5 polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a polypeptide selected from the group consisting of SEQ ID NOS: 50, 51, and 52.

In some embodiments, ActRIIB polypeptides of the disclosure comprise, consist, or consist essentially of, at least one extracellular ActRIIB variant polypeptide having the sequence of SEQ ID NO: 282 shown below:

(SEQ ID NO: 282)
GRGEAETRECIFYNANWEKDRTNQSGLEPCYGDQDKRRHCFASWKNSSG
TIELVKQGCWLDDINCYDRQECVAKKDSPEVYFCCCEGNFCNERFTHLP
EAGGPEVTYEPPPTAPT.

In some embodiments, ActRIIB polypeptides of the disclosure comprise, consist, or consist essentially of, at least one extracellular ActRIIB variant polypeptide having the sequence of any one of SEQ ID NOs: 282, 289, or 290-302. In some embodiments, ActRIIB polypeptides of the disclosure comprise, consist, or consist essentially of, at least one extracellular ActRIIB variant polypeptide having the sequence of any one of SEQ ID NOs: 282 or 290-302 (Table 3).

In some embodiments, ActRIIB polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the processed (mature) extracellular ActRIIB polypeptide sequence (SEQ ID NO: 2).

Polypeptides described herein include an extracellular ActRIIB variant having at least one amino acid substitution relative to the processed (mature) extracellular ActRIIB polypeptide sequence having the sequence of SEQ ID NO: 2. Possible amino acid substitutions at 28 different positions may be introduced to an extracellular ActRIIB variant (Table 1). An extracellular ActRIIB variant may have one or more (e.g., 1-28, 1-25, 1-23, 1-21, 1-19, 1-17, 1-15, 1-13, 1-11, 1-9, 1-7, 1-5, 1-3, or 1-2; e.g., 1, 2, 3, 4, 5, 6, 7, 8, 9, 10, 11, 12, 13, 14, 15, 16, 17, 18, 19, 20, 21, 22, 23, 24, 25, 26, or 27) amino acid substitutions relative the sequence of a processed (mature) extracellular ActRIIB polypeptide sequence (SEQ ID NO: 2). In some embodiments, an extracellular ActRIIB variant (e.g., an extracellular ActRIIB variant having a sequence of SEQ ID NO: 289) may include amino acid substitutions at all of the 28 positions as listed in Table 1. In some embodiments, an extracellular ActRIIB variant may include amino acid substitutions at a number of positions, e.g., at 3, 4, 5, 6, 7, 8, 9, 10, 12, 15, 16, 18, 20, 22, 24, 26, or 27 out of the 28 positions, as listed in Table 1. In some embodiments, the substitutions are substitutions of an amino acid from an ActRIIA polypeptide sequence into the same position in an ActRIIB polypeptide sequence. In some embodiments, the substitutions are novel changes (e.g., substitutions of amino acids that are not in the corresponding position of ActRIIA, e.g., S48T, 151 L, Q69D, or E70T).

Amino acid substitutions can worsen or improve the activity and/or binding affinity of the ActRIIB variants disclosed herein (e.g., an extracellular ActRIIB variant having the sequence of any one of SEQ ID NOs: 282, 289, and 290-30 (e.g., SEQ ID NOs: 282 and 290-302)). In some embodiments, the amino acid substitutions worsen the binding affinity of the ActRIIB variants to BMP9 (e.g., the variants have reduced binding to BMP9 relative to wild-type extracellular ActRIIB, or have lower binding to BMP9 than to other ActRIIB ligands (e.g., activin A or B, myostatin, or GDF-11)). In some embodiments, the ActRIIB variants have reduced or no substantial binding to BMP9. In some embodiments, the amino acid substitutions improve the binding affinity of ActRIIB to myostatin, activin A or B, and/or GDF-11 (e.g., the variants have improved binding affinity relative to wild-type extracellular ActRIIB, or bind more strongly to myostatin, activin A or B, or GDF-11 than to BMP9). In some embodiments, the amino acid substitutions reduce the binding affinity of ActRIIB to myostatin, activin A or B, and/or GDF-11 (e.g., the variants have decreased binding affinity relative to wild-type extracellular ActRIIB, or have reduced binding to myostatin, activin A or B, or GDF-11 as compared to BMP9). In some embodiments, the amino acid substitutions do not substantially change extracellular ActRIIB function (e.g., the ActRIIB variants increase lean mass, muscle, mass, or bone mineral density, or reduce or prevent fibrosis, by a similar amount as wild-type extracellular ActRIIB, e.g., the ActRIIB variants are functionally equivalent to the wild-type extracellular ActRIIB). In some embodiments, the amino acid substitutions confer a property or activity of an ActRIIA polypeptide on an ActRIIB variant polypeptide (e.g., the ActRIIB variant polypeptide has a longer half-life than wild-type extracellular ActRIIB). In some embodiments, the ActRIIB variant polypeptides have one or more, two or more, or three or more of the above properties (e.g., reduced BMP9 binding and improved binding to activin A or B, myostatin, and/or GDF-11, or reduced BMP9 binding and functional equivalence to wild-type ActRIIB).

In some embodiments, ActRIIB polypeptides of the disclosure (e.g., an extracellular ActRIIB variant having the sequence of any one of SEQ ID NOs: 282, 289, and 290-30 (e.g., SEQ ID NOs: 282 and 290-302)) have one or more amino acid substitutions that reduce BMP9 binding. In some embodiments, the amino acid substitution that reduces BMP9 binding is E75K (e.g., X₂₄ is K in SEQ ID NO: 289). In some embodiments, the amino acid substitutions that reduce BMP9 binding are Q69T and E70D (e.g., X₂₁ is T and X₂₂ is D in SEQ ID NO: 289). In some embodiments, the amino acid substitutions that reduce BMP9 binding are Q69D and E70T (e.g., X₂₁ is D and X₂₂ is T in SEQ ID NO: 289). In some embodiments, the amino acid substitutions that reduce BMP9 binding are T74K, E75K, E76D, N77S, and Q79E (e.g., X₂₃, X₂₄, X₂₅, X₂₆, and X₂₈ are K, K, D, S, and E, respectively, in SEQ ID NO: 289). In some embodiments, the ActRIIB variants have more than one of the aforementioned amino acid substitutions that reduce BMP9 binding (e.g., substitution E75K and substitutions Q69D and E70T, or substitution E75K and substitutions Q69T and E70D). In some embodiments, the ActRIIB variants disclosed herein have one or more amino acid substitutions that reduce BMP9 binding, and one or more additional amino acid substitutions. The additional amino acid substitutions may confer other beneficial properties, such as altered binding to activins or myostatin or improved activity. For example, amino acid substitutions T74K, E75K, E76D, N77S, and Q79E lead to a reduction in ActRIIB variant activity, but including additional substitutions S25T and S47I; E31Y, E33D, and Q34K; or Y41F, R45K, and K56Q improves the ActRIIB variant activity. The additional amino acid substitutions may include one or more of substitutions I11L, Y12F, L19K, E20D, S25T, L27V, R29P, E31Y, E33D, Q34K, L38R, Y41F, R45K, S47I, S48T, T50S, I51L, L53I, K56Q, F631, T74K, E76D, N77S, Q79E, or F89M.

In some embodiments, variant ActRIIB polypeptides of the disclosure comprise one or more amino acid substitutions relative to the sequence of SEQ ID NO: 2, in which the variant contains one or more amino acid substitutions that impart reduced BMP9 binding relative to wild type extracellular ActRIIB, and one or more additional amino acid substitutions, wherein the substitutions that reduce BMP9 binding are one or more of: (a) amino acid substitution E75K; (b) amino acid substitutions Q69T and E70D; or (c) amino acid substitutions Q69D and E70T. In some embodiments, the one or more additional amino acid substitutions are selected from the group consisting of 11IL, Y12F, L19K, E20D, S25T, L27V, R29P, E31Y, E33D, Q34K, L38R, Y41F, R45K, S47I, S48T, T50S, I51L, L53I, K56Q, F631, T74K, E76D, N77S, Q79E, and F89M. In some embodiments, the variant contains amino acid substitution E75K and additional amino acid substitutions E20D and F631. In some embodiments, the variant polypeptide further comprises amino acid substitution E75K. In some embodiments, the variant contains amino acid substitution E75K and additional amino acid substitutions that reduce BMP9 binding. In some embodiments of any of the above embodiments, the additional amino acid substitutions that reduce BMP9 binding are T74K, E76D, N77S, and Q79E. In some embodiments, the variant further contains one or more additional amino acid substitutions. In some embodiments, the variant contains additional amino acid substitutions Y41F, R45K, and K56Q. In some embodiments, the variant further contains additional amino acid substitutions Y12F, L19K, E20D, R29P, E31Y, E33D, L38R, and F63I. In some embodiments, the variant contains additional amino acid substitutions S25T and S47I. In some embodiments, the variant contains additional amino acid substitution S48T. In some embodiments, the variant contains additional amino acid substitution R29P. In some embodiments, the variant contains additional amino acid substitutions E31Y, E33D, and Q34K. In some embodiments, the variant contains additional amino acid substitutions Y12F, L19K, and E20D. In some embodiments, the variant contains additional amino acid substitutions E31Y, E33D, and L38R. In some embodiments, the variant contains amino acid substitutions Q69T and E70D, and additional amino acid substitutions I11L, L27V, Q34K, T50S, I51L, L531, and F89M. In some embodiments, the variant contains amino acid substitutions Q69D and E70T, and additional amino acid substitutions I11L, L27V, Q34K, T50S, I51L, L531, and F89M. In some embodiments, the variant further contains amino acid substitution E75K. In some embodiments, the variant polypeptide comprises the sequence of any one of SEQ ID NOs: 282 or 290-302. See, e.g., Table 3.

In some embodiments, a polypeptide described herein includes an extracellular ActRIIB variant having the sequence of SEQ ID NO: 289.

TABLE 1
Amino acid substitutions in an extracellular
ActRIIB variant having a sequence of SEQ ID NO: 289
GRGEAETRECX₁X₂YNANWEX₃X₄RTNQX₅GX₆EX₇CX₈GX₉X₁₀DKRX₁₁HC
X₁₂ASWX₁₃NX₁₄X₁₅GX₁₆X₁₇EX₁₈VKX₁₉GCWLDDX₂₀NCYDRX₂₁X₂₂CVA
X₂₃X₂₄X₂₅X₂₆PX₂₇VYFCCCEGNX₂₈CNERFTHLPEAGGPEVTYEPPPTAPT
(SEQ ID NO: 289)

| | |
|---|---|
| X₁ | I or L |
| X₂ | F, Y, A, V, I, L, M, |
| | or W |
| X₃ | L or K |
| X₄ | D, E, or A |
| X₅ | T, S, N, or Q |
| X₆ | L, V, A, I, M, F, Y, |
| | or W |
| X₇ | Por R |
| X₈ | Y or E |
| X₉ | D, E, or A |
| X₁₀ | K or Q |
| X₁₁ | R or L |
| X₁₂ | A, V, I, L, M, Y, W |
| | or F |
| X₁₃ | R, H, or K |
| X₁₄ | Sor I |
| X₁₅ | S, N, Q, or T |
| X₁₆ | S, N, Q, or T |
| X₁₇ | L, V, A, I, M, F, Y, |
| | or W |
| X₁₈ | L, V, A, I, M, F, Y, |
| | or W |
| X₁₉ | Kor Q |
| X₂₀ | L, V, A, I, M, F, Y, |
| | or W |
| X₂₁ | Q, T, S, N or D |
| X₂₂ | E, D, A, or T |
| X₂₃ | K or T |
| X₂₄ | K or E |
| X₂₅ | D, E, or A |
| X₂₆ | T, S, N, or Q |
| X₂₇ | E or Q |
| X₂₈ | L, V, A, I, M, F, Y, |
| | or W |

TABLE 2
Compositions that can be administered to a subject according to the methods
described herein.

| Row | Composition |
|---|---|
| 1 | A polypeptide containing an ActRIIB variant, the variant having a |
| | sequence of GRGEAETRECX₁X₂YNANWEX₃X₄RTNQX₅GX₆EX₇CX₈GX₉X₁₀DKRX₁₁HCX₁₂AS |
| | WX₁₃NX₁₄X₁₅GX₁₆X₁₇EX₁₈VKX₁₉GCWLDDX₂₀NCYDRX₂₁X₂₂CVAX₂₃X₂₄X₂₅ |
| | X₂₆PX27VYFCCCEGNX28CNERFTHLPEAGGPEVTYEPPPTAPT (SEQ ID NO: 289), |
| | wherein X₁ is I or L; X₂ is For Y; X₃ is L or K; X₄ is D or E; X₅ is T or S; X₆ is L or V; |
| | X₇ is Por R; X₈ is Y or E; X₉ is D or E; X₁₀ is K or Q; X₁₁ is R or L; X₁₂ is Y or F; X₁₃ |
| | is R or K; X₁₄ is S or I; X₁₅ is S or T; X₁₆ is S or T; X₁₇ is I or L; X₁₈ is I or L; X₁₉ |
| | is K or Q; X₂₀ is For I; X₂₁ is Q, T, or D; X₂₂ is E, D, or T; X₂₃ is K or T; X₂₄ is K or |
| | E; X₂₅ is D or E; X₂₆ is S or N; X₂₇ is E or Q; and X₂₈ is F or M, and wherein X₂₄ is E |
| | and/or either X₂₁ is T and X₂₂ is D or X₂₁ is D and X₂₂ is T. |
| 2 | A polypeptide containing an ActRIIB variant, the variant having a |
| | sequence of GRGEAETRECX₁X₂YNANWEX₃X₄RTNQX₅GX₆EX₇CX₈GX₉X₁₀DKRX₁₁HCX₁₂AS |
| | WX₁₃NX₁₄X₁₅GX₁₆X₁₇EX₁₈VKX₁₉GCWLDDX₂₀NCYDRX₂₁X₂₂CVAX₂₃X₂₄X₂₅ |
| | X₂₆PX₂₇VYFCCCEGNX₂₈CNERFTHLPEAGGPEVTYEPPPTAPT (SEQ ID NO: 289), |
| | wherein X₁ is I or L; X₂ is F, Y, A, V, I, L, M, or W; X₃ is L or K; X₄ is D, E, or A; X₅ |
| | is T, S, N, or Q; X₆ is L, V, A, I, M, F, Y, or W; X₇ is P or R; X₈ is Y or E; X₉ is D, E, |
| | or A; X₁₀ is K or Q; X₁₁ is R or L; X₁₂ is A, V, I, L, M, Y, W or F; X₁₃ is R, H, or K; |
| | X₁₄ is S or I; X₁₅ is S, N, Q, or T; X₁₆ is S, N, Q, or T; X₁₇ is L, V, A, I, M, F, Y, or W; |
| | X₁₈ is L, V, A, I, M, F, Y, or W; X₁₉ is K or Q; X₂₀ is L, V, A, I, M, F, Y, or W; X₂₁ is |
| | Q, T, S, N or D; X₂₂ is E, D, A, or T; X₂₃ is K or T; X₂₄ is K or E; X₂₅ is D, A, or E; X₂₆ |
| | is T, S, N, or Q; X₂₇ is E or Q; and X₂₈ is L, V, A, I, M, F, Y, or W, and wherein X₂₄ is |
| | E and/or either X₂₁ is T and X₂₂ is D or X₂₁ is D and X₂₂ is T. |
| 3 | The variant of row 1 or 2, wherein X₁ is I. |
| 4 | The variant of row 1 or 2, wherein X₁ is L. |
| 5 | The variant of any one of rows 1-4, wherein X₂ is F. |
| 6 | The variant of any one of rows 1-4, wherein X₂ is Y. |
| 7 | The variant of any one of rows 1-6, wherein X₃ is L. |
| 8 | The variant of any one of rows 1-6, wherein X₃ is K. |
| 9 | The variant of any one of rows 1-8, wherein X₄ is D. |
| 10 | The variant of any one of rows 1-8, wherein X₄ is E. |
| 11 | The variant of any one of rows 1-10, wherein X₅ is T. |
| 12 | The variant of any one of rows 1-10, wherein X₅ is S. |
| 13 | The variant of any one of rows 1-12, wherein X₆ is L. |
| 14 | The variant of any one of rows 1-12, wherein X₆ is V. |
| 15 | The variant of any one of rows 1-14, wherein X₇ is P. |
| 16 | The variant of any one of rows 1-14, wherein X₇ is R. |
| 17 | The variant of any one of rows 1-16, wherein X₈ is Y. |
| 18 | The variant of any one of rows 1-16, wherein X₈ is E. |
| 19 | The variant of any one of rows 1-18, wherein X₉ is D. |
| 20 | The variant of any one of rows 1-18, wherein X₉ is E. |
| 21 | The variant of any one of rows 1-20, wherein X₁₀ is K. |
| 22 | The variant of any one of rows 1-20, wherein X₁₀ is Q. |
| 23 | The variant of any one of rows 1-22, wherein X₁₁ is R. |
| 24 | The variant of any one of rows 1-22, wherein X₁₁ is L. |
| 25 | The variant of any one of rows 1-24, wherein X₁₂ is Y. |
| 26 | The variant of any one of rows 1-24, wherein X₁₂ is F. |
| 27 | The variant of any one of rows 1-26, wherein X₁₃ is R. |
| 28 | The variant of any one of rows 1-26, wherein X₁₃ is K. |
| 29 | The variant of any one of rows 1-28, wherein X₁₄ is S. |
| 30 | The variant of any one of rows 1-28, wherein X₁₄ is I. |
| 31 | The variant of any one of rows 1-30, wherein X₁₅ is S. |
| 32 | The variant of any one of rows 1-30, wherein X₁₅ is T. |
| 33 | The variant of any one of rows 1-32, wherein X₁₆ is S. |
| 34 | The variant of any one of rows 1-32, wherein X₁₆ is T. |
| 35 | The variant of any one of rows 1-34, wherein X₁₇ is I. |
| 36 | The variant of any one of rows 1-34, wherein X₁₇ is L. |
| 37 | The variant of any one of rows 1-36, wherein X₁₈ is I. |
| 38 | The variant of any one of rows 1-36, wherein X₁₈ is L. |
| 39 | The variant of any one of rows 1-38, wherein X₁₉ is K. |
| 40 | The variant of any one of rows 1-38, wherein X₁₉ is Q. |
| 41 | The variant of any one of rows 1-40, wherein X₂₀ is F. |
| 42 | The variant of any one of rows 1-40, wherein X₂₀ is I. |
| 43 | The variant of any one of rows 1-42, wherein X₂₁ is Q. |
| 44 | The variant of any one of rows 1-42, wherein X₂₁ is T. |
| 45 | The variant of any one of rows 1-42, wherein X₂₁ is D. |
| 46 | The variant of any one of rows 1-43, wherein X₂₂ is E. |
| 47 | The variant of any one of rows 1-42 and row 45, wherein X₂₂ is D. |
| 48 | The variant of any one of rows 1-42 and row 45, wherein X₂₂ is T. |
| 49 | The variant of any one of rows 1-48, wherein X₂₃ is K. |
| 50 | The variant of any one of rows 1-48, wherein X₂₃ is T. |
| 51 | The variant of any one of rows 1-50, wherein X₂₄ is K. |
| 52 | The variant of any one of rows 1-42, row 44, row 45, row 47, and |
| | row 48-50, wherein X₂₄ is E. |
| 53 | The variant of any one of rows 1-52, wherein X₂₅ is D. |
| 54 | The variant of any one of rows 1-52, wherein X₂₅ is E. |
| 55 | The variant of any one of rows 1-54, wherein X₂₆ is S. |
| 56 | The variant of any one of rows 1-54, wherein X₂₆ is N. |
| 57 | The variant of any one of rows 1-56, wherein X₂₇ is E. |
| 58 | The variant of any one of rows 1-56, wherein X₂₇ is Q. |
| 59 | The variant of any one of rows 1-58, wherein X₂₈ is F. |
| 60 | The variant of any one of rows 1-58, wherein X₂₈ is M. |

In some embodiments, a polypeptide described herein includes an extracellular ActRIIB variant having a sequence of any one of SEQ ID NOs: 282 and 290-302 (Table 3).

TABLE 3
Extracellular ActRIIB variants having the sequences
of SEQ ID NOs: 282 and 290-302

| SEQ ID | |
|---|---|
| NO | Amino Acid Sequence |
| 282 | GRGEAETRECIFYNANWEKDRTNQSGLEPCYGDQDKRRHCFASWKNSSGTI |
| | ELVKQGCWLDDINCYDRQECVAKKDSPEVYFCCCEGNFCNERFTHLPEAG |
| | GPEVTYEPPPTAPT |
| 290 | GRGEAETRECIYYNANWELDRTNQSGLERCEGEQDKRLHCYASWRNSSGTI |
| | ELVKKGCWLDDINCYDRQECVATKENPQVYFCCCEGNFCNERFTHLPEAG |
| | GPEVTYEPPPTAPT |
| 291 | GRGEAETRECIYYNANWELERTNQSGLERCEGEQDKRLHCYASWRNSSGTI |
| | ELVKKGCWLDDFNCYDRQECVAKKDSPEVYFCCCEGNFCNERFTHLPEAG |
| | GPEVTYEPPPTAPT |
| 292 | GRGEAETRECIYYNANWELERTNQTGLERCEGEQDKRLHCYASWRNISGTI |
| | ELVKKGCWLDDFNCYDRQECVAKKDSPEVYFCCCEGNFCNERFTHLPEAG |
| | GPEVTYEPPPTAPT |
| 293 | GRGEAETRECIYYNANWELERTNQTGLERCEGEQDKRLHCYASWRNITGTI |
| | ELVKKGCWLDDFNCYDRQECVAKKDSPEVYFCCCEGNFCNERFTHLPEAG |
| | GPEVTYEPPPTAPT |
| 294 | GRGEAETRECIYYNANWELERTNQSGLEPCEGEQDKRLHCYASWRNSSGTI |
| | ELVKKGCWLDDFNCYDRQECVAKKDSPEVYFCCCEGNFCNERFTHLPEAG |
| | GPEVTYEPPPTAPT |
| 295 | GRGEAETRECIYYNANWELERTNQSGLERCYGDKDKRLHCFASWKNSSGTI |
| | ELVKQGCWLDDFNCYDRQECVAKKDSPEVYFCCCEGNFCNERFTHLPEAG |
| | GPEVTYEPPPTAPT |
| 296 | GRGEAETRECIYYNANWELERTNQSGLERCEGEQDKRLHCYASWRNSSGTI |
| | ELVKKGCWLDDENCYDRQECVAKKDSPEVYFCCCEGNFCNERFTHLPEAG |
| | GPEVTYEPPPTAPT |
| 297 | GRGEAETRECIFYNANWEKDRTNQSGLERCYGDQDKRRHCYASWRNSSGT |
| | IELVKKGCWLDDFNCYDRQECVAKKDSPEVYFCCCEGNFCNERFTHLPEAG |
| | GPEVTYEPPPTAPT |
| 298 | GRGEAETRECIYYNANWELERTNQSGLERCEGEQDKRLHCYASWRNSSGTI |
| | ELVKKGCWLDDFNCYDRQECVAKKDSPEVYFCCCEGNFCNERFTHLPEAG |
| | GPEVTYEPPPTAPT |
| 299 | GRGEAETRECLYYNANWELERTNQSGVERCEGEKDKRLHCYASWRNSSGS |
| | LEIVKKGCWLDDFNCYDRTDCVATEENPQVYFCCCEGNMCNERFTHLPEA |
| | GGPEVTYEPPPTAPT |
| 300 | GRGEAETRECLYYNANWELERTNQSGVERCEGEKDKRLHCYASWRNSSGS |
| | LEIVKKGCWLDDENCYDRDTCVATEENPQVYFCCCEGNMCNERFTHLPEA |
| | GGPEVTYEPPPTAPT |
| 301 | GRGEAETRECLYYNANWELERTNQSGVERCEGEKDKRLHCYASWRNSSGS |
| | LEIVKKGCWLDDFNCYDRTDCVATKENPQVYFCCCEGNMCNERFTHLPEA |
| | GGPEVTYEPPPTAPT |
| 302 | GRGEAETRECLYYNANWELERTNQSGVERCEGEKDKRLHCYASWRNSSGS |
| | LEIVKKGCWLDDFNCYDRDTCVATKENPQVYFCCCEGNMCNERFTHLPEA |
| | GGPEVTYEPPPTAPT |

In one aspect, the present disclosure provides isolated variant ActRIIB polypeptides comprising hybrid soluble ActRIIB polypeptides which retain myostatin- and activin A-neutralizing activities, but demonstrate dramatically reduced BMP9-neutralization. In various embodiments, the variant ActRIIB polypeptides comprise a hybrid soluble ActRIIB polypeptide having the amino acid sequence of SEQ ID NO: 303 wherein at least one of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least two of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least three of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least four of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least five of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least six of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least seven of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least eight of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least nine of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least ten of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least fifteen of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least twenty of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least twenty-five of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide. In various embodiments, the variant ActRIIB polypeptides comprise a hybrid soluble ActRIIB polypeptide having the amino acid sequence of SEQ ID NO: 303 wherein at least thirty of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with another amino acid, and wherein the hybrid ActRIIB polypeptide is capable of binding myostatin and activin A, but demonstrates a decreased binding affinity for BMP9 relative to a wild-type ActRIIB polypeptide.

In various embodiments, the variant ActRIIB polypeptides comprise hybrid soluble ActRIIB polypeptides having an amino acid sequence set forth in any one of SEQ ID NOS: 305-339 (see, e.g., Table 8), wherein the hybrid ActRIIB polypeptide is capable of binding myostatin and activin A, but demonstrates a decreased binding affinity for BMP9 relative to a wild-type ActRIIB polypeptide. In various embodiments, the hybrid soluble ActRIIB polypeptides are hybrid soluble ActRIIB polypeptides having an amino acid sequence that is at least 70%, 75%, 80%, 85%, 90%, 91%, 92%, 93%, 94%, 95%, 96%, 97%, 98%, 99% or 100% identical to an amino acid sequence selected from SEQ ID NOs: 305-339 (see, e.g., Table 8), wherein the hybrid ActRIIB polypeptide is capable of binding myostatin and activin A, but demonstrates a decreased binding affinity for BMP9 relative to a wild-type ActRIIB polypeptide.

In various embodiments, the variant ActRIIB polypeptide comprises an amino acid sequence set forth in any one of SEQ ID NOs: 340-406, wherein the variant ActRIIB polypeptide is capable of binding myostatin and activin A, but demonstrates a decreased binding affinity for BMP9 relative to a wild-type ActRIIB polypeptide. In various embodiments, the variant ActRIIB polypeptides are hybrid soluble ActRIIB polypeptides having an amino acid sequence that is at least 70%, 75%, 80%, 85%, 90%, 91%, 92%, 93%, 94%, 95%, 96%, 97%, 98%, 99% or 100% identical to an amino acid sequence selected from SEQ ID NOs: 340-406, wherein the hybrid ActRIIB polypeptide is capable of binding myostatin and activin A, but demonstrates a decreased binding affinity for BMP9 relative to a wild-type ActRIIB polypeptide.

In another aspect, the present disclosure provides isolated nucleic acid molecules comprising a polynucleotide encoding a hybrid soluble ActRIIB polypeptide of the present disclosure. In various embodiments, the polynucleotides encodes one of the polypeptide sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide. In various embodiments, the polynucleotides encode a polypeptide having an amino acid sequence at least 70%, 75%, 80%, 85%, 90%, 91%, 92%, 93%, 94%, 95%, 96%, 97%, 98%, 99% or 100% identity to any one of the polypeptides sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide. In various embodiments, the polynucleotides encode a polypeptide having at least 90% identity to any one of the polypeptides sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide. In various embodiments, the polynucleotides encode a polypeptide having an amino acid sequence at least 95% identity to any one of the polypeptides sequences set forth in SEQ ID NOs: 305-406, wherein the hybrid ActRIIB polypeptide is capable of binding myostatin and activin A, but demonstrates a decreased binding affinity for BMP9 relative to a wild-type ActRIIB polypeptide.

In some embodiments, an ActRIIB polypeptide of the disclosure comprises a hybrid soluble ActRIIB polypeptide that is derived from wild-type ActRIIB and wild-type ActRIIA. The hybrid soluble ActRIIB polypeptides are specifically engineered by replacing one or more amino acids of a truncated wild-type ActRIIB polypeptide with the amino acids from a truncated wild-type ActRIIA polypeptide at corresponding positions based on sequence alignment between the two truncated ActRII polypeptide extracellular domains at the amino acid level. The one or more amino acid replacements are specifically selected for purposes of providing hybrid soluble ActRIIB polypeptides which demonstrate a reduction of BMP9-neutralization as compared to wild-type ActRIIB polypeptide, while retaining myostatin- and activin A-neutralization.

In various embodiments, the truncated extracellular domain of ActRIIB used to prepare the hybrid soluble ActRIIB polypeptides has the 110 amino acid sequence set forth in SEQ ID NO: 303:

(SEQ ID NO: 303)
ETRECIYYNANWELERTNQSGLERCEGEQDKRLHCYASWRNSS
GTIELVKKGCWLDDENCYDRQECVATEENPQVYFCCCEGNFCN
ERFTHLPEAGGPEVTYEPPPTAPT

In various embodiments, the truncated extracellular domain of ActRIIA used to prepare the hybrid soluble ActRIIB polypeptides has the 110 amino acid sequence set forth in SEQ ID NO: 304:

(SEQ ID NO: 304)
ETQECLFFNANWEKDRTNQTGVEPCYGDKDKRRHCFATWKNIS
GSIEIVKQGCWLDDINCYDRTDCVEKKDSPEVYFCCCEGNMCN
EKFSYFPEMEVTQPTSNPVTPKPP

In various embodiments, the variant ActRIIB polypeptides comprise a hybrid soluble ActRIIB polypeptide having the amino acid sequence of SEQ ID NO: 303 wherein at least one of amino acid residues R3, 16, Y7, Y8, L14, E15, S20, L22, R24, E26, E28, Q29, L33, L48, Y36, S38, R40, S42, T45, K51, F58, Q64, E65, A68, T69, E70, E71, N72, Q74, F84, R88, T90, H91, L92, E94, A95, G96, G97, P98, E99, V100, Y102, E103, P105, P106, T107, A108, or T110 is substituted with the amino acid at the corresponding position of wild-type ActRIIA sequence (SEQ ID NO: 304), and wherein the hybrid soluble ActRIIB polypeptide is capable of binding myostatin and activin A, but demonstrates a decreased binding affinity for BMP9 relative to a wild-type ActRIIB polypeptide.

In various embodiments, the hybrid soluble ActRIIB polypeptide comprises the amino acid sequence of SEQ ID NO: 305, wherein amino acid residues E26, E28, Q29, L33, F58, Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 306, wherein amino acid residues E26, E28, Q29, L33, Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 307, wherein amino acid residues F58, Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 308, wherein amino acid residues F58, Q64, E65, A68, T69, E70, E71, and N72 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 309, wherein amino acid residues Q64, E65, A68, T69, E70, E71, and N72 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 310, wherein amino acid residues Q64, E65, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 311, wherein amino acid residues A68, T69, E70, E71, N72 and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 312, wherein amino acid residues A68, T69, E70, E71, and N72 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 313, wherein amino acid residues F58, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 314, wherein amino acid residues Q64, E65, A68, T69, E70, E71, N72, Q74, and F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 315, wherein amino acid residues A68, T69, E70, E71, N72, Q74, and F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 316, wherein amino acid residues R3, L14, E15, S20, L22, R24, E26, E28, Q29, and L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 317, wherein amino acid residues R3, L14, E15, S20, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 318, wherein amino acid residues E26, E28, Q29, and L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 319, wherein amino acid residues L14, E15, S20, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 320, wherein amino acid residues R3, L14, E15, S20, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 321, wherein amino acid residues R3, L14, E15, and S20 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 322, wherein amino acid residues R3, L14, and E15 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 323, wherein amino acid residues L14 and E15 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 324, wherein amino acid residue R3 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 325, wherein amino acid residues Y36, S38, and K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 326, wherein amino acid residues E26, E28, Q29, L33, and F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 327, wherein amino acid residue E70 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 328, wherein amino acid residue F58 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 329, wherein amino acid residues F58 and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 330, wherein amino acid residues E28, Q29, F58, and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 331, wherein amino acid residues E28, F58, and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 332, wherein amino acid residues E28 and E70 of SEQ ID NO: 303 have been replaced by the amino acid residues in the corresponding positions of SEQ ID NO: 304. In some embodiments, the hybrid soluble ActRIIB polypeptide has decreased binding affinity for BMP9.

In some embodiments, the hybrid soluble ActRIIB polypeptide binds myostatin and/or activin A. In various embodiments, the hybrid soluble ActRIIB polypeptide comprises the amino acid sequence of SEQ ID NO: 333, wherein amino acid residue E28 of SEQ ID NO: 303 has been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 334, wherein amino acid residues E26, E28, Q29, L33, A68, T69, E70, E71, N72, and Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 335, wherein amino acid residues Y7, Y8, L14, E15, S20, L22, and R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 336, wherein amino acid residues Y36, S38, R40, S42, T45, and K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 337, wherein amino acid residues Q64 and E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 338, wherein amino acid residue F84 of SEQ ID NO: 303 have been replaced by the amino acid residue in the corresponding position of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 339, wherein amino acid residues E28 and F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 340, wherein amino acid residues R3, 16, Y7, Y8, L14, E15, L22, R24, E26, E28, Q29, L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 341, wherein amino acid residues R3, 16, Y7, Y8, L14, E15, L22, R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 342, wherein amino acid residues 16, Y7, Y8, L14, E15, L22, R24 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 343, wherein amino acid residues 16, Y7, Y8, L14, E15, L22, R24, E26 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 344, wherein amino acid residues 16, Y7, Y8, L14, E15, L22, R24, E26, E28, Q29, L33 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 345, wherein amino acid residues 16, Y7, Y8, L14, E15, L22, R24, E26, E28, Q29, L33, Y36, S38, R40, S42, T45, L48, K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 346, wherein amino acid residues 16, Y7, Y8, L14, E15, L22, R24, E26, E28, Q29, L33, Y36, S38, R40, S42, T45, L48, K51, F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 347, wherein amino acid residues 16, Y7, Y8, L14, E15, L22, R24, E26, E28, Q29, L33, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 348, wherein amino acid residues R3, E26, E28, Q29, L33, Y36, S38, R40, S42, T45, L48, K51, F58 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 349, wherein amino acid residues E26, E28, Q29, L33, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 350, wherein amino acid residues E26, E28, Q29, L33, Y36, S38, R40, S42, T45, L48, K51, F58, Q64, E65, A68, T69, E70, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 351, wherein amino acid residues Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 352, wherein amino acid residues Y36, S38, R40, S42, T45, L48, K51, F58, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 353, wherein amino acid residues Y36, S38, R40, S42, T45, L48, K51, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 354, wherein amino acid residues Y36, S38, R40, S42, T45, L48, K51 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 355, wherein amino acid residues R3, E26, E28, Q29, L33, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 356, wherein amino acid residues R3, E26, E28, Q29, L33, F58, Q64, E65, A68, T69, E70, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 357, wherein amino acid residues R3, E26, E28, Q29, L33, Y36, S38, R40, S42, T45, L48, K51, F58, Q64, E65, A68, T69, E70, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 358, wherein amino acid residues R3, E26, E28, Q29, L33, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 359, wherein amino acid residues 16, Y7, Y8, L14, E15, L22, R24, Y36, S38, R40, S42, T45, L48, K51, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 360, wherein amino acid residues 16, Y7, Y8, L14, E15, L22, R24, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 361, wherein amino acid residues 16, Y7, Y8, L14, E15, L22, R24, E26, E28, Q29, L33, F58, Q64, E65, A68, T69, E70, E71, N72, Q74 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 362, wherein amino acid residues E26, E28, Q29, L33, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 363, wherein amino acid residues E26, E28, Q29, L33, K51, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 364, wherein amino acid residues E26, E28, Q29, L33, L48, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 365, wherein amino acid residues E26, E28, Q29, L33, T45, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 366, wherein amino acid residues E26, E28, Q29, L33, T45, L48, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 367, wherein amino acid residues E26, E28, Q29, L33, T45, L48, K51, Q64, E65 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 368, wherein amino acid residues Q64, E65, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 369, wherein amino acid residues R88, T90, H91, L92, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 370, wherein amino acid residues R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 371, wherein amino acid residues E26, E28, Q29, L33, F58, Q64, E65, A68, T69, E70, E71, N72, Q74, R88, T90, H91, L92, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 372, wherein amino acid residues E26, E28, Q29, L33, Q64, E65, A68, T69, E70, E71, N72, Q74, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 373, wherein amino acid residues E26, E28, Q29, L33, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 374, wherein amino acid residues E26, E28, Q29, L33, K51, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 375, wherein amino acid residues E26, E28, Q29, L33, L48, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 376, wherein amino acid residues E26, E28, Q29, L33, T45, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 377, wherein amino acid residues T45, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 378, wherein amino acid residues L48, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 379, wherein amino acid residues K51, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 380, wherein amino acid residues A68, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 381, wherein amino acid residues A68, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 382, wherein amino acid residues E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 383, wherein amino acid residues E71, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 384, wherein amino acid residues N72, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 385, wherein amino acid residues Q74, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 386, wherein amino acid residues E28, Q29, A68, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 387, wherein amino acid residues Q29, T69, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 388, wherein amino acid residues E28, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 389, wherein amino acid residues E28, Q29, K51, T69, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 390, wherein amino acid residues E28, Q29, L48, K51, T69E, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 391, wherein amino acid residues E26, E28, T45, L48, K51, T69, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 392, wherein amino acid residues Q29, L48, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 393, wherein amino acid residues E26, E28, L33, Q70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 394, wherein amino acid residues L33, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 395, wherein amino acid residues E26, T45, L48, Q64, E65, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 396, wherein amino acid residues L33, T45, T69, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 397, wherein amino acid residues L33, L48, T69, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 398, wherein amino acid residues L33, T45, L48, E70, R88, T90, H91, L92, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 399, wherein amino acid residues E28, L48, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 400, wherein amino acid residues E28, T45, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 401, wherein amino acid residues E28, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 402, wherein amino acid residues L48, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 403, wherein amino acid residues E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 404, wherein amino acid residues E28, L48, T79, E70, R88, T90, H91, L92, E94, A95, G96, G97, P98, E99, V100, Y102, E103, P105, P106, T107, A108, T110 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 405, wherein amino acid residues R3, 16, Y7, Y8, L14, E15, S20, L22, R24, E26, E28, Q29, L33, Y36, S38, R40, S42, T45, L48, K51, F58, Q64, E65, A68, T69, E71, N72, Q74, F84 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In various embodiments, the hybrid soluble ActRIIB polypeptide comprises the amino acid sequence of SEQ ID NO: 406, wherein amino acid residues E26, E28, Q29, L33, F56, E68 of SEQ ID NO: 303 have been replaced by the amino acid residues in the corresponding positions of SEQ ID NO: 304. In some embodiments, the hybrid soluble ActRIIB polypeptide has decreased binding affinity for BMP9. In some embodiments, the hybrid soluble ActRIIB polypeptide binds myostatin and/or activin A.

In certain embodiments, the present disclosure relates to ActRIIA polypeptides. As used herein, the term “ActRIIA” refers to a family of activin receptor type IIA (ActRIIA) proteins from any species and variants derived from such ActRIIA proteins by mutagenesis or other modification. Reference to ActRIIA herein is understood to be a reference to any one of the currently identified forms. Members of the ActRIIA family are generally transmembrane proteins, composed of a ligand-binding extracellular domain comprising a cysteine-rich region, a transmembrane domain, and a cytoplasmic domain with predicted serine/threonine kinase activity.

The term “ActRIIA polypeptide” includes polypeptides comprising any naturally occurring polypeptide of an ActRIIA family member as well as any variants thereof (including mutants, fragments, fusions, and peptidomimetic forms) that retain a useful activity. Examples of such variant ActRIIA polypeptides are provided throughout the present disclosure as well as in International Patent Application Publication Nos. WO 2006/012627, WO 2007/062188, WO2018/089706, WO2018/089715, and WO2019/094751 which are incorporated herein by reference in their entirety. Numbering of amino acids for all ActRIIA-related polypeptides described herein is based on the numbering of the human ActRIIA precursor protein sequence provided below (SEQ ID NO: 9), unless specifically designated otherwise.

The canonical human ActRIIA precursor protein sequence is as follows:

```
(SEQ ID NO: 9)
    1 MGAAAKLAFA VFLISCSSGA ILGRSETQEC LFFNANWEKD RTNQTGVEPC
  51 YGDKDKRRHC FATWKNISGS IEIVKQGCWL DDINCYDRTD CVEKKDSPEV
101 YFCCCEGNMC NEKFSYFPEM EVTQPTSNPV TPKPPYYNIL LYSLVPLMLI
151 AGIVICAFWV YRHHKMAYPP VLVPTQDPGP PPPSPLLGLK PLQLLEVKAR
201 GREGCVWKAQ LLNEYVAVKI FPIQDKQSWQ NEYEVYSLPG MKHENILQFI
251 GAEKRGTSVD VDLWLITAFH EKGSLSDFLK ANVVSWNELC HIAETMARGL
301 AYLHEDIPGL KDGHKPAISH RDIKSKNVLL KNNLTACIAD FGLALKFEAG
351 KSAGDTHGQV GTRRYMAPEV LEGAINFQRD AFLRIDMYAM GLVLWELASR
401 CTAADGPVDE YMLPFEEEIG QHPSLEDMQE VVVHKKKRPV LRDYWQKHAG
451 MAMLCETIEE CWDHDAEARL SAGCVGERIT QMQRLINIIT TEDIVTVVTM
501 VINVDFPPKE SSL
```

The signal peptide is indicated by a single underline; the extracellular domain is indicated in bold font; and the potential, endogenous N-linked glycosylation sites are indicated by a double underline.

A processed (mature) extracellular human ActRIIA polypeptide sequence is as follows:

(SEQ ID NO: 10)
ILGRSETQECLFFNANWEKDRINQTGVEPCYGDKDKRRHC
FATWKNISGSIEIVKQGCWLDDINCYDRTDCVEKKDSPEV
YFCCCEGNMCNEKFSYFPEMEVTQPTSNPVTPKPP

The C-terminal “tail” of the extracellular domain is indicated by single underline. The sequence with the “tail” deleted (a 415 sequence) is as follows:

(SEQ ID NO: 11)
ILGRSETQECLFFNANWEKDRINQTGVEPCYGDKDKRR
HCFATWKNISGSIEIVKQGCWLDDINCYDRTDCVEKKD
SPEVYFCCCEGNMCNEKFSYFPEM

A nucleic acid sequence encoding the human ActRIIA precursor protein (SEQ ID NO: 9) is shown below (SEQ ID NO: 12), as follows nucleotides 159-1700 of Genbank Reference Sequence NM_001616.4. The signal sequence is underlined.

```
(SEQ ID NO: 12)
      1 ATGGGAGCTG CTGCAAAGTT GGCGTTTGCC GTCTTTCTTA TCTCCTGTTC
    51 TTCAGGTGCT ATACTTGGTA GATCAGAAAC TCAGGAGTGT CTTTTCTTTA
  101 ATGCTAATTG GGAAAAAGAC AGAACCAATC AAACTGGTGT TGAACCGTGT
  151 TATGGTGACA AAGATAAACG GCGGCATTGT TTTGCTACCT GGAAGAATAT
  201 TTCTGGTTCC ATTGAAATAG TGAAACAAGG TTGTTGGCTG GATGATATCA
  251 ACTGCTATGA CAGGACTGAT TGTGTAGAAA AAAAAGACAG CCCTGAAGTA
  301 TATTTTTGTT GCTGTGAGGG CAATATGTGT AATGAAAAGT TTTCTTATTT
  351 TCCGGAGATG GAAGTCACAC AGCCCACTTC AAATCCAGTT ACACCTAAGC
  401 CACCCTATTA CAACATCCTG CTCTATTCCT TGGTGCCACT TATGTTAATT
  451 GCGGGGATTG TCATTTGTGC ATTTTGGGTG TACAGGCATC ACAAGATGGC
  501 CTACCCTCCT GTACTTGTTC CAACTCAAGA CCCAGGACCA CCCCCACCTT
  551 CTCCATTACT AGGTTTGAAA CCACTGCAGT TATTAGAAGT GAAAGCAAGG
  601 GGAAGATTTG GTTGTGTCTG GAAAGCCCAG TTGCTTAACG AATATGTGGC
  651 TGTCAAAATA TTTCCAATAC AGGACAAACA GTCATGGCAA AATGAATACG
  701 AAGTCTACAG TTTGCCTGGA ATGAAGCATG AGAACATATT ACAGTTCATT
  751 GGTGCAGAAA AACGAGGCAC CAGTGTTGAT GTGGATCTTT GGCTGATCAC
  801 AGCATTTCAT GAAAAGGGTT CACTATCAGA CTTTCTTAAG GCTAATGTGG
  851 TCTCTTGGAA TGAACTGTGT CATATTGCAG AAACCATGGC TAGAGGATTG
  901 GCATATTTAC ATGAGGATAT ACCTGGCCTA AAAGATGGCC ACAAACCTGC
  951 CATATCTCAC AGGGACATCA AAAGTAAAAA TGTGCTGTTG AAAAACAACC
1001 TGACAGCTTG CATTGCTGAC TTTGGGTTGG CCTTAAAATT TGAGGCTGGC
1051 AAGTCTGCAG GCGATACCCA TGGACAGGTT GGTACCCGGA GGTACATGGC
1101 TCCAGAGGTA TTAGAGGGTG CTATAAACTT CCAAAGGGAT GCATTTTTGA
1151 GGATAGATAT GTATGCCATG GGATTAGTCC TATGGGAACT GGCTTCTCGC
1201 TGTACTGCTG CAGATGGACC TGTAGATGAA TACATGTTGC CATTTGAGGA
1251 GGAAATTGGC CAGCATCCAT CTCTTGAAGA CATGCAGGAA GTTGTTGTGC
1301 ATAAAAAAAA GAGGCCTGTT TTAAGAGATT ATTGGCAGAA ACATGCTGGA
1351 ATGGCAATGC TCTGTGAAAC CATTGAAGAA TGTTGGGATC ACGACGCAGA
1401 AGCCAGGTTA TCAGCTGGAT GTGTAGGTGA AAGAATTACC CAGATGCAGA
1451 GACTAACAAA TATTATTACC ACAGAGGACA TTGTAACAGT GGTCACAATG
1501 GTGACAAATG TTGACTTTCC TCCCAAAGAA TCTAGTCTA
```

A nucleic acid sequence encoding the processed soluble (extracellular) human ActRIIA polypeptide (SEQ ID NO: 10) is as follows:

| | (SEQ ID NO: 13) |
|---|---|
| 1 | ATACTTGGTA GATCAGAAAC TCAGGAGTGT CTTTTCTTTA ATGCTAATTG |
| 51 | GGAAAAAGAC AGAACCAATC AAACTGGTGT TGAACCGTGT TATGGTGACA |
| 101 | AAGATAAACG GCGGCATTGT TTTGCTACCT GGAAGAATAT TTCTGGTTCC |
| 151 | ATTGAAATAG TGAAACAAGG TTGTTGGCTG GATGATATCA ACTGCTATGA |
| 201 | CAGGACTGAT TGTGTAGAAA AAAAAGACAG CCCTGAAGTA TATTTTTGTT |
| 251 | GCTGTGAGGG CAATATGTGT AATGAAAAGT TTTCTTATTT TCCGGAGATG |
| 301 | GAAGTCACAC AGCCCACTTC AAATCCAGTT ACACCTAAGC CACCC |

In some embodiments, the ActRIIA polypeptide sequence comprises the amino acid sequence set forth at accession number UniProtKB/Swiss-Prot P27037.1 (SEQ ID NO: 408 herein), and variants thereof. In some embodiments, the term “wild-type ActRIIA polypeptide” refers to the extracellular domain of ActRIIA, amino acids 1 to 135 (with signal sequence), or amino acids 20 through 135 of SEQ ID NO: 407 (without signal sequence) (referred to herein as SEQ ID NO: 409).

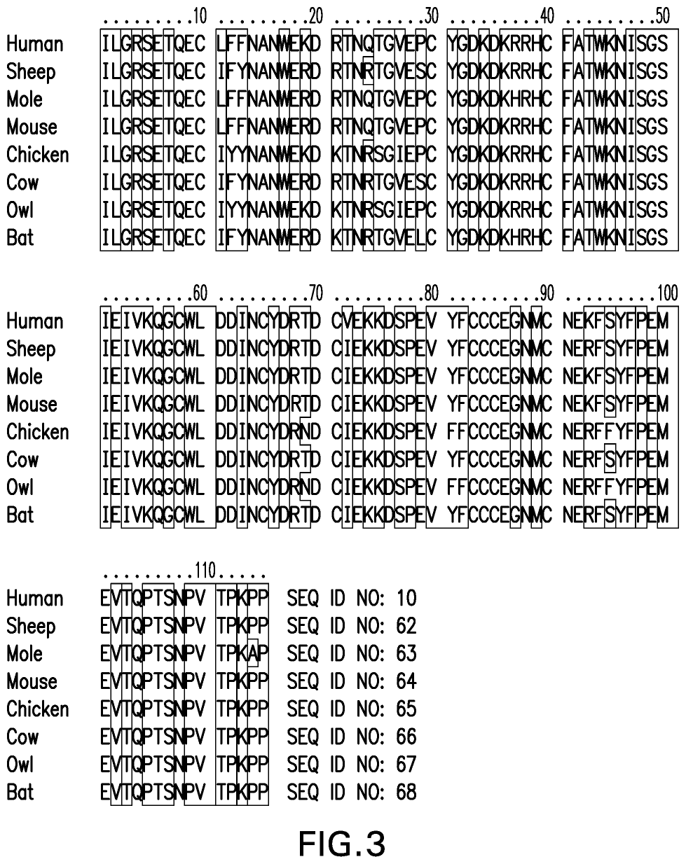

ActRIIA is well-conserved among vertebrates, with large stretches of the extracellular domain completely conserved. For example, FIG. 3 depicts a multi-sequence alignment of a human ActRIIA extracellular domain compared to various ActRIIA orthologs. Many of the ligands that bind to ActRIIA are also highly conserved. Accordingly, from these alignments, it is possible to predict key amino acid positions within the ligand-binding domain that are important for normal ActRIIA-ligand binding activities as well as to predict amino acid positions that are likely to be tolerant to substitution without significantly altering normal ActRIIA-ligand binding activities. Therefore, an active, human ActRIIA variant polypeptide useful in accordance with the presently disclosed methods may include one or more amino acids at corresponding positions from the sequence of another vertebrate ActRIIA, or may include a residue that is similar to that in the human or other vertebrate sequences.

Without meaning to be limiting, the following examples illustrate this approach to defining an active ActRIIA variant. As illustrated in FIG. 3, F13 in the human extracellular domain (SEQ ID NO: 10) is Y in Ovis aries (SEQ ID NO: 62), Gallus gallus (SEQ ID NO: 65), Bos Taurus (SEQ ID NO: 66), Tyto alba (SEQ ID NO: 67), and Myotis davidii (SEQ ID NO: 68) ActRIIA, indicating that aromatic residues are tolerated at this position, including F, W, and Y. Q24 in the human extracellular domain (SEQ ID NO: 10) is R in Bos Taurus ActRIIA, indicating that charged residues will be tolerated at this position, including D, R, K, H, and E. S95 in the human extracellular domain (SEQ ID NO: 10) is F in Gallus gallus and Tyto alba ActRIIA, indicating that this site may be tolerant of a wide variety of changes, including polar residues, such as E, D, K, R, H, S, T, P, G, Y, and probably hydrophobic residue such as L, I, or F. E52 in the human extracellular domain (SEQ ID NO: 10) is D in Ovis aries ActRIIA, indicating that acidic residues are tolerated at this position, including D and E. P29 in the human extracellular (SEQ ID NO: 10) domain is relatively poorly conserved, appearing as S in Ovis aries ActRIIA and L in Myotis davidii ActRIIA, thus essentially any amino acid should be tolerated at this position.

Moreover, as discussed above, ActRII proteins have been characterized in the art in terms of structural/functional characteristics, particularly with respect to ligand binding [Attisano et al. (1992) Cell 68 (1): 97-108; Greenwald et al. (1999) Nature Structural Biology 6 (1): 18-22; Allendorph et al. (2006) PNAS 103 (20:7643-7648; Thompson et al. (2003) The EMBO Journal 22 (7): 1555-1566; as well as U.S. Pat. Nos. 7,709,605, 7,612,041, and 7,842,663]. In addition to the teachings herein, these references provide ample guidance for how to generate ActRII variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRIIA, as demarcated by the outermost of these conserved cysteines, corresponds to positions 30-110 of SEQ ID NO: 9 (ActRIIA precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRIIA extracellular domains truncations include SEQ ID NOs: 10 and 11.

Accordingly, a general formula for an active portion (e.g., ligand binding) of ActRIIA is a polypeptide that comprises, consists essentially of, or consists of amino acids 30-110 of SEQ ID NO: 9. Therefore ActRIIA polypeptides may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to any one of amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 9. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), and 24-30 (e.g., beginning at any one of amino acids 24, 25, 26, 27, 28, 29, or 30) of SEQ ID NO: 9, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), and 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 9. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 9. Thus, in some embodiments, an ActRIIA polypeptide may comprise, consists essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9. Optionally, ActRIIA polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9, and comprising no more than 1, 2, 5, 10 or 15 conservative amino acid changes in the ligand-binding pocket.

In certain embodiments, the disclosure relates to ActRII antagonists (inhibitors) that comprise an ActRIIA polypeptide, which includes fragments, functional variants, and modified forms thereof as well as uses thereof (e.g., increasing an immune response in a patient in need thereof and treating cancer). Preferably, ActRIIA polypeptides are soluble (e.g., an extracellular domain of ActRIIA). In some embodiments, ActRIIA polypeptides inhibit (e.g., SMAD signaling) of one or more ligands (e.g., GDF11, GDF8, activin (activin A, activin B, activin AB, activin AC, BMP6, BMP7, BMP10, GDF3, GDF8, and/or GDF11). In some embodiments, ActRIIA polypeptides bind to one or more ligands (e.g., activin A, activin B, activin AB, activin AC, BMP6, BMP7, BMP10, GDF3, GDF8, and/or GDF11). In some embodiments, ActRIIA polypeptides of the disclosure demonstrate a decreased binding affinity for BMP9. In some embodiments, ActRIIA polypeptides of the disclosure do not bind BMP9. In some embodiments, ActRIIA polypeptide of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134 or 135) of SEQ ID NO: 9. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 9. In certain embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 9. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 9, 10, 11, 36, 39, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 283, 304, 408, and 409. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 9. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 10. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 11. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 36. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 39. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 139. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 140. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 141. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 142. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 143. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 144. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 145. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 146. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 147. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 148. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 149. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 150. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 151. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 152. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 153. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 154. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 155. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 156. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 157. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 158. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 159. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 160. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 161. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 162. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 163. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 164. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 165. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 166. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 167. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 168. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 169. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 170. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 171. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 172. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 173. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 174. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 175. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 176. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 177. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 178. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 179. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 180. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 181. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 182. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 183. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 184. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 185. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 186. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 187. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 188. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 189. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 190. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 191. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 192. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 193. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 194. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 195. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 196. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 197. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 198. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 199. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 200. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 201. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 202. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 203. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 204. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 205. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 206. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 207. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 208. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 209. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 210. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 211. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 283. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 304. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 408. In some embodiments, ActRIIA polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 409.

In some embodiments, an extracellular ActRIIA variant polypeptide may have a sequence of any one of SEQ ID NOs: 139-210. In some embodiments, an extracellular ActRIIA variant polypeptide has a sequence of any one of SEQ ID NOs: 144-210 (Table 5). In some embodiments, an extracellular ActRIIA variant polypeptide may, for example, comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the sequence of a wild-type extracellular ActRIIA polypeptide (SEQ ID NO: 211).

In some embodiments, polypeptides described herein include an extracellular ActRIIA variant having at least one amino acid substitution relative to the wild-type extracellular ActRIIA having the sequence of SEQ ID NO: 211 or the extracellular ActRIIA having any one of the sequences of SEQ ID NOs: 212-232. Possible amino acid substitutions at 27 different positions may be introduced to an extracellular ActRIIA variant (Table 4). An extracellular ActRIIA variant may have one or more (e.g., 1-27, 1-25, 1-23, 1-21, 1-19, 1-17, 1-15, 1-13, 1-11, 1-9, 1-7, 1-5, 1-3, or 1-2; e.g., 1, 2, 3, 4, 5, 6, 7, 8, 9, 10, 11, 12, 13, 14, 15, 16, 17, 18, 19, 20, 21, 22, 23, 24, 25, 26, or 27) amino acid substitutions relative the sequence of a wild-type extracellular ActRIIA (SEQ ID NO: 211). In some embodiments, an extracellular ActRIIA variant (e.g., an extracellular ActRIIA variant having a sequence of SEQ ID NO: 139) may include amino acid substitutions at all of the 27 positions as listed in Table 4. In some embodiments, an extracellular ActRIIA variant may include amino acid substitutions at a number of positions, e.g., at 2, 4, 6, 8, 10, 12, 14, 16, 18, 20, 22, 24, or 26 out of the 27 positions, as listed in Table 4.

Amino acid substitutions can worsen or improve the activity and/or binding affinity of the ActRIIA variants disclosed herein. In some embodiments, to maintain polypeptide function, it is important that the lysine (K) at position X₁₇ in the sequences shown in Tables 3 and 4 (SEQ ID NOs: 139-210 (e.g., SEQ ID NOs: 144-210)) be retained. Substitutions at that position can lead to a loss of activity. For example, an ActRIIA variant having the sequence GAILGRSETQECLFYNANWELERTNQTGVERCEGEKDKRLHCYATWRNISGSIEIVA KGCWLDDFNCYDRTDCVETEENPQVYFCCCEGNMCNEKFSYFPEMEVTQPTS (SEQ ID NO: 283) has reduced activity in vivo, indicating that the substitution of alanine (A) for lysine (K) at X₁₇ is not tolerated. ActRIIA variants disclosed herein, including variants in Tables 3 and 4 (e.g., SEQ ID NOs: 139-210 (e.g., SEQ ID NOs: 144-210), therefore, may retain amino acid K at the position corresponding to X₁₇ in SEQ ID NO: 139 or SEQ ID NO: 140.

In some embodiments, the ActRIIA variants disclosed herein have reduced or no substantial binding to BMP9. In some embodiments, BMP9 binding is reduced in ActRIIA variants containing the amino acid sequence TEEN at positions X₂₃, X₂₄, X₂₅, and X₂₆, as well as in variants that maintain the amino acid K at position X₂₄ and have the amino acid sequence TKEN at positions X₂₃, X₂₄, X₂₅, and X₂₆. The sequences TEEN and TKEN can be employed interchangeably in the ActRIIA variants (e.g., the variants in Tables 3 and 4, e.g., SEQ ID NOs: 139-210 (e.g., SEQ ID NOs: 144-210)) disclosed herein to provide reduced BMP9 binding.

In some embodiments, the ActRIIA variants disclosed herein may further include a C-terminal extension (e.g., additional amino acids at the C-terminus). The C-terminal extension can add one to six additional amino acids at the C-terminus (e.g., 1, 2, 3, 4, 5, 6 or more additional amino acids) to any of the variant polypeptides shown in Tables 3 and 4 (e.g., SEQ ID NOs: 139-208 (e.g., SEQ ID NOs: 144-208)). One potential C-terminal extension that can be included in the ActRIIA variant polypeptides disclosed herein is amino acid sequence NP. For example, the sequence including the C-terminal extension is SEQ ID NO: 209 (e.g., SEQ ID NO: 207 with a C-terminal extension of NP). Another exemplary C-terminal extension that can be included in the ActRIIA variant polypeptides disclosed herein is amino acid sequence NPVTPK (SEQ ID NO: 288). For example, the sequence including the C-terminal extension is SEQ ID NO: 210 (e.g., SEQ ID NO: 207 with a C-terminal extension of NPVTPK).

TABLE 4
Amino acid substitutions in an extracellular ActRIIA variant
having a sequence of any one of SEQ ID NOs: 139-143

GAILGRSETQECLX₁X₂NANWX₃X₄X₅X₆TNQTGVEX₇CX₈GX₉X₁₀X₁₁X₁₂X₁₃X₁₄HC
X₁₅ATWX₁₆NISGSIEIVX₁₇X₁₈GCX₁₉X₂₀X₂₁DX₂₂NCYDRTDCVEX₂₃X₂₄X₂₅X₂₆
PX₂₇VYFCCCEGNMCNEKFSYFPEMEVTQPTS (SEQ ID NO: 139)
GAILGRSETQECLFX₂NANWX₃X₄X₅X₆TNQTGVEX₇CX₈GX₉KX₁₁X₁₂X₁₃X₁₄HCX₁₅
ATWX₁₆NISGSIEIVX₁₇X₁₈GCX₁₉X₂₀X₂₁DX₂₂NCYDRTDCVEX₂₃X₂₄X₂₅X₂₆PX₂₇
VYFCCCEGNMCNEKFSYFPEMEVTQPTS (SEQ ID NO: 140)
GAILGRSETQECLFX₂NANWEX₄X₅RTNQTGVEX₇CX₈GX₉KDKRX₁₄HCX₁₅ATWX₁₆
NISGSIEIVKX₁₈GCWLDDX₂₂NCYDRTDCVEX₂₃X₂₄X₂₅X₂₆PX₂₇VYFCCCEGNMC
NEKFSYFPEMEVTQPTS (SEQ ID NO: 141)
GAILGRSETQECLFX₂NANWEX₄DRTNQTGVEX₈CX₉GX₉KDKRX₁₄HCX₁₅ATWX₁₆
NISGSIEIVKX₁₈GCWLDDX₂₂NCYDRTDCVEX₂₃KX₂₅X₂₆PX₂₇VYFCCCEGNMCN
EKFSYFPEMEVTQPTS (SEQ ID NO: 142)
GAILGRSETQECLFX₂NANWEX₄DRTNQTGVEPCX₈GX₉KDKRX₁₄HCFATWKNISG
SIEIVKX₁₈GCWLDDINCYDRTDCVEX₂₃KX₂₅X₂₆PX₂₇VYFCCCEGNMCNEKFSYF
PEMEVTQPTS (SEQ ID NO: 143)

| | |
|---|---|
| X₁ | F, A, V, I, L, M, F, |
| | Y or W |
| X₂ | F, A, V, I, L, M, F, |
| | Y or W |
| X₃ | D, E or A |
| X₄ | K or L |
| X₅ | D, E, or A |
| X₆ | R, H, K, or A |
| X₇ | P or R |
| X₈ | Y or E |
| X₉ | D, E, or A |
| X₁₀ | K or Q |
| X₁₁ | D, E, or A |
| X₁₂ | K, R, H, or A |
| X₁₃ | R, H, K, or A |
| X₁₄ | R or L |
| X₁₅ | F, A, V, I, L, M, F, |
| | Y or W |
| X₁₆ | K, R, H, or A |
| X₁₇ | K, A, Y, F, I, V, L, |
| | M, or W |
| X₁₈ | Q or K |
| X₁₉ | W or A |
| X₂₀ | L or A |
| X₂₁ | D, K, R, H, A, F, G, |
| | M, N, or I |
| X₂₂ | I, F, A, V, L, M, Y |
| | or W |
| X₂₃ | K or T |
| X₂₄ | K, R, H, D, or E |
| X₂₅ | D, E, or A |
| X₂₆ | S, N, T, or Q |
| X₂₇ | E or Q |

In some embodiments, an extracellular ActRIIA variant comprising the sequence of SEQ ID NO: 140 has the following amino acid substitutions: X₃ is E, X₆ is R, X₁₁ is D, X₁₂ is K, X₁₃ is R, X₁₆ is K or R, X₁₇ is K, X₁₉ is W, X₂₀ is L, X₂₁ is D, and X₂₂ is I or F. In some embodiments, an extracellular ActRIIA variant comprising the sequence of SEQ ID NO: 139 or 140 has the following amino acid substitutions: X₁₇ is K. In some embodiments, an extracellular ActRIIA variant comprising the sequence of SEQ ID NOs: 139-141 has the following amino acid substitutions: X₁₇ is K, X₂₃ is T, X₂₄ is E, X₂₅ is E, and X₂₆ is N. In some embodiments, an extracellular ActRIIA variant comprising the sequence of any one of SEQ ID NOs: 139-143 has the following amino acid substitutions: X₁₇ is K, X₂₃ is T, X₂₄ is K, X₂₅ is E, and X₂₆ is N.

In some embodiments, a polypeptide described herein includes an extracellular ActRIIA variant having a sequence of any one of SEQ ID NOs: 144-210 (Table 5).

TABLE 5
Extracellular ActRIIA variant polypeptides having
the sequences of SEQ ID NOs: 144-210

| SEQ ID | |
|---|---|
| NO | Amino Acid Sequence |
| 144 | GAILGRSETQECLFYNANWELDRTNQTGVEPCEGEKDKRLHCFATWKNIS |
| | GSIEIVKKGCWLDDINCYDRTDCVETKENPQVYFCCCEGNMCNEKFSYFPE |
| | MEVTQPTS |
| 145 | GAILGRSETQECLFYNANWELERTNQTGVEPCEGEKDKRLHCFATWKNIS |
| | GSIEIVKKGCWLDDINCYDRTDCVETKENPQVYFCCCEGNMCNEKFSYFPE |
| | MEVTQPTS |
| 146 | GAILGRSETQECLFYNANWELDRTNQTGVERCEGEKDKRLHCFATWKNIS |
| | GSIEIVKKGCWLDDINCYDRTDCVETKENPQVYFCCCEGNMCNEKFSYFPE |
| | MEVTQPTS |
| 147 | GAILGRSETQECLFYNANWELDRTNQTGVEPCEGEKDKRLHCYATWKNIS |
| | GSIEIVKKGCWLDDINCYDRTDCVETKENPQVYFCCCEGNMCNEKFSYFPE |
| | MEVTQPTS |
| 148 | GAILGRSETQECLFYNANWELDRTNQTGVEPCEGEKDKRLHCFATWRNIS |
| | GSIEIVKKGCWLDDINCYDRTDCVETKENPQVYFCCCEGNMCNEKFSYFPE |
| | MEVTQPTS |
| 149 | GAILGRSETQECLFYNANWELDRTNQTGVEPCEGEKDKRLHCFATWKNIS |
| | GSIEIVKKGCWLDDFNCYDRTDCVETKENPQVYFCCCEGNMCNEKFSYFP |
| | EMEVTQPTS |
| 150 | GAILGRSETQECLFYNANWELDRTNQTGVEPCEGEKDKRLHCFATWKNIS |
| | GSIEIVKKGCWLDDINCYDRTDCVETEENPQVYFCCCEGNMCNEKFSYFPE |
| | MEVTQPTS |
| 151 | GAILGRSETQECLFYNANWELERTNQTGVERCEGEKDKRLHCFATWKNIS |
| | GSIEIVKKGCWLDDINCYDRTDCVETKENPQVYFCCCEGNMCNEKFSYFPE |
| | MEVTQPTS |
| 152 | GAILGRSETQECLFYNANWELERTNQTGVEPCEGEKDKRLHCYATWKNIS |
| | GSIEIVKKGCWLDDINCYDRTDCVETKENPQVYFCCCEGNMCNEKFSYFPE |
| | MEVTQPTS |
| 153 | GAILGRSETQECLFYNANWELERTNQTGVEPCEGEKDKRLHCFATWRNIS |
| | GSIEIVKKGCWLDDINCYDRTDCVETKENPQVYFCCCEGNMCNEKFSYFPE |
| | MEVTQPTS |
| 154 | GAILGRSETQECLFYNANWELERTNQTGVEPCEGEKDKRLHCFATWKNIS |
| | GSIEIVKKGCWLDDFNCYDRTDCVETKENPQVYFCCCEGNMCNEKFSYFP |
| | EMEVTQPTS |
| 155 | GAILGRSETQECLFYNANWELERTNQTGVEPCEGEKDKRLHCFATWKNIS |
| | GSIEIVKKGCWLDDINCYDRTDCVETEENPQVYFCCCEGNMCNEKFSYFPE |
| | MEVTQPTS |
| 156 | GAILGRSETQECLFYNANWELDRTNQTGVERCEGEKDKRLHCYATWKNIS |
| | GSIEIVKKGCWLDDINCYDRTDCVETKENPQVYFCCCEGNMCNEKFSYFPE |
| | MEVTQPTS |
| 157 | GAILGRSETQECLFYNANWELDRTNQTGVERCEGEKDKRLHCFATWRNIS |
| | GSIEIVKKGCWLDDINCYDRTDCVETKENPQVYFCCCEGNMCNEKFSYFPE |
| | MEVTQPTS |
| 158 | GAILGRSETQECLFYNANWELDRTNQTGVERCEGEKDKRLHCFATWKNIS |
| | GSIEIVKKGCWLDDFNCYDRTDCVETKENPQVYFCCCEGNMCNEKFSYFP |
| | EMEVTQPTS |
| 159 | GAILGRSETQECLFYNANWELDRTNQTGVERCEGEKDKRLHCFATWKNIS |
| | GSIEIVKKGCWLDDINCYDRTDCVETEENPQVYFCCCEGNMCNEKFSYFPE |
| | MEVTQPTS |
| 160 | GAILGRSETQECLFYNANWELDRTNQTGVEPCEGEKDKRLHCYATWRNIS |
| | GSIEIVKKGCWLDDINCYDRTDCVETKENPQVYFCCCEGNMCNEKFSYFPE |
| | MEVTQPTS |
| 161 | GAILGRSETQECLFYNANWELDRTNQTGVEPCEGEKDKRLHCYATWKNIS |
| | GSIEIVKKGCWLDDFNCYDRTDCVETKENPQVYFCCCEGNMCNEKFSYFP |
| | EMEVTQPTS |
| 162 | GAILGRSETQECLFYNANWELDRTNQTGVEPCEGEKDKRLHCYATWKNIS |
| | GSIEIVKKGCWLDDINCYDRTDCVETEENPQVYFCCCEGNMCNEKFSYFPE |
| | MEVTQPTS |
| 163 | GAILGRSETQECLFYNANWELDRTNQTGVEPCEGEKDKRLHCFATWRNIS |
| | GSIEIVKKGCWLDDFNCYDRTDCVETKENPQVYFCCCEGNMCNEKFSYFP |
| | EMEVTQPTS |
| 164 | GAILGRSETQECLFYNANWELDRTNQTGVEPCEGEKDKRLHCFATWRNIS |
| | GSIEIVKKGCWLDDINCYDRTDCVETEENPQVYFCCCEGNMCNEKFSYFPE |
| | MEVTQPTS |
| 165 | GAILGRSETQECLFYNANWELDRTNQTGVEPCEGEKDKRLHCFATWKNIS |
| | GSIEIVKKGCWLDDFNCYDRTDCVETEENPQVYFCCCEGNMCNEKFSYFPE |
| | MEVTQPTS |
| 166 | GAILGRSETQECLFYNANWELERTNQTGVERCEGEKDKRLHCYATWKNIS |
| | GSIEIVKKGCWLDDINCYDRTDCVETKENPQVYFCCCEGNMCNEKFSYFPE |
| | MEVTQPTS |
| 167 | GAILGRSETQECLFYNANWELERTNQTGVERCEGEKDKRLHCFATWRNIS |
| | GSIEIVKKGCWLDDINCYDRTDCVETKENPQVYFCCCEGNMCNEKFSYFPE |
| | MEVTQPTS |
| 168 | GAILGRSETQECLFYNANWELERTNQTGVERCEGEKDKRLHCFATWKNIS |
| | GSIEIVKKGCWLDDFNCYDRTDCVETKENPQVYFCCCEGNMCNEKFSYFP |
| | EMEVTQPTS |
| 169 | GAILGRSETQECLFYNANWELERTNQTGVERCEGEKDKRLHCFATWKNIS |
| | GSIEIVKKGCWLDDINCYDRTDCVETEENPQVYFCCCEGNMCNEKFSYFPE |
| | MEVTQPTS |
| 170 | GAILGRSETQECLFYNANWELERTNQTGVEPCEGEKDKRLHCYATWRNIS |
| | GSIEIVKKGCWLDDINCYDRTDCVETKENPQVYFCCCEGNMCNEKFSYFPE |
| | MEVTQPTS |
| 171 | GAILGRSETQECLFYNANWELERTNQTGVEPCEGEKDKRLHCYATWKNIS |
| | GSIEIVKKGCWLDDFNCYDRTDCVETKENPQVYFCCCEGNMCNEKFSYFP |
| | EMEVTQPTS |
| 172 | GAILGRSETQECLFYNANWELERTNQTGVEPCEGEKDKRLHCYATWKNIS |
| | GSIEIVKKGCWLDDINCYDRTDCVETEENPQVYFCCCEGNMCNEKFSYFPE |
| | MEVTQPTS |
| 173 | GAILGRSETQECLFYNANWELERTNQTGVEPCEGEKDKRLHCFATWRNIS |
| | GSIEIVKKGCWLDDFNCYDRTDCVETKENPQVYFCCCEGNMCNEKFSYFP |
| | EMEVTQPTS |
| 174 | GAILGRSETQECLFYNANWELERTNQTGVEPCEGEKDKRLHCFATWRNIS |
| | GSIEIVKKGCWLDDINCYDRTDCVETEENPQVYFCCCEGNMCNEKFSYFPE |
| | MEVTQPTS |
| 175 | GAILGRSETQECLFYNANWELERTNQTGVEPCEGEKDKRLHCFATWKNIS |
| | GSIEIVKKGCWLDDFNCYDRTDCVETEENPQVYFCCCEGNMCNEKFSYFPE |
| | MEVTQPTS |
| 176 | GAILGRSETQECLFYNANWELDRTNQTGVERCEGEKDKRLHCYATWRNIS |
| | GSIEIVKKGCWLDDINCYDRTDCVETKENPQVYFCCCEGNMCNEKFSYFPE |
| | MEVTQPTS |
| 177 | GAILGRSETQECLFYNANWELDRTNQTGVERCEGEKDKRLHCYATWKNIS |
| | GSIEIVKKGCWLDDFNCYDRTDCVETKENPQVYFCCCEGNMCNEKFSYFP |
| | EMEVTQPTS |
| 178 | GAILGRSETQECLFYNANWELDRTNQTGVERCEGEKDKRLHCYATWKNIS |
| | GSIEIVKKGCWLDDINCYDRTDCVETEENPQVYFCCCEGNMCNEKFSYFPE |
| | MEVTQPTS |
| 179 | GAILGRSETQECLFYNANWELDRTNQTGVERCEGEKDKRLHCFATWRNIS |
| | GSIEIVKKGCWLDDFNCYDRTDCVETKENPQVYFCCCEGNMCNEKFSYFP |
| | EMEVTQPTS |
| 180 | GAILGRSETQECLFYNANWELDRTNQTGVERCEGEKDKRLHCFATWRNIS |
| | GSIEIVKKGCWLDDINCYDRTDCVETEENPQVYFCCCEGNMCNEKFSYFPE |
| | MEVTQPTS |
| 181 | GAILGRSETQECLFYNANWELDRTNQTGVERCEGEKDKRLHCFATWKNIS |
| | GSIEIVKKGCWLDDENCYDRTDCVETEENPQVYFCCCEGNMCNEKFSYFPE |
| | MEVTQPTS |
| 182 | GAILGRSETQECLFYNANWELDRTNQTGVEPCEGEKDKRLHCYATWRNIS |
| | GSIEIVKKGCWLDDFNCYDRTDCVETKENPQVYFCCCEGNMCNEKFSYFP |
| | EMEVTQPTS |
| 183 | GAILGRSETQECLFYNANWELDRTNQTGVEPCEGEKDKRLHCYATWRNIS |
| | GSIEIVKKGCWLDDINCYDRTDCVETEENPQVYFCCCEGNMCNEKFSYFPE |
| | MEVTQPTS |
| 184 | GAILGRSETQECLFYNANWELDRTNQTGVEPCEGEKDKRLHCYATWKNIS |
| | GSIEIVKKGCWLDDFNCYDRTDCVETEENPQVYFCCCEGNMCNEKFSYFPE |
| | MEVTQPTS |
| 185 | GAILGRSETQECLFYNANWELDRTNQTGVEPCEGEKDKRLHCFATWRNIS |
| | GSIEIVKKGCWLDDFNCYDRTDCVETEENPQVYFCCCEGNMCNEKFSYFPE |
| | MEVTQPTS |
| 186 | GAILGRSETQECLFYNANWELERTNQTGVERCEGEKDKRLHCYATWRNIS |
| | GSIEIVKKGCWLDDINCYDRTDCVETKENPQVYFCCCEGNMCNEKFSYFPE |
| | MEVTQPTS |
| 187 | GAILGRSETQECLFYNANWELERTNQTGVERCEGEKDKRLHCYATWKNIS |
| | GSIEIVKKGCWLDDFNCYDRTDCVETKENPQVYFCCCEGNMCNEKFSYFP |
| | EMEVTQPTS |
| 188 | GAILGRSETQECLFYNANWELERTNQTGVERCEGEKDKRLHCYATWKNIS |
| | GSIEIVKKGCWLDDINCYDRTDCVETEENPQVYFCCCEGNMCNEKFSYFPE |
| | MEVTQPTS |
| 189 | GAILGRSETQECLFYNANWELERTNQTGVERCEGEKDKRLHCFATWRNIS |
| | GSIEIVKKGCWLDDFNCYDRTDCVETKENPQVYFCCCEGNMCNEKFSYFP |
| | EMEVTQPTS |
| 190 | GAILGRSETQECLFYNANWELERTNQTGVERCEGEKDKRLHCFATWRNIS |
| | GSIEIVKKGCWLDDINCYDRTDCVETEENPQVYFCCCEGNMCNEKFSYFPE |
| | MEVTQPTS |
| 191 | GAILGRSETQECLFYNANWELERTNQTGVERCEGEKDKRLHCFATWKNIS |
| | GSIEIVKKGCWLDDFNCYDRTDCVETEENPQVYFCCCEGNMCNEKFSYFPE |
| | MEVTQPTS |
| 192 | GAILGRSETQECLFYNANWELERTNQTGVEPCEGEKDKRLHCYATWRNIS |
| | GSIEIVKKGCWLDDFNCYDRTDCVETKENPQVYFCCCEGNMCNEKFSYFP |
| | EMEVTQPTS |
| 193 | GAILGRSETQECLFYNANWELERTNQTGVEPCEGEKDKRLHCYATWRNIS |
| | GSIEIVKKGCWLDDINCYDRTDCVETEENPQVYFCCCEGNMCNEKFSYFPE |
| | MEVTQPTS |
| 194 | GAILGRSETQECLFYNANWELERTNQTGVEPCEGEKDKRLHCYATWKNIS |
| | GSIEIVKKGCWLDDFNCYDRTDCVETEENPQVYFCCCEGNMCNEKFSYFPE |
| | MEVTQPTS |
| 195 | GAILGRSETQECLFYNANWELERTNQTGVEPCEGEKDKRLHCFATWRNIS |
| | GSIEIVKKGCWLDDFNCYDRTDCVETEENPQVYFCCCEGNMCNEKFSYFPE |
| | MEVTQPTS |
| 196 | GAILGRSETQECLFYNANWELDRTNQTGVERCEGEKDKRLHCYATWRNIS |
| | GSIEIVKKGCWLDDFNCYDRTDCVETKENPQVYFCCCEGNMCNEKFSYFP |
| | EMEVTQPTS |
| 197 | GAILGRSETQECLFYNANWELDRTNQTGVERCEGEKDKRLHCYATWRNIS |
| | GSIEIVKKGCWLDDINCYDRTDCVETEENPQVYFCCCEGNMCNEKFSYFPE |
| | MEVTQPTS |
| 198 | GAILGRSETQECLFYNANWELDRTNQTGVERCEGEKDKRLHCYATWKNIS |
| | GSIEIVKKGCWLDDFNCYDRTDCVETEENPQVYFCCCEGNMCNEKFSYFPE |
| | MEVTQPTS |
| 199 | GAILGRSETQECLFYNANWELDRTNQTGVERCEGEKDKRLHCFATWRNIS |
| | GSIEIVKKGCWLDDFNCYDRTDCVETEENPQVYFCCCEGNMCNEKFSYFPE |
| | MEVTQPTS |
| 200 | GAILGRSETQECLFYNANWELDRTNQTGVEPCEGEKDKRLHCYATWRNIS |
| | GSIEIVKKGCWLDDFNCYDRTDCVETEENPQVYFCCCEGNMCNEKFSYFPE |
| | MEVTQPTS |
| 201 | GAILGRSETQECLFYNANWELERTNQTGVERCEGEKDKRLHCYATWRNIS |
| | GSIEIVKKGCWLDDFNCYDRTDCVETKENPQVYFCCCEGNMCNEKFSYFP |
| | EMEVTQPTS |
| 202 | GAILGRSETQECLFYNANWELERTNQTGVERCEGEKDKRLHCYATWRNIS |
| | GSIEIVKKGCWLDDINCYDRTDCVETEENPQVYFCCCEGNMCNEKFSYFPE |
| | MEVTQPTS |
| 203 | GAILGRSETQECLFYNANWELERTNQTGVERCEGEKDKRLHCYATWKNIS |
| | GSIEIVKKGCWLDDFNCYDRTDCVETEENPQVYFCCCEGNMCNEKFSYFPE |
| | MEVTQPTS |
| 204 | GAILGRSETQECLFYNANWELERTNQTGVERCEGEKDKRLHCFATWRNIS |
| | GSIEIVKKGCWLDDFNCYDRTDCVETEENPQVYFCCCEGNMCNEKFSYFPE |
| | MEVTQPTS |
| 205 | GAILGRSETQECLFYNANWELDRTNQTGVERCEGEKDKRLHCYATWRNIS |
| | GSIEIVKKGCWLDDFNCYDRTDCVETEENPQVYFCCCEGNMCNEKFSYFPE |
| | MEVTQPTS |
| 206 | GAILGRSETQECLFYNANWELERTNQTGVEPCEGEKDKRLHCYATWRNIS |
| | GSIEIVKKGCWLDDFNCYDRTDCVETEENPQVYFCCCEGNMCNEKFSYFPE |
| | MEVTQPTS |
| 207 | GAILGRSETQECLFYNANWELERTNQTGVERCEGEKDKRLHCYATWRNIS |
| | GSIEIVKKGCWLDDFNCYDRTDCVETEENPQVYFCCCEGNMCNEKFSYFPE |
| | MEVTQPTS |
| 208 | GAILGRSETQECLYYNANWELERTNQTGVERCEGEQDKRLHCYATWRNIS |
| | GSIEIVKKGCWLDDFNCYDRTDCVETEENPQVYFCCCEGNMCNEKFSYFPE |
| | MEVTQPTS |
| 209 | GAILGRSETQECLFYNANWELERTNQTGVERCEGEKDKRLHCYATWRNIS |
| | GSIEIVKKGCWLDDFNCYDRTDCVETEENPQVYFCCCEGNMCNEKFSYFPE |
| | MEVTQPTSNP |
| 210 | GAILGRSETQECLFYNANWELERTNQTGVERCEGEKDKRLHCYATWRNIS |
| | GSIEIVKKGCWLDDFNCYDRTDCVETEENPQVYFCCCEGNMCNEKFSYFPE |
| | MEVTQPTSNPVTPK |

In some embodiments, a polypeptide disclosed herein comprises an extracellular ActRIIA variant polypeptide (e.g., any one of SEQ ID NOs: 139-210 (e.g., SEQ ID NOs: 144-210)) having an amino acid K at the position corresponding to X₁₇ in SEQ ID NO: 139 or SEQ ID NO: 140. In some embodiments, altering the amino acid at position X₁₇ can result in reduced activity. For example, an ActRIIA variant having the sequence GAILGRSETQECLFYNANWELERTNQTGVERCEGEKDKRLHCYATWRNISGSIEIVAKGC WLDDFNCYDRTDCVETEENPQVYFCCCEGNMCNEKFSYFPEMEVTQPTS

(SEQ ID NO: 283) has reduced activity in vivo, indicating that the substitution of A for K at X₁₇ is not tolerated.

In some embodiments, a polypeptide disclosed herein including an extracellular ActRIIA variant (e.g., any one of SEQ ID NOs: 139-210 (e.g., SEQ ID NOs: 144-210)) with the sequence TEEN at positions X₂₃, X₂₄, X₂₅, and X₂₆ can have a substitution of the amino acid K for the amino acid E at position X₂₄. In some embodiments, a polypeptide disclosed herein including an extracellular ActRIIA variant (e.g., any one of SEQ ID NOs: 139-210 (e.g., SEQ ID NOs: 144-210)) with the sequence TKEN at positions X₂₃, X₂₄, X₂₅, and X₂₆ can have a substitution of the amino acid E for the amino acid K at position X₂₄. In some embodiments, polypeptides having the sequence TEEN or TKEN at positions X₂₃, X₂₄, X₂₅, and X₂₆ have reduced binding to BMP9.

In some embodiments, a polypeptide disclosed herein including an extracellular ActRIIA variant (e.g., any one of SEQ ID NOs: 139-208 (e.g., SEQ ID NOs: 144-208)) may further include a C-terminal extension (e.g., additional amino acids at the C-terminus). In some embodiments, the C-terminal extension is amino acid sequence NP. For example, the sequence including the C-terminal extension is SEQ ID NO: 209 (e.g., SEQ ID NO: 207 with a C-terminal extension of NP). In some embodiments, the C-terminal extension is amino acid sequence NPVTPK (SEQ ID NO: 288). For example, the sequence including the C-terminal extension is SEQ ID NO: 210 (e.g., SEQ ID NO: 207 with a C-terminal extension of NPVTPK). The C-terminal extension can add one to six additional amino acids at the C-terminus (e.g., 1, 2, 3, 4, 5, 6 or more additional amino acids).

In some embodiments, Compositions that can be administered to a subject according to the methods described herein are provided in Table 6, below.

TABLE 6
Compositions that can be administered to a subject according to the methods
described herein.

| Row | Composition |
|---|---|
| 1 | A polypeptide comprising an extracellular activin receptor type IIa (ActRIIA) variant, |
| | the variant having a sequence of |
| | GAILGRSETQECLX₁X₂NANWX₃X₄X₅X₆TNQTGVEX₇CX₈GX₉X₁₀X₁₁X₁₂X₁₃X₁₄HC |
| | X₁₅ATWX₁₆NISGSIEIVX₁₇X₁₈GCX₁₉X₂₀X₂₁DX₂₂NCYDRTDCVEX₂₃X₂₄X₂₅X₂₆PX₂₇V |
| | YFCCCEGNMCNEKFSYFPEMEVTQPTS (SEQ ID NO: 139), wherein X₁ is F or Y; |
| | X₂ is For Y; X₃ is E or A; X₄ is K or L; X₅ is D or E; X₆ is R or A; X₇ is P or R; X₈ is Y |
| | or E; X₉ is D or E; X₁₀ is K or Q; X₁₁ is D or A; X₁₂ is K or A; X₁₃ is R or A; X₁₄ is R or |
| | L; X₁₅ is For Y; X₁₆ is K, R, or A; X₁₇ is K, A, Y, F, or I; X₁₈ is Q or K; X₁₉ is W or A; |
| | X₂₀ is L or A; X₂₁ is D, K, R, A, F, G, M, N, or I; X₂₂ is I, F, or A; X₂₃ is K or T; X₂₄ is |
| | K or E; X₂₅ is D or E; X₂₆ is S or N; and X₂₇ is E or Q, and wherein the variant has at |
| | least one amino acid substitution relative to a wild-type extracellular ActRIIA having |
| | the sequence of SEQ ID NO: 211 or an extracellular ActRIIA having any one of the |
| | sequences of SEQ ID NOs: 212-232. |
| 2 | A polypeptide comprising an extracellular activin receptor type IIa (ActRIIA) variant, |
| | the variant having a sequence of |
| | GAILGRSETQECLX₁X₂NANWX₃X₄X₅X₆TNQTGVEX₇CX₈GX₉X₁₀X₁₁X₁₂X₁₃X₁₄HC |
| | X₁₅ATWX₁₆NISGSIEIVX₁₇X₁₈GCX₁₉X₂₀X₂₁DX₂₂NCYDRTDCVEX₂₃X₂₄X₂₅X₂₆PX₂₇V |
| | YFCCCEGNMCNEKFSYFPEMEVTQPTS (SEQ ID NO: 139), wherein X₁ is F, A, V, |
| | I, L, M, F, Y or W; X₂ is F, A, V, I, L, M, F, Y or W; X₃ is D, E or A; X₄ is K or L; X₅ |
| | is D, E, or A; X₆ is R, H, K, or A; X₇ is Por R; X₈ is Y or E; X₉ is D, E, or A; X₁₀ is K |
| | or Q; X₁₁ is D, E, or A; X₁₂ is K, R, H, or A; X₁₃ is R, H, K, or A; X₁₄ is R or L; X₁₅ is |
| | F, A, V, I, L, M, F, Y or W; X₁₆ is K, R, H, or A; X₁₇ is K, A, Y, F, I, V, L, M, or W; |
| | X₁₈ is Q or K; X₁₉ is W or A; X₂₀ is L or A; X₂₁ is D, K, R, H, A, F, G, M, N, or I; X₂₂ is |
| | I, F, A, V, L, M, Y or W; X₂₃ is K or T; X₂₄ is K, R, H, D, or E; X₂₅ is D, E, or A; X₂₆ is |
| | S, N, T, or Q; and X₂₇ is E or Q, and wherein the variant has at least one amino acid |
| | substitution relative to a wild-type extracellular ActRIIA having the sequence of SEQ |
| | ID NO: 211 or an extracellular ActRIIA having any one of the sequences of SEQ ID |
| | NOs: 212-232. |
| 3 | The polypeptide of row 1 or 2, wherein the variant has a sequence of |
| | GAILGRSETQECLFX₂NANWX₃X₄X₅X₆TNQTGVEX₇CX₈GX₉KX₁₁X₁₂X₁₃X₁₄HCX₁₅ |
| | ATWX₁₆NISGSIEIVX₁₇X₁₈GCX₁₉X₂₀X₂₁DX₂₂NCYDRTDCVEX₂₃X₂₄X₂₅X₂₆PX₂₇VYF |
| | CCCEGNMCNEKFSYFPEMEVTQPTS (SEQ ID NO: 140). |
| 4 | The polypeptide of rows 1-3, wherein the variant has a sequence of |
| | GAILGRSETQECLFX₂NANWEX₄X₅RTNQTGVEX₇CX₈GX₉KDKRX₁₄HCX₁₅ATW |
| | X₁₆NISGSIEIVKX₁₈GCWLDDX₂₂NCYDRTDCVEX₂₃X₂₄X₂₅X₂₆PX₂₇VYFCCCEGNM |
| | CNEKFSYFPEMEVTQPTS (SEQ ID NO: 141). |
| 5 | The polypeptide of any one of rows 1-4, wherein the variant has a sequence of |
| | GAILGRSETQECLFX₂NANWEX₄DRTNQTGVEX₇CX₈GX₉KDKRX₁₄HCX₁₅ATWX |
| | 16NISGSIEIVKX₁₈GCWLDDX₂₂NCYDRTDCVEX₂₃KX₂₅X₂₆PX₂₇VYFCCCEGNMCN |
| | EKFSYFPEMEVTQPTS (SEQ ID NO: 142). |
| 6 | The polypeptide of any one of rows 1-5, wherein the variant has a sequence of |
| | GAILGRSETQECLFX₂NANWEX₄DRTNQTGVEPCX₈GX₉KDKRX₁₄HCFATWKNI |
| | SGSIEIVKX₁₈GCWLDDINCYDRTDCVEX₂₃KX₂₅X₂₆PX₂₇VYFCCCEGNMCNEKFS |
| | YFPEMEVTQPTS (SEQ ID NO: 143). |
| 7 | The polypeptide of row 1 or 2, wherein X₁ is F. |
| 8 | The polypeptide of row 1 or 2, wherein X₁ is Y. |
| 9 | The polypeptide of row 1, 2, 7, or 8 wherein X₁₀ is K. |
| 10 | The polypeptide of row 1, 2, 7, or 8 wherein X₁₀ is Q. |
| 11 | The polypeptide of any one of rows 1-10, wherein X₂ is F. |
| 12 | The polypeptide of any one of rows 1-10, wherein X₂ is Y. |
| 13 | The polypeptide of any one of rows 1, 2, 3, and 7-12, wherein X₃ is E. |
| 14 | The polypeptide of any one of rows 1, 2, 3, and 7-12, wherein X₃ is A. |
| 15 | The polypeptide of any one of rows 1-14, wherein X₄ is K. |
| 16 | The polypeptide of any one of rows 1-14, wherein X₄ is L. |
| 17 | The polypeptide of any one of rows 1, 2, 3, 4, and 7-16, wherein X₅ is D. |
| 18 | The polypeptide of any one of rows 1, 2, 3, 4, and 7-16, wherein X₅ is E. |
| 19 | The polypeptide of any one of rows 1, 2, 3, and 7-18, wherein X₆ is R. |
| 20 | The polypeptide of any one of rows 1, 2, 3, and 7-18, wherein X₆ is A. |
| 21 | The polypeptide of any one of rows 1-5 and 7-20, wherein X₇ is P. |
| 22 | The polypeptide of any one of rows 1-5 and 7-20, wherein X₇ is R. |
| 23 | The polypeptide of any one of rows 1-22, wherein X₈ is Y. |
| 24 | The polypeptide of any one of rows 1-22, wherein X₈ is E. |
| 25 | The polypeptide of any one of rows 1-24, wherein X₉ is D. |
| 26 | The polypeptide of any one of rows 1-24, wherein X₉ is E |
| 27 | The polypeptide of any one of rows 1, 2, 3, and 7-26, wherein X₁₁ is D. |
| 28 | The polypeptide of any one of rows 1, 2, 3, and 7-26, wherein X₁₁ is A. |
| 29 | The polypeptide of any one of rows 1, 2, 3, and 7-28, wherein X₁₂ is K. |
| 30 | The polypeptide of any one of rows 1, 2, 3, and 7-28, wherein X₁₂ is A. |
| 31 | The polypeptide of any one of rows 1, 2, 3, and 7-30, wherein X₁₃ is R. |
| 32 | The polypeptide of any one of rows 1, 2, 3, and 7-30, wherein X₁₃ is A. |
| 33 | The polypeptide of any one of rows 1-32, wherein X₁₄ is R. |
| 34 | The polypeptide of any one of rows 1-32, wherein X₁₄ is L. |
| 35 | The polypeptide of any one of rows 1-5 and 7-34, wherein X₁₅ is F. |
| 36 | The polypeptide of any one of rows 1-5 and 7-34, wherein X₁₅ is Y. |
| 37 | The polypeptide of any one of rows 1-5 and 7-36, wherein X₁₆ is K. |
| 38 | The polypeptide of any one of rows 1-5 and 7-36, wherein X₁₆ is R. |
| 39 | The polypeptide of any one of rows 1-5 and 7-36, wherein X₁₆ is A. |
| 40 | The polypeptide of any one of rows 1, 2, 3, and 7-39, wherein X₁₇ is K. |
| 41 | The polypeptide of any one of rows 1, 2, 3, and 7-39, wherein X₁₇ is A. |
| 42 | The polypeptide of any one of rows 1, 2, 3, and 7-39, wherein X₁₇ is Y. |
| 43 | The polypeptide of any one of rows 1, 2, 3, and 7-39, wherein X₁₇ is F. |
| 44 | The polypeptide of any one of rows 1, 2, 3, and 7-39, wherein X₁₇ is I. |
| 45 | The polypeptide of any one of rows 1-44, wherein X₁₈ is Q. |
| 46 | The polypeptide of any one of rows 1-44, wherein X₁₈ is K. |
| 47 | The polypeptide of any one of rows 1-3 and 7-46, wherein X₁₉ is W. |
| 48 | The polypeptide of any one of rows 1-3 and 7-46, wherein X₁₉ is A. |
| 49 | The polypeptide of any one of rows 1-3 and 7-48, wherein X₂₀ is L. |
| 50 | The polypeptide of any one of rows 1-3 and 7-48, wherein X₂₀ is A. |
| 51 | The polypeptide of any one of rows 1-3 and 7-50, wherein X₂₁ is D. |
| 52 | The polypeptide of any one of rows 1-3 and 7-50, wherein X₂₁ is K. |
| 53 | The polypeptide of any one of rows 1-3 and 7-50, wherein X₂₁ is R. |
| 54 | The polypeptide of any one of rows 1-3 and 7-50, wherein X₂₁ is A. |
| 55 | The polypeptide of any one of rows 1-3 and 7-50, wherein X₂₁ is F. |
| 56 | The polypeptide of any one of rows 1-3 and 7-50, wherein X₂₁ is G. |
| 57 | The polypeptide of any one of rows 1-3 and 7-50, wherein X₂₁ is M. |
| 58 | The polypeptide of any one of rows 1-3 and 7-50, wherein X₂₁ is N. |
| 59 | The polypeptide of any one of rows 1-3 and 7-50, wherein X₂₁ is I. |
| 60 | The polypeptide of any one of rows 1-5 and 7-59, wherein X₂₂ is I. |
| 61 | The polypeptide of any one of rows 1-5 and 7-59, wherein X₂₂ is F. |
| 62 | The polypeptide of any one of rows 1-5 and 7-59, wherein X₂₂ is A. |
| 63 | The polypeptide of any one of rows 1-62, wherein X₂₃ is K. |
| 64 | The polypeptide of any one of rows 1-62, wherein X₂₃ is T. |
| 65 | The polypeptide of any one of rows 1-4 and 7-64, wherein X₂₄ is K. |
| 66 | The polypeptide of any one of rows 1-4 and 7-64, wherein X₂₄ is E. |
| 67 | The polypeptide of any one of rows 1-66, wherein X₂₅ is D. |
| 68 | The polypeptide of any one of rows 1-66, wherein X₂₅ is E. |
| 69 | The polypeptide of any one of rows 1-68, wherein X₂₆ is S. |
| 70 | The polypeptide of any one of rows 1-68, wherein X₂₆ is N. |
| 71 | The polypeptide of any one of rows 1-70, wherein X₂₇ is E. |
| 72 | The polypeptide of any one of rows 1-70, wherein X₂₇ is Q. |
| 73 | The polypeptide of any one of rows 1-72, wherein X₂₃ is T, X₂₄ is E, X₂₅ is E, and X₂₆ is |
| | N. |
| 74 | The polypeptide of any one of rows 1-72, wherein X₂₃ is T, X₂₄ is E, X₂₅ is E, and X₂₆ is |
| | N. |
| 75 | The polypeptide of any one of rows 1-74, wherein X₁₇ is K. |
| 76 | The polypeptide of row 1 or 2, wherein the variant has the sequence of any one of SEQ |
| | ID NOs: 144-210. |
| 77 | The polypeptide of row 76, wherein the variant has the sequence of SEQ ID NO: 207. |
| 78 | The polypeptide of row 76, wherein the variant has the sequence of SEQ ID NO: 196. |
| 79 | The polypeptide of row 76, wherein the variant has the sequence of SEQ ID NO: 144. |
| 80 | The polypeptide of row 76, wherein the variant has the sequence of SEQ ID NO: 176. |
| 81 | The polypeptide of row 76, wherein the variant has the sequence of SEQ ID NO: 179. |
| 82 | The polypeptide of row 76, wherein the variant has the sequence of SEQ ID NO: 182. |
| 83 | The polypeptide of row 76, wherein the variant has the sequence of SEQ ID NO: 208. |
| 84 | The polypeptide of row 76, wherein the variant has the sequence of SEQ ID NO: 209. |
| 85 | The polypeptide of row 76, wherein the variant has the sequence of SEQ ID NO: 210. |
| 86 | The polypeptide of any one of rows 1-85, wherein the amino acid at position X₂₄ is |
| | replaced with the amino acid K. |
| 87 | The polypeptide of any one of rows 1-86, wherein the amino acid at position X₂₄ is |
| | replaced with the amino acid E. |
| 88 | The polypeptide of any one of rows 1-87, further comprising a C-terminal extension of |
| | one or more amino acids. |
| 89 | The polypeptide of row 88, wherein the C-terminal extension is NP. |
| 90 | The polypeptide of row 88, wherein the C-terminal extension is NPVTPK. |
| 91 | The polypeptide of any one of rows 1-90, further comprising an Fc domain monomer |
| | fused to the C-terminus of the polypeptide by way of a linker. |
| 92 | The polypeptide of row 91, wherein the Fc domain monomer comprises the sequence of |
| | SEQ ID NO: 233. |
| 93 | The polypeptide of any one of rows 1-90, further comprising a wild-type Fc domain |
| | fused to the C-terminus of the polypeptide by way of a linker. |
| 94 | The polypeptide of row 93, wherein the wild-type Fc domain comprises the sequence of |
| | SEQ ID NO: 284. |
| 95 | The polypeptide of any one of rows 1-90, further comprising an Fc domain with amino |
| | acid substitutions fused to the C-terminus of the polypeptide by way of a linker. |
| 96 | The polypeptide of row 95, wherein the Fc domain does not form a dimer. |
| 97 | The polypeptide of any one of rows 1-90, further comprising an albumin-binding |
| | peptide fused to the C-terminus of the polypeptide by way of a linker. |
| 98 | The polypeptide of row 97, wherein the albumin-binding peptide comprises the |
| | sequence of SEQ ID NO: 285. |
| 99 | The polypeptide of any one of rows 1-90, further comprising a fibronectin domain fused |
| | to the C-terminus of the polypeptide by way of a linker. |
| 100 | The polypeptide of row 99, wherein the fibronectin domain comprises the sequence of |
| | SEQ ID NO: 286. |
| 101 | The polypeptide of any one of rows 1-90, further comprising a human serum albumin |
| | fused to the C-terminus of the polypeptide by way of a linker. |
| 102 | The polypeptide of row 101, wherein the human serum albumin comprises the sequence |
| | of SEQ ID NO: 287. |
| 103 | The polypeptide of row 91 or 92, wherein the polypeptide forms a dimer. |
| 104 | The polypeptide of any one of rows 91-103, wherein the linker is an amino acid spacer. |
| 105 | The polypeptide of row 104, wherein the amino acid spacer is GGG, GGGA (SEQ ID |
| | NO: 234), GGGG (SEQ ID NO: 20), GGGAG (SEQ ID NO: 263), GGGAGG (SEQ ID |
| | NO: 264), or GGGAGGG (SEQ ID NO: 265). |

In some embodiments, an extracellular ActRIIA variant described herein does not have the sequence of any one of SEQ ID NOs: 212-232 shown in Table 7 below.

TABLE 7
Excluded Extracellular ActRIIA Variant polypeptides.

| SEQ ID NO: | Amino Acid Sequence |
|---|---|
| 212 | GAILGRSETQECLFFNANWEKDRTNQTGVEPCYGDKDKRRHCFATWAN |
| | ISGSIEIVKQGCWLDDINCYDRTDCVEKKDSPEVYFCCCEGNMCNEKF |
| | SYFPEMEVTQPTS |
| 213 | GAILGRSETQECLFFNANWAKDRTNQTGVEPCYGDKDKRRHCFATWKN |
| | ISGSIEIVKQGCWLDDINCYDRTDCVEKKDSPEVYFCCCEGNMCNEKF |
| | SYFPEMEVTQPTS |
| 214 | GAILGRSETQECLFFNANWEKDATNQTGVEPCYGDKDKRRHCFATWKN |
| | ISGSIEIVKQGCWLDDINCYDRTDCVEKKDSPEVYFCCCEGNMCNEKF |
| | SYFPEMEVTQPTS |
| 215 | GAILGRSETQECLFFNANWEKDRTNQTGVEPCYGDKAKRRHCFATWKN |
| | ISGSIEIVKQGCWLDDINCYDRTDCVEKKDSPEVYFCCCEGNMCNEKF |
| | SYFPEMEVTQPTS |
| 216 | GAILGRSETQECLFFNANWEKDRTNQTGVEPCYGDKDARRHCFATWKN |
| | ISGSIEIVKQGCWLDDINCYDRTDCVEKKDSPEVYFCCCEGNMCNEKF |
| | SYFPEMEVTQPTS |
| 217 | GAILGRSETQECLFFNANWEKDRTNQTGVEPCYGDKDKARHCFATWKN |
| | ISGSIEIVKQGCWLDDINCYDRTDCVEKKDSPEVYFCCCEGNMCNEKF |
| | SYFPEMEVTQPTS |
| 218 | GAILGRSETQECLFFNANWEKDRTNQTGVEPCYGDKDKRRHCFATWKN |
| | ISGSIEIVAQGCWLDDINCYDRTDCVEKKDSPEVYFCCCEGNMCNEKF |
| | SYFPEMEVTQPTS |
| 219 | GAILGRSETQECLFFNANWEKDRTNQTGVEPCYGDKDKRRHCFATWKN |
| | ISGSIEIVYQGCWLDDINCYDRTDCVEKKDSPEVYFCCCEGNMCNEKF |
| | SYFPEMEVTQPTS |
| 220 | GAILGRSETQECLFFNANWEKDRTNQTGVEPCYGDKDKRRHCFATWKN |
| | ISGSIEIVFQGCWLDDINCYDRTDCVEKKDSPEVYFCCCEGNMCNEKF |
| | SYFPEMEVTQPTS |
| 221 | GAILGRSETQECLFFNANWEKDRTNQTGVEPCYGDKDKRRHCFATWKN |
| | ISGSIEIVIQGCWLDDINCYDRTDCVEKKDSPEVYFCCCEGNMCNEKF |
| | SYFPEMEVTQPTS |
| 222 | GAILGRSETQECLFFNANWEKDRTNQTGVEPCYGDKDKRRHCFATWKN |
| | ISGSIEIVKQGCALDDINCYDRTDCVEKKDSPEVYFCCCEGNMCNEKF |
| | SYFPEMEVTQPTS |
| 223 | GAILGRSETQECLFFNANWEKDRTNQTGVEPCYGDKDKRRHCFATWK |
| | NISGSIEIVKQGCWADDINCYDRTDCVEKKDSPEVYFCCCEGNMCNE |
| | KFSYFPEMEVTQPTS |
| 224 | GAILGRSETQECLFFNANWEKDRTNQTGVEPCYGDKDKRRHCFATWK |
| | NISGSIEIVKQGCWLKDINCYDRTDCVEKKDSPEVYFCCCEGNMCNE |
| | KFSYFPEMEVTQPTS |
| 225 | GAILGRSETQECLFFNANWEKDRTNQTGVEPCYGDKDKRRHCFATWK |
| | NISGSIEIVKQGCWLRDINCYDRTDCVEKKDSPEVYFCCCEGNMCNE |
| | KFSYFPEMEVTQPTS |
| 226 | GAILGRSETQECLFFNANWEKDRTNQTGVEPCYGDKDKRRHCFATWK |
| | NISGSIEIVKQGCWLADINCYDRTDCVEKKDSPEVYFCCCEGNMCNE |
| | KFSYFPEMEVTQPTS |
| 227 | GAILGRSETQECLFFNANWEKDRTNQTGVEPCYGDKDKRRHCFATWK |
| | NISGSIEIVKQGCWLFDINCYDRTDCVEKKDSPEVYFCCCEGNMCNE |
| | KFSYFPEMEVTQPTS |
| 228 | GAILGRSETQECLFFNANWEKDRTNQTGVEPCYGDKDKRRHCFATWK |
| | NISGSIEIVKQGCWLGDINCYDRTDCVEKKDSPEVYFCCCEGNMCNE |
| | KFSYFPEMEVTQPTS |
| 229 | GAILGRSETQECLFFNANWEKDRTNQTGVEPCYGDKDKRRHCFATWK |
| | NISGSIEIVKQGCWLMDINCYDRTDCVEKKDSPEVYFCCCEGNMCNE |
| | KFSYFPEMEVTQPTS |
| 230 | GAILGRSETQECLFFNANWEKDRTNQTGVEPCYGDKDKRRHCFATWK |
| | NISGSIEIVKQGCWLNDINCYDRTDCVEKKDSPEVYFCCCEGNMCNE |
| | KFSYFPEMEVTQPTS |
| 231 | GAILGRSETQECLFFNANWEKDRTNQTGVEPCYGDKDKRRHCFATWK |
| | NISGSIEIVKQGCWLIDINCYDRTDCVEKKDSPEVYFCCCEGNMCNE |
| | KFSYFPEMEVTQPTS |
| 232 | GAILGRSETQECLFFNANWEKDRTNQTGVEPCYGDKDKRRHCFATWK |
| | NISGSIEIVKQGCWLDDANCYDRTDCVEKKDSPEVYFCCCEGNMCNE |
| | KFSYFPEMEVTQPTS |

Furthermore, in some embodiments, a polypeptide described herein has a serum half-life of at least 7 days in humans. In some embodiments, the polypeptide may bind to bone morphogenetic protein 9 (BMP9) with a K_{D} of 200 pM or higher. In some embodiments, the polypeptide may bind to activin A with a K_{D} of 10 pM or higher. In some embodiments, the polypeptide does not bind to BMP9 or activin A. In some embodiments, the polypeptide binds to activin and/or myostatin and exhibits reduced binding to BMP9. In some embodiments, the polypeptide that has reduced binding to BMP9 has the sequence TEEN or TKEN at positions X₂₃, X₂₄, X₂₅, and X₂₆.

Additionally, in some embodiments, the polypeptide may bind to human BMP9 with a K_{D} of about 200 pM or higher (e.g., a K_{D} of about 200, 300, 400, 500, 600, 700, 800, or 900 pM or higher, e.g., a K_{D} of about 1, 2, 3, 4, 5, 6, 7, 8, 9, 10, 20, 30, 40, or 50 nM or higher, e.g., a K_{D} of between about 200 pM and about 50 nM). In some embodiments, the polypeptide does not substantially bind to human BMP9. In some embodiments, the polypeptide may bind to human activin A with a K_{D} of about 800 pM or less (e.g., a K_{D} of about 800, 700, 600, 500, 400, 300, 200,100, 90, 80, 70, 60, 50, 40, 30, 20, 10, 9, 8, 7, 6, 5, 4, 3, 2, or 1 pM or less, e.g., a K_{D} of between about 800 pM and about 200 pM). In some embodiments, the polypeptide may bind to human activin B with a K_{D} of 800 pM or less (e.g., a K_{D} of about 800, 700, 600, 500, 400, 300, 200, 100, 90, 80, 70, 60, 50, 40, 30, 20, 10, 9, 8, 7, 6, 5, 4, 3, 2, or 1 pM or less, e.g., a K_{D} of between about 800 pM and about 200 pM). In some embodiments, the polypeptide may also bind to growth and differentiation factor 11 (GDF-11) with a K_{D} of approximately 5 pM or higher (e.g., a K_{D} of about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 pM or higher).

To illustrate, one or more mutations may be selected that increase the selectivity of the altered ligand-binding domain for GDF11 and/or GDF8 over one or more ActRII-binding ligands such as activins (activin A, activin B, activin AB, activin C, and/or activin E), particularly activin A. Optionally, the altered ligand-binding domain has a ratio of K_{d} for activin binding to K_{d} for GDF11 and/or GDF8 binding that is at least 2-, 5-, 10-, 20-, 50-, 100- or even 1000-fold greater relative to the ratio for the wild-type ligand-binding domain. Optionally, the altered ligand-binding domain has a ratio of IC₅₀ for inhibiting activin to IC₅₀ for inhibiting GDF11 and/or GDF8 that is at least 2-, 5-, 10-, 20-, 50-, 100- or even 1000-fold greater relative to the wild-type ligand-binding domain. Optionally, the altered ligand-binding domain inhibits GDF11 and/or GDF8 with an IC₅₀ at least 2-, 5-, 10-, 20-, 50-, 100- or even 1000-times less than the IC₅₀ for inhibiting activin.

Amino acid residues of the ActRIIB proteins (e.g., E39, K55, Y60, K74, W78, L79, D80, and F101 with respect to SEQ ID NO: 1) are in the ActRIIB ligand-binding pocket and help mediate binding to its ligands including, for example, activin A, GDF11, and GDF8. Thus, the present disclosure provides polypeptides comprising an altered-ligand binding domain (e.g., a GDF8/GDF11-binding domain) of an ActRIIB receptor which comprises one or more mutations at those amino acid residues.

As a specific example, the positively-charged amino acid residue Asp (D80) of the ligand-binding domain of ActRIIB can be mutated to a different amino acid residue to produce a polypeptide that preferentially binds to GDF8, but not activin. In some embodiments, the D80 residue with respect to SEQ ID NO: 1 is changed to an amino acid residue selected from the group consisting of: an uncharged amino acid residue, a negative amino acid residue, and a hydrophobic amino acid residue. As a further specific example, the hydrophobic residue L79 of SEQ ID NO: 1 can be altered to confer altered activin-GDF11/GDF8 binding properties. For example, an L79P substitution reduces GDF11 binding to a greater extent than activin binding. In contrast, replacement of L79 with an acidic amino acid (an aspartic acid or glutamic acid; an L79D or an L79E substitution) greatly reduces activin A binding affinity while retaining GDF11 binding affinity. In exemplary embodiments, the methods described herein utilize a polypeptide which is a variant ActRIIB polypeptide comprising an acidic amino acid (e.g., D or E) at the position corresponding to position 79 of SEQ ID NO: 1, optionally in combination with one or more additional amino acid substitutions, additions, or deletions.

In some embodiments, the present disclosure contemplates making functional variants by modifying the structure of an ActRII polypeptide for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide or to a reference variant polypeptide, or to bind to one or more TGF-beta ligands including, for example, activin A, activin B, activin AB, activin AC, BMP6, BMP7, BMP9, BMP10, GDF3, GDF8, and GDF11.

In certain embodiments, the present disclosure contemplates specific mutations of an ActRII polypeptide so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, polypeptides of the present disclosure for use in humans may be expressed in a mammalian cell line that provides proper glycosylation, such as HEK293 or CHO cell lines, although other mammalian expression cell lines are expected to be useful as well.

The present disclosure further contemplates a method of generating mutants, particularly sets of combinatorial mutants of an ActRII polypeptide as well as truncation mutants. Pools of combinatorial mutants are especially useful for identifying functionally active (e.g., ligand binding) ActRII sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, ActRII variants may be screened for ability to bind to one or more TGF-beta ligands (e.g., activin A, activin B, activin AB, activin AC, BMP6, BMP7, BMP9, BMP10, GDF3, GDF8, and GDF11), to prevent binding of a TGF-beta ligand to an ActRII polypeptide and/or to interfere with signaling caused by a TGF-beta ligand.

The activity of ActRII polypeptides may also be tested in a cell-based or in vivo assay. For example, the effect of an ActRII polypeptide on the expression of genes involved in renal diseases or conditions (e.g., Alport syndrome, focal segmental glomerulosclerosis (FSGS), polycystic kidney diseases, chronic kidney disease) may be assessed. This may, as needed, be performed in the presence of one or more recombinant TGF-beta ligand proteins (e.g., activin A, activin B, activin AB, activin AC, BMP6, BMP7, BMP9, BMP10, GDF3, GDF8, and GDF11), and cells may be transfected so as to produce an ActRII polypeptide and optionally, a TGF-beta family ligand. Likewise, an ActRII polypeptide may be administered to a mouse or other animal and effects on renal diseases or conditions (e.g., Alport syndrome, focal segmental glomerulosclerosis (FSGS), polycystic kidney diseases, chronic kidney disease) may be assessed using art-recognized methods. Similarly, the activity of an ActRII polypeptide or variant thereof may be tested in blood cell precursor cells for any effect on growth of these cells, for example, by the assays as described herein and those of common knowledge in the art. A SMAD-responsive reporter gene may be used in such cell lines to monitor effects on downstream signaling.

Combinatorial-derived variants can be generated which have increased selectivity or generally increased potency relative to a reference ActRII polypeptide. Such variants, when expressed from recombinant DNA constructs, can be used in gene therapy protocols. Likewise, mutagenesis can give rise to variants which have intracellular half-lives dramatically different than the corresponding unmodified ActRII polypeptide. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter the half-life of the ActRII polypeptide.

A combinatorial library may be produced by way of a degenerate library of genes encoding a library of polypeptides which each include at least a portion of potential ActRII polypeptide sequences. For instance, a mixture of synthetic oligonucleotides can be enzymatically ligated into gene sequences such that the degenerate set of potential ActRII encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art [Narang, S A (1983) Tetrahedron 39:3; Itakura et al. (1981) Recombinant DNA, Proc. 3rd Cleveland Sympos. Macromolecules, ed. A G Walton, Amsterdam: Elsevier pp 273-289; Itakura et al. (1984) Annu. Rev. Biochem. 53:323; Itakura et al. (1984) Science 198:1056; and Ike et al. (1983) Nucleic Acid Res. 11:477]. Such techniques have been employed in the directed evolution of other proteins [Scott et al., (1990) Science 249:386-390; Roberts et al. (1992) PNAS USA 89:2429-2433; Devlin et al. (1990) Science 249:404-406; Cwirla et al., (1990) PNAS USA 87:6378-6382; as well as U.S. Pat. Nos. 5,223,409, 5,198,346, and 5,096,815].

Alternatively, other forms of mutagenesis can be utilized to generate a combinatorial library. For example, ActRII polypeptides of the disclosure can be generated and isolated from a library by screening using, for example, alanine scanning mutagenesis [Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [Gustin et al. (1993) Virology 193:653-660; and Brown et al. (1992) Mol. Cell Biol. 12:2644-2652; Mcknight et al. (1982) Science 232:316], by saturation mutagenesis [Meyers et al., (1986) Science 232:613]; by PCR mutagenesis [Leung et al. (1989) Method Cell Mol Biol 1:11-19]; or by random mutagenesis, including chemical mutagenesis [Miller et al. (1992) A Short Course in Bacterial Genetics, CSHL Press, Cold Spring Harbor, NY; and Greener et al. (1994) Strategies in Mol Biol 7:32-34]. Linker scanning mutagenesis, particularly in a combinatorial setting, is an attractive method for identifying truncated (bioactive) forms of ActRII polypeptides.

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of ActRII polypeptides. The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Exemplary assays include TGF-beta ligand (e.g., activin A, activin B, activin AB, activin AC, BMP6, BMP7, BMP9, BMP10, GDF3, GDF8, and GDF11) binding assays and/or ligand-mediated cell signaling assays.

As will be recognized by one of skill in the art, most of the described mutations, variants or modifications described herein may be made at the nucleic acid level or, in some cases, by post-translational modification or chemical synthesis. Such techniques are well known in the art and some of which are described herein. In part, the present disclosure identifies functionally active portions (fragments) and variants of ActRII polypeptides that can be used as guidance for generating and using other variant ActRII polypeptides within the scope described herein.

In certain embodiments, functionally active fragments of ActRII polypeptides of the present disclosure can be obtained by screening polypeptides recombinantly produced from the corresponding fragment of the nucleic acid encoding an ActRII polypeptide. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (inhibitors) of ActRII receptors and/or one or more TGF-beta ligands (e.g., activin A, activin B, activin AB, activin AC, BMP6, BMP7, BMP9, BMP10, GDF3, GDF8, and GDF11).

In certain embodiments, ActRII polypeptides of the present disclosure may further comprise post-translational modifications in addition to any that are naturally present in the ActRII polypeptide. Such modifications include, but are not limited to, acetylation, carboxylation, glycosylation, phosphorylation, lipidation, and acylation. As a result, the ActRII polypeptide may contain non-amino acid elements, such as polyethylene glycols, lipids, polysaccharide or monosaccharide, and phosphates. Effects of such non-amino acid elements on the functionality of a ligand trap polypeptide may be tested as described herein for other ActRII variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the ActRII polypeptides.

In certain aspects, ActRII polypeptides of the present disclosure include fusion proteins having at least a portion (domain) of an ActRII polypeptide and one or more heterologous portions (domains). Well-known examples of such fusion domains include, but are not limited to, polyhistidine, Glu-Glu, glutathione S-transferase (GST), thioredoxin, protein A, protein G, an immunoglobulin heavy-chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) (SEQ ID NO: 137) fusion partners. As another example, a fusion domain may be selected so as to facilitate detection of the ActRII polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagglutinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation. Other types of fusion domains that may be selected include multimerizing (e.g., dimerizing, tetramerizing) domains and functional domains (that confer an additional biological function) including, for example constant domains from immunoglobulins (e.g., Fc domains).

In certain aspects, ActRII polypeptides of the present disclosure contain one or more modifications that are capable of “stabilizing” the polypeptides. By “stabilizing” is meant anything that increases the in vitro half-life, serum half-life, regardless of whether this is because of decreased destruction, decreased clearance by the kidney, or other pharmacokinetic effect of the agent. For example, such modifications enhance the shelf-life of the polypeptides, enhance circulatory half-life of the polypeptides, and/or reduce proteolytic degradation of the polypeptides. Such stabilizing modifications include, but are not limited to, fusion proteins (including, for example, fusion proteins comprising an ActRII polypeptide domain and a stabilizer domain), modifications of a glycosylation site (including, for example, addition of a glycosylation site to a polypeptide of the disclosure), and modifications of carbohydrate moiety (including, for example, removal of carbohydrate moieties from a polypeptide of the disclosure). As used herein, the term “stabilizer domain” not only refers to a fusion domain (e.g., an immunoglobulin Fc domain) as in the case of fusion proteins, but also includes non-proteinaceous modifications such as a carbohydrate moiety, or non-proteinaceous moiety, such as polyethylene glycol. In certain embodiments, an ActRII polypeptide is fused with a heterologous domain that stabilizes the polypeptide (a “stabilizer” domain), preferably a heterologous domain that increases stability of the polypeptide in vivo. Fusions with a constant domain of an immunoglobulin (e.g., a Fc domain) are known to confer desirable pharmacokinetic properties on a wide range of proteins. Likewise, fusions to human serum albumin can confer desirable properties.

An example of a native amino acid sequence that may be used for the Fc portion of human IgG1 (G1Fc) is shown below (SEQ ID NO: 14). Dotted underline indicates the hinge region, and solid underline indicates positions with naturally occurring variants. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 14. Naturally occurring variants in G1Fc would include E134D and M136L according to the numbering system used in SEQ ID NO: 14 (see Uniprot P01857).

| (SEQ ID NO: 14) | | | | |
|---|---|---|---|---|
| 1 | 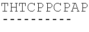 | ELLGGPSVFL | FPPKPKDTLM | ISRTPEVTCV VVDVSHEDPE |
| 51 | VKFNWYVDGV | EVHNAKTKPR | EEQYNSTYRV | VSVLTVLHQD WLNGKEYKCK |
| 101 | VSNKALPAPI | EKTISKAKGQ | PREPQVYTLP | PSREEMTKNQ VSLTCLVKGF |
| 151 | YPSDIAVEWE | SNGOPENNYK | TTPPVLDSDG | SFFLYSKLTV DKSRWQQGNV |
| 201 | FSCSVMHEAL | HNHYTOKSLS | LSPGK | |

Optionally, the IgG1 Fc domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcγ receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain. In certain cases, the mutant IgG1 Fc domain lacks the N-terminal lysine (K).

In some embodiments, the sequence that may be used for the Fc portion of human IgG1 (G1Fc) is shown below:

(SEQ ID NO: 233)
THTCPPCPAPELLGGPSVFLFPPKPKDTLMISRTPEVTCVVV
DVSHEDPEVKFNWYVDGVEVHNAKTKPREEQYNSTYRVVSVL
TVLHQDWLNGKEYKCKVSNKALPVPIEKTISKAKGQPREPQV
YTLPPSREEMTKNQVSLTCLVKGFYPSDIAVEWESNGQPENN
YKTTPPVLDSDGPFFLYSKLTVDKSRWQQGNVFSCSVMHEAL
HNHYTQKSLSLSPGK

In some embodiments, an Fc domain is from an IgG1 antibody and includes amino acid substitutions L12A, L13A, and G15A, relative to the sequence of SEQ ID NO: 233. In some embodiments, an Fc domain is from an IgG1 antibody and includes amino acid substitutions D43A, KIODA, and N212A, relative to the sequence of SEQ ID NO: 233. In certain cases, the mutant IgG1 Fc domain lacks the N-terminal lysine (K). In some embodiments, a polypeptide described herein (e.g., an ActRII polypeptide)) may be fused to the N- or C-terminus of an Fc domain monomer (e.g., SEQ ID NO: 233) through conventional genetic or chemical means, e.g., chemical conjugation. If desired, a linker (e.g., a spacer) can be inserted between the polypeptide and the Fc domain monomer. In some embodiments, the Fc domain monomer can be fused to the N- or C-terminus (e.g., C-terminus) of the polypeptide.

In some embodiments, an Fc domain includes one or more of the following amino acid substitutions: T366W, T366Y, T394W, F405W, Y349T, Y349E, Y349V, L351T, L351H, L351N, L352K, P353S, S354D, D356K, D356R, D356S, E357K, E357R, E357Q, S364A, T366E, L368T, L368Y, L368E, K370E, K370D, K370Q, K392E, K392D, T394N, P395N, P396T, V397T, V397Q, L398T, D399K, D399R, D399N, F405T, F405H, F405R, Y407T, Y407H, Y407I, K409E, K409D, K409T, and K409I, relative to the sequence of human IgG1. In some embodiments, an Fc domain includes the amino acid substitution T366W, relative to the sequence of human IgG1. The sequence of a wild-type Fc domain is shown in SEQ ID NO: 284.

An example of a native amino acid sequence that may be used for the Fc portion of human IgG2 (G2Fc) is shown below (SEQ ID NO: 15). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 15. In certain cases, the IgG2 Fc domain lacks the N-terminal lysine (K).

| (SEQ ID NO: 15) | | | | | |
|---|---|---|---|---|---|
| 1 | 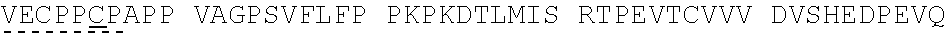 | | | | |
| 51 | FNWYVDGVEV | HNAKTKPREE | QFNSTFRVVS | VLTVVHODWL | NGKEYKCKVS |
| 101 | NKGLPAPIEK | TISKTKGQPR | EPQVYTLPPS | REEMTKNQVS | LTCLVKGFYP |
| 151 | SDIAVEWESN | GOPENNYKTT | PPMLDSDGSF | FLYSKLTVDK | SRWOOGNVES |
| 201 | CSVMHEALHN | HYTOKSLSLS | PGK | | |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 16) contains a short hinge region consisting of a single 15-residue segment, whereas the second G3Fc sequence (SEQ ID NO: 17) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NOs: 16 and 17. In certain cases, the IgG3 Fc domain lacks the N-terminal lysine (K).

| (SEQ ID NO: 16) | | | | | |
|---|---|---|---|---|---|
| 1 | 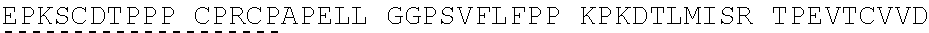 | | | | |
| 51 | VSHEDPEVOF | KWYVDGVEVH | NAKTKPREEQ | YNSTFRVVSV | LTVLHQDWLN |
| 101 | GKEYKCKVSN | KALPAPIEKT | ISKTKGOPRE | PQVYTLPPSR | EEMTKNQVSL |
| 151 | TCLVKGFYPS | DIAVEWESSG | QPENNYNTTP | PMLDSDGSFF | LYSKLTVDKS |
| 201 | RWQQGNIFSC | SVMHEALHNR | FTQKSLSLSP | GK | |
| (SEQ ID NO: 17) | | | | | |
| 1 | 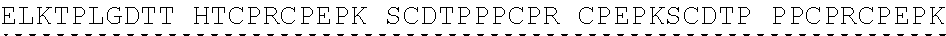 | | | | |
| 51 | 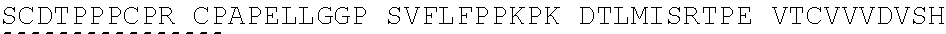 | | | | |
| 101 | EDPEVOFKWY | VDGVEVHNAK | TKPREEQYNS | TFRVVSVLIV | LHQDWLNGKE |
| 151 | YKCKVSNKAL | PAPIEKTISK | TKGOPREPQV | YTLPPSREEM | TKNQVSLTCL |
| 201 | VKGFYPSDIA | VEWESSGOPE | NNYNTTPPML | DSDGSFFLYS | KLTVDKSRWQ |
| 251 | QGNIFSCSVM | HEALHNRFTQ | KSLSLSPGK | | |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 16, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 18). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 18. In certain cases, the IgG4 Fc domain lacks the N-terminal lysine (K).

| (SEQ ID NO: 18) | |
|---|---|
| 1 | 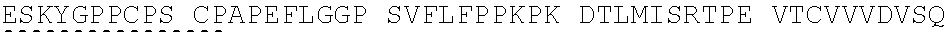 |
| 51 | EDPEVQFNWY VDGVEVHNAK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHYTQ KSLSLSLGK |

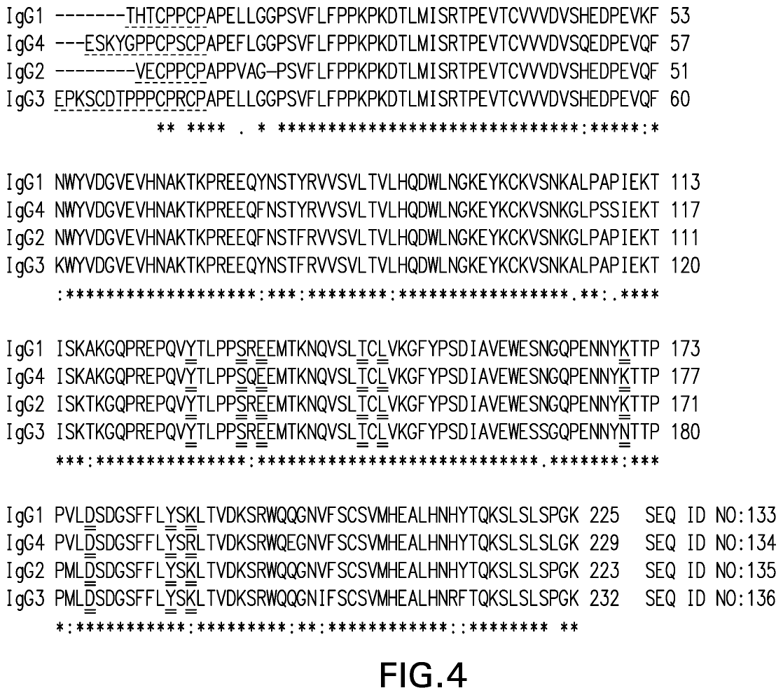

A variety of engineered mutations in the Fc domain are presented herein with respect to the G1Fc sequence (SEQ ID NO: 14), and analogous mutations in G2Fc, G3Fc, and G4Fc can be derived from their alignment with G1Fc in FIG. 4. Due to unequal hinge lengths, analogous Fc positions based on isotype alignment (FIG. 4) possess different amino acid numbers in SEQ ID NOs: 14, 15, 16, 17, and 18. It can also be appreciated that a given amino acid position in an immunoglobulin sequence consisting of hinge, C_{H}2, and C_{H}3 regions (e.g., SEQ ID NOs: 14, 15, 16, 17, and 18) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 14), the human IgG1 heavy chain constant domain (Uniprot P01857), and the human IgG1 heavy chain is as follows.

Correspondence of C_{H}3 Positions in
Different Numbering Systems

| G1Fc | IgG1 heavy chain | IgG1 heavy chain |
|---|---|---|
| (Numbering begins | constant domain | EU numbering |
| at first threonine | (Numbering | scheme of Kabat |
| in hinge region) | begins at C_{H}1) | et al., 1991*) |
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

*Kabat et al. (eds) 1991; pp. 688-696 in Sequences of Proteins of Immunological Interest, 5^{th} ed., Vol. 1, NIH, Bethesda, MD.

The application further provides Fc fusion proteins with engineered or variant Fc regions. Such Fc fusion proteins may be useful, for example, in modulating effector functions, such as, antigen-dependent cytotoxicity (ADCC) and complement-dependent cytotoxicity (CDC). Additionally, the modifications may improve the stability of the Fc fusion proteins. Amino acid sequence variants of the Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the Fc fusion proteins, such as changing the number or position of glycosylation sites. In some embodiments, Fc polypeptide domains of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a polypeptide selected from the group consisting of SEQ ID NOs: 14, 15, 16, 17, 18, 133, 134, 135, 136, 233, and 284.

In some embodiments, a polypeptide disclosed herein (e.g., an ActRIIA variant polypeptide or an ActRIIB variant polypeptide) may further include a moiety (e.g., Fc domain monomer, a wild-type Fc domain, an Fc domain with amino acid substitutions (e.g., one or more substitutions that reduce dimerization), an albumin-binding peptide, a fibronectin domain, or a human serum albumin), which may be fused to the N- or C-terminus (e.g., C-terminus) of the polypeptide by way of a linker or other covalent bonds. A polypeptide (e.g., an ActRIIA variant polypeptide or an ActRIIB variant polypeptide) fused to an Fc domain monomer may form a dimer (e.g., homodimer or heterodimer) through the interaction between two Fc domain monomers, which combine to form an Fc domain in the dimer.

Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26) and the P329G/L234A/L235A (P329G LALA) mutation described by Schlothauer et al. (see Schlothauer T., et al. Protein Eng Des Sel. 2016 October; 29 (10): 457-466). Thus, in certain embodiments, Fc fusion proteins of the disclosure with mutations within the constant region including the Ala-Ala mutation or the P329G LALA mutation may be used to reduce or abolish effector function. According to these embodiments, Fc fusion proteins may comprise a mutation to an alanine at position 234 or a mutation to an alanine at position 235, or a combination thereof. In one embodiment, the Fc fusion protein comprises an IgG4 framework, wherein the Ala-Ala mutation would describe a mutation(s) from phenylalanine to alanine at position 234 and/or a mutation from leucine to alanine at position 235. In some embodiments, Fc fusion proteins may further comprise mutation from proline to glycine at position 329. In another embodiment, the Fc fusion protein comprises an IgG1 framework, wherein the Ala-Ala mutation would describe a mutation(s) from leucine to alanine at position 234 and/or a mutation from leucine to alanine at position 235. In some embodiments, the Fc fusion protein comprising an IgG1 framework further comprises a mutation from proline to glycine at position 329. The Fc fusion protein may alternatively or additionally carry other mutations, including the point mutation K322A in the CH2 domain (Hezareh et al. 2001 J Virol. 75:12161-8).

In some embodiments, the Fc fusion protein may be modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively, or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. Sec Caron et al., J. Exp Med. 176:1191-1195 (1992) and Shopes, B. J. Immunol. 148:2918-2922 (1992), WO99/51642, Duncan & Winter Nature 322:738-40 (1988); U.S. Pat. Nos. 5,648,260; 5,624,821; and WO94/29351.

In some embodiments, a polypeptide described herein may include an extracellular ActRIIA variant fused to a serum protein-binding peptide. Binding to serum protein peptides can improve the pharmacokinetics of protein pharmaceuticals.

As one example, albumin-binding peptides that can be used in the methods and compositions described here are generally known in the art. In one embodiment, the albumin binding peptide includes the sequence DICLPRWGCLW (SEQ ID NO: 285).

In some embodiments, albumin-binding peptides may be joined to the N- or C-terminus (e.g., C-terminus) of a polypeptide described herein (e.g., an ActRIIA or ActRIIB polypeptide)) to increase the serum half-life of the extracellular ActRIIA variant. In some embodiments, an albumin-binding peptide is joined, either directly or through a linker, to the N- or C-terminus of the polypeptide.

In some embodiments, a polypeptide described herein (e.g., an ActRIIA or ActRIIB polypeptide) may be fused to the N- or C-terminus of albumin-binding peptide (e.g., SEQ ID NO: 285) through conventional genetic or chemical means, e.g., chemical conjugation. If desired, a linker (e.g., a spacer) can be inserted between the polypeptide and the albumin-binding peptide. Without being bound to a theory, it is expected that inclusion of an albumin-binding peptide in an extracellular ActRIIA or ActRIIB variant described herein may lead to prolonged retention of the therapeutic protein through its binding to serum albumin.

In some embodiments, a polypeptide described herein may include a polypeptide (e.g., an ActRIIA or ActRIIB polypeptide) fused to fibronectin domains. Binding to fibronectin domains can improve the pharmacokinetics of protein pharmaceuticals.

A fibronectin domain is a high molecular weight glycoprotein of the extracellular matrix, or a fragment thereof, that binds to, e.g., membrane-spanning receptor proteins such as integrins and extracellular matrix components such as collagens and fibrins. In some embodiments, a fibronectin domain is joined to the N- or C-terminus (e.g., C-terminus) of a polypeptide described herein (e.g., an ActRIIA or ActRIIB polypeptide) to increase the serum half-life of the polypeptide. A fibronectin domain can be joined, either directly or through a linker, to the N- or C-terminus of a polypeptide.

As one example, fibronectin domains that can be used in the methods and compositions described here are generally known in the art. In one embodiment, the fibronectin domain is a fibronectin type III domain (SEQ ID NO: 286) having amino acids 610-702 of the sequence of UniProt ID NO: P02751. In another embodiment, the fibronectin domain is an adnectin protein.

In some embodiments, a polypeptide described herein (e.g., an ActRIIA or ActRIIB polypeptide) may be fused to the N- or C-terminus of a fibronectin domain (e.g., SEQ ID NO: 286) through conventional genetic or chemical means, e.g., chemical conjugation. If desired, a linker (e.g., a spacer) can be inserted between the polypeptide and the fibronectin domain. Without being bound to a theory, it is expected that inclusion of a fibronectin domain in a polypeptide described herein may lead to prolonged retention of the therapeutic protein through its binding to integrins and extracellular matrix components such as collagens and fibrins.

In some embodiments, a polypeptide described herein may include an ActRIIA or ActRIIB polypeptide fused to serum albumin. Binding to serum albumins can improve the pharmacokinetics of protein pharmaceuticals.

Serum albumin is a globular protein that is the most abundant blood protein in mammals. Serum albumin is produced in the liver and constitutes about half of the blood serum proteins. It is monomeric and soluble in the blood. Some of the most crucial functions of serum albumin include transporting hormones, fatty acids, and other proteins in the body, buffering pH, and maintaining osmotic pressure needed for proper distribution of bodily fluids between blood vessels and body tissues. In some embodiments, serum albumin is human serum albumin. In some embodiments, a human serum albumin is joined to the N- or C-terminus (e.g., C-terminus) of a polypeptide described herein (e.g., an ActRIIA or ActRIIB polypeptide) to increase the serum half-life of the polypeptide. In some embodiments, the human serum albumin can be joined, either directly or through a linker, to the N- or C-terminus of a polypeptide disclosed herein.

As one example, serum albumins that can be used in the methods and compositions described herein are generally known in the art. In one embodiment, the serum albumin includes the sequence of UniProt ID NO: P02768 (SEQ ID NO: 287).

In some embodiments, a polypeptide described herein (e.g., an ActRIIA or ActRIIB polypeptide) may be fused to the N- or C-terminus of a human serum albumin (e.g., SEQ ID NO: 287) through conventional genetic or chemical means, e.g., chemical conjugation. If desired, a linker (e.g., a spacer) can be inserted between the polypeptide and the human serum albumin. Without being bound to a theory, it is expected that inclusion of a human serum albumin in an ActRIIA or ActRIIB polypeptide described herein may lead to prolonged retention of the therapeutic protein.

It is understood that different elements of the fusion proteins (e.g., immunoglobulin Fc fusion proteins) may be arranged in any manner that is consistent with desired functionality. For example, an ActRII polypeptide domain may be placed C-terminal to a heterologous domain, or alternatively, a heterologous domain may be placed C-terminal to an ActRII polypeptide domain. The ActRII polypeptide domain and the heterologous domain need not be adjacent in a fusion protein, and additional domains or amino acid sequences may be included C- or N-terminal to either domain or between the domains.

For example, an ActRII receptor fusion protein may comprise an amino acid sequence as set forth in the formula A-B-C. The B portion corresponds to an ActRII polypeptide domain. The A and C portions may be independently zero, one, or more than one amino acid, and both the A and C portions when present are heterologous to B. The A and/or C portions may be attached to the B portion via a linker sequence. In certain embodiments, an ActRII fusion protein comprises an amino acid sequence as set forth in the formula A-B-C, wherein A is a leader (signal) sequence, B consists of an ActRII polypeptide domain, and C is a polypeptide portion that enhances one or more of in vivo stability, in vivo half-life, uptake/administration, tissue localization or distribution, formation of protein complexes, and/or purification. In certain embodiments, an ActRII fusion protein comprises an amino acid sequence as set forth in the formula A-B-C, wherein A is a TPA leader sequence, B consists of an ActRII receptor polypeptide domain, and C is an immunoglobulin Fc domain. Exemplary fusion proteins comprise the amino acid sequence set forth in any one of SEQ ID NOs: 36, 39, 40, 42, 45, 46, 48, 69, 74, 77, 78, 139, 140, 141, 142, 143, 144, 318, or 331.

In some embodiments, ActRII polypeptides to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of purity are well known in the art [see, e.g., Flatman et al., (2007) J. Chromatogr. B 848:79-87]. In some embodiments, ActRII polypeptide to be used in accordance with the methods described herein are recombinant polypeptides.

ActRII polypeptides of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the polypeptides of the disclosure, including fragments or variants thereof, may be recombinantly produced using various expression systems (e.g., E. coli, Chinese Hamster Ovary (CHO) cells, COS cells, baculovirus) as is well known in the art. In a further embodiment, the modified or unmodified polypeptides of the disclosure may be produced by digestion of recombinantly produced full-length ActRII polypeptides by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites. Alternatively, such polypeptides may be produced from recombinantly generated full-length ActRII polypeptides using chemical cleavage (e.g., cyanogen bromide, hydroxylamine, etc.).

### 3. Linkers

The disclosure provides for ActRII polypeptides and variants thereof (e.g., ActRIIA polypeptides or ActRIIB polypeptides), and in these embodiments, the polypeptide portion (e.g., ActRIIA polypeptide) is connected to the heterologous portion (e.g., Fc portion) by means of a linker. In some embodiments, the linkers are glycine and serine rich linkers. In some embodiments, the linker may be rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 19), GGGG (SEQ ID NO: 20), TGGGG (SEQ ID NO: 21), SGGGG (SEQ ID NO: 22), TGGG (SEQ ID NO: 23), SGGG (SEQ ID NO: 24), or GGGGS (SEQ ID NO: 25) singlets, or repeats. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In some embodiments, the linker comprises the amino acid sequence GlyGlyGlyGlySer (GGGGS) (SEQ ID NO: 25), or repetitions thereof (GGGGS) n, where n≥2. In particular embodiments n≥3, or n=3-10. In some embodiments, n≥4, or n=4-10. In some embodiments, n is not greater than 4 in a (GGGGS) n linker. In some embodiments, n=4-10, 4-9, 4-8, 4-7, 4-6, 4-5, 5-8, 5-7, or 5-6. In some embodiments, n=3, 4, 5, 6, or 7. In particular embodiments, n=4. In some embodiments, a linker comprising a (GGGGS), sequence also comprises an N-terminal threonine. In some embodiments, the linker is any one of the following:

(SEQ ID NO: 85)
GGGGSGGGGS
(SEQ ID NO: 86)
TGGGGSGGGGS
(SEQ ID NO: 87)
TGGGGSGGGGSGGGGS
(SEQ ID NO: 88)
TGGGGSGGGGSGGGGSGGGGS
(SEQ ID NO: 89)
TGGGGSGGGGSGGGGSGGGGSGGGGS
(SEQ ID NO: 90)
TGGGGSGGGGSGGGGSGGGGSGGGGSGGGGS
or
(SEQ ID NO: 91)
TGGGGSGGGGSGGGGSGGGGSGGGGSGGGGSGGGGS.

In some embodiments, the linker comprises the amino acid sequence of TGGGPKSCDK (SEQ ID NO: 92). In some embodiments, the linker is any one of SEQ ID NOs: 85-92 lacking the N-terminal threonine. In some embodiments, the linker does not comprise the amino acid sequence of SEQ ID NO: 90 or 91.

In some embodiments, a polypeptide described (e.g., ActRIIA polypeptides or ActRIIB polypeptides) herein may include a polypeptide fused to a moiety by way of a linker. In some embodiments, the moiety increases stability of the polypeptide. In some embodiments, the moiety is selected from the group consisting of an Fc domain monomer, a wild-type Fc domain, an Fc domain with amino acid substitutions (e.g., one or more substitutions that reduce dimerization), an albumin-binding peptide, a fibronectin domain, and a human serum albumin. In some embodiments, a linker between a moiety (e.g., an Fc domain monomer (e.g., the sequence of SEQ ID NO: 233), a wild-type Fc domain (e.g., SEQ ID NO: 284), an Fc domain with amino acid substitutions (e.g., one or more substitutions that reduce dimerization), an albumin-binding peptide (e.g., SEQ ID NO: 285), a fibronectin domain (e.g., SEQ ID NO: 286), or a human serum albumin (e.g., SEQ ID NO: 287)) and a polypeptide (e.g., ActRIIA polypeptides or ActRIIB polypeptides), can be an amino acid linker including 1-200 amino acids. Suitable peptide linkers are known in the art, and include, for example, peptide linkers containing flexible amino acid residues such as glycine, alanine, and serine. In some embodiments, a linker can contain motifs, e.g., multiple or repeating motifs, of GA, GS, GG, GGA, GGS, GGG, GGGA (SEQ ID NO: 234), GGGS (SEQ ID NO: 235), GGGG (SEQ ID NO: 20), GGGGA (SEQ ID NO: 236), GGGGS (SEQ ID NO: 25), GGGGG (SEQ ID NO: 237), GGAG (SEQ ID NO: 238), GGSG (SEQ ID NO: 239), AGGG (SEQ ID NO: 240), or SGGG (SEQ ID NO: 24). In some embodiments, a linker can contain 2 to 12 amino acids including motifs of GA or GS, e.g., GA, GS, GAGA (SEQ ID NO: 241), GSGS (SEQ ID NO: 242), GAGAGA (SEQ ID NO: 243), GSGSGS (SEQ ID NO: 244), GAGAGAGA (SEQ ID NO: 245), GSGSGSGS (SEQ ID NO: 246), GAGAGAGAGA (SEQ ID NO: 247), GSGSGSGSGS (SEQ ID NO: 248), GAGAGAGAGAGA (SEQ ID NO: 249), and GSGSGSGSGSGS (SEQ ID NO: 250). In some embodiments, a linker can contain 3 to 12 amino acids including motifs of GGA or GGS, e.g., GGA, GGS, GGAGGA (SEQ ID NO: 251), GGSGGS (SEQ ID NO: 252), GGAGGAGGA (SEQ ID NO: 253), GGSGGSGGS (SEQ ID NO: 254), GGAGGAGGAGGA (SEQ ID NO: 255), and GGSGGSGGSGGS (SEQ ID NO: 256). In some embodiments, a linker can contain 4 to 12 amino acids including motifs of GGAG (SEQ ID NO: 238), GGSG (SEQ ID NO: 239), GGAGGGAG (SEQ ID NO: 257), GGSGGGSG (SEQ ID NO: 258), GGAGGGAGGGAG (SEQ ID NO: 259), and GGSGGGSGGGSG (SEQ ID NO: 260). In some embodiments, a linker can contain motifs of GGGGA (SEQ ID NO: 236) or GGGGS (SEQ ID NO: 25), e.g., GGGGAGGGGAGGGGA (SEQ ID NO: 261) and GGGGSGGGGSGGGGS (SEQ ID NO: 262). In some embodiments, an amino acid linker between a moiety (e.g., an Fc domain monomer, a wild-type Fc domain, an Fc domain with amino acid substitutions (e.g., one or more substitutions that reduce dimerization), an albumin-binding peptide, a fibronectin domain, or a human serum albumin) and a polypeptide (e.g., ActRIIA polypeptides or ActRIIB polypeptides) may be GGG, GGGA (SEQ ID NO: 234), GGGG (SEQ ID NO: 20), GGGAG (SEQ ID NO: 263), GGGAGG (SEQ ID NO: 264), or GGGAGGG (SEQ ID NO: 265).

In some embodiments, a linker can also contain amino acids other than glycine, alanine, and serine, e.g., AAAL (SEQ ID NO: 266), AAAK (SEQ ID NO: 267), AAAR (SEQ ID NO: 268), EGKSSGSGSESKST (SEQ ID NO: 269), GSAGSAAGSGEF (SEQ ID NO: 270), AEAAAKEAAAKA (SEQ ID NO: 271), KESGSVSSEQLAQFRSLD (SEQ ID NO: 272), GENLYFQSGG (SEQ ID NO: 273), SACYCELS (SEQ ID NO: 274), RSIAT (SEQ ID NO: 275), RPACKIPNDLKQKVMNH (SEQ ID NO: 276), GGSAGGSGSGSSGGSSGASGTGTAGGTGSGSGTGSG (SEQ ID NO: 277), AAANSSIDLISVPVDSR (SEQ ID NO: 278), or GGSGGGSEGGGSEGGGSEGGGSEGGGSEGGGSGGGS (SEQ ID NO: 279). In some embodiments, a linker can contain motifs, e.g., multiple or repeating motifs, of EAAAK (SEQ ID NO: 280). In some embodiments, a linker can contain motifs, e.g., multiple or repeating motifs, of praline-rich sequences such as (XP) n, in which X may be any amino acid (e.g., A, K, or E) and n is from 1-5, and PAPAP (SEQ ID NO: 281).

The length of the peptide linker and the amino acids used can be adjusted depending on the two proteins involved and the degree of flexibility desired in the final protein fusion polypeptide. The length of the linker can be adjusted to ensure proper protein folding and avoid aggregate formation.

### 4. Nucleic Acids Encoding ActRII Polypeptides and Variants Thereof

In certain embodiments, the present disclosure provides isolated and/or recombinant nucleic acids encoding ActRII polypeptides (including fragments, functional variants, and fusion proteins thereof). For example, SEQ ID NO: 7 encodes a naturally occurring human ActRIIB precursor polypeptide (the R64 variant described above), while SEQ ID NO: 8 encodes the processed extracellular domain of ActRIIB (the R64 variant described above). The subject nucleic acids may be single-stranded or double-stranded. Such nucleic acids may be DNA or RNA molecules. These nucleic acids may be used, for example, in methods for making ActRII-based variant polypeptides as described herein.

As used herein, isolated nucleic acid(s) refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

In certain embodiments, nucleic acids encoding ActRII polypeptides of the disclosure are understood to include nucleic acids that are variants of any one of SEQ ID NOs: 7, 8, 12, 13, 37, 43, 49, 70, 71, 72, 73, 75, 76, 80, 81, 82, 83, 84, and 94. Variant nucleotide sequences include sequences that differ by one or more nucleotide substitutions, additions, or deletions including allelic variants, and therefore, will include coding sequence that differ from the nucleotide sequence designated in any one of SEQ ID NOs: 7, 8, 12, 13, 37, 43, 49, 70, 71, 72, 73, 75, 76, 80, 81, 82, 83, 84, and 94.

In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to any one of SEQ ID NOs: 7, 8, 12, 13, 37, 43, 49, 70, 71, 72, 73, 75, 76, 80, 81, 82, 83, 84, and 94. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 7. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 8. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 12. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 13. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 37. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 43. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 49. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 70. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 71. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 72. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 73. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 75. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 76. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 80. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 81. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 82. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 83. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 84. In certain embodiments, ActRII polypeptides of the disclosure are encoded by isolated and/or recombinant nucleic acid sequences that are at least 70%, 75%, 80%, 85%, 90%, 91%, 92%, 93%, 94% 95%, 96%, 97%, 98%, 99%, or 100% identical to SEQ ID NO: 94. One of ordinary skill in the art will appreciate that nucleic acid sequences that are at least 70%, 75%, 80%, 85%, 90%, 91%, 92%, 93%, 94% 95%, 96%, 97%, 98%, 99%, or 100% identical to the sequences complementary to SEQ ID NOs: 7, 8, 12, 13, 37, 43, 49, 70, 71, 72, 73, 75, 76, 80, 81, 82, 83, and 84, and variants thereof, are also within the scope of the present disclosure. In further embodiments, the nucleic acid sequences of the disclosure can be isolated, recombinant, and/or fused with a heterologous nucleotide sequence, or in a DNA library.

In other embodiments, nucleic acids of the present disclosure also include nucleotide sequences that hybridize under highly stringent conditions to the nucleotide sequence designated in SEQ ID NOs: 7, 8, 12, 13, 37, 43, 49, 70, 71, 72, 73, 75, 76, 80, 81, 82, 83, and 84, complement sequences of SEQ ID NOs: 7, 8, 12, 13, 37, 43, 49, 70, 71, 72, 73, 75, 76, 80, 81, 82, 83, and 84, or fragments thereof. As discussed above, one of ordinary skill in the art will understand readily that appropriate stringency conditions which promote DNA hybridization can be varied. One of ordinary skill in the art will understand readily that appropriate stringency conditions which promote DNA hybridization can be varied. For example, one could perform the hybridization at 6.0× sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in SEQ ID NOS: 7, 8, 12, 13, 37, 43, 49, 70, 71, 72, 73, 75, 76, 80, 81, 82, 83, and 84 to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the protein. However, it is expected that DNA sequence polymorphisms that do lead to changes in the amino acid sequences of the subject proteins will exist among mammalian cells. One skilled in the art will appreciate that these variations in one or more nucleotides (up to about 3-5% of the nucleotides) of the nucleic acids encoding a particular protein may exist among individuals of a given species due to natural allelic variation. Any and all such nucleotide variations and resulting amino acid polymorphisms are within the scope of this disclosure.

In certain embodiments, the recombinant nucleic acids of the present disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art and can be used in a variety of host cells. Typically, one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In some embodiments, the expression vector contains a selectable marker gene to allow the selection of transformed host cells. Selectable marker genes are well known in the art and can vary with the host cell used.

In certain aspects, the subject nucleic acid disclosed herein is provided in an expression vector comprising a nucleotide sequence encoding an ActRII polypeptide and operably linked to at least one regulatory sequence. Regulatory sequences are art-recognized and are selected to direct expression of the ActRII polypeptide. Accordingly, the term “regulatory sequence” includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding an ActRII polypeptide. Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other protein encoded by the vector, such as antibiotic markers, should also be considered.

A recombinant nucleic acid of the present disclosure can be produced by ligating the cloned gene, or a portion thereof, into a vector suitable for expression in either prokaryotic cells, eukaryotic cells (yeast, avian, insect or mammalian), or both. Expression vehicles for production of a recombinant ActRII polypeptide include plasmids and other vectors. For instance, suitable vectors include plasmids of the following types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of proteins in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, e.g., Molecular Cloning A Laboratory Manual, 3rd Ed., ed. by Sambrook, Fritsch and Maniatis (Cold Spring Harbor Laboratory Press, 2001). In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

In one embodiment, a vector will be designed for production of the subject ActRII polypeptides in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the subject ActRII polypeptides in cells propagated in culture, e.g., to produce proteins, including fusion proteins or variant proteins, for purification.

This disclosure also pertains to a host cell transfected with a recombinant gene including a coding sequence for one or more of the subject ActRII polypeptides. The host cell may be any prokaryotic or eukaryotic cell. For example, an ActRII polypeptide of the disclosure may be expressed in bacterial cells such as E. coli, insect cells (e.g., using a baculovirus expression system), yeast, or mammalian cells (e.g., a Chinese hamster ovary (CHO) cell line]) Other suitable host cells are known to those skilled in the art.

Accordingly, the present disclosure further pertains to methods of producing the subject ActRII polypeptides. For example, a host cell transfected with an expression vector encoding an ActRII polypeptide can be cultured under appropriate conditions to allow expression of the ActRII polypeptide to occur. The polypeptide may be secreted and isolated from a mixture of cells and medium containing the polypeptide. Alternatively, the ActRII polypeptide may be retained cytoplasmically or in a membrane fraction and the cells harvested, lysed and the protein isolated. A cell culture includes host cells, media and other byproducts. Suitable media for cell culture are well known in the art. The subject polypeptides can be isolated from cell culture medium, host cells, or both, using techniques known in the art for purifying proteins, including ion-exchange chromatography, gel filtration chromatography, ultrafiltration, electrophoresis, immunoaffinity purification with antibodies specific for particular epitopes of the ActRII polypeptides, and affinity purification with an agent that binds to a domain fused to the ActRII polypeptide (e.g., a protein A column may be used to purify an ActRII-Fc fusion proteins). In some embodiments, the ActRII polypeptide is a fusion protein containing a domain which facilitates its purification.

In some embodiments, purification is achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. An ActRII polypeptide may be purified to a purity of >90%, >95%, >96%, >98%, or >99% as determined by size exclusion chromatography and >90%, >95%, >96%, >98%, or >99% as determined by SDS PAGE. The target level of purity should be one that is sufficient to achieve desirable results in mammalian systems, particularly non-human primates, rodents (mice), and humans.

In another embodiment, a fusion gene coding for a purification leader sequence, such as a poly-(His)/enterokinase cleavage site sequence at the N-terminus of the desired portion of the recombinant ActRII polypeptide, can allow purification of the expressed fusion protein by affinity chromatography using a Ni²⁺ metal resin. The purification leader sequence can then be subsequently removed by treatment with enterokinase to provide the purified ActRII polypeptide. See, e.g., Hochuli et al. (1987) J. Chromatography 411:177; and Janknecht et al. (1991) PNAS USA 88:8972.

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence. See, e.g., Current Protocols in Molecular Biology, eds. Ausubel et al., John Wiley & Sons: 1992.

### 5. Screening Assays

In certain aspects, the present disclosure relates to the use of the subject ActRII antagonists (e.g., ActRII polypeptides) to identify compounds (agents) which may be used to treat, prevent, or reduce the progression rate and/or severity of renal diseases or conditions (e.g., Alport syndrome, focal segmental glomerulosclerosis (FSGS), polycystic kidney diseases, chronic kidney disease).

There are numerous approaches to screening for therapeutic agents for treating renal diseases or conditions (e.g., Alport syndrome, focal segmental glomerulosclerosis (FSGS), polycystic kidney diseases, chronic kidney disease) by targeting signaling (e.g., SMAD signaling) of one or more ligands. In certain embodiments, high-throughput screening of compounds can be carried out to identify agents that perturb ligand-mediated effects on a selected cell line. In certain embodiments, the assay is carried out to screen and identify compounds that specifically inhibit or reduce binding of a TGF-beta ligand (e.g., activin A, activin B, activin AB, activin AC, BMP6, BMP7, BMP9, BMP10, GDF3, GDF8, and/or GDF11) to its binding partner, such as a type II receptor (e.g., ActRIIA and/or ActRIIB). Alternatively, the assay can be used to identify compounds that enhance binding of a ligand to its binding partner such as a type II receptor. In a further embodiment, the compounds can be identified by their ability to interact with a type II receptor.

A variety of assay formats will suffice and, in light of the present disclosure, those not expressly described herein will nevertheless be comprehended by one of ordinary skill in the art. As described herein, the test compounds (agents) disclosed herein may be created by any combinatorial chemical method. Alternatively, the subject compounds may be naturally occurring biomolecules synthesized in vivo or in vitro. Compounds (agents) to be tested for their ability to act as modulators of tissue growth can be produced, for example, by bacteria, yeast, plants or other organisms (e.g., natural products), produced chemically (e.g., small molecules, including peptidomimetics), or produced recombinantly. Test compounds contemplated by the present invention include non-peptidyl organic molecules, peptides, polypeptides, peptidomimetics, sugars, hormones, and nucleic acid molecules. In certain embodiments, the test agent is a small organic molecule having a molecular weight of less than about 2,000 Daltons.

The test compounds of the disclosure can be provided as single, discrete entities, or provided in libraries of greater complexity, such as made by combinatorial chemistry. These libraries can comprise, for example, alcohols, alkyl halides, amines, amides, esters, aldehydes, ethers and other classes of organic compounds. Presentation of test compounds to the test system can be in either an isolated form or as mixtures of compounds, especially in initial screening steps. Optionally, the compounds may be optionally derivatized with other compounds and have derivatizing groups that facilitate isolation of the compounds. Non-limiting examples of derivatizing groups include biotin, fluorescein, digoxygenin, green fluorescent protein, isotopes, polyhistidine, magnetic beads, glutathione S-transferase (GST), photoactivatable crosslinkers or any combinations thereof.

In many drug-screening programs which test libraries of compounds and natural extracts, high-throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity or bioavailability of the test compound can be generally ignored in the in vitro system, the assay instead being focused primarily on the effect of the drug on the molecular target as may be manifest in an alteration of binding affinity between a TGF-beta ligand (e.g., activin A, activin B, activin AB, activin AC, BMP6, BMP7, BMP9, BMP10, GDF3, GDF8, and/or GDF11) to its binding partner, such as a type II receptor (e.g., ActRIIA and/or ActRIIB).

Merely to illustrate, in an exemplary screening assay of the present disclosure, the compound of interest is contacted with an isolated and purified ActRIIB polypeptide which is ordinarily capable of binding to an ActRIIB ligand, as appropriate for the intention of the assay. To the mixture of the compound and ActRIIB polypeptide is then added to a composition containing an ActRIIB ligand (e.g., GDF11). Detection and quantification of ActRIIB/ActRIIB-ligand complexes provides a means for determining the compound's efficacy at inhibiting (or potentiating) complex formation between the ActRIIB polypeptide and its binding protein. The efficacy of the compound can be assessed by generating dose-response curves from data obtained using various concentrations of the test compound. Moreover, a control assay can also be performed to provide a baseline for comparison. For example, in a control assay, isolated and purified ActRIIB ligand is added to a composition containing the ActRIIB polypeptide, and the formation of ActRIIB/ActRIIB ligand complex is quantitated in the absence of the test compound. It will be understood that, in general, the order in which the reactants may be admixed can be varied, and can be admixed simultaneously. Moreover, in place of purified proteins, cellular extracts and lysates may be used to render a suitable cell-free assay system.

Complex formation between a ligand and its binding protein may be detected by a variety of techniques. For instance, modulation of the formation of complexes can be quantitated using, for example, detectably labeled proteins such as radiolabeled (e.g., 32P, 35S, 14C or 3H), fluorescently labeled (e.g., FITC), or enzymatically labeled ActRIIB polypeptide and/or its binding protein, by immunoassay, or by chromatographic detection.

In certain embodiments, the present disclosure contemplates the use of fluorescence polarization assays and fluorescence resonance energy transfer (FRET) assays in measuring, either directly or indirectly, the degree of interaction between a ligand and its binding protein. Further, other modes of detection, such as those based on optical waveguides (see, e.g., PCT Publication WO 96/26432 and U.S. Pat. No. 5,677,196), surface plasmon resonance (SPR), surface charge sensors, and surface force sensors, are compatible with many embodiments of the disclosure.

Moreover, the present disclosure contemplates the use of an interaction trap assay, also known as the “two-hybrid assay,” for identifying agents that disrupt or potentiate interaction between a ligand and its binding partner. See, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; and Iwabuchi et al. (1993) Oncogene 8:1693-1696). In a specific embodiment, the present disclosure contemplates the use of reverse two-hybrid systems to identify compounds (e.g., peptides) that dissociate interactions between a ligand and its binding protein [see, e.g., Vidal and Legrain, (1999) Nucleic Acids Res 27:919-29; Vidal and Legrain, (1999) Trends Biotechnol 17:374-81; and U.S. Pat. Nos. 5,525,490; 5,955,280; and 5,965,368].

In certain embodiments, the subject compounds are identified by their ability to interact with a particular ligand. The interaction between the compound and the ligand may be covalent or non-covalent. For example, such interaction can be identified at the protein level using in vitro biochemical methods, including photo-crosslinking, radiolabeled ligand binding, and affinity chromatography [see, e.g., Jakoby W B et al. (1974) Methods in Enzymology 46:1]. In certain cases, the compounds may be screened in a mechanism-based assay, such as an assay to detect compounds which bind to a particular ligand. This may include a solid-phase or fluid-phase binding event. Alternatively, the gene encoding a ligand can be transfected with a reporter system (e.g., β-galactosidase, luciferase, or green fluorescent protein) into a cell and screened against the library preferably by high-throughput screening or with individual members of the library. Other mechanism-based binding assays may be used; for example, binding assays which detect changes in free energy. Binding assays can be performed with the target fixed to a well, bead or chip or captured by an immobilized antibody or resolved by capillary electrophoresis. The bound compounds may be detected usually using colorimetric endpoints or fluorescence or surface plasmon resonance.

### 6. Methods of Use

In part, the present disclosure relates to methods of treating renal diseases or conditions (e.g., Alport syndrome, focal segmental glomerulosclerosis (FSGS), polycystic kidney diseases, chronic kidney disease), comprising administering to a patient in need thereof an effective amount of an ActRII antagonist of the present disclosure. In some embodiments, an ActRII antagonist of the present disclosure can be used to treat or prevent a disease or condition that is associated with abnormal activity of a ActRIIA or ActRIIB polypeptide, and/or an ActRIIA or ActRIIB ligand (e.g., Activin A, activin B, GDF11, GDF8, GDF3, BMP5, BMP6, and BMP10).

In certain embodiments, the present invention provides methods of treating an individual in need thereof through administering to the individual a therapeutically effective amount of an ActRII antagonist, as described herein, optionally in combination with one or more additional active agents and/or supportive therapies.

The terms “renal” and “kidney” are used interchangeably herein.

The terms “treatment”, “treating”, “alleviation” and the like are used herein to generally mean obtaining a desired pharmacologic and/or physiologic effect, and may also be used to refer to improving, alleviating, and/or decreasing the severity of one or more clinical complication of a condition being treated. The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample. A patient, subject, or individual “in need of treatment” includes those suffering from, suspected of having, diagnosed with, or predisposed to one or more renal diseases or conditions (e.g., Alport syndrome, focal segmental glomerulosclerosis (FSGS), polycystic kidney diseases, chronic kidney disease), or those in which these diseases or conditions are to be prevented.

In general, treatment or prevention of a disease or condition as described in the present disclosure is achieved by administering an ActRII antagonist in an effective amount. An effective amount of an agent refers to an amount effective, at dosages and for periods of time necessary, to achieve the desired therapeutic or prophylactic result. A therapeutically effective amount of an agent of the present disclosure may vary according to factors such as the disease state, age, sex, and weight of the individual, and the ability of the agent to elicit a desired response in the individual. A prophylactically effective amount refers to an amount effective, at dosages and for periods of time necessary, to achieve the desired prophylactic result.

The terms “patient”, “subject”, or “individual” are used interchangeably herein and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The term “baseline” as used herein refers to an initial measurement that can be compared to. In some instances, a baseline measurement can be a measurement made while a subject is administered only standard of care (SOC). In some instances, a baseline measurement can be made without a subject being administered SOC. A baseline measurement can also be a measurement made prior to administration of an ActRII antagonist of the present disclosure and/or SOC.

In certain aspects, the disclosure contemplates the use of an ActRII antagonist, in combination with one or more additional active agents or other supportive therapy for treating or preventing a disease or condition (e.g., Alport syndrome, focal segmental glomerulosclerosis (FSGS), polycystic kidney disease, chronic kidney disease). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. One or more an ActRII antagonists of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of an ActRII antagonist of the present disclosure with the additional active agent or therapy and/or the desired effect.

In some embodiments, the disclosure contemplates methods of treating one or more complications of a renal disease or condition comprising administering to a subject in need thereof an effective amount of an ActRII antagonist. In some embodiments, the disclosure contemplates methods of preventing one or more complications of a renal disease or condition comprising administering to a subject in need thereof an effective amount of an ActRII antagonist. In some embodiments, the disclosure contemplates methods of reducing the progression rate of a renal disease or condition comprising administering to a subject in need thereof an effective amount of an ActRII antagonist. In some embodiments, the disclosure contemplates methods of reducing the progression rate of one or more complications of a renal disease or condition comprising administering to a subject in need thereof an effective amount of an ActRII antagonist. In some embodiments, the disclosure contemplates methods of reducing the severity of a renal disease or condition comprising administering to a subject in need thereof an effective amount of an ActRII antagonist. In some embodiments, the disclosure contemplates methods of reducing the severity of one or more complications of a renal disease or condition comprising administering to a subject in need thereof an effective amount of an ActRII antagonist. In some embodiments, a renal disease or condition is selected from the group consisting of Alport syndrome, focal segmental glomerulosclerosis (FSGS), polycystic kidney disease, and chronic kidney disease. In some embodiments, a renal disease or condition is Alport syndrome. In some embodiments, a renal disease or condition is focal segmental glomerulosclerosis (FSGS). In some embodiments, a renal disease or condition is polycystic kidney disease. In some embodiments, a renal disease or condition is chronic kidney disease. In some embodiments, a subject has a decline in kidney function. In some embodiments, methods of the present disclosure slow kidney function decline.

In certain aspects, the disclosure relates to methods of treating, preventing, or reducing the progression rate and/or severity of a renal disease or condition comprising administering to a subject in need thereof an effective amount of an ActRII antagonist. In some embodiments, the method relates to treating a subject with Alport Syndrome. In some embodiments, the method relates to treating a subject with a confirmatory genetic diagnosed Alport Syndrome.

Alport syndrome, also known as hereditary nephritis, is a genetically heterogeneous disease that results from mutations in genes encoding alpha-3, alpha-4, and alpha-5 chains of type IV collagen. Type IV collagen alpha chains are normally located in various basement membranes throughout the body, including the kidneys. Abnormalities in these chains can result in defective basement membranes at these sites, which in turn lead to clinical features of Alport syndrome (e.g., progressive glomerular disease).

Transmission of Alport syndrome can be X-linked, autosomal recessive, or autosomal dominant. In some embodiments, a subject has X-linked Alport syndrome. In some embodiments, the disclosure relates to methods of treating a subject that has X-linked Alport syndrome. X-linked transmission accounts for the majority of affected patients and arises from mutations in the COL4A5 gene on the X chromosome. In some embodiments, a subject has genetic defects in the COL4A5 gene. In some embodiments, the disclosure relates to methods of treating a subject that has one or more genetic defects in the COL4A5 gene. Autosomal recessive variant accounts for approximately 15 percent of patients with Alport syndrome and arises from genetic defects in either the COL4A3 or COL4A4 genes. In some embodiments, a subject has autosomal recessive Alport syndrome. Autosomal dominant disease appears to account for between about 20 to about 30 percent of patients with Alport syndrome and arises from heterozygous mutations in the COL4A3 or COL4A4 genes. In some embodiments, a subject has autosomal dominant Alport syndrome. In some embodiments, a subject has heterozygous mutations in the COL4A3 gene. In some embodiments, a subject has heterozygous mutations in the COL4A4 gene. In some embodiments, a subject has genetic defects in the COL4A3 gene. In some embodiments, the disclosure relates to methods of treating a subject that has one or more genetic defects in the COL4A3 gene. In some embodiments, a subject has genetic defects in the COL4A4 gene. In some embodiments, the disclosure relates to methods of treating a subject that has one or more genetic defects in the COL4A4 gene. In some embodiments, a subject has genetic defects in the COL4A3 and COLA4A genes. In some embodiments, the disclosure relates to methods of treating a subject that has one or more genetic defects in the COL4A3 and COL4A4 genes. Some families exhibit digenic inheritance due to transmission of mutations in two of the three genes (COL4A3, COL4A4, COL4A5). In some embodiments, a subject has mutations in two of the three genes (COL4A3, COL4A4, COL4A5). In some embodiments, the disclosure relates to methods of treating a subject that has one or more genetic defects in the COL4A3, COL4A4, and/or COL4A5 genes.

The classical presentation of Alport syndrome is based upon clinical manifestations of affected males with X-linked disease. In some embodiments, a subject with X-linked disease has a glomerular disease that progresses to end-stage renal disease (ESRD). Clinical presentation and course in patients with autosomal recessive disease is similar to those with X-linked disease. Patients with autosomal dominant disease generally exhibit more gradual loss of renal function.

Initially, renal manifestation of Alport syndrome is typically asymptomatic persistent microscopic hematuria (e.g., presence of blood in the urine), which is usually present in early childhood in affected patients. Since screening urinalysis is seldom performed in routine pediatric primary care, microscopic hematuria may not be detected unless the patient is screened because of an affected family member or found as an incidental finding for another issue. Gross hematuria may be the initial presenting finding and often occurs after an upper respiratory infection. However, recurrent episodes of gross hematuria are not uncommon especially during childhood. In some embodiments, the disclosure relates to methods of treating a subject that has asymptomatic persistent microscopic hematuria. In some embodiments, the disclosure relates to methods of treating a subject that has gross hematuria. In some embodiments, the disclosure relates to methods of treating a subject that has recurring episodes of gross hematuria. In some embodiments, the disclosure relates to methods of reducing the severity, occurrence, and/or duration of asymptomatic persistent microscopic hematuria, gross hematuria, or persistent microscopic hematuria in a subject in need thereof (e.g., a subject with Alport syndrome).

Patients with Alport syndrome typically have normal C3 levels, which is a component of the complement pathway that plays an integral role in the body's immune defenses. Decreased C3 may be associated with acute glomerulonephritis, membranoproliferative glomerulonephritis, immune complex disease, active systemic lupus erythematosus, septic shock, and end-stage liver disease, among other conditions. In early childhood, serum creatinine and blood pressure measurements are usually at normal levels as well. In some embodiments, the disclosure relates to methods of treating a subject with Alport syndrome that has normal levels of C3. In some embodiments, the disclosure relates to methods of treating a subject with Alport syndrome that has decreased levels of C3 compared to a baseline measurement. In some embodiments, the disclosure relates to methods of increasing C3 levels in a subject in need thereof (e.g., a subject with Alport syndrome).

Proteinuria, hypertension, and progressive renal insufficiency may develop in a subject with Alport syndrome. Proteinuria comprises a presence of excess proteins in urine. Albumin is a protein produced by the liver which makes up roughly 50%-60% of the proteins in the blood. Due to this, the concentration of albumin in the urine is one of the most sensitive indicators of any kidney disease, particularly for subjects with diabetes or hypertension, compared to a routine proteinuria examination. This measurement is often referred to as albuminuria. In some embodiments, the disclosure relates to methods of treating a subject that has proteinuria. In some embodiments, the disclosure relates to methods of treating a subject that has hypertension. In some embodiments, the disclosure relates to methods of treating a subject that has progressive renal insufficiency. In some embodiments, the disclosure relates to methods of reducing the severity, occurrence, and/or duration of one or more of proteinuria, hypertension, and progressive renal insufficiency in a subject in need thereof (e.g., a subject with Alport syndrome).

Subjects with Alport syndrome may develop end-stage renal disease (ESRD). ESRD usually occurs between the ages of 16 and 35 years in patients with X-linked or autosomal recessive Alport syndrome, among many other renal diseases and conditions. In some families, the course is more indolent with kidney failure being delayed until age 45 to 60, especially in those with autosomal dominant Alport syndrome. Females with X-linked Alport syndrome may have recurrent episodes of gross hematuria, proteinuria, and diffuse glomerular basement membrane (GBM) thickening are associated with more severe kidney dysfunction and ESRD at an earlier age. In some embodiments, the disclosure relates to methods of treating subjects with Alport syndrome that have ESRD. In some embodiments, the disclosure relates to methods of treating females with X-linked Alport syndrome. In some embodiments, the disclosure relates to methods of reducing severity, occurrence and/or duration of one or more of gross hematuria, proteinuria, and diffuse GBM thickening are associated with more severe kidney dysfunction and ESRD in a subject in need thereof (e.g., a subject with Alport syndrome).

A diagnosis of Alport syndrome may be made by molecular genetic testing, or by skin or renal biopsy. Molecular genetic next generation analysis is one method of making a diagnosis for patients with a positive family history for persistent hematuria and/or end-stage renal disease (ESRD) and for patients with chronic kidney disease (CKD), regardless of family history. Alport syndrome can be distinguished from other glomerular diseases by presence of a characteristic finding of lamination of the glomerular basement membrane (GBM) in samples from a renal biopsy, or abnormalities of type IV collagen by immunostaining, or by identification of one or more mutations in COL4A3, COL4A4, or COL4A5. Thin glomerular basement membranes in a subject with a COL4A3, COL4A4, or COL4A5 mutation, with or without the manifestation of FSGS, is properly diagnosed as Alport syndrome. In some embodiments, the disclosure relates to methods of treating subjects with Alport syndrome that have a positive family history for persistent hematuria and/or ESRD and/or for patients with CKD.

In certain aspects, the disclosure relates to methods of treating, preventing, or reducing the progression rate and/or severity of a renal disease or condition comprising administering to a subject in need thereof an effective amount of an ActRII antagonist to subjects that have focal segmental glomerulosclerosis (FSGS). In some embodiments, the subject has primary FSGS. In some embodiments, the subject has genetic FSGS.

FSGS is a glomerular scarring disease characterized by an effacement of the podocyte foot on a kidney biopsy. When urine samples from subjects suffering FSGS are analyzed, a massive urine protein loss is typically observed, which can progress to a renal failure. FSGS is a common histopathologic lesion among adults with idiopathic nephrotic syndrome in the United States, accounting for about 35 percent of all cases. FSGS is also the most common primary glomerular disease identified in patients with end-stage renal disease (ESRD) in the United States. Prevalence of FSGS as a lesion associated with ESRD has risen. FSGS is characterized by the presence of sclerosis in parts (segmental) of at least one glomerulus (focal) of a kidney biopsy specimen, when examined by light microscopy (LM), immunofluorescence (IF), or electron microscopy (EM). In some embodiments, the disclosure relates to methods of reducing severity, occurrence and/or duration of urine protein loss in a subject in need thereof (e.g., a subject with FSGS). In some embodiments, the disclosure relates to methods of reducing severity, occurrence and/or duration of renal failure in a subject in need thereof (e.g., a subject with FSGS). In some embodiments, the disclosure relates to methods of reducing severity, occurrence and/or duration of end stage renal disease (ESRD) in a subject in need thereof (e.g., a subject with FSGS). In some embodiments, the disclosure relates to methods of reducing severity, occurrence and/or duration of sclerosis in a glomerulus of a kidney in a subject in need thereof (e.g., a subject with FSGS).

FSGS arises as a consequence of multiple pathways either individually or collectively resulting in injury to a podocyte, which is a cell in the Bowman's capsule in the kidneys that wraps around capillaries of the glomerulus. There are five known etiologies, and a suggested sixth etiology, associated with FSGS. Etiologies of FSGS comprise primary (e.g., idiopathic), secondary (e.g., adaptive), genetic, virus-associated, medication-associated, and APOL1 risk allele-associated. Primary or idiopathic FSGS is associated with a plasma factor with responsiveness to immunosuppressive therapy and a risk of recurrence after kidney transplant. In primary FSGS, a putative circulating factor that is toxic to a podocyte causes generalized podocyte dysfunction. Primary FSGS most often presents with the nephrotic system. Secondary (e.g., adaptive) FSGS is associated with excessive nephron workload due to increased body size, reduced nephron capacity, or single glomerular hyperfiltration associated with certain diseases. Secondary FSGS generally occurs as an adaptive phenomenon that results from a reduction in nephron mass, or can be considered as medicated-induced by direct toxicity from drugs (e.g., heroin, interferon, and pamidronate) or virus-induced by viral infections (e.g., HIV). Secondary FSGS often presents with non-nephrotic proteinuria, and/or with some degree of renal insufficiency. Secondary FSGS most commonly refers to FSGS that develops as an adaptive response to glomerular hypertrophy or hyperfiltration. Additional etiologies are recognized as drivers of FSGS, including high-penetrance genetic FSGS due to mutations in one of nearly 40 genes (genetic FSGS), virus-associated FSGS, and medication-associated FSGS. Emerging data support the identification of a sixth etiology: APOL1 risk allele-associated FSGS in individuals with sub-Saharan ancestry. Sometimes, secondary FSGS encompasses virus-associated FSGS and/or medication-associated FSGS. In some embodiments, the disclosure relates to methods of treating a subject with primary or idiopathic FSGS. In some embodiments, the disclosure relates to methods of treating a subject with secondary or adaptive FSGS. In some embodiments, the disclosure relates to methods of treating a subject with genetic FSGS. In some embodiments, the disclosure relates to methods of treating a subject with virus-associated FSGS. In some embodiments, the disclosure relates to methods of treating a subject with medication-associated FSGS. In some embodiments, the disclosure relates to methods of treating a subject with APOL1 risk allele-associated FSGS.

Primary FSGS comprises several prototypical characteristics. Primary FSGS is the most common form of FSGS in adolescents and young adults, and is commonly associated with nephrotic-range proteinuria (sometimes massive proteinuria, e.g., >10 g protein/day in the urine), reduced plasma albumin levels, and/or hyperlipidemia. In some embodiments, nephrotic-range proteinuria comprises proteinuria >3.5 g protein/day, and/or hypoalbuminemia <3.5 g albumin/dL urine (<35 g/L), and/or other manifestations of the nephrotic syndrome (e.g., edema, hyperlipidemia). In some embodiments, the disclosure relates to methods of reducing severity, occurrence and/or duration of one or more of nephrotic range proteinuria, reduced plasma albumin levels, or hyperlipidemia in a subject in need thereof (e.g., a subject with primary FSGS). In some embodiments, the disclosure relates to methods of reducing severity, occurrence and/or duration of proteinuria in a subject in need thereof (e.g., a subject with primary FSGS).

A subject with secondary or adaptive FSGS typically presents with slowly increasing proteinuria and renal insufficiency over time. Proteinuria in subjects with secondary FSGS often presents in the non-nephrotic range (e.g., nephrotic range is typically a loss of 3 grams or more of protein in the urine per day, and/or presence of 2 grams of protein per gram of creatinine in the urine). Sometimes, proteinuria in subjects with secondary FSGS comprises serum albumin levels that are normal. A subject with secondary FSGS may have a glomerular filtration rate (GFR) that is elevated, which is a measurement of the flow rate of filtered fluid through the kidney. In some embodiments, a subject with secondary FSGS and an increase in GFR may have one or more additional and/or associated conditions selected from the group consisting of congenital cyanotic heart disease, sickle cell anemia, obesity, androgen abuse, sleep apnea, and high-protein diet. In some embodiments, the disclosure relates to methods of treating a subject with secondary FSGS with a normal GFR. In some embodiments, the disclosure relates to methods of treating a subject with secondary FSGS that has a decreased GFR. In some embodiments, the disclosure relates to methods of reducing severity, occurrence and/or duration of proteinuria and/or renal insufficiency in a subject in need thereof (e.g., a subject with primary FSGS).

Viruses have been implicated in causing FSGS. HIV-1 may be associated with FSGS, particularly the collapsing glomerulopathy variant. Other viruses that have been implicated in causing FSGS include, but are not limited to, cytomegalovirus, parvovirus B19, and Epstein-Barr virus. Parasites have also been associated with FSGS, which include, but are not limited to, Plasmodium (malaria), Schistosoma mansoni, and filiariasis. In some embodiments, the disclosure relates to methods of treating a subject with FSGS associated with HIV-1. In some embodiments, the disclosure relates to methods of treating a subject with FSGS associated with one or more of HIV-1, cytomegalovirus, parvovirus B19, and Epstein-Barr virus. In some embodiments, the disclosure relates to methods of treating a subject with FSGS associated with parasites including, but not limited to, Plasmodium (malaria), Schistosoma mansoni, and filiariasis. In some embodiments, the disclosure relates to methods of treating subjects with FSGS associated with an infection including, but not limited to, HIV, cytomegalovirus, parvovirus B19, Epstein-Barr virus, pulmonary tuberculosis, leishmaniasis, and malaria.

In some embodiments, the disclosure relates to methods of treating subjects with FSGS associated with autoimmune disorders implicated in causing FSGS including, but not limited to Adult Still's disease, systemic lupus erythematosus, and mixed connective tissue disorder.

In some embodiments, the disclosure relates to methods of treating subjects with FSGS associated with malignancies implicated in causing FSGS including, but not limited to hemophagocytic lymphohistiocytosis, multiple myeloma, and acute monoblastic leukemia.

In some embodiments, the disclosure relates to methods of treating subjects with FSGS associated with acute glomerular ischemias implicated in causing FSGS including, but not limited to thrombotic microangiopathy, renal infarction, atheroembolism, and hydrophilic polymer embolism.

In some embodiments, the disclosure relates to methods of treating subjects with FSGS associated with genetic disorders implicated in causing FSGS including, but not limited to APOL1 high-risk alleles, sickle cell disease, mitochondrial disorders (coenzyme Q deficiency), acute myoclonus-renal failure syndrome, and Galloway-Mowat syndrome.

In some embodiments, the disclosure relates to methods of treating subjects with FSGS associated with post transplantation events implicated in causing FSGS including, but not limited to Arteriopathy/thrombotic microangiopathy, acute rejection, and viral infection (cytomegalovirus, Epstein-Barr virus, BK polyomavirus).

In some embodiments, the disclosure relates to methods of treating subjects with FSGS associated with certain medications. In some embodiments, IFN-α, -β, or -γ therapy has been associated with development of collapsing glomerulopathy. In some embodiments, the disclosure relates to methods of treating subjects with FSGS associated with one or more of podocyte injury, including MCD, FSGS, and particularly, collapsing FSGS (collapsing glomerulopathy) who have taken and/or are still taking bisphosphonates. In some embodiments, the disclosure relates to methods of treating subjects with FSGS who have been on and/or are currently on lithium therapy. In some embodiments, the disclosure relates to methods of treating subjects with FSGS who have taken and/or are still taking sirolimus. In some embodiments, the disclosure relates to methods of treating subjects with FSGS who have taken and/or are still taking anthracycline medications, including doxorubicin (Adriamcyin) and daunomycin. In some embodiments, the disclosure relates to methods of treating subjects with FSGS who have taken and/or are still taking medications implicated in causing FSGS including, but not limited to bisphosphonates, interferons (alpha, beta, or gamma), anabolic steroids, calcineurin inhibitors, and mammalian (mechanistic) target of rapamycin (mTOR) inhibitors.

Genetic FSGS takes two forms. In some embodiments, the disclosure relates to methods of treating subjects with genetic FSGS associated with one or more variants in susceptibility genes (i.e., some individuals with a particular variant will develop FSGS, and other individuals will not). In some embodiments, the disclosure relates to methods of treating subjects with FSGS associated with one or more susceptibility genes including, but not limited to APOL1 and PDSS1. In some embodiments, the disclosure relates to methods of treating subjects with genetic FSGS associated with one or more high-penetrance mutations that manifest either Mendelian inheritance (for nuclear genes) or maternal inheritance (for genes encoded by mitochondrial DNA). The number of genes associated with FSGS rises every year, in large part because of the dissemination of whole-exome sequencing. At least 38 genes have been identified in relation to genetic FSGS. In some embodiments, the disclosure relates to methods of treating subjects with FSGS associated with one or more genes involved in genetic FSGS comprising COL4A3, COL4A4, COL4A5, ITGB4, LAMB2, NPHS, NPHS2, CD2AP, PTPRO, MYO1E, ACTN4, INF2, AHRGP24, AHRGDIA, MYH9, INF1, MT-TL1, MT-TL2, MT-TY, COQ2, COQ6, PDSS2, ADCK, WT1, NUP95, NUP203, XP05, NXF5, PAX2, LMX1B, SMARCAL1, NXF5, EYA1, WDR73, LMNA, PLCE1, TRPC6, KANK4, SCARB2, and TTC21B.

In some embodiments, a subject suspected of FSGS is administered a kidney biopsy. A kidney biopsy may be analyzed by light microscopy to determine one or more of glomerular size, histologic variant of FSGS, microcystic tubular changes, and tubular hypertrophy. Further, a kidney biopsy may be analyzed by immunofluorescence to rule out other primary glomerulopathies and/or by electron microscopy to determine one or more of an extent of podocyte foot process effacement, podocyte microvillous transformation, and tubuloreticular inclusions. A complete assessment of renal histology is important for establishing the parenchymal setting of segmental glomerulosclerosis, distinguishing FSGS associated with one of many other glomerular diseases from the clinical-pathologic syndrome of FSGS. In some embodiments, genetic testing is used to further analyze a subject for a genetic FSGS etiology.

Traditionally, FSGS was classified based upon the Columbia classification, which defined five morphologic variants of FSGS lesions based upon LM examination. This classification system was designed to rely solely on pathologic criteria and does not integrate these findings with clinical and/or genetic information. In general, morphologic characteristics seen on kidney biopsy cannot distinguish between genetic and nongenetic forms of FSGS. Exceptions include distinctive features associated with NPHS1 and actinin alpha 4 gene mutations and the disease-specific lesions of Fabry disease, Alport syndrome, and lecithin-cholesterol acyl transferase deficiency. Histologic variants of FSGS comprise FSGS not otherwise specified (NOS) (formerly called classic FSGS, which is the most common form); collapsing variant, tip variant; perihilar variant; and cellular variant. Although the appearance of a glomerulus on LM, by definition, differs among these forms, they all share ultrastructural findings of podocyte alterations. Tip lesions affect the portion of the glomerular tuft juxtaposed to the tubular pole, and a tip lesion abnormality includes one or more of adhesion to Bowman's capsule at the tip, hypercellularity, presence of foam cells, and/or sclerosis. A collapsing variant shows segmental or global mesangial consolidation and loss of endocapillary patency in association with extracapillary epithelial hypertrophy and/or proliferation. Perihilar and NOS variants are determined by whether the segmental sclerosis/segmental obliteration of capillary loops with matrix increase (with or without hyalinosis) involves the segment near the hilum or the specific segment cannot be determined, respectively. A cellular lesion is the most difficult lesion to identify reproducibly. A cellular lesion shows segmental endocapillary hypercellularity occluding lumens with or without foam cells and karyorrhexis.

In certain aspects, the disclosure relates to methods of treating, preventing, or reducing the progression rate and/or severity of a renal disease or condition comprising administering to a subject in need thereof an effective amount of an ActRII antagonist, wherein the renal disease or condition is polycystic kidney disease (PKD).

Polycystic Kidney Disease occurs in two forms: autosomal recessive (ARPKD) and autosomal dominant (ADPKD). The two forms of the disease have distinct genetic basis, and two genes involved in ADPKD have been identified, and one gene involved in ARPKD has been identified. The manifestations of the two different types of disease are very similar, and both result from a hyperproliferation of tubule epithelial cells that ultimately results in destruction of tubular structure with cyst formation leading to chronic renal failure. In some embodiments, the disclosure relates to methods of treating subjects with autosomal recessive polycystic kidney disease (ARPKD). In some embodiments, the disclosure relates to methods of treating subjects with autosomal dominant polycystic kidney disease (ADPKD).

Autosomal dominant polycystic kidney disease (ADPKD) is a hereditary disorder of the kidneys characterized by markedly enlarged kidneys with extensive cyst formation throughout. These cysts progressively enlarge with age, as kidney function gradually declines. A diagnosis of ADPKD is based on family history and ultrasonographic evaluation. In as many as 25% of patients with ADPKD, no family history is identified, which may be related to subclinical disease or a new genetic mutation in about 5% of such cases. A defining feature of ADPKD is marked bilateral, renal enlargement. Patients with ADPKD typically progress to end-stage renal disease (ESRD) by the fifth or sixth decade of life. The rate of progression of ADPKD is related directly to kidney volume, and therapies aim to slow the decline in renal volume to delay progression. In some embodiments, the disclosure relates to methods of reducing severity, occurrence and/or duration of cysts on the kidney in a subject in need thereof. In some embodiments, the disclosure relates to methods of reducing severity, occurrence and/or duration of renal enlargement in a subject in need thereof. In some embodiments, the disclosure relates to methods of reducing severity, occurrence and/or duration of an increase in kidney volume (e.g., total kidney volume) in a subject in need thereof.

ADPKD can be attributed to an abnormality on chromosome 16 (PKD1 locus) or chromosome 4 (PKD2 locus). PKD1 mutations comprise about 78% of ADPKD cases, while PKD2 mutations comprise about 14% of cases. PKD1 patients tend to progress to ESRD at an earlier age than PKD2 patients. In some embodiments, the disclosure relates to methods of treating a subject with ADPKD that has a mutation in the PKD1 locus. In some embodiments, the disclosure relates to methods of treating a subject with ADPKD that has a mutation in the PKD2 locus.

The PKD1 and PKD2 genes encode the proteins polycystin-1 and polycystin-2, respectively. These polycystins are integral membrane proteins and are found in renal tubular epithelia. It is postulated that abnormalities in polycystin-1 impair cell-cell and cell-matrix interactions in the renal tubular epithelia, while abnormalities in polycystin-2 impair calcium signaling in the cells.

Resultant changes in renal pathophysiology due to PKD include, but are not limited to, hematuria (often gross), a concentrating defect (resulting in polyuria and increased thirst), mild proteinuria, nephrolithiasis (in about 25% of ADPKD patients), flank pain, and abdominal pain. Furthermore, cyst rupture, hemorrhage, and infection are common complications. Progressive renal decline often results in end-stage renal disease. Hypertension is the most prevalent initial clinical presentation, occurring in about 50% to about 70% of cases, and is the most common feature directly associated with the rate of decline to ESRD and cardiovascular complications. In some embodiments, the disclosure relates to methods of reducing severity, occurrence and/or duration of one or more of hematuria, a concentrating defect, proteinuria, nephrolithiasis, flank pain, and abdominal pain in a subject in need thereof. In some embodiments, the disclosure relates to methods of reducing severity, occurrence and/or duration of one or more of cyst rupture, hemorrhage, and infection in a subject in need thereof. In some embodiments, the disclosure relates to methods of reducing severity, occurrence and/or duration of end-stage renal disease (ESRD) in a subject in need thereof. In some embodiments, the disclosure relates to methods of reducing severity, occurrence and/or duration of hypertension in a subject in need thereof.

Multiple extra-renal manifestations are often present in a subject with polycystic kidney disease. Cerebral aneurysms occur in about 5% of young adults, and as many as 20% of patients over the age of 60. Risk of a cerebral aneurysm or subarachnoid hemorrhage is highest in subjects with a family history of the same. Extrarenal cysts are common in ADPKD. Hepatic cysts are often noted in these patients, and prevalence increases with age. As many as 94% of patients over the age of 35 have been reported to have hepatic cysts. Total cyst prevalence and volume is higher in women versus men. Hepatic cysts in ADPKD patients rarely cause liver dysfunction. Rarely, patients develop pain from an acute cyst infection or hemorrhage. In addition, between about 7% and about 36% of ADPKD patients develop pancreatic cysts, with a higher prevalence in ADPKD patients with PKD2 mutations. Cardiac valvular disease has been noted in 25% to 30% of ADPKD patients. Cardiovascular complications, particularly cardiac hypertrophy and coronary artery disease are leading causes of death in patients with ADPKD. In some embodiments, the disclosure relates to methods of reducing severity, occurrence and/or duration of a cerebral aneurysm in a subject in need thereof. In some embodiments, the disclosure relates to methods of reducing severity, occurrence and/or duration of extrarenal cysts in a subject in need thereof. In some embodiments, the disclosure relates to methods of reducing severity, occurrence and/or duration of hepatic cysts in a subject in need thereof. In some embodiments, the disclosure relates to methods of reducing severity, occurrence and/or duration of pancreatic cysts in a subject in need thereof. In some embodiments, the disclosure relates to methods of reducing severity, occurrence and/or duration of cardiovascular complications (e.g., cardiac hypertrophy, coronary artery disease) in a subject. in need thereof.

Autosomal recessive polycystic kidney disease (ARPKD) is a cause of significant renal and liver-related morbidity and mortality in children. A majority of subjects with ARPKD present in the neonatal period with enlarged echogenic kidneys. Renal disease is characterized by nephromegaly, hypertension, and varying degrees of renal dysfunction. More than 50% of affected individuals with ARPKD progress to end-stage renal disease (ESRD) within the first decade of life, and subjects with ARPKD whom progressed to ESRD may require kidney transplantation. In some embodiments, the disclosure relates to methods of reducing severity, occurrence and/or duration of one or more nephromegaly, hypertension, and renal dysfunction in a subject in need thereof. In some embodiments, the disclosure relates to methods of reducing severity, occurrence and/or duration of end stage renal disease in a subject in need thereof, preventing a need for kidney transplantation.

ARPKD can be attributed to mutations in the PKHD1 gene located on chromosome 6p21, which contains at least 66 exons and encodes fibrocystin (also referred to as polyductin), a large integral membrane protein. Although the function of fibrocystin is presently unknown, it is found in the cortical and medullary collecting ducts and the thick ascending limb of the kidney, and in the epithelial cells of the hepatic bile duct. In some embodiments, the disclosure relates to methods of treating a subject with ARPKD that is associated with one or more mutations in PKHD1.

Because of the diversity of PKHD1 mutations, it can be challenging to correlate genotype with phenotype in cases of ARPKD. Subjects with two truncation mutations may have more severe renal involvement and are possibly at risk for early neonatal death. Subjects who are homozygotes for a missense mutation, or who have a missense mutation paired with a truncating mutation, may also have a severe phenotype. Subjects who are heterozygotes with two missense mutations typically have milder disease. Subjects who survive the neonatal period most often have at least one missense mutation. In some embodiments, the present disclosure relates to methods of treating a subject with ARPKD comprising two truncation mutations. In some embodiments, the present disclosure relates to methods of treating a subject with ARPKD comprising one or more missense mutations.

Two primary organ systems affected in ARPKD are the kidney and hepatobiliary tract. Kidneys may increase in size and/or have microcysts (usually less than 2 mm in size), which radiate from the medulla to the cortex, and are visible as pinpoint dots on the capsular surface. Severity of renal disease is proportional to the percentage of nephrons affected by cysts. Larger renal cysts (up to 1 cm) and interstitial fibrosis develop, which contribute to the progressive deterioration of renal function seen in subjects who survive beyond the neonatal period. ARPKD is associated with biliary dysgenesis due to a developmental defect comprising varying degrees of dilatation of the intrahepatic bile ducts and hepatic fibrosis. In some embodiments, the disclosure relates to methods of reducing severity, occurrence and/or duration of an increase in kidney size and/or presence of cysts.

Clinical presentation of ARPKD varies based on the age of onset of symptoms and the predominance of hepatic or renal involvement. ARPKD is often detected by routine antenatal ultrasonography in fetuses after 24 weeks of gestation. A presumptive diagnosis is based on the presence of characteristic findings of markedly enlarged echogenic kidneys with poor corticomedullary differentiation. Discrete cysts ranging in size from 5 to 7 mm in diameter may be detected; however, larger cysts are unusual, especially those >10 mm in diameter. Subjects with ARPKD are typically monitored for blood pressure changes, renal function, serum electrolyte concentrations, hydration status, nutritional status, and growth. In some embodiments, the disclosure relates to methods of treating a subject with ARPKD further comprising monitoring one or more of blood pressure, renal function, serum electrolyte concentration, hydration status, nutritional status, and growth.

During the neonatal period, infants can present with renal manifestations, which may or may not be accompanied by respiratory distress. An infant with ARPKD may present with bilateral markedly enlarged kidneys, which may impact pulmonary function or lead to difficulty in feeding due to renal compression of the stomach. An infant with ARPKD may present with renal function impairment reflected by increased serum/plasma concentrations of creatinine and blood urea nitrogen (BUN). Neonates with end-stage renal disease (ESRD) may require renal replacement therapy (RRT) for survival. An infant with ARPKD may present with one or more of hypertension and hyponatremia (due to the inability to dilute urine maximally). In some embodiments, the disclosure relates to methods of reducing severity, occurrence and/or duration of renal function impairment reflected by increased serum/plasma concentrations of creatinine and blood urea nitrogen (BUN) in a subject in need thereof. In some embodiments, the disclosure relates to methods of reducing severity, occurrence and/or duration of hypertension and/or hyponatremia in a subject in need thereof.

For patients who survive beyond the neonatal period, there can be improvement of renal function due to continued renal maturation. However, over time, progressive deterioration of renal function can develop, which may be rapid or slow and may result in ESRD. An adolescent subject with ARPKD may have one or more of progressive deterioration of renal function (usually beginning with signs of tubular dysfunction or injury, polyuria and/or polydipsia due to a reduced concentrating ability, a maximal urine osmolality below 500 mosmol/kg, metabolic acidosis due to decreased urinary acidification capacity, hypertension, recurrent episodes of urinary tract infections, urinary abnormalities (including, but not limited to, mild proteinuria, glucosuria, hyperphosphaturia, and/or increased urinary excretion of magnesium), progressive renal impairment, and decreased kidney growth rate and/or kidney size. In some embodiments, the present disclosure relates to methods of reducing severity, occurrence and/or duration of one or more of progressive deterioration of renal function, progressive renal impairment, and decreased kidney growth rate and/or kidney size in a subject in need thereof.

Ultrasound findings of ARPKD are characterized by bilateral large echogenic kidneys with poor corticomedullary differentiation. In patients with only medullary involvement, standard-resolution ultrasonography may be normal; however, high-resolution ultrasonography is able to detect ductal dilations confined to the medulla. Macrocysts, typically seen in subjects with autosomal dominant disease, are not usually present during infancy in patients with ARPKD, but may appear in older children. As a result, in older subjects, it may be more challenging to differentiate ARPKD from autosomal dominant polycystic kidney disease (ADPKD) by ultrasound. In some embodiments, the present disclosure relates to methods of treating a subject with ARPKD or ADPKD, further comprising differentiation of disease by ultrasound.

In certain aspects, the disclosure relates to methods of treating, preventing, or reducing the progression rate and/or severity of a renal disease or condition comprising administering to a subject in need thereof an effective amount of an ActRII antagonist to a subject that has chronic kidney disease (CKD).

Chronic kidney disease (CKD) is a condition in which the kidneys are damaged and cannot filter blood as well as healthy kidneys. A subject with CKD typically has excess fluid and waste from blood remaining in the body. In some embodiments, the disclosure provides methods of treating a subject with CKD. In some embodiments, the disclosure relates to treating a subject with CKD, wherein the subject also has one or more other health conditions selected from the group consisting of anemia or low number of red blood cells, increased occurrence of infections, low calcium levels, high potassium levels, and high phosphorus levels in the blood, loss of appetite or eating less, depression and lower quality of life.

CKD has varying levels of seriousness and typically gets worse over time, though treatment has been shown to slow progression. If left untreated, CKD can progress to kidney failure, end stage renal disease (ESRD), and/or early cardiovascular disease, potentially leading to dialysis or kidney transplant for survival. In some embodiments, the present disclosure relates to methods of reducing severity, occurrence and/or duration of kidney failure, end stage renal disease (ESRD), and/or early cardiovascular disease in a subject in need thereof.

Diagnosis of CKD is typically accomplished by blood tests to measure the estimated glomerular filtration rate (eGFR), and/or a urine test to measure albumin and/or overall protein in the urine. Typically, an increase in protein in the urine indicates CKD. Ultrasound or kidney biopsy may be performed to determine an underlying cause.

In some embodiments, CKD manifests initially without symptoms, and is usually detected on routine screening blood work by either an increase in serum creatinine, and/or protein in the urine. As kidney function of a subject with CKD decreases, blood pressure increases due to fluid overload and production of vasoactive hormones created by the kidney via the renin-angiotensin system, thereby increasing the risk of developing hypertension and heart failure. As urea accumulates in a subject with CKD, azotemia and ultimately uremia (symptoms ranging from lethargy to pericarditis and encephalopathy) may arise. Due to its high systemic concentration, urea is excreted in eccrine sweat at high concentrations and crystallizes on skin as the sweat evaporates (e.g., “uremic frost”). In a subject with CKD, potassium may accumulate in the blood (e.g., hyperkalemia with a range of symptoms including malaise and potentially fatal cardiac arrhythmias). Hyperkalemia usually does not develop in a subject with CKD until the glomerular filtration rate (GFR) falls to less than about 20 to about 25 ml/min/1.73 m², at which point the kidneys have decreased ability to excrete potassium. Hyperkalemia in CKD can be exacerbated by acidemia (which leads to extracellular shift of potassium) and from lack of insulin. A subject with CKD may have hyperphosphatemia, which can result from poor phosphate elimination in the kidney. Hyperphosphatemia contributes to increased cardiovascular risk by causing vascular calcification. A subject with CKD may have hypocalcemia. A subject with CKD may have one or more changes in mineral and bone metabolism that may cause abnormalities of calcium, phosphorus (phosphate), parathyroid hormone, or vitamin D metabolism; abnormalities in bone turnover, mineralization, volume, linear growth, or strength (kidney osteodystrophy); and/or vascular or other soft-tissue calcification. A subject with CKD may have metabolic acidosis that may result from decreased capacity to generate enough ammonia from the cells of the proximal tubule. A subject with CKD may have anemia. In later stages of CKD, a subject may develop cachexia, leading to unintentional weight loss, muscle wasting, weakness and anorexia. Subjects with CKD are more likely than the general population to develop atherosclerosis with consequent cardiovascular disease. In some embodiments, the present disclosure relates to methods of reducing severity, occurrence and/or duration of one or more conditions or complications of CKD selected from the group consisting of blood pressure increase, hypertension and/or heart failure, azotemia, uremia, “uremic frost”, hyperkalemia, decreased ability of the kidney to excrete potassium, acidemia, hyperphosphatemia, vascular calcification, hypocalcemia, changes in mineral and bone metabolism (particularly changes that may cause abnormalities of calcium, phosphorus (phosphate), parathyroid hormone, or vitamin D metabolism), abnormalities in bone turnover, mineralization, volume, linear growth, or strength (kidney osteodystrophy), vascular or other soft-tissue calcification, metabolic acidosis, anemia, cachexia (particularly cachexia that may lead to unintentional weight loss, muscle wasting, weakness and anorexia), and atherosclerosis (which may lead to cardiovascular disease).

Common causes of CKD are diabetes mellitus, hypertension, and glomerulonephritis. About one of five adults with hypertension and one of three adults with diabetes have CKD. CKD may also be caused by one or more of vascular diseases (including but not limited to, large vessel disease such as bilateral kidney artery stenosis and small vessel disease such as ischemic nephropathy, hemolytic-uremic syndrome, vasculitis), primary glomerular disease (focal segmental glomerulosclerosis (FSGS) and/or IgA nephropathy (or nephritis)), secondary glomerular disease (such as diabetic nephropathy and lupus nephritis), tubulointerstitial disease (which includes drug- and toxin-induced chronic tubulointerstitial nephritis, and reflux nephropathy), obstructive nephropathy (as exemplified by bilateral kidney stones and benign prostatic hyperplasia of the prostate gland), and congenital disease (such as polycystic kidney disease). Rarely, pinworms infecting the kidney can cause obstructive nephropathy. In some embodiments, the present disclosure relates to methods of treating a subject with CKD caused by one or more of diabetes mellitus, hypertension, and glomerulonephritis, vascular diseases (including but not limited to, large vessel disease such as bilateral kidney artery stenosis and small vessel disease such as ischemic nephropathy, hemolytic-uremic syndrome, vasculitis), primary glomerular disease (focal segmental glomerulosclerosis (FSGS) and/or IgA nephropathy (or nephritis)), secondary glomerular disease (such as diabetic nephropathy and lupus nephritis), tubulointerstitial disease (which includes drug- and toxin-induced chronic tubulointerstitial nephritis, and reflux nephropathy), obstructive nephropathy (as exemplified by bilateral kidney stones and benign prostatic hyperplasia of the prostate gland), and congenital disease (such as polycystic kidney disease). Rarely, pinworms infecting the kidney can cause obstructive nephropathy.

In some embodiments, the disclosure relates to methods of monitoring a subject with a renal disease or condition (e.g., Alport syndrome, focal segmental glomerulosclerosis (FSGS), polycystic kidney disease, chronic kidney disease) for albuminuria and/or proteinuria. Elevated protein levels in urine is a hallmark of many renal diseases or conditions. Annual monitoring for albuminuria and proteinuria are initiated beginning at one year of age for at-risk children. Proteinuria comprises a presence of abnormal quantities of protein in the urine. The most sensitive marker of proteinuria is elevated urine albumin (e.g., albuminuria). Albumin typically circulates in the blood, and only a trace of albumin is found in urine of subjects without a renal disease or condition. Moderate albuminuria is typically called microalbuminuria, while severe albuminuria is typically called macroalbuminuria. An albumin level above the upper limit value is called severe albuminuria or macroalbuminuria. In some embodiments, the present disclosure provides methods of treating a subject with one or more of albuminuria, proteinuria, microalbuminuria, and macroalbuminuria. In some embodiments, the present disclosure relates to methods of reducing severity, occurrence and/or duration of one or more of albuminuria, proteinuria, microalbuminuria, and macroalbuminuria in a subject in need thereof.

Measurements of albumin can have different units depending on how such measurements were taken. In some embodiments, albumin in urine is measured as a mass of albumin per time period of urine collected (e.g., mg/24 hr). In some embodiments, albumin in urine is measured as a mass of albumin per volume of urine collected (e.g., mg/L). In some embodiments, albumin in urine is measured as a mass of albumin per mass of creatinine in the urine (e.g., μg/mg of creatinine, termed albumin-creatine ratio, or ACR).

In some embodiments, a subject is administered a urine test to determine presence of a kidney disease or condition (e.g., Alport syndrome, focal segmental glomerulosclerosis (FSGS), polycystic kidney disease, chronic kidney disease). In some embodiments, a urine test comprises collection of urine over a specific time period (e.g., 24 hours). Moderate albuminuria or microalbuminuria comprises a level of albumin detected in the urine from a 24-hour urine collection that is between about 30 and about 300 mg albumin/24 hours and/or a level of albumin detected in the urine from a one minute urine collection that is between about 20 and about 200 μg albumin/1 minute. Severe albuminuria or macroalbuminuria comprises a level of albumin detected in the urine from a 24-hour urine collection that is above about 300 mg albumin/24 hours and/or a level of albumin detected in the urine from a 1 minute urine collection that is above about 200 μg albumin/1 minute. In some embodiments, the disclosure relates to methods of treating a subject with moderate albuminuria or microalbuminuria comprising a level of albumin detected in the urine from a 24-hour urine collection that is between about 30 and about 300 mg albumin/24 hours. In some embodiments, the disclosure relates to methods of treating a subject with moderate albuminuria or microalbuminuria comprising a level of albumin detected in the urine from a one minute urine collection that is between about 20 and about 200 μg albumin/1 minute. In some embodiments, the disclosure relates to methods of treating a subject with severe albuminuria or macroalbuminuria comprising a level of albumin detected in the urine from a 24-hour urine collection that is above about 300 mg albumin/24 hours. In some embodiments, the disclosure relates to methods of treating a subject with severe albuminuria or macroalbuminuria comprising a level of albumin detected in the urine from a 1 minute urine collection that is above about 200 μg albumin/1 minute.

In some embodiments, a urine test comprises a spot test using a single sample of urine. In some embodiments, a urine test comprises a dipstick test. In some embodiments, a urine dipstick test may provide an estimate of the level of albuminuria. In some embodiments, moderate albuminuria or microalbuminuria comprises a level of albumin detected in the urine from a spot sample that is between about 20 and about 200 mg albumin/L urine. In some embodiments, severe albuminuria or macroalbuminuria comprises a level of albumin detected in the urine from a spot sample that is above about 200 mg albumin/L urine. In some embodiments, the disclosure relates to methods of treating a subject with moderate albuminuria or microalbuminuria comprising a level of albumin detected in a urine from a spot sample that is between about 20 and about 200 mg albumin/L urine. In some embodiments, the disclosure relates to methods of treating a subject with severe albuminuria or macroalbuminuria comprising a level of albumin detected in the urine from a spot sample that is above about 200 mg albumin/L urine.

To compensate for variations in urine concentration in spot-check samples (versus a larger sample collection and/or a sample collection over time), comparing the amount of albumin in the sample against the urine concentration of creatinine is useful. This is called the albumin/creatinine ratio (ACR). In some embodiments, presence and/or severity of albuminuria is determined by a ratio of albumin to creatinine in the urine (e.g., albumin-creatinine ratio, ACR, sometimes referred to as urinary albumin-creatinine ratio, or uACR). ACR lower and upper limits can vary between men and women. ACR is measured as a unit of mass of albumin per a unit of mass of creatinine in the urine. In some embodiments, the disclosure provides methods of treating a subject with moderate albuminuria or microalbuminuria comprising an ACR of between about 30 and about 300 mg albumin/g of creatinine. In some embodiments, the disclosure provides methods of treating a subject with severe albuminuria or macroalbuminuria comprising an ACR of above about 300 mg albumin/g of creatinine. In some embodiments, a normal ACR is typically below 30 mg albumin/g creatinine. It is important to note that the units of measure for any albuminuria measurement can differ. For example, ACR may be measured as μg of albumin per mg of creatinine. ACR may also be measured as g of albumin/g creatinine. Units of mg albumin/g creatinine are interchangeable with units of μg albumin/mg creatinine. ACR is sometimes provided without units, if both albumin and creatinine are provided as measurements of mass.

ACR can be measured as mass of albumin per concentration of creatinine in the urine. In some embodiments, the disclosure provides methods of treating moderate albuminuria or microalbuminuria comprising an ACR of between about 2.5 and about 35 mg albumin/mmol of creatinine in a subject in need thereof. In some embodiments, the disclosure provides methods of treating severe albuminuria or macroalbuminuria comprising an ACR of above about 35 mg albumin/mmol of creatinine in a subject in need thereof.

Disease stages describing the extent of renal damage and loss of function in a subject are typically assigned to subjects with renal diseases or conditions. Albuminuria stages are typically measured in terms of an ACR. In some embodiments, the present disclosure relates to methods of treating a subject with Stage A1 albuminuria. Stage A1 albuminuria comprises normal to moderately increased levels of albumin in the urine, with an ACR of less than 30 mg albumin/g creatinine (or less than 3 mg albumin/mmol creatinine). In some embodiments, the present disclosure relates to methods of treating a subject with Stage A2 albuminuria. Stage A2 comprises moderate albuminuria or microalbuminuria, with an ACR of between about 30 and about 300 mg albumin/g creatinine (or between about 3 and about 30 mg albumin/mmol creatinine). In some embodiments, the present disclosure relates to methods of treating a subject with Stage A3 albuminuria. Stage A3 comprises severe albuminuria or macroalbuminuria, with an ACR of greater than 300 mg albumin/g creatinine (or greater than 30 mg albumin/mmol creatinine). In some embodiments, administration of therapy to a subject with a renal disease or condition will delay or prevent development of end stage renal disease. In some embodiments, administration of therapy to a subject with a renal disease or condition will lower said subject's albuminuria stage. In some embodiments, the present disclosure relates to methods of reducing severity, occurrence and/or duration of Stage A1 albuminuria. In some embodiments, the present disclosure relates to methods of reducing severity, occurrence and/or duration of Stage A2 albuminuria. In some embodiments, the present disclosure relates to methods of reducing severity, occurrence and/or duration of Stage A3 albuminuria. In some embodiments, the present disclosure provides methods of treating a subject with Stage A1 albuminuria that delay or prevent progression to Stage A2 albuminuria. In some embodiments, the present disclosure provides methods of treating a subject with Stage A2 albuminuria that delay or prevent progression to Stage A3 albuminuria. In some embodiments, the present disclosure provides methods of delaying and/or preventing worsening of albuminuria stage progression in a subject in need thereof. In some embodiments, the present disclosure provides an improvement in renal damage and/or a downgrade in albuminuria stage classification in a subject in need thereof. In some embodiments, the present disclosure provides methods of improving albuminuria classification in a subject by one or more stages.

In some embodiments, a subject has proteinuria in the nephrotic range prior to treatment with the methods of the invention. In some embodiments, proteinuria in the nephrotic range comprises between about 3 and about 3.5 g of protein in the urine per 24 hours per 1.73 m² body surface area. In some embodiments, a subject with nephrotic syndrome has proteinuria of greater than 3.5 g/24 hrs/1.73 m².

In some embodiments, the disclosure relates to methods of reducing an ACR in a subject with a renal disease or condition, comprising administering to a subject in need thereof an effective amount of an ActRII antagonist. In some embodiments, the method relates to reducing the subject's ACR by between about 0.1 and about 2.5 mg albumin/g creatinine compared to a baseline measurement. In some embodiments, the method relates to reducing the subject's ACR by between about 2.5 and about 3.5 mg albumin/g creatinine compared to a baseline measurement. In some embodiments, the method relates to reducing the subject's ACR by between about 3.5 and about 5.0 mg albumin/g creatinine compared to a baseline measurement. In some embodiments, the method relates to reducing the subject's ACR by between about 5.0 and about 7.5 mg albumin/g creatinine compared to a baseline measurement. In some embodiments, the method relates to reducing the subject's ACR by between about 7.5 and about 10.0 mg albumin/g creatinine compared to a baseline measurement. In some embodiments, the method relates to reducing the subject's ACR by between about 10.0 and about 15.0 mg albumin/g creatinine compared to a baseline measurement. In some embodiments, the method relates to reducing the subject's ACR by between about 15.0 and about 20.0 mg albumin/g creatinine compared to a baseline measurement. In some embodiments, the method relates to reducing the subject's ACR by between about 20.0 and about 25.0 mg albumin/g creatinine compared to a baseline measurement. In some embodiments, the method relates to reducing the subject's ACR by between about 30.0 and about 35.0 mg albumin/g creatinine compared to a baseline measurement. In some embodiments, the method relates to reducing the subject's ACR by between about 40.0 and about 45.0 mg albumin/g creatinine compared to a baseline measurement. In some embodiments, the method relates to reducing the subject's ACR by between about 45.0 and about 50.0 mg albumin/g creatinine compared to a baseline measurement. In some embodiments, the method relates to reducing the subject's ACR by between about 50.0 and about 60.0 mg albumin/g creatinine compared to a baseline measurement. In some embodiments, the method relates to reducing the subject's ACR by between about 60.0 and about 70.0 mg albumin/g creatinine compared to a baseline measurement. In some embodiments, the method relates to reducing the subject's ACR by between about 70.0 and about 80.0 mg albumin/g creatinine compared to a baseline measurement. In some embodiments, the method relates to reducing the subject's ACR by between about 80.0 and about 90.0 mg albumin/g creatinine compared to a baseline measurement. In some embodiments, the method relates to reducing the subject's ACR by between about 90.0 and about 100.0 mg albumin/g creatinine compared to a baseline measurement.

In some embodiments, the disclosure relates to methods of reducing an ACR in a subject with a renal disease or condition, comprising administering to a subject in need thereof an effective amount of an ActRII antagonist. In some embodiments, the method relates to reducing the subject's ACR by greater than or equal to 0.5 g albumin/g creatinine compared to a baseline measurement. In some embodiments, the method relates to reducing the subject's absolute ACR to less than 0.5 g albumin/g creatinine compared to a baseline measurement. In some embodiments, the method relates to reducing the subject's absolute ACR to less than 0.3 g albumin/g creatinine compared to a baseline measurement. In some embodiments, the method relates to reducing the subject's ACR by between about 0.1 and about 2.5 g albumin/g creatinine compared to a baseline measurement. In some embodiments, the method relates to reducing the subject's ACR by between about 0.3 and about 2.5 g albumin/g creatinine compared to a baseline measurement. In some embodiments, the method relates to reducing the subject's ACR by between about 0.5 and about 2.5 g albumin/g creatinine compared to a baseline measurement. In some embodiments, the method relates to reducing the subject's ACR by between about 0.5 and about 3.0 g albumin/g creatinine compared to a baseline measurement. In some embodiments, the method relates to reducing the subject's ACR by between about 2.5 and about 3.5 g albumin/g creatinine compared to a baseline measurement. In some embodiments, the method relates to reducing the subject's ACR by between about 3.5 and about 5.0 g albumin/g creatinine compared to a baseline measurement. In some embodiments, the method relates to reducing the subject's ACR by between about 5.0 and about 7.5 g albumin/g creatinine compared to a baseline measurement. In some embodiments, the method relates to reducing the subject's ACR by between about 7.5 and about 10.0 g albumin/g creatinine compared to a baseline measurement. In some embodiments, the method relates to reducing the subject's ACR by between about 10.0 and about 15.0 g albumin/g creatinine compared to a baseline measurement. In some embodiments, the method relates to reducing the subject's ACR by between about 15.0 and about 20.0 g albumin/g creatinine compared to a baseline measurement. In some embodiments, the method relates to reducing the subject's ACR by between about 20.0 and about 25.0 g albumin/g creatinine compared to a baseline measurement. In some embodiments, the method relates to reducing the subject's ACR by between about 30.0 and about 35.0 g albumin/g creatinine compared to a baseline measurement. In some embodiments, the method relates to reducing the subject's ACR by between about 40.0 and about 45.0 g albumin/g creatinine compared to a baseline measurement. In some embodiments, the method relates to reducing the subject's ACR by between about 45.0 and about 50.0 g albumin/g creatinine compared to a baseline measurement. In some embodiments, the method relates to reducing the subject's ACR by between about 50.0 and about 60.0 g albumin/g creatinine compared to a baseline measurement. In some embodiments, the method relates to reducing the subject's ACR by between about 60.0 and about 70.0 g albumin/g creatinine compared to a baseline measurement. In some embodiments, the method relates to reducing the subject's ACR by between about 70.0 and about 80.0 g albumin/g creatinine compared to a baseline measurement. In some embodiments, the method relates to reducing the subject's ACR by between about 80.0 and about 90.0 g albumin/g creatinine compared to a baseline measurement. In some embodiments, the method relates to reducing the subject's ACR by between about 90.0 and about 100.0 g albumin/g creatinine compared to a baseline measurement.

In some embodiments, the method relates to reducing the subject's ACR by at least 2.5% compared to a baseline measurement. In some embodiments, the method relates to reducing the subject's ACR by at least 5% compared to a baseline measurement (e.g., SOC). In some embodiments, the method relates to reducing the subject's ACR by at least 10% compared to a baseline measurement. In some embodiments, the method relates to reducing the subject's ACR by at least 15% compared to a baseline measurement. In some embodiments, the method relates to reducing the subject's ACR by at least 20% compared to a baseline measurement. In some embodiments, the method relates to reducing the subject's ACR by at least 25% compared to a baseline measurement. In some embodiments, the method relates to reducing the subject's ACR by at least 30% compared to a baseline measurement. In some embodiments, the method relates to reducing the subject's ACR by greater than or equal to 30% compared to a baseline measurement. In some embodiments, the method relates to reducing the subject's ACR by at least 40% compared to a baseline measurement. In some embodiments, the method relates to reducing the subject's ACR by greater than or equal to 30% compared to a baseline measurement. In some embodiments, the method relates to reducing the subject's ACR by at least 50% compared to a baseline measurement. In some embodiments, the method relates to reducing the subject's ACR by greater than or equal to 50% compared to a baseline measurement. In some embodiments, the method relates to reducing the subject's ACR by at least 60% compared to a baseline measurement. In some embodiments, the method relates to reducing the subject's ACR by at least 70% compared to a baseline measurement. In some embodiments, the method relates to reducing the subject's ACR by at least 80% compared to a baseline measurement. In some embodiments, the method relates to reducing the subject's ACR by at least 90% compared to a baseline measurement. In some embodiments, the method relates to reducing the subject's ACR by at least 95% compared to a baseline measurement. In some embodiments, the method relates to reducing the subject's ACR by at least 99% compared to a baseline measurement.

In some embodiments, total urine protein may be measured and compared against creatinine presence in the urine (e.g., UPCR). In some embodiments, UPCR is a measurement of proteinuria. In some embodiments, proteinuria comprises a urinary protein-creatinine ratio (UPCR) of greater than 0.2 mg/mg. In some embodiments, proteinuria comprises a urinary protein excretion of greater than 4 mg/m² per hour. In some embodiments, complete remission (CR) of a renal disease or condition is defined as a consistent UPCR measurement of less than 0.2 g protein/g creatinine. In some embodiments, a partial remission (PR) of a renal disease or condition is defined as having about a 50% reduction from baseline proteinuria and a consistent UPCR of less than about 2 g protein/g creatinine.

In some embodiments, the disclosure relates to methods of reducing an UPCR in a subject with a renal disease or condition, comprising administering to a subject in need thereof an effective amount of an ActRII antagonist. In some embodiments, the method relates to reducing the subject's UPCR by between about 0.2 and about 1 mg urinary protein/mg creatinine. In some embodiments, the method relates to reducing the subject's UPCR by less than 0.5 mg urinary protein/mg creatinine. In some embodiments, the method relates to reducing the subject's UPCR by between about by between about 0.1 and about 100.0 mg urinary protein/mg creatinine. In some embodiments, the method relates to reducing the subject's UPCR by between about 0.1 and about 2.5 mg urinary protein/mg creatinine. In some embodiments, the method relates to reducing the subject's UPCR by between about 2.5 and about 3.5 mg urinary protein/mg creatinine. In some embodiments, the method relates to reducing the subject's UPCR by between about 3.5 and about 5.0 mg urinary protein/mg creatinine. In some embodiments, the method relates to reducing the subject's UPCR by between about 5.0 and about 7.5 mg urinary protein/mg creatinine. In some embodiments, the method relates to reducing the subject's UPCR by between about 7.5 and about 10.0 mg urinary protein/mg creatinine. In some embodiments, the method relates to reducing the subject's UPCR by between about 10.0 and about 15.0 mg urinary protein/mg creatinine. In some embodiments, the method relates to reducing the subject's UPCR by between about 15.0 and about 20.0 mg urinary protein/mg creatinine. In some embodiments, the method relates to reducing the subject's UPCR by between about 20.0 and about 25.0 mg urinary protein/mg creatinine. In some embodiments, the method relates to reducing the subject's UPCR by between about 30.0 and about 35.0 mg urinary protein/mg creatinine. In some embodiments, the method relates to reducing the subject's UPCR by between about 40.0 and about 45.0 mg urinary protein/mg creatinine. In some embodiments, the method relates to reducing the subject's UPCR by between about 45.0 and about 50.0 mg urinary protein/mg creatinine. In some embodiments, the method relates to reducing the subject's UPCR by between about 50.0 and about 60.0 mg urinary protein/mg creatinine. In some embodiments, the method relates to reducing the subject's UPCR by between about 60.0 and about 70.0 mg urinary protein/mg creatinine. In some embodiments, the method relates to reducing the subject's UPCR by between about 70.0 and about 80.0 mg urinary protein/mg creatinine. In some embodiments, the method relates to reducing the subject's UPCR by between about 80.0 and about 90.0 mg urinary protein/mg creatinine. In some embodiments, the method relates to reducing the subject's UPCR by between about 90.0 and about 100.0 mg urinary protein/mg creatinine.

In some embodiments, the disclosure relates to methods of reducing an UPCR in a subject with a renal disease or condition, comprising administering to a subject in need thereof an effective amount of an ActRII antagonist. In some embodiments, the method relates to reducing the subject's UPCR by between about 0.2 and about 1 g urinary protein/g creatinine. In some embodiments, the method relates to reducing the subject's UPCR by less than 0.5 g urinary protein/g creatinine. In some embodiments, the method relates to reducing the subject's UPCR by greater than or equal to 0.5 g urinary protein/g creatinine compared to a baseline measurement. In some embodiments, the method relates to reducing the subject's absolute UPCR to less than 0.5 g urinary protein/g creatinine compared to a baseline measurement. In some embodiments, the method relates to reducing the subject's absolute UPCR to less than 0.3 g urinary protein/g creatinine compared to a baseline measurement. In some embodiments, the method relates to reducing the subject's UPCR by between about by between about 0.1 and about 100.0 g urinary protein/g creatinine. In some embodiments, the method relates to reducing the subject's UPCR by between about 0.1 and about 2.5 g urinary protein/g creatinine. In some embodiments, the method relates to reducing the subject's UPCR by between about 2.5 and about 3.5 g urinary protein/g creatinine. In some embodiments, the method relates to reducing the subject's UPCR by between about 3.5 and about 5.0 g urinary protein/g creatinine. In some embodiments, the method relates to reducing the subject's UPCR by between about 5.0 and about 7.5 g urinary protein/g creatinine. In some embodiments, the method relates to reducing the subject's UPCR by between about 7.5 and about 10.0 g urinary protein/g creatinine. In some embodiments, the method relates to reducing the subject's UPCR by between about 10.0 and about 15.0 g urinary protein/g creatinine. In some embodiments, the method relates to reducing the subject's UPCR by between about 15.0 and about 20.0 g urinary protein/g creatinine. In some embodiments, the method relates to reducing the subject's UPCR by between about 20.0 and about 25.0 g urinary protein/g creatinine. In some embodiments, the method relates to reducing the subject's UPCR by between about 30.0 and about 35.0 g urinary protein/g creatinine. In some embodiments, the method relates to reducing the subject's UPCR by between about 40.0 and about 45.0 g urinary protein/g creatinine. In some embodiments, the method relates to reducing the subject's UPCR by between about 45.0 and about 50.0 g urinary protein/g creatinine. In some embodiments, the method relates to reducing the subject's UPCR by between about 50.0 and about 60.0 g urinary protein/g creatinine. In some embodiments, the method relates to reducing the subject's UPCR by between about 60.0 and about 70.0 g urinary protein/g creatinine. In some embodiments, the method relates to reducing the subject's UPCR by between about 70.0 and about 80.0 g urinary protein/g creatinine. In some embodiments, the method relates to reducing the subject's UPCR by between about 80.0 and about 90.0 g urinary protein/g creatinine. In some embodiments, the method relates to reducing the subject's UPCR by between about 90.0 and about 100.0 g urinary protein/g creatinine.

In some embodiments, administration of therapy decreases urinary protein excretion. In some embodiments, the method relates to reducing the subject's UPCR by at least 2.5% compared to a baseline measurement. In some embodiments, the method relates to reducing the subject's UPCR by at least 5% compared to a baseline measurement. In some embodiments, the method relates to reducing the subject's UPCR by at least 10% compared to a baseline measurement. In some embodiments, the method relates to reducing the subject's UPCR by at least 15% compared to a baseline measurement. In some embodiments, the method relates to reducing the subject's UPCR by at least 20% compared to a baseline measurement. In some embodiments, the method relates to reducing the subject's UPCR by at least 25% compared to a baseline measurement. In some embodiments, the method relates to reducing the subject's UPCR by at least 30% compared to a baseline measurement. In some embodiments, the method relates to reducing the subject's UPCR by greater than or equal to 30% compared to a baseline measurement. In some embodiments, the method relates to reducing the subject's UPCR by at least 40% compared to a baseline measurement. In some embodiments, the method relates to reducing the subject's UPCR by greater than or equal to 40% compared to a baseline measurement. In some embodiments, the method relates to reducing the subject's UPCR by at least 50% compared to a baseline measurement. In some embodiments, the method relates to reducing the subject's UPCR by greater than or equal to 50% compared to a baseline measurement. In some embodiments, the method relates to reducing the subject's UPCR by at least 60% compared to a baseline measurement. In some embodiments, the method relates to reducing the subject's UPCR by at least 70% compared to a baseline measurement. In some embodiments, the method relates to reducing the subject's UPCR by at least 80% compared to a baseline measurement. In some embodiments, the method relates to reducing the subject's UPCR by at least 90% compared to a baseline measurement. In some embodiments, the method relates to reducing the subject's UPCR by at least 95% compared to a baseline measurement. In some embodiments, the method relates to reducing the subject's UPCR by at least 99% compared to a baseline measurement.

A subject may be administered a blood test to determine presence of a kidney disease or condition (e.g., Alport syndrome, focal segmental glomerulosclerosis (FSGS), polycystic kidney disease, chronic kidney disease), by determining how well the kidney is filtering the blood. Typically, a glomerular filtration rate (GFR) is determined, which measures the flow rate of filtered fluid (e.g., blood) through the kidney into the Bowman's capsule. GFR is equal to the clearance rate of when any solute is freely filtered and is neither reabsorbed nor secreted by the kidneys. GFR is therefore a measurement of the quantity of the substance in the urine that originated from a calculable volume of blood, and is typically recorded in units of volume per time, e.g., milliliters per minute (mL/min). A normal range of GFR, adjusted for body surface area, is between about 100 and about 130 mL/min/1.73 m² in men, with an average GFR of 125 mL/min/1.73 m² in men. A normal range of GFR, adjusted for body surface area, is between about 90 and about 120 mL/min/1.73 m² in women younger than age 40. GFR measured by insulin clearance in children under 2 years old is about 110 mL/min/1.73 m², which progressively decreases. After age 40, GFR decreases progressively with age, by between about 0.4 and about 1.2 mL/min per year. GFR may also be calculated by comparative measurements of substances in the blood and urine, estimated using a blood test result (e.g., eGFR). In some embodiments, eGFR is measured using serum creatinine, age, ethnicity, and gender variables. In some embodiments, eGFR is measured using one or more of Cockcroft-Gault formula, Modification of Diet in Renal Disease (MDRD) formula, CKD-EPI formula, Mayo quadratic formula, and Schwartz formula.

A glomerular filtration rate (GFR) ≥60 ml/min/1.73 m² is considered normal in a subject without chronic kidney disease if there is no kidney damage present, which comprises signs of damage seen in blood, urine, or imaging studies which includes lab albumin/creatinine ratio (ACR) ≥30. Subjects with a GFR <60 ml/min/1.73 m² for at least 3 months are diagnosed as having chronic kidney disease.

In general, protein in the urine is regarded as an independent marker for decline of kidney function and cardiovascular disease, and the stages of chronic kidney disease (often used for renal diseases and/or conditions in general) is determined by measuring a subject's GFR. In some embodiments, the present disclosure provides methods of treating stage 1 CKD. Stage 1 CKD comprises normal kidney function, kidney damage with normal or relatively high GFR (e.g., >90 ml/min/1.73 m²), and lower creatinine levels. Kidney damage may be defined as pathological abnormalities or markers of damage, including abnormalities in blood or urine tests or imaging studies. In some embodiments, the present disclosure provides methods of treating stage 2 CKD. Stage 2 CKD comprises mild reduction in kidney function and GFR (e.g., between about 60 and about 89 ml/min/1.73 m²) with kidney damage. In some embodiments, the present disclosure provides methods of treating stage 3 CKD. Stage 3 CKD comprises mild to moderate reduction in kidney function and GFR (e.g., between about 30 and about 59 ml/min/1.73 m²). Stage 3 CKD may be split into stages 3a (e.g., mild to moderate reduction in kidney function and GFR between about 45 and about 59 ml/min/1.73 m² and 3b (e.g., moderate to severe reduction in kidney function and GFR between about 30 and about 44 ml/min/1.73 m². In some embodiments, the present disclosure provides methods of treating stage 3a CKD. In some embodiments, the present disclosure provides methods of treating stage 3b CKD. In some embodiments, the present disclosure provides methods of treating stage 4 CKD. Stage 4 CKD comprises severe reduction in kidney function and GFR (e.g., between about 15 and about 29 ml/min/1.73 m²). In some embodiments, the present disclosure provides methods of treating stage 5 CKD. Stage 5 CKD comprises established kidney failure (e.g., GFR about <15 ml/min/1.73 m²), permanent kidney replacement therapy, end-stage renal disease. (ESRD), and high creatinine levels. In some embodiments, the present disclosure relates to methods of reducing severity, occurrence and/or duration of stage 1 CKD. In some embodiments, the present disclosure relates to methods of reducing severity, occurrence and/or duration of stage 2 CKD. In some embodiments, the present disclosure relates to methods of reducing severity, occurrence and/or duration of stage 3 CKD. In some embodiments, the present disclosure relates to methods of reducing severity, occurrence and/or duration of stage 3a CKD. In some embodiments, the present disclosure relates to methods of reducing severity, occurrence and/or duration of stage 3b CKD. In some embodiments, the present disclosure relates to methods of reducing severity, occurrence and/or duration of stage 4 CKD. In some embodiments, the present disclosure relates to methods of reducing severity, occurrence and/or duration of stage 5 CKD. In some embodiments, the present disclosure provides methods of treating a subject with Stage 1 CKD that delay or prevent progression to Stage 2 CKD. In some embodiments, the present disclosure provides methods of treating a subject with Stage 2 CKD that delay or prevent progression to Stage 3 CKD. In some embodiments, the present disclosure provides methods of treating a subject with Stage 2 CKD that delay or prevent progression to Stage 3a CKD. In some embodiments, the present disclosure provides methods of treating a subject with Stage 2 CKD that delay or prevent progression to Stage 3b CKD. In some embodiments, the present disclosure provides methods of treating a subject with Stage 3a CKD that delay or prevent progression to Stage 3b CKD. In some embodiments, the present disclosure provides methods of treating a subject with Stage 3 CKD that delay or prevent progression to Stage 4 CKD. In some embodiments, the present disclosure provides methods of treating a subject with Stage 3a CKD that delay or prevent progression to Stage 4 CKD. In some embodiments, the present disclosure provides methods of treating a subject with Stage 3b CKD that delay or prevent progression to Stage 4 CKD. In some embodiments, the present disclosure provides methods of treating a subject with Stage 4 CKD that delay or prevent progression to Stage 5 CKD. In some embodiments, the present disclosure provides methods of delaying and/or preventing worsening of CKD stage progression in a subject in need thereof. In some embodiments, the present disclosure provides an improvement in renal damage and/or a downgrade in CKD stage classification in a subject in need thereof. In some embodiments, the present disclosure provides methods of improving CKD classification in a subject by one or more stages.

In certain aspects, the disclosure relates to methods of treating, preventing, or reducing the progression rate and/or severity of a renal disease or condition (e.g., Alport syndrome, focal segmental glomerulosclerosis (FSGS), polycystic kidney diseases, chronic kidney disease) comprising administering to a subject in need thereof an effective amount of an ActRII antagonist. In some embodiments, the subject has stage 1 CKD. In some embodiments, the subject has stage 2 CKD. In some embodiments, the subject has stage 3 CKD. In some embodiments, the subject has stage 3a CKD. In some embodiments, the subject has stage 3b CKD. In some embodiments, the subject has stage 4 CKD. In some embodiments, the subject has stage 5 CKD.

In some embodiments, the disclosure relates to methods of increasing GFR and/or eGFR in a subject with a renal disease or condition, comprising administering to a subject in need thereof an effective amount of an ActRII antagonist. In some embodiments, the method relates to increasing the subject's GFR and/or eGFR by at least 2.5% compared to a baseline measurement. In some embodiments, the method relates to increasing the subject's GFR and/or eGFR by at least 5% compared to a baseline measurement. In some embodiments, the method relates to increasing the subject's GFR and/or eGFR by at least 10% compared to a baseline measurement. In some embodiments, the method relates to increasing the subject's GFR and/or eGFR by at least 15% compared to a baseline measurement. In some embodiments, the method relates to increasing the subject's GFR and/or eGFR by at least 20% compared to a baseline measurement. In some embodiments, the method relates to increasing the subject's GFR and/or eGFR by at least 25% compared to a baseline measurement. In some embodiments, the method relates to increasing the subject's GFR and/or eGFR by at least 30% compared to a baseline measurement. In some embodiments, the method relates to increasing the subject's GFR and/or eGFR by greater than or equal to 30% compared to a baseline measurement. In some embodiments, the method relates to increasing the subject's GFR and/or eGFR by at least 40% compared to a baseline measurement. In some embodiments, the method relates to increasing the subject's GFR and/or eGFR by greater than or equal to 40% compared to a baseline measurement. In some embodiments, the method relates to increasing the subject's GFR and/or eGFR by at least 50% compared to a baseline measurement. In some embodiments, the method relates to increasing the subject's GFR and/or eGFR by at least 60% compared to a baseline measurement. In some embodiments, the method relates to increasing the subject's GFR and/or eGFR by at least 70% compared to a baseline measurement. In some embodiments, the method relates to increasing the subject's GFR and/or eGFR by at least 80% compared to a baseline measurement. In some embodiments, the method relates to increasing the subject's GFR and/or eGFR by at least 90% compared to a baseline measurement. In some embodiments, the method relates to increasing the subject's GFR and/or eGFR by at least 95% compared to a baseline measurement. In some embodiments, the method relates to increasing the subject's GFR and/or eGFR by at least 99% compared to a baseline measurement.

In some embodiments, the method relates to increasing the subject's eGFR and/or GFR by about 1 mL/min/1.73 m² compared to a baseline measurement. In some embodiments, the method relates to increasing the subject's eGFR and/or GFR by about 3 mL/min/1.73 m² compared to a baseline measurement. In some embodiments, the method relates to increasing the subject's eGFR and/or GFR by about 5 mL/min/1.73 m² compared to a baseline measurement. In some embodiments, the method relates to increasing the subject's eGFR and/or GFR by about 7 mL/min/1.73 m² compared to a baseline measurement. In some embodiments, the method relates to increasing the subject's eGFR and/or GFR by about 9 mL/min/1.73 m² compared to a baseline measurement. In some embodiments, the method relates to increasing the subject's eGFR and/or GFR by about 10 mL/min/1.73 m² compared to a baseline measurement. In some embodiments, the method relates to increasing the subject's eGFR and/or GFR by about 15 mL/min/1.73 m² compared to a baseline measurement. In some embodiments, the method relates to increasing the subject's eGFR and/or GFR by about 20 mL/min/1.73 m² compared to a baseline measurement. In some embodiments, the method relates to increasing the subject's eGFR and/or GFR by about 25 mL/min/1.73 m² compared to a baseline measurement. In some embodiments, the method relates to increasing the subject's eGFR and/or GFR by about 30 mL/min/1.73 m² compared to a baseline measurement. In some embodiments, the method relates to increasing the subject's eGFR and/or GFR by about 35 mL/min/1.73 m² compared to a baseline measurement. In some embodiments, the method relates to increasing the subject's eGFR and/or GFR by about 40 mL/min/1.73 m² compared to a baseline measurement. In some embodiments, the method relates to increasing the subject's eGFR and/or GFR by about 45 mL/min/1.73 m² compared to a baseline measurement. In some embodiments, the method relates to increasing the subject's eGFR and/or GFR by about 50 mL/min/1.73 m² compared to a baseline measurement. In some embodiments, the method relates to increasing the subject's eGFR and/or GFR by about 55 mL/min/1.73 m² compared to a baseline measurement. In some embodiments, the method relates to increasing the subject's eGFR and/or GFR by about 60 mL/min/1.73 m² compared to a baseline measurement. In some embodiments, the method relates to increasing the subject's eGFR and/or GFR by about 65 mL/min/1.73 m² compared to a baseline measurement. In some embodiments, the method relates to increasing the subject's eGFR and/or GFR by about 70 mL/min/1.73 m² compared to a baseline measurement. In some embodiments, the method relates to increasing the subject's eGFR and/or GFR by about 75 mL/min/1.73 m² compared to a baseline measurement. In some embodiments, the method relates to increasing the subject's eGFR and/or GFR by about 80 mL/min/1.73 m² compared to a baseline measurement. In some embodiments, the method relates to increasing the subject's eGFR and/or GFR by about 85 mL/min/1.73 m² compared to a baseline measurement. In some embodiments, the method relates to increasing the subject's eGFR and/or GFR by about 90 mL/min/1.73 m² compared to a baseline measurement. In some embodiments, the method relates to increasing the subject's eGFR and/or GFR by about 95 mL/min/1.73 m² compared to a baseline measurement. In some embodiments, the method relates to increasing the subject's eGFR and/or GFR by about 100 mL/min/1.73 m² compared to a baseline measurement.

In some embodiments, the method relates to increasing the subject's eGFR and/or GFR by about 1 mL/min/year compared to a baseline measurement (e.g., SOC). In some embodiments, the method relates to increasing the subject's eGFR and/or GFR by greater than or equal to 1 mL/min/year compared to a baseline measurement. In some embodiments, the method relates to increasing the subject's eGFR and/or GFR by about 2 mL/min/year compared to a baseline measurement. In some embodiments, the method relates to increasing the subject's eGFR and/or GFR by about 3 mL/min/year compared to a baseline measurement. In some embodiments, the method relates to increasing the subject's eGFR and/or GFR by greater than or equal to 3 mL/min/year compared to a baseline measurement. In some embodiments, the method relates to increasing the subject's eGFR and/or GFR by about 5 mL/min/year compared to a baseline measurement. In some embodiments, the method relates to increasing the subject's eGFR and/or GFR by about 7 mL/min/year compared to a baseline measurement. In some embodiments, the method relates to increasing the subject's eGFR and/or GFR by about 9 mL/min/year compared to a baseline measurement. In some embodiments, the method relates to increasing the subject's eGFR and/or GFR by about 10 mL/min/year compared to a baseline measurement. In some embodiments, the method relates to increasing the subject's eGFR and/or GFR by about 15 mL/min/year compared to a baseline measurement. In some embodiments, the method relates to increasing the subject's eGFR and/or GFR by about 20 mL/min/year compared to a baseline measurement. In some embodiments, the method relates to increasing the subject's eGFR and/or GFR by about 25 mL/min/year compared to a baseline measurement. In some embodiments, the method relates to increasing the subject's eGFR and/or GFR by about 30 mL/min/year compared to a baseline measurement. In some embodiments, the method relates to increasing the subject's eGFR and/or GFR by about 35 mL/min/year compared to a baseline measurement. In some embodiments, the method relates to increasing the subject's eGFR and/or GFR by about 40 mL/min/year compared to a baseline measurement. In some embodiments, the method relates to increasing the subject's eGFR and/or GFR by about 45 mL/min/year compared to a baseline measurement. In some embodiments, the method relates to increasing the subject's eGFR and/or GFR by about 50 mL/min/year compared to a baseline measurement. In some embodiments, the method relates to increasing the subject's eGFR and/or GFR by about 55 ml/min/year compared to a baseline measurement. In some embodiments, the method relates to increasing the subject's eGFR and/or GFR by about 60 mL/min/year compared to a baseline measurement. In some embodiments, the method relates to increasing the subject's eGFR and/or GFR by about 65 mL/min/year compared to a baseline measurement. In some embodiments, the method relates to increasing the subject's eGFR and/or GFR by about 70 mL/min/year compared to a baseline measurement. In some embodiments, the method relates to increasing the subject's eGFR and/or GFR by about 75 mL/min/year compared to a baseline measurement. In some embodiments, the method relates to increasing the subject's eGFR and/or GFR by about 80 mL/min/year compared to a baseline measurement. In some embodiments, the method relates to increasing the subject's eGFR and/or GFR by about 85 mL/min/year compared to a baseline measurement. In some embodiments, the method relates to increasing the subject's eGFR and/or GFR by about 90 mL/min/year compared to a baseline measurement. In some embodiments, the method relates to increasing the subject's eGFR and/or GFR by about 95 mL/min/year compared to a baseline measurement. In some embodiments, the method relates to increasing the subject's eGFR and/or GFR by about 100 mL/min/year compared to a baseline measurement. In some embodiments, the method relates to maintaining the subject's eGFR and/or GFR to a level at or near a baseline measurement (e.g., SOC).

In some embodiments, GFR and/or eGFR can be determined by injecting insulin or the insulin-analog sinistrin into plasma. Since both insulin and sinistrin are neither reabsorbed nor secreted by the kidney after glomerular filtration, their rate of excretion is directly proportional to the rate of filtration of water and solutes across the glomerular filter. In some embodiments, GFR and/or eGFR is measured using radioactive substances. In some embodiments, GFR and/or eGFR is measured using chromium-51. In some embodiments, GFR and/or eGFR is measured using renal or plasma clearance of 51Cr-EDTA. In some embodiments, GFR and/or eGFR is measured using technetium-99m. In some embodiments, GFR and/or eGFR is measured using 99mTc-DTPA. A benefit of using radioactive substances is they come close to the ideal properties of insulin (undergoing only glomerular filtration) but can be measured more practically with only a few urine or blood samples. Renal and plasma clearance 51Cr-EDTA has been shown to be accurate in comparison with insulin. In some embodiments, insulin clearance slightly overestimates glomerular function. In early stage renal disease, insulin clearance may remain normal due to hyperfiltration in the remaining nephrons. Incomplete urine collection is an important source of error in insulin clearance measurement.

Creatinine clearance rate (CCr or CrCl) is the volume of blood plasma that is cleared of creatinine per unit time and is a useful measure for approximating the GFR. Creatinine clearance exceeds GFR due to creatinine secretion, which can be blocked by cimetidine. Both GFR and CCr may be accurately calculated by comparative measurements of substances in the blood and urine, or estimated by formulas using just a blood test result (eGFR and eCCr).

In some embodiments, the disclosure relates to methods of reducing total kidney volume in subject with a renal disease or condition, comprising administering to a subject in need thereof an effective amount of an ActRII antagonist. In some embodiments, total kidney volume is measured by ultrasound. In some embodiments, total kidney volume is measured by magnetic resonance imaging (MRI). In some embodiments, total kidney volume reflects a sum volume of the kidney and cysts in renal diseases or disorders (e.g., ADPKD). In some embodiments, the method relates to reducing total kidney volume in the subject by at least 2.5% compared to a baseline measurement. In some embodiments, the method relates to reducing total kidney volume in the subject by at least 5% compared to a baseline measurement. In some embodiments, the method relates to reducing total kidney volume in the subject by at least 10% compared to a baseline measurement. In some embodiments, the method relates to reducing total kidney volume in the subject by at least 15% compared to a baseline measurement. In some embodiments, the method relates to reducing total kidney volume in the subject by at least 20% compared to a baseline measurement. In some embodiments, the method relates to reducing total kidney volume in the subject by at least 25% compared to a baseline measurement. In some embodiments, the method relates to reducing total kidney volume in the subject by at least 30% compared to a baseline measurement. In some embodiments, the method relates to reducing total kidney volume in the subject by at least 40% compared to a baseline measurement. In some embodiments, the method relates to reducing total kidney volume in the subject by at least 50% compared to a baseline measurement. In some embodiments, the method relates to reducing total kidney volume in the subject by at least 60% compared to a baseline measurement. In some embodiments, the method relates to reducing total kidney volume in the subject by at least 70% compared to a baseline measurement. In some embodiments, the method relates to reducing total kidney volume in the subject by at least 80% compared to a baseline measurement. In some embodiments, the method relates to reducing total kidney volume in the subject by at least 90% compared to a baseline measurement. In some embodiments, the method relates to reducing total kidney volume in the subject by at least 95% compared to a baseline measurement. In some embodiments, the method relates to reducing total kidney volume in the subject by at least 99% compared to a baseline measurement.

In some embodiments, blood urea nitrogen (BUN) is measured. In some embodiments, a BUN test measures the amount of urea nitrogen in blood. In some embodiments, if kidneys are impaired, the amount of urea nitrogen can be higher. In some embodiments, the disclosure relates to methods of reducing BUN in a subject with a renal disease or condition, comprising administering to a subject in need thereof an effective amount of an ActRII antagonist. In some embodiments, a normal BUN level for a human is between about 7 mg/dL and about 20 mg/dL. In some embodiments, the method relates to reducing BUN in the subject by at least 2.5% compared to a baseline measurement. In some embodiments, the method relates to reducing BUN in the subject by at least 5% compared to a baseline measurement. In some embodiments, the method relates to reducing BUN in the subject by at least 10% compared to a baseline measurement. In some embodiments, the method relates to reducing BUN in the subject by at least 15% compared to a baseline measurement. In some embodiments, the method relates to reducing BUN in the subject by at least 20% compared to a baseline measurement. In some embodiments, the method relates to reducing BUN in the subject by at least 25% compared to a baseline measurement. In some embodiments, the method relates to reducing BUN in the subject by at least 30% compared to a baseline measurement. In some embodiments, the method relates to reducing BUN in the subject by at least 40% compared to a baseline measurement. In some embodiments, the method relates to reducing BUN in the subject by at least 50% compared to a baseline measurement. In some embodiments, the method relates to reducing BUN in the subject by at least 60% compared to a baseline measurement. In some embodiments, the method relates to reducing BUN in the subject by at least 70% compared to a baseline measurement. In some embodiments, the method relates to reducing BUN in the subject by at least 80% compared to a baseline measurement. In some embodiments, the method relates to reducing BUN in the subject by at least 90% compared to a baseline measurement. In some embodiments, the method relates to reducing BUN in the subject by at least 95% compared to a baseline measurement. In some embodiments, the method relates to reducing BUN in the subject by at least 99% compared to a baseline measurement.

Urine Neutrophil Gelatinase-Associated Lipocalin (NGAL) concentration is an early biomarker of acute kidney injury that is highly sensitive to early injury and is known as a marker of tubular-specific damage. Urine NGAL (or uNGAL) tends to be elevated before serum creatinine levels, allowing for prediction of renal tubular injury. uNGAL increases quantitatively and proportionally according to the severity of renal structural acute kidney injury. In some embodiments, a uNGAL measurement of <50 ng/ml is an indication of low risk of acute kidney injury. In some embodiments, a uNGAL measurement of between about 50 and about 149 ng/mL indicates equivocal risk of acute kidney injury. In some embodiments, a uNGAL measurement of between about 150 and about 300 ng/ml indicates moderate risk of acute kidney injury. In some embodiments, a uNGAL measurement of >300 ng/mL indicates high risk of acute kidney injury.

In some embodiments, the disclosure relates to methods of reducing uNGAL in a subject with a renal disease or condition, comprising administering to a subject in need thereof an effective amount of an ActRII antagonist. In some embodiments, the method relates to reducing the subject's uNGAL by between about 0.1 and about 50 ng/ml. In some embodiments, the method relates to reducing the subject's uNGAL by between about 0.1 and about 100.0 ng/ml. In some embodiments, the method relates to reducing the subject's uNGAL by between about 0.1 and about 150.0 ng/ml. In some embodiments, the method relates to reducing the subject's uNGAL by between about 0.1 and about 200.0 ng/ml. In some embodiments, the method relates to reducing the subject's uNGAL by between about 0.1 and about 250.0 ng/mL. In some embodiments, the method relates to reducing the subject's uNGAL by between about 0.1 and about 300.0 ng/ml. In some embodiments, the method relates to reducing the subject's uNGAL by between about 0.1 and about 25 ng/ml. In some embodiments, the method relates to reducing the subject's uNGAL by between about 25 and about 50 ng/ml. In some embodiments, the method relates to reducing the subject's uNGAL by between about 50 and about 100 ng/mL. In some embodiments, the method relates to reducing the subject's uNGAL by between about 100 and about 150 ng/ml. In some embodiments, the method relates to reducing the subject's uNGAL by between about 150 and about 200 ng/mL. In some embodiments, the method relates to reducing the subject's uNGAL by between about 200 and about 250 ng/mL. In some embodiments, the method relates to reducing the subject's uNGAL by between about 250 and about 300 ng/ml. In some embodiments, the method relates to reducing the subject's uNGAL by more than 300 ng/mL. In some embodiments, the method relates to reducing the subject's uNGAL by between about 0.1 and about 300 ng/mL.

In some embodiments, the disclosure relates to methods of reducing uNGAL in a subject with a renal disease or condition, comprising administering to a subject in need thereof an effective amount of an ActRII antagonist. In some embodiments, the method relates to reducing the subject's uNGAL by at least 2.5% compared to a baseline measurement. In some embodiments, the method relates to reducing the subject's uNGAL by at least 5% compared to a baseline measurement. In some embodiments, the method relates to reducing the subject's uNGAL by at least 10% compared to a baseline measurement. In some embodiments, the method relates to reducing the subject's uNGAL by at least 15% compared to a baseline measurement. In some embodiments, the method relates to reducing the subject's uNGAL by at least 20% compared to a baseline measurement. In some embodiments, the method relates to reducing the subject's uNGAL by at least 25% compared to a baseline measurement. In some embodiments, the method relates to reducing the subject's uNGAL by at least 30% compared to a baseline measurement. In some embodiments, the method relates to reducing the subject's uNGAL by at least 40% compared to a baseline measurement. In some embodiments, the method relates to reducing the subject's uNGAL by at least 50% compared to a baseline measurement. In some embodiments, the method relates to reducing the subject's uNGAL by at least 60% compared to a baseline measurement. In some embodiments, the method relates to reducing the subject's uNGAL by at least 70% compared to a baseline measurement. In some embodiments, the method relates to reducing the subject's uNGAL by at least 80% compared to a baseline measurement. In some embodiments, the method relates to reducing the subject's uNGAL by at least 90% compared to a baseline measurement. In some embodiments, the method relates to reducing the subject's uNGAL by at least 95% compared to a baseline measurement. In some embodiments, the method relates to reducing the subject's uNGAL by at least 99% compared to a baseline measurement.

Optionally, methods disclosed herein for treating, preventing, or reducing the progression rate and/or severity of a renal disease or condition (e.g., Alport syndrome, focal segmental glomerulosclerosis (FSGS), polycystic kidney disease, chronic kidney disease), particularly treating, preventing, or reducing the progression rate and/or severity of one or more complications of a renal disease or condition, may further comprise administering to the subject one or more additional active agents and/or supportive therapies for treating a renal disease or condition. In some embodiments, a subject is administered an additional active agent and/or supportive therapy for treating a renal disease or condition (e.g., Alport syndrome, FSGS, polycystic kidney disease, chronic kidney disease). In some embodiments, ARBs and ACE inhibitors are mainstays of therapy for renal diseases and conditions (e.g., Alport syndrome, FSGS, polycystic kidney disease, chronic kidney disease), with beta-blockade and calcium-channel blockers as second-line therapy. In some embodiments, as third-line therapy, thiazides are used in subjects with normal renal function, while loop diuretics are used in subjects with impaired renal function.

In some embodiments, a subject with a renal disease or condition (e.g., Alport syndrome, focal segmental glomerulosclerosis (FSGS), polycystic kidney disease, chronic kidney disease) is administered an antagonist of the Renin-angiotensin-aldosterone system (RAAS). In some embodiments, RAAS inhibitors include, but are not limited to, angiotensin antagonists (e.g., angiotensin blockade therapy, angiotensin system inhibitor, renin-angiotensin system inhibitor, angiotensin II blockade, angiotensin II type 1 receptor blocker, ARB, angiotensin II receptor antagonist, AT₁ receptor antagonist, or a sartan) and an angiotensin-converting enzyme (ACE) inhibitor. In some embodiments, RAAS antagonism and particularly, the combination of an ACE inhibitor and ARB, will lower GFR by reducing efferent arteriolar vascular tone and thus, reducing intraglomerular capillary pressure, the driving force for glomerular filtration. Thus, a modest decrease in GFR may be tolerated, providing evidence that RAAS antagonism has been achieved.

In some embodiments, a subject is administered an angiotensin antagonist (e.g., angiotensin receptor blocker, ARB), when the subject shows signs of proteinuria. In some embodiments, an ARB reduces proteinuria in subjects with a renal disease or condition. In some embodiments, an angiotensin antagonist diminishes the rate of glomerulosclerosis in subjects with a renal disease or condition. In some embodiments, administration of an ARB decreases renal disease progression. In some embodiments, a subject is administered one or more ARBs selected from the group consisting of losartan, irbesartan, olmesartan, candesartan, valsartan, fimasartan, azilsartan, salprisartan, and telmisartan. In some embodiments a subject is administered losartan. In some embodiments, a subject is administered irbesartan. In some embodiments, a subject is administered olmesartan. In some embodiments, a subject is administered candesartan. In some embodiments, a subject is administered valsartan. In some embodiments, a subject is administered fimasartan. In some embodiments, a subject is administered azilsartan. In some embodiments, a subject is administered salprisartan. In some embodiments, a subject is administered telmisartan.

In some embodiments, a subject with a renal disease or condition is administered an ACE inhibitor. In some embodiments, an ACE inhibitor is selected from the group consisting of benazepril, captopril, enalapril, lisinopril, perindopril, ramipril (e.g., ramipen), trandolapril, and zofenopril. In some embodiments, a subject is administered benazepril. In some embodiments, a subject is administered captopril. In some embodiments, a subject is administered enalapril. In some embodiments, a subject is administered lisinopril. In some embodiments, a subject is administered perindopril. In some embodiments, a subject is administered ramipril. In some embodiments, a subject is administered trandolapril. In some embodiments, a subject is administered zofenopril. In some embodiments, administration of an ACE inhibitor delays dialysis in a subject with proteinuria and normal kidney function. In some embodiments, administration of an ACE inhibitor slows decline in renal function in a subject. In some embodiments, administration of an ACE inhibitor reduces proteinuria in a subject. In some embodiments, administration of an ACE inhibitor decreases kidney damage in a subject.

In some embodiments, a subject with a renal disease or condition is administered an ARB and an ACE inhibitor. In some embodiments, a subject with a renal disease or condition comprising proteinuria and/or microalbuminuria is administered an ARB and an ACE inhibitor.

In some embodiments, an alternative approach to angiotensin antagonism is to combine an ACE inhibitor and/or ARB with an aldosterone antagonist.

In some embodiments, a subject with a renal disease or condition (e.g., primary FSGS) is administered an immunosuppressive treatment. In some embodiments, subjects with a renal disease or condition are treated with immunosuppressive medications. In some embodiments, immunosuppression is not administered to subjects with secondary FSGS. In some embodiments, immunosuppressants are not administered to subjects that do not have primary FSGS. In some embodiments, an immunosuppressant is selected from the group consisting of corticosteroids, calcineurin inhibitors, janus kinase inhibitors, mammalian target of rapamycin (mTOR) inhibitors, IMDH inhibitors, and biologics (including, but not limited to monoclonal antibodies).

In some embodiments, a subject with a renal disease or condition is administered a corticosteroid. In some embodiments, a glucocorticoid is a corticosteroid. In some embodiments, a subject with a renal disease or condition is administered one or more glucocorticoids. In some embodiments, administration of a glucocorticoid is an initial therapy. In some embodiments, a glucocorticoid is selected from the group consisting of beclomethasone, betamethasone, budesonide, cortisone, dexamethasone, hydrocortisone, methylprednisolone, prednisolone, methylprednisone, prednisone, and triamcinolone. In some embodiments, a subject with a renal disease or condition is administered prednisone. In some embodiments, a subject with a renal disease or condition is administered prednisolone.

In some embodiments, a calcineurin inhibitor is selected from the group consisting of cyclosporine (e.g., cyclosporin, ciclosporin, ciclosporine, Neoral, Sandimmune, SangCya) and tacrolimus (e.g., Astagraf XL, Envarsus XR, Prograf). In some embodiments, calcineurin inhibitors are administered to steroid-sensitive subjects who cannot tolerate continued steroid therapy, and/or to subjects with steroid-resistant renal disease (e.g., steroid-resistant FSGS). In some embodiments, a subject with a renal disease or condition is administered cyclosporine. In some embodiments, a subject with a renal disease or condition is administered tacrolimus.

In some embodiments, a subject with a renal disease or condition maybe administered a combination of one or more corticosteroids and/or calcineurin inhibitors. In some embodiments, a subject with a kidney disease or condition may be administered cyclosporine and prednisone. In some embodiments, a subject with a renal disease or condition is administered tacrolimus and prednisone. In some embodiments, cyclosporine and prednisone are administered to preserve renal function assessed as creatinine clearance.

In some embodiments, treatment with mycophenolate mofetil (MMF) combined with glucocorticoids may be beneficial in subjects who cannot take calcineurin inhibitors. In some embodiments, a subject with a renal disease or condition is administered mycophenolate mofetil (MMF) in combination with one or more glucocorticoids. In some embodiments, a subject with a renal disease or condition is administered MMF and prednisone. In some embodiments, a subject with a renal disease or condition is administered prednisolone and MMF.

In some embodiments, a subject with a renal disease or condition is administered cyclophosphamide and/or prednisone. In some embodiments, a subject with a renal disease or condition is administered prednisolone and/or chlorambucil. In some embodiments, a subject with a renal disease or condition is administered cyclophosphamide. In some embodiments, a subject with a renal disease or condition is administered chlorambucil.

In some embodiments, a janus kinase inhibitor is tofacitinib (e.g., Xeljanz).

In some embodiments, an mTOR inhibitor is selected from the group consisting of sirolimus (e.g., Rapamune) and everolimus (e.g., Afinitor, Zortress).

In some embodiments, an IMDH inhibitor is selected from the group consisting of azathioprinc (e.g., Azasan, Imuran), leflunomide (e.g., Arava), and mycophenolate (e.g., CellCept, Myfortic).

In some embodiments, a biologic is selected from the group consisting of abatacept (e.g., Orencia), adalimumab (e.g., Humira), anakinra (e.g., Kineret), basiliximab (e.g., Simulect), certolizumab (e.g., Cimzia), daclizumab (e.g., Zinbryta), etanercept (e.g., Enbrel), fresolimumab, golimumab (e.g., Simponi), infliximab (e.g., Remicade), ixckizumab (e.g., Taltz), natalizumab (e.g., Tysabri), rituximab (e.g., Rituxan), secukinumab (e.g., Cosentyx), tocilizumab (e.g., Actemra), ustekinumab (e.g., Stelara), and vedolizumab (e.g., Entyvio).

In some embodiments, a subject with a renal disease or condition (e.g., Alport syndrome, focal segmental glomerulosclerosis (FSGS), polycystic kidney disease, chronic kidney disease) is administered a statin (e.g., benazepril, valsartan, Fluvastatin, pravastatin).

In some embodiments, a subject with a renal disease or condition (e.g., Alport syndrome, focal segmental glomerulosclerosis (FSGS), polycystic kidney disease, chronic kidney disease) is administered lademirsen. Lademirsen is an anti-miRNA-21.

In some embodiments, a subject with a renal disease or condition (e.g., Alport syndrome, FSGS, polycystic kidney disease, chronic kidney disease) is administered bardoxolone methyl. Bardoxolone methyl is an activator of the KEAPI-Nrf2 pathway and bardoxolone methyl also inhibits the pro-inflammatory transcription factor NF-κB.

In some embodiments, a subject with a renal disease or condition (e.g., Alport syndrome, FSGS, polycystic kidney disease, chronic kidney disease) is administered Achtar gel. Achtar gel was approved in the 1950s by the US Food and Drug Administration for nephrotic syndrome under criteria that were less stringent than required today. In some embodiments, some case studies suggest limited efficacy of Aethar in some subjects with FSGS. In some embodiments, a subject with FSGS is administered Achtar gel.

In some embodiments, a subject with ADPKD is administered Tolvaptan (e.g., OPC-41061). In some embodiments, Tolvaptan has demonstrated a slower decline than placebo in the eGFR over a one year period in patients with late-stage chronic kidney disease but is associated with elevations of bilirubin and alanine aminotransferase levels.

In some embodiments, a subject a renal disease or condition (e.g., Alport syndrome, FSGS, polycystic kidney disease, chronic kidney disease) is administered one or more of abatacept in combination with sparsentan, aliskiren, allopurinol, ANG-3070, atorvastatin, bleselumab, bosutinib, CCX140-B, CXA-10, D6-25-hydroxyvitamin D3, dapagliflozin, dexamethasone in combination with MMF, emodin, FG-3019, FK506, FK-506 and MMF, FT-011, galactose, GC1008, GFB-887, isotretinoin, lanreotide, levamisole, lixivaptan, losmapimod, metformin, mizorbine, N-acetylmannosamine, octreotide, paricalcitol, PF-06730512, pioglitazone, propagermanium, propagermanium and irbesartan, rapamune, rapamycin, RE-021 (e.g., sparsentan), RG012, rosiglitazone (e.g., Avandia), saquinivir, SAR339375, somatostatin, spironolactone, tesevatinib (KD019), tetracosactin, tripterygium wilfordii (TW), valproic acid, VAR-200, venglustat (GZ402671), verinurad, voclosporin, and VX-147.

In some embodiments, a subject with a renal disease or condition (e.g., Alport syndrome, FSGS, polycystic kidney disease, chronic kidney disease) undergoes kidney dialysis. In some embodiments, a subject with a renal disease or condition (e.g., Alport syndrome, focal segmental glomerulosclerosis (FSGS), polycystic kidney disease, chronic kidney disease) undergoes a kidney transplant. In some embodiments, a subject with ESRD undergoes a kidney transplantation. In some embodiments, a subject with a kidney transplant does not experience recurrent renal disease. In some embodiments, a subject with a kidney transplant contracts anti-glomerular basement membrane antibody disease. In some embodiments, anti-glomerular basement membrane antibody disease occurs within one year after kidney transplantation. In some embodiments, a subject with anti-glomerular basement membrane antibody disease is administered methylprednisone and/or cyclophosphamide. In some embodiments, a subject with anti-glomerular basement membrane antibody disease undergoes plasmapheresis.

In some embodiments, a subject with a renal disease or condition (e.g., Alport syndrome, FSGS, polycystic kidney disease, chronic kidney disease) is administered mesenchymal stem cell therapy. In some embodiments, a subject with a renal disease or condition (e.g., Alport syndrome, FSGS, polycystic kidney disease, chronic kidney disease) is administered bone marrow stem cells. In some embodiments, a subject with a renal disease or condition (e.g., Alport syndrome, FSGS, polycystic kidney disease, chronic kidney disease) undergoes lipoprotein removal. In some embodiments, a subject with a renal disease or condition (e.g., Alport syndrome, FSGS, polycystic kidney disease, chronic kidney disease) is administered a Liposorber LA-15 device. In some embodiments, a subject with a renal disease or condition (e.g., Alport syndrome, FSGS, polycystic kidney disease, chronic kidney disease) undergoes plasmapheresis. In some embodiments, a subject with a renal disease or condition (e.g., Alport syndrome, FSGS, polycystic kidney disease, chronic kidney disease) undergoes plasma exchange. In some embodiments, a subject with a renal disease or condition (e.g., Alport syndrome, FSGS, polycystic kidney disease, chronic kidney disease) undergoes a change in diet (e.g., dietary sodium intake).

In some embodiments, methods of the present disclosure delay clinical worsening of a renal disease or condition (e.g., Alport syndrome, FSGS, polycystic kidney disease, chronic kidney disease) in a subject. In some embodiments, methods of the present disclosure reduce the risk of hospitalization for one or more complications associated with a renal disease or condition (e.g., Alport syndrome, FSGS, polycystic kidney disease, chronic kidney disease).

### 7. Pharmaceutical Compositions & Modes of Administration

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than the ActRII antagonists which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more ActRII antagonists in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind described herein.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Further, the composition may be encapsulated or injected in a form for delivery to a target tissue site. In certain embodiments, compositions of the present invention may include a matrix capable of delivering one or more therapeutic compounds (e.g., ActRII antagonists) to a target tissue site, providing a structure for the developing tissue and optimally capable of being resorbed into the body. For example, the matrix may provide slow release of the ActRII antagonist. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments, methods of the invention can be administered for orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the present invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., ActRII antagonists). The various factors include, but are not limited to, the patient's age, sex, and diet, the severity disease, time of administration, and other clinical factors. Optionally, the dosage may vary with the type of matrix used in the reconstitution and the types of compounds in the composition. The addition of other known growth factors to the final composition, may also affect the dosage. Progress can be monitored by periodic assessment of bone growth and/or repair, for example, X-rays (including DEXA), histomorphometric determinations, and tetracycline labeling. In some embodiments, ActRII antagonists of the disclosure are administered at 0.1-2.0 mg/kg. In some embodiments, ActRII antagonists of the disclosure are administered at 0.1 mg/kg. In some embodiments, ActRII antagonists of the disclosure are administered at 0.2 mg/kg. In some embodiments, ActRII antagonists of the disclosure are administered at 0.3 mg/kg. In some embodiments, ActRII antagonists of the disclosure are administered at 0.4 mg/kg. In some embodiments, ActRII antagonists of the disclosure are administered at 0.5 mg/kg. In some embodiments, ActRII antagonists of the disclosure are administered at 0.6 mg/kg. In some embodiments, ActRII antagonists of the disclosure are administered at 0.7 mg/kg. In some embodiments, ActRII antagonists of the disclosure are administered at 0.8 mg/kg. In some embodiments, ActRII antagonists of the disclosure are administered at 0.9 mg/kg. In some embodiments, ActRII antagonists of the disclosure are administered at 1.0 mg/kg. In some embodiments, ActRII antagonists of the disclosure are administered at 1.1 mg/kg. In some embodiments, ActRII antagonists of the disclosure are administered at 1.2 mg/kg. In some embodiments, ActRII antagonists of the disclosure are administered at 1.3 mg/kg. In some embodiments, ActRII antagonists of the disclosure are administered at 1.4 mg/kg. In some embodiments, ActRII antagonists of the disclosure are administered at 1.5 mg/kg. In some embodiments, ActRII antagonists of the disclosure are administered at 1.6 mg/kg. In some embodiments, ActRII antagonists of the disclosure are administered at 1.7 mg/kg. In some embodiments, ActRII antagonists of the disclosure are administered at 1.8 mg/kg. In some embodiments, ActRII antagonists of the disclosure are administered at 1.9 mg/kg. In some embodiments, ActRII antagonists of the disclosure are administered at 2.0 mg/kg. In some embodiments, ActRII antagonists of the disclosure are administered at 2.25 mg/kg. In some embodiments, ActRII antagonists of the disclosure are administered at 2.50 mg/kg. In some embodiments, ActRII antagonists of the disclosure are administered at 2.75 mg/kg. In some embodiments, ActRII antagonists of the disclosure are administered at 3.00 mg/kg. In some embodiments, ActRII antagonists of the disclosure are administered at 3.25 mg/kg. In some embodiments, ActRII antagonists of the disclosure are administered at 3.50 mg/kg. In some embodiments, ActRII antagonists of the disclosure are administered at 3.75 mg/kg. In some embodiments, ActRII antagonists of the disclosure are administered at 4.00 mg/kg. In some embodiments, ActRII antagonists of the disclosure are administered at 4.25 mg/kg. In some embodiments, ActRII antagonists of the disclosure are administered at 4.50 mg/kg. In some embodiments, ActRII antagonists of the disclosure are administered at 4.75 mg/kg. In some embodiments, ActRII antagonists of the disclosure are administered at 5.00 mg/kg. In some embodiments, ActRII antagonists of the disclosure are administered at 5.00 mg/kg. In some embodiments, ActRII antagonists of the disclosure are administered at 6.00 mg/kg. In some embodiments, ActRII antagonists of the disclosure are administered at 7.00 mg/kg. In some embodiments, ActRII antagonists of the disclosure are administered at 8.00 mg/kg. In some embodiments, ActRII antagonists of the disclosure are administered at 9.00 mg/kg. In some embodiments, ActRII antagonists of the disclosure are administered at 10.00 mg/kg. In some embodiments, ActRII antagonists of the disclosure are administered at 20.00 mg/kg. In some embodiments, ActRII antagonists of the disclosure are administered at 30.00 mg/kg. In some embodiments, ActRII antagonists of the disclosure are administered at least 0.25 mg/kg. In some embodiments, a dose of one or more ActRII antagonists comprises between about 0.25 mg/to about 30.00 mg/kg. The various factors include, but are not limited to, the patient's age, sex, and diet, the severity disease, time of administration, and other clinical factors. Optionally, the dosage may vary with the type of matrix used in the reconstitution and the types of compounds in the composition.

In some embodiments, a dose of one or more ActRII antagonists of the present disclosure is administered once every day. In some embodiments, a dose of one or more ActRII antagonists of the present disclosure is administered once every two days. In some embodiments, a dose of one or more ActRII antagonists of the present disclosure is administered once every three days. In some embodiments, a dose of one or more ActRII antagonists of the present disclosure is administered once every four days. In some embodiments, a dose of one or more ActRII antagonists of the present disclosure is administered once every five days. In some embodiments, a dose of one or more ActRII antagonists of the present disclosure is administered once every six days. In some embodiments, a dose of one or more ActRII antagonists of the present disclosure is administered once every week. In some embodiments, a dose of one or more ActRII antagonists of the present disclosure is administered once every two weeks. In some embodiments, a dose of one or more ActRII antagonists of the present disclosure is administered once every three weeks. In some embodiments, a dose of one or more ActRII antagonists of the present disclosure is administered once every four weeks. In some embodiments, a dose of one or more ActRII antagonists of the present disclosure is administered once every other week. In some embodiments, a dose of one or more ActRII antagonists of the present disclosure is administered once every month. In some embodiments, a dose of one or more ActRII antagonists of the present disclosure is administered once every two months. In some embodiments, a dose of one or more ActRII antagonists of the present disclosure is administered once every three months. In some embodiments, a dose of one or more ActRII antagonists of the present disclosure is administered once every four months. In some embodiments, a dose of one or more ActRII antagonists of the present disclosure is administered once every five months. In some embodiments, a dose of one or more ActRII antagonists of the present disclosure is administered once every six months. In some embodiments, a dose of one or more ActRII antagonists of the present disclosure is administered once every year.

In some embodiments, a dose of one or more ActRII antagonists of the present disclosure is administered twice every day. In some embodiments, a dose of one or more ActRII antagonists of the present disclosure is administered twice every two days. In some embodiments, a dose of one or more ActRII antagonists of the present disclosure is administered twice every three days. In some embodiments, a dose of one or more ActRII antagonists of the present disclosure is administered twice every four days. In some embodiments, a dose of one or more ActRII antagonists of the present disclosure is administered twice every five days. In some embodiments, a dose of one or more ActRII antagonists of the present disclosure is administered twice every six days. In some embodiments, a dose of one or more ActRII antagonists of the present disclosure is administered twice every week. In some embodiments, a dose of one or more ActRII antagonists of the present disclosure is administered twice every two weeks. In some embodiments, a dose of one or more ActRII antagonists of the present disclosure is administered twice every three weeks. In some embodiments, a dose of one or more ActRII antagonists of the present disclosure is administered twice every four weeks. In some embodiments, a dose of one or more ActRII antagonists of the present disclosure is administered twice every other week. In some embodiments, a dose of one or more ActRII antagonists of the present disclosure is administered twice every month. In some embodiments, a dose of one or more ActRII antagonists of the present disclosure is administered twice every two months. In some embodiments, a dose of one or more ActRII antagonists of the present disclosure is administered twice every three months. In some embodiments, a dose of one or more ActRII antagonists of the present disclosure is administered twice every four months. In some embodiments, a dose of one or more ActRII antagonists of the present disclosure is administered twice every five months. In some embodiments, a dose of one or more ActRII antagonists of the present disclosure is administered twice every six months. In some embodiments, a dose of one or more ActRII antagonists of the present disclosure is administered twice every year.

In some embodiments, a dose of one or more ActRII antagonists of the present disclosure is administered three times every day. In some embodiments, a dose of one or more ActRII antagonists of the present disclosure is administered three times every two days. In some embodiments, a dose of one or more ActRII antagonists of the present disclosure is administered three times every three days. In some embodiments, a dose of one or more ActRII antagonists of the present disclosure is administered three times every four days. In some embodiments, a dose of one or more ActRII antagonists of the present disclosure is administered three times every five days. In some embodiments, a dose of one or more ActRII antagonists of the present disclosure is administered three times every six days. In some embodiments, a dose of one or more ActRII antagonists of the present disclosure is administered three times every week. In some embodiments, a dose of one or more ActRII antagonists of the present disclosure is administered three times every two weeks. In some embodiments, a dose of one or more ActRII antagonists of the present disclosure is administered three times every three weeks. In some embodiments, a dose of one or more ActRII antagonists of the present disclosure is administered three times every four weeks. In some embodiments, a dose of one or more ActRII antagonists of the present disclosure is administered three times every other week. In some embodiments, a dose of one or more ActRII antagonists of the present disclosure is administered three times every month. In some embodiments, a dose of one or more ActRII antagonists of the present disclosure is administered three times every two months. In some embodiments, a dose of one or more ActRII antagonists of the present disclosure is administered three times every three months. In some embodiments, a dose of one or more ActRII antagonists of the present disclosure is administered three times every four months. In some embodiments, a dose of one or more ActRII antagonists of the present disclosure is administered three times every five months. In some embodiments, a dose of one or more ActRII antagonists of the present disclosure is administered three times every six months. In some embodiments, a dose of one or more ActRII antagonists of the present disclosure is administered three times every year.

In some embodiments, the present disclosure provides methods of treating renal diseases or conditions, comprising administering an ActRII antagonist to a subject in need thereof, wherein the ActRII antagonist is administered in a dose of between about 0.25 mg/kg and about 30.00 mg/kg to a subject in need thereof. In some embodiments, the ActRII antagonist is administered at least once every week. In some embodiments, the ActRII antagonist is administered at least once every three weeks. In some embodiments, the ActRII antagonist is administered at least once every four weeks. In some embodiments, the ActRII antagonist is administered subcutaneously.

In certain embodiments, the present invention also provides gene therapy for the in vivo production of ActRII antagonists (e.g., ActRII polypeptides). Such therapy would achieve its therapeutic effect by introduction of the ActRII antagonist polynucleotide sequences into cells or tissues having the disorders as listed above. Delivery of ActRII antagonist polynucleotide sequences can be achieved using a recombinant expression vector such as a chimeric virus or a colloidal dispersion system. In some embodiments, targeted liposomes are used for therapeutic delivery of ActRII antagonist polynucleotide sequences.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. In some embodiments, targeting is accomplished by using an antibody. Those of skill in the art will recognize that specific polynucleotide sequences can be inserted into the retroviral genome or attached to a viral envelope to allow target specific delivery of the retroviral vector containing the ActRII antagonist. In one embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for ActRII antagonist polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. One colloidal system useful for this invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art. The disclosure provides formulations that may be varied to include acids and bases to adjust the pH; and buffering agents to keep the pH within a narrow range.

### 8. Kits

The present disclosure provides a kit comprising a lyophilized polypeptide and an injection device. In certain embodiments, the lyophilized polypeptide comprises an ActRII antagonist (e.g., an antagonist of one or more of activin A, activin B, activin AB, activin AC, BMP6, BMP7, BMP9, BMP10, GDF3, GDF8, GDF11, and one or more SMAD proteins), or fragments, functional variants, or modified forms thereof. In certain embodiments, the polypeptide binds to one or more ligands selected from the group consisting of activin A, activin B, and GDF11. In certain such embodiments, the polypeptide further binds to one or more ligands selected from the group consisting of BMP10, GDF8, and BMP6. In certain embodiments, the polypeptide binds to activin and/or GDF11.

In some embodiments, the lyophilized polypeptide comprises a polypeptide that comprises, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of any one of SEQ ID NOs: 9, 10, 11, 36, 39, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 283, 304, 408, and 409.

In some embodiments, the lyophilized polypeptide comprises a polypeptide that comprises, consists essentially of, or consists of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 4, 5, 6, 40, 42, 45, 46, 47, 48, 69, 74, 77, 78, 79, 138, 282, 289, 290, 291, 292, 293, 294, 295, 296, 297, 298, 299, 300, 301, 302, 303, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, and 407.

In certain embodiments of the foregoing, the lyophilized polypeptide comprises a fusion protein further comprising an Fc domain of an immunoglobulin. In certain such embodiments, the Fc domain of the immunoglobulin is an Fc domain of an IgG1 immunoglobulin. In other embodiments, the fusion protein further comprises a linker domain positioned between the polypeptide domain and the Fc domain of the immunoglobulin. In certain embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In certain embodiments, the linker domain comprises TGGG (SEQ ID NO: 23).

In certain embodiments, the lyophilized polypeptide is part of a homodimer protein complex. In certain embodiments, the lyophilized polypeptide is part of a heterodimer protein complex.

In certain embodiments, the polypeptide is glycosylated.

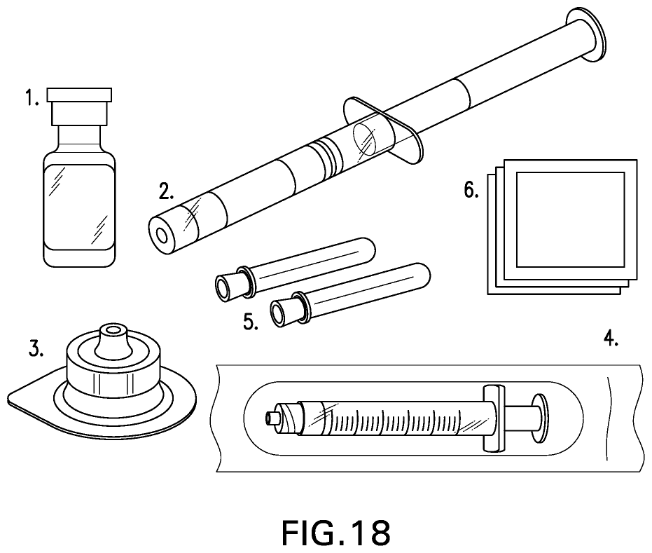

The present disclosure provides a kit comprising a sterile powder comprising a lyophilized polypeptide as disclosed herein and an injection device. In some embodiments of the kits disclosed herein, the sterile powder comprising a lyophilized polypeptide is pre-filled in one or more containers, such as one or more vials (FIG. 18 (1)).

In certain embodiments, the pH range for the sterile powder comprising a lyophilized polypeptide is from 7 to 8. In some embodiments, the sterile powder comprising a lyophilized polypeptide further comprises a buffering agent. In some embodiments, the buffering agent may be added in an amount of at least 10 mM. In some embodiments, the buffering agent may be added in an amount in the range of between about 10 mM to about 200 mM. In some embodiments, the buffering agent comprises citric acid monohydrate and/or trisodium citrate dehydrate.

In some embodiments, the sterile powder comprising a lyophilized polypeptide further comprises a surfactant. In some embodiments, the surfactant comprises a polysorbate. In some embodiments, the surfactant comprises polysorbate 80.

In some embodiments, the sterile powder comprising a lyophilized polypeptide further comprises a lyoprotectant. In some embodiments, the lyoprotectant comprises a sugar, such as disaccharides (e.g., sucrose). In some embodiments, the lyoprotectant comprises sucrose, trehalose, mannitol, polyvinylpyrrolidone (PVP), dextrose, and/or glycine. In some embodiments, the lyoprotectant comprises sucrose. In some embodiments, the sterile powder comprises the lyoprotectant and lyophilized polypeptide in a weight ratio of at least 1:1 lyophilized polypeptide to lyoprotectant. In some embodiments, the sterile powder comprises the lyoprotectant and lyophilized polypeptide in a weight ratio of from 1:1 to 1:10 lyophilized polypeptide to lyoprotectant. In some embodiments, the sterile powder comprises the lyoprotectant and lyophilized polypeptide in a weight ratio of 1:1, 1:2, 1:3, 1:4, 1:5, 1:6, 1:7, 1:8, 1:9, or 1:10 lyophilized polypeptide to lyoprotectant. In some embodiments, the sterile powder comprises the lyoprotectant and lyophilized polypeptide in a weight ratio of 1:6 lyophilized polypeptide to lyoprotectant. In certain embodiments of the foregoing, the sterile powder comprises lyoprotectant in an amount sufficient to stabilize the lyophilized polypeptide.

In certain embodiments of the kits disclosed herein, the injection device comprises a syringe (FIG. 18 (2)). In certain such embodiments, the syringe is pre-filled with a reconstitution solution. In some embodiments, the reconstitution solution comprises a pharmaceutically acceptable carrier and/or excipient. In some embodiments, the pharmaceutically acceptable carrier comprises aqueous solutions such as water, physiologically buffered saline, or other solvents or vehicles such as glycols, glycerol, oils or injectable organic esters. In some embodiments, the pharmaceutically acceptable excipient comprises a pharmaceutically acceptable excipient selected from calcium phosphates, calcium carbonates, calcium sulfates, halites, metallic oxides, sugars, sugar alcohols, starch, glycols, povidones, mineral hydrocarbons, acrylic polymers, fatty alcohols, mineral stearates, glycerin, and/or lipids. In certain embodiments, the reconstitution solution comprises pharmaceutically acceptable sterile isotonic aqueous or nonaqueous solutions, dispersions, suspensions or emulsions. In certain such embodiments, the reconstitution solution comprises antioxidants, buffers, bacteriostats, and/or solutes which render the formulation isotonic with the blood of the intended recipient. In other embodiments, the reconstitution solution comprises suspending or thickening agents.

In certain embodiments of the kits disclosed herein, the kit further comprises a vial adapter (FIG. 18 (3)). In some embodiments, the vial pre-filled with sterile powder comprising a lyophilized polypeptide attaches to one end of the vial adapter. In some embodiments, the syringe pre-filled with a reconstitution solution as disclosed herein attaches to an end of the vial adapter. In some embodiments, the syringe pre-filled with a reconstitution solution as disclosed herein and the vial pre-filled with sterile powder comprising a lyophilized polypeptide are attached to opposite ends of the vial adapter. In some embodiments, the reconstitution solution is transferred from the pre-filled syringe to the vial. In some embodiments, transfer of the reconstitution solution to the vial pre-filled with sterile powder comprising a lyophilized polypeptide reconstitutes the lyophilized polypeptide into a sterile injectable solution. In some embodiments, the lyophilized polypeptide is reconstituted into a sterile injectable solution. In some embodiments, the lyophilized polypeptide is reconstituted into a sterile injectable solution prior to use.

In other embodiments of the kits disclosed herein, the kit further comprises a pump apparatus. In certain embodiments, the pump apparatus comprises an electromechanical pumping assembly. In certain embodiments, the pump apparatus comprises a reservoir for holding a sterile injectable solution. In certain embodiments, the reservoir holds 1 mL of sterile injectable solution. In certain embodiments, the pump apparatus comprises one or more vials or cartridges comprising a sterile injectable solution. In certain embodiments, the vials or cartridges are prefilled with sterile injectable solution. In certain embodiments, the vials or cartridges comprise sterile injectable solution reconstituted from a lyophilized polypeptide. In certain embodiments, the reservoir is coupled to the vial or cartridge. In certain embodiments, the vial or cartridge holds 1-20 mL of sterile injectable solution. In certain embodiments, the electromechanical pumping assembly comprises a pump chamber. In certain embodiments, the electromechanical pumping assembly is coupled to the reservoir. In certain embodiments, the sterile injectable solution is received from the reservoir into the pump chamber. In some embodiments, the electromechanical pumping assembly comprises a plunger that is disposed such that sterile injectable solution in the pump chamber is in direct contact with the plunger. In certain embodiments, a sterile injectable solution is received from the reservoir into the pump chamber during a first pumping phase, and is delivered from the pump chamber to a subject during a second pumping phase. In certain embodiments, the electromechanical pumping assembly comprises control circuitry. In certain embodiments, control circuitry drives the plunger to (a) draw the sterile injectable solution into the pump chamber during the first pumping phase and (b) deliver the sterile injectable solution from the pump chamber in a plurality of discrete motions of the plunger during the second pumping phase, thereby delivering the therapeutic substance to the subject in a plurality of controlled and discrete dosages throughout the second pumping phase. In certain embodiments, a cycle of alternating the first and second pumping phases may be repeated until a desired dose is administered. In certain embodiments, the pump apparatus is coupled to a wearable patch. In certain embodiments, the pump apparatus is a wearable pump apparatus. In some embodiments, the pump apparatus administers a dose every 3 weeks. In some embodiments, the pump apparatus administers the dose via subcutaneous injection.

The present disclosure provides a kit used for reconstituting a lyophilized polypeptide into a sterile injectable solution. In certain embodiments, the resulting sterile injectable solution is useful in the methods disclosed herein.

In certain embodiments of the kits disclosed herein, the kit further comprises an injectable device for use in administering the sterile injectable solution parenterally (FIGS. 18 (1, 2, 3, 4, and 5)). In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection. In some embodiments, the sterile injectable solution is self-administered. In some embodiments, the sterile injectable solution comprises a therapeutically effective dose. In some embodiments, the therapeutically effective dose comprises a weight based dose. In some embodiments, the weight based dose is 0.3 mg/kg. In some embodiments, the weight based dose is 0.7 mg/kg.

In some embodiments of the kits disclosed herein, the kit further comprises one or more vials or cartridges containing the lyophilized polypeptide. In some embodiments, the kit comprises at least two vials or cartridges containing the lyophilized polypeptide. In some embodiments, the kit comprises at least three vials or cartridges containing the lyophilized polypeptide. In some embodiments, the two vials can contain the same or different amounts of the lyophilized polypeptide. In some embodiments, the vials or cartridges comprise a vial or cartridge containing between 25 mg to 60 mg of lyophilized polypeptide. In some embodiments, at least one of the vials or cartridges comprise a vial or cartridge containing 60 mg of lyophilized polypeptide. In some embodiments, at least one of the vials or cartridges comprise a vial or cartridge containing 45 mg of lyophilized polypeptide. In some embodiments, at least one of the vials or cartridges comprise a vial or cartridge containing 30 mg of lyophilized polypeptide. In some embodiments, at least one of the vials or cartridges comprise a vial or cartridge containing 25 mg of lyophilized polypeptide. In some embodiments, a first vial or cartridge contains 45 mg of lyophilized polypeptide and a second vial or cartridge contains 60 mg of lyophilized polypeptide. In some embodiments, a first vial or cartridge contains 30 mg of lyophilized polypeptide and a second vial or cartridge contains 60 mg of lyophilized polypeptide. In some embodiments, a first vial or cartridge contains 30 mg of lyophilized polypeptide, a second vial or cartridge contains 45 mg of lyophilized polypeptide, and a third vial or cartridge contains 60 mg of lyophilized polypeptide. In some embodiments, a first vial or cartridge contains 25 mg of lyophilized polypeptide, a second vial or cartridge contains 45 mg of lyophilized polypeptide, and a third vial or cartridge contains 60 mg of lyophilized polypeptide. In some embodiments, the one or more vials or cartridges are refrigerated at 2-8° C.

### 9. Additional Sequences

TABLE 8

| SEQ | |
|---|---|
| ID | |
| NO | Amino Acid Sequence |
| 211 | GAILGRSETQECLFFNANWEKDRTNQTGVEPCYGD |
| | KDKRRHCFATWKNISGSIEIVKQGCWLDDINCYDR |
| | TDCVEKKDSPEVYFCCCEGNMCNEKFSYFPEMEVT |
| | QPTS |
| 284 | MEWSWVFLFFLSVTTGVHSDKTHTCPPCPAPELLG |
| | GPSVFLFPPKPKDTLMISRTPEVTCVVVDVSHEDP |
| | EVKFNWYVDGVEVHNAKTKPREEQYNSTYRVVSVL |
| | TVLHQDWLNGKEYKCKVSNKALPAPIEKTISKAKG |
| | QPREPQVYTLPPSRDELTKNQVSLTCLVKGFYPSD |
| | IAVEWESNGQPENNYKTTPPVLDSDGSFFLYSKLT |
| | VDKSRWQQGNVFSCSVMHEALHNHYTQKSLSLSPG |
| | K |
| 286 | GPVEVFITETPSQPNSHPIQWNAPQPSHISKYILR |
| | WRPKNSVGRWKEATIPGHLNSYTIKGLKPGVVYEG |
| | QLISIQQYGHQEVTRFDFTTTST |
| 287 | MKWVTFISLLFLFSSAYSRGVFRRDAHKSEVAHRF |
| | KDLGEENFKALVLIAFAQYLQQCPFEDHVKLVNEV |
| | TEFAKTCVADESAENCDKSLHTLFGDKLCTVATLR |
| | ETYGEMADCCAKQEPERNECFLQHKDDNPNLPRLV |
| | RPEVDVMCTAFHDNEETFLKKYLYEIARRHPYFYA |
| | PELLFFAKRYKAAFTECCQAADKAACLLPKLDELR |
| | DEGKASSAKQRLKCASLQKFGERAFKAWAVARLSQ |
| | RFPKAEFAEVSKLVTDLTKVHTECCHGDLLECADD |
| | RADLAKYICENQDSISSKLKECCEKPLLEKSHCIA |
| | EVENDEMPADLPSLAADFVESKDVCKNYAEAKDVF |
| | LGMFLYEYARRHPDYSVVLLLRLAKTYETTLEKCC |
| | AAADPHECYAKVFDEFKPLVEEPQNLIKQNCELFE |
| | QLGEYKFQNALLVRYTKKVPQVSTPTLVEVSRNLG |
| | KVGSKCCKHPEAKRMPCAEDYLSVVLNQLCVLHEK |
| | TPVSDRVTKCCTESLVNRRPCFSALEVDETYVP |
| | KEFNAETFTFHADICTLSEKERQIKKQTALVELVK |
| | HKPKATKEQLKAVMDDFAAFVEKCCKADDKETCFA |
| | EEGKKLVAASQAALGL |
| 305 | ETRECIYYNANWELERTNQSGLERCYGDKDKRRHC |
| | YASWRNSSGTIELVKKGCWLDDINCYDRTDCVEKK |
| | DSPEVYFCCCEGNFCNERFTHLPEAGGPEVTYEPP |
| | PTAPT |
| 306 | ETRECIYYNANWELERTNQSGLERCYGDKDKRRHC |
| | YASWRNSSGTIELVKKGCWLDDFNCYDRTDCVEKK |
| | DSPEVYFCCCEGNFCNERFTHLPEAGGPEVTYEPP |
| | PTAPT |
| 307 | ETRECIYYNANWELERTNQSGLERCEGEQDKRLHC |
| | YASWRNSSGTIELVKKGCWLDDINCYDRTDCVEKK |
| | DSPEVYFCCCEGNFCNERFTHLPEAGGPEVTYEPP |
| | PTAPT |
| 308 | ETRECIYYNANWELERTNQSGLERCEGEQDKRLHC |
| | YASWRNSSGTIELVKKGCWLDDINCYDRTDCVEKK |
| | DSPQVYFCCCEGNFCNERFTHLPEAGGPEVTYEPP |
| | PTAPT |
| 309 | ETRECIYYNANWELERTNQSGLERCEGEQDKRLHC |
| | YASWRNSSGTIELVKKGCWLDDFNCYDRTDCVEKK |
| | DSPQVYFCCCEGNFCNERFTHLPEAGGPEVTYEPP |
| | PTAPT |
| 310 | ETRECIYYNANWELERTNQSGLERCEGEQDKRLHC |
| | YASWRNSSGTIELVKKGCWLDDFNCYDRTDCVEKK |
| | DSPEVYFCCCEGNFCNERFTHLPEAGGPEVTYEPP |
| | PTAPT |
| 311 | ETRECIYYNANWELERTNQSGLERCEGEQDKRLHC |
| | YASWRNSSGTIELVKKGCWLDDFNCYDRQECVEKK |
| | DSPEVYFCCCEGNFCNERFTHLPEAGGPEVTYEPP |
| | PTAPT |
| 312 | ETRECIYYNANWELERTNQSGLERCEGEQDKRLHC |
| | YASWRNSSGTIELVKKGCWLDDFNCYDRQECVEKK |
| | DSPQVYFCCCEGNFCNERFTHLPEAGGPEVTYEPP |
| | PTAPT |
| 313 | ETRECIYYNANWELERTNQSGLERCEGEQDKRLHC |
| | YASWRNSSGTIELVKKGCWLDDINCYDRQECVEKK |
| | DSPEVYFCCCEGNFCNERFTHLPEAGGPEVTYEPP |
| | PTAPT |
| 314 | ETRECIYYNANWELERTNQSGLERCEGEQDKRLHC |
| | YASWRNSSGTIELVKKGCWLDDFNCYDRTDCVEKK |
| | DSPEVYFCCCEGNMCNERFTHLPEAGGPEVTYEPP |
| | PTAPT |
| 315 | ETRECIYYNANWELERTNQSGLERCEGDQDKRLHC |
| | YASWRNSSGTIELVKKGCWLDDFNCYDRQECVEKK |
| | DSPEVYFCCCEGNMCNERFTHLPEAGGPEVTYEPP |
| | PTAPT |
| 316 | ETQECIYYNANWEKDRTNQTGVEPCYGDKDKRRHC |
| | YASWRNSSGTIELVKKGCWLDDFNCYDRQECVATE |
| | ENPQVYFCCCEGNFCNERFTHLPEAGGPEVTYEPP |
| | PTAPT |
| 317 | ETQECIYYNANWEKDRTNQTGVEPCEGDQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 318 | ETRECIYYNANWELERTNQSGLERCYGDKDKRRHC |
| | YASWRNSSGTIELVKKGCWLDDENCYDRQECVATE |
| | ENPQVYFCCCEGNFCNERFTHLPEAGGPEVTYEPP |
| | PTAPT |
| 319 | ETRECIYYNANWEKDRTNQTGVEPCEGDQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 320 | ETQECIYYNANWEKDRTNQTGVEPCEGDQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 321 | ETQECIYYNANWEKDRTNQTGLERCEGEQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 322 | ETQECIYYNANWEKDRTNQSGLERCEGEQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 323 | ETRECIYYNANWEKDRTNQSGLERCEGEQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 324 | ETQECIYYNANWELERTNQSGLERCEGEQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 325 | ETRECIYYNANWELERTNQSGLERCEGEQDKRLHC |
| | FATWRNSSGTIELVKQGCWLDDENCYDRQECVATE |
| | ENPQVYFCCCEGNFCNERFTHLPEAGGPEVTYEPP |
| | PTAPT |
| 326 | ETRECIYYNANWELERTNQSGLERCYGDKDKRRHC |
| | YASWRNSSGTIELVKKGCWLDDINCYDRQECVATE |
| | ENPQVYFCCCEGNFCNERFTHLPEAGGPEVTYEPP |
| | PTAPT |
| 327 | ETRECIYYNANWELERTNQSGLERCEGEQDKRLHC |
| | YASWRNSSGTIELVKKGCWLDDFNCYDRQECVATK |
| | ENPQVYFCCCEGNFCNERFTHLPEAGGPEVTYEPP |
| | PTAPT |
| 328 | ETRECIYYNANWELERTNQSGLERCEGEQDKRLHC |
| | YASWRNSSGTIELVKKGCWLDDINCYDRQECVATE |
| | ENPQVYFCCCEGNFCNERFTHLPEAGGPEVTYEPP |
| | PTAPT |
| 329 | ETRECIYYNANWELERTNQSGLERCEGEQDKRLHC |
| | YASWRNSSGTIELVKKGCWLDDINCYDRQECVATK |
| | ENPQVYFCCCEGNFCNERFTHLPEAGGPEVTYEPP |
| | PTAPT |
| 330 | ETRECIYYNANWELERTNQSGLERCEGDKDKRLHC |
| | YASWRNSSGTIELVKKGCWLDDINCYDRQECVATK |
| | ENPQVYFCCCEGNFCNERFTHLPEAGGPEVTYEPP |
| | PTAPT |
| 331 | ETRECIYYNANWELERTNQSGLERCEGDQDKRLHC |
| | YASWRNSSGTIELVKKGCWLDDINCYDRQECVATK |
| | ENPQVYFCCCEGNFCNERFTHLPEAGGPEVTYEPP |
| | PTAPT |
| 332 | ETRECIYYNANWELERTNQSGLERCEGDQDKRLHC |
| | YASWRNSSGTIELVKKGCWLDDFNCYDRQECVATK |
| | ENPQVYFCCCEGNFCNERFTHLPEAGGPEVTYEPP |
| | PTAPT |
| 333 | ETRECIYYNANWELERTNQSGLERCEGDQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 334 | ETRECIYYNANWELERTNQSGLERCYGDKDKRRHC |
| | YASWRNSSGTIELVKKGCWLDDINCYDRQECVEKK |
| | DSPEVYFCCCEGNFCNERFTHLPEAGGPEVTYEPP |
| | PTAPT |
| 335 | ETRECIFFNANWEKDRTNQTGVEPCEGEQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 336 | ETRECIYYNANWELERTNQSGLERCEGEQDKRLHC |
| | FATWKNISGSIELVKQGCWLDDFNCYDRQECVATE |
| | ENPQVYFCCCEGNFCNERFTHLPEAGGPEVTYEPP |
| | PTAPT |
| 337 | ETRECIYYNANWELERTNQSGLERCEGEQDKRLHC |
| | YASWRNSSGTIELVKKGCWLDDENCYDRTDCVATE |
| | ENPQVYFCCCEGNFCNERFTHLPEAGGPEVTYEPP |
| | PTAPT |
| 338 | ETRECIYYNANWELERTNQSGLERCEGEQDKRLHC |
| | YASWRNSSGTIELVKKGCWLDDFNCYDRQECVATE |
| | ENPQVYFCCCEGNMCNERFTHLPEAGGPEVTYEPP |
| | PTAPT |
| 339 | ETRECIYYNANWELERTNQSGLERCEGDQDKRLHC |
| | YASWRNSSGTIELVKKGCWLDDINCYDRQECVATE |
| | ENPQVYFCCCEGNFCNERFTHLPEAGGPEVTYEPP |
| | PTAPT |
| 340 | ETQECLFFNANWEKDRTNQSGVEPCYGDKDKRRHC |
| | YASWRNSSGTIELVKKGCWLDDFNCYDRQECVATE |
| | ENPQVYFCCCEGNFCNERFTHLPEAGGPEVTYEPP |
| | PTAPT |
| 341 | ETQECLFFNANWEKDRTNQSGVEPCEGEQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 342 | ETRECLFFNANWEKDRTNQSGVEPCEGEQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 343 | ETQECLFFNANWEKDRTNQSGVEPCYGEQDKRLHC |
| | YASWRNSSGTIELVKKGCWLDDFNCYDRQECVATE |
| | ENPQVYFCCCEGNFCNERFTHLPEAGGPEVTYEPP |
| | PTAPT |
| 344 | ETRECLFFNANWEKDRTNQSGVEPCYGDKDKRRHC |
| | YASWRNSSGTIELVKKGCWLDDENCYDRQECVATE |
| | ENPQVYFCCCEGNFCNERFTHLPEAGGPEVTYEPP |
| | PTAPT |
| 345 | ETRECLFFNANWEKDRTNQTGVEPCYGDKDKRRHC |
| | FATWKNISGSIEIVKQGCWLDDFNCYDRQECVATE |
| | ENPQVYFCCCEGNFCNERFTHLPEAGGPEVTYEPP |
| | PTAPT |
| 346 | ETRECLFFNANWEKDRTNQTGVEPCYGDKDKRRHC |
| | FATWKNISGSIEIVKQGCWLDDINCYDRQECVATE |
| | ENPQVYFCCCEGNFCNERFTHLPEAGGPEVTYEPP |
| | PTAPT |
| 347 | ETRECLFFNANWEKDRTNQTGVEPCYGDKDKRRHC |
| | FATWKNISGSIEIVKQGCWLDDINCYDRTDCVEKK |
| | DSPEVYFCCCEGNFCNERFTHLPEAGGPEVTYEPP |
| | PTAPT |
| 348 | ETQECIYYNANWELERTNQSGLERCYGDKDKRRHC |
| | FATWKNISGSIEIVKQGCWLDDINCYDRQECVATE |
| | ENPQVYFCCCEGNFCNERFTHLPEAGGPEVTYEPP |
| | PTAPT |
| 349 | ETRECIYYNANWELERTNQSGLERCYGDKDKRRHC |
| | FATWKNISGSIEIVKQGCWLDDINCYDRTDCVEKK |
| | DSPEVYFCCCEGNFCNERFTHLPEAGGPEVTYEPP |
| | PTAPT |
| 350 | ETRECIYYNANWELERTNQSGLERCYGDKDKRRHC |
| | FATWKNISGSIEIVKQGCWLDDINCYDRTDCVEKK |
| | DSPEVYFCCCEGNMCNERFTHLPEAGGPEVTYEPP |
| | PTAPT |
| 351 | ETRECIYYNANWELERTNQSGLERCEGEQDKRLHC |
| | FATWKNISGSIEIVKQGCWLDDINCYDRTDCVEKK |
| | DSPEVYFCCCEGNFCNERFTHLPEAGGPEVTYEPP |
| | PTAPT |
| 352 | ETRECIYYNANWELERTNQSGLERCEGEQDKRLHC |
| | FATWKNISGSIEIVKQGCWLDDINCYDRTDCVATE |
| | ENPQVYFCCCEGNFCNERFTHLPEAGGPEVTYEPP |
| | PTAPT |
| 353 | ETRECIYYNANWELERTNQSGLERCEGEQDKRLHC |
| | FATWKNISGSIEIVKQGCWLDDFNCYDRTDCVATE |
| | ENPQVYFCCCEGNFCNERFTHLPEAGGPEVTYEPP |
| | PTAPT |
| 354 | ETRECIYYNANWELERTNQSGLERCEGEQDKRLHC |
| | FATWKNISGSIEIVKQGCWLDDFNCYDRQECVATE |
| | ENPQVYFCCCEGNFCNERFTHLPEAGGPEVTYEPP |
| | PTAPT |
| 355 | ETQECIYYNANWELERTNQSGLERCYGDKDKRRHC |
| | YASWRNSSGTIELVKKGCWLDDINCYDRTDCVEKK |
| | DSPEVYFCCCEGNFCNERFTHLPEAGGPEVTYEPP |
| | PTAPT |
| 356 | ETQECIYYNANWELERTNQSGLERCYGDKDKRRHC |
| | YASWRNSSGTIELVKKGCWLDDINCYDRTDCVEKK |
| | DSPEVYFCCCEGNMCNERFTHLPEAGGPEVTYEPP |
| | PTAPT |
| 357 | ETQECIYYNANWELERTNQSGLERCYGDKDKRRHC |
| | FATWKNISGSIEIVKQGCWLDDINCYDRTDCVEKK |
| | DSPEVYFCCCEGNMCNERFTHLPEAGGPEVTYEPP |
| | PTAPT |
| 358 | ETQECIYYNANWELERTNQSGLERCYGDKDKRRHC |
| | FATWKNISGSIEIVKQGCWLDDINCYDRTDCVEKK |
| | DSPEVYFCCCEGNFCNERFTHLPEAGGPEVTYEPP |
| | PTAPT |
| 359 | ETRECLFFNANWEKDRTNQTGVEPCEGEQDKRLHC |
| | FATWKNISGSIEIVKQGCWLDDINCYDRTDCVEKK |
| | DSPEVYFCCCEGNFCNERFTHLPEAGGPEVTYEPP |
| | PTAPT |
| 360 | ETRECLFFNANWEKDRTNQSGVEPCEGEQDKRLHC |
| | YASWRNSSGTIELVKKGCWLDDINCYDRTDCVEKK |
| | DSPEVYFCCCEGNFCNERFTHLPEAGGPEVTYEPP |
| | PTAPT |
| 361 | ETRECLFFNANWEKDRTNQSGVEPCYGDKDKRRHC |
| | YASWRNSSGTIELVKKGCWLDDINCYDRTDCVEKK |
| | DSPEVYFCCCEGNFCNERFTHLPEAGGPEVTYEPP |
| | PTAPT |
| 362 | ETRECIYYNANWELERTNQSGLERCYGDKDKRRHC |
| | YASWRNSSGTIELVKKGCWLDDFNCYDRTDCVATE |
| | ENPQVYFCCCEGNFCNERFTHLPEAGGPEVTYEPP |
| | PTAPT |
| 363 | ETRECIYYNANWELERTNQSGLERCYGDKDKRRHC |
| | YASWRNSSGTIELVKQGCWLDDENCYDRTDCVATE |
| | ENPQVYFCCCEGNFCNERFTHLPEAGGPEVTYEPP |
| | PTAPT |
| 364 | ETRECIYYNANWELERTNQSGLERCYGDKDKRRHC |
| | YASWRNSSGTIEIVKKGCWLDDENCYDRTDCVATE |
| | ENPQVYFCCCEGNFCNERFTHLPEAGGPEVTYEPP |
| | PTAPT |
| 365 | ETRECIYYNANWELERTNQSGLERCYGDKDKRRHC |
| | YASWRNSSGSIELVKKGCWLDDFNCYDRTDCVATE |
| | ENPQVYFCCCEGNFCNERFTHLPEAGGPEVTYEPP |
| | PTAPT |
| 366 | ETRECIYYNANWELERTNQSGLERCYGDKDKRRHC |
| | YASWRNSSGSIEIVKKGCWLDDFNCYDRTDCVATE |
| | ENPQVYFCCCEGNFCNERFTHLPEAGGPEVTYEPP |
| | PTAPT |
| 367 | ETRECIYYNANWELERTNQSGLERCYGDKDKRRHC |
| | YASWRNSSGSIEIVKQGCWLDDFNCYDRTDCVATE |
| | ENPQVYFCCCEGNFCNERFTHLPEAGGPEVTYEPP |
| | PTAPT |
| 368 | ETRECIYYNANWELERTNQSGLERCEGEQDKRLHC |
| | YASWRNSSGTIELVKKGCWLDDENCYDRTDCVATE |
| | ENPQVYFCCCEGNFCNERFTHLPEAGGPEVTYEPP |
| | PTAPT |
| 369 | ETRECIYYNANWELERTNQSGLERCEGEQDKRLHC |
| | YASWRNSSGTIELVKKGCWLDDFNCYDRQECVATE |
| | ENPQVYFCCCEGNFCNEKFSYFPEMEVTQPTSNPV |
| | TPKPP |
| 370 | ETRECIYYNANWELERTNQSGLERCEGEQDKRLHC |
| | YASWRNSSGTIELVKKGCWLDDFNCYDRQECVATE |
| | ENPQVYFCCCEGNFCNEKFSYFPQMEVTQPTSNPV |
| | TPKPP |
| 371 | ETRECIYYNANWELERTNQSGLERCYGDKDKRRHC |
| | YASWRNSSGTIELVKKGCWLDDINCYDRTDCVEKK |
| | DSPEVYFCCCEGNFCNEKFSYFPEMEVTQPTSNPV |
| | TPKPP |
| 372 | ETRECIYYNANWELERTNQSGLERCYGDKDKRRHC |
| | YASWRNSSGTIELVKKGCWLDDINCYDRTDCVEKK |
| | DSPEVYFCCCEGNFCNEKFSYFPQMEVTQPTSNPV |
| | TPKPP |
| 373 | ETRECIYYNANWELERTNQSGLERCYGDKDKRRHC |
| | YASWRNSSGTIELVKKGCWLDDFNCYDRQECVATE |
| | ENPQVYFCCCEGNFCNEKFSYFPQMEVTQPTSNPV |
| | TPKPP |
| 374 | ETRECIYYNANWELERTNQSGLERCYGDKDKRRHC |
| | YASWRNSSGTIELVKQGCWLDDFNCYDRQECVATE |
| | ENPQVYFCCCEGNFCNEKFSYFPQMEVTQPTSNPV |
| | TPKPP |
| 375 | ETRECIYYNANWELERTNQSGLERCYGDKDKRRHC |
| | YASWRNSSGTIEIVKKGCWLDDFNCYDRQECVATK |
| | ENPQVYFCCCEGNFCNEKFSYFPQMEVTQPTSNPV |
| | TPKPP |
| 376 | ETRECIYYNANWELERTNQSGLERCYGDKDKRRHC |
| | YASWRNSSGSIELVKKGCWLDDFNCYDRQECVATK |
| | ENPQVYFCCCEGNFCNEKFSYFPQMEVTQPTSNPV |
| | TPKPP |
| 377 | ETRECIYYNANWELERTNQSGLERCEGEQDKRLHC |
| | YASWRNSSGSIELVKKGCWLDDFNCYDRQECVATE |
| | ENPQVYFCCCEGNFCNEKFSYFPQMEVTQPTSNPV |
| | TPKPP |
| 378 | ETRECIYYNANWELERTNQSGLERCEGEQDKRLHC |
| | YASWRNSSGTIEIVKKGCWLDDFNCYDRQECVATE |
| | ENPQVYFCCCEGNFCNEKFSYFPQMEVTQPTSNPV |
| | TPKPP |
| 379 | ETRECIYYNANWELERTNQSGLERCEGEQDKRLHC |
| | YASWRNSSGTIELVKQGCWLDDFNCYDRQECVATE |
| | ENPQVYFCCCEGNFCNEKFSYFPQMEVTQPTSNPV |
| | TPKPP |
| 380 | ETRECIYYNANWELERTNQSGLERCEGEQDKRLHC |
| | YASWRNSSGTIELVKKGCWLDDFNCYDRQECVETE |
| | ENPQVYFCCCEGNFCNEKFSYFPQMEVTQPTSNPV |
| | TPKPP |
| 381 | ETRECIYYNANWELERTNQSGLERCEGEQDKRLHC |
| | YASWRNSSGTIELVKKGCWLDDFNCYDRQECVAKE |
| | ENPQVYFCCCEGNFCNEKFSYFPQMEVTQPTSNPV |
| | TPKPP |
| 382 | ETRECIYYNANWELERTNQSGLERCEGEQDKRLHC |
| | YASWRNSSGTIELVKKGCWLDDFNCYDRQECVATK |
| | ENPQVYFCCCEGNFCNEKFSYFPQMEVTQPTSNPV |
| | TPKPP |
| 383 | ETRECIYYNANWELERTNQSGLERCEGEQDKRLHC |
| | YASWRNSSGTIELVKKGCWLDDFNCYDRQECVATE |
| | DNPQVYFCCCEGNFCNEKFSYFPQMEVTQPTSNPV |
| | TPKPP |
| 384 | ETRECIYYNANWELERTNQSGLERCEGEQDKRLHC |
| | YASWRNSSGTIELVKKGCWLDDFNCYDRQECVATE |
| | ESPQVYFCCCEGNFCNEKFSYFPQMEVTQPTSNPV |
| | TPKPP |
| 385 | ETRECIYYNANWELERTNQSGLERCEGEQDKRLHC |
| | YASWRNSSGTIELVKKGCWLDDFNCYDRQECVATE |
| | ENPEVYFCCCEGNFCNEKFSYFPQMEVTQPTSNPV |
| | TPKPP |
| 386 | ETRECIYYNANWELERTNQSGLERCEGDKDKRLHC |
| | YASWRNSSGTIELVKKGCWLDDFNCYDRQECVETE |
| | ENPQVYFCCCEGNFCNEKFSYFPQMEVTQPTSNPV |
| | TPKPP |
| 387 | ETRECIYYNANWELERTNQSGLERCEGEKDKRLHC |
| | YASWRNSSGTIELVKKGCWLDDFNCYDRQECVAKE |
| | ENPQVYFCCCEGNFCNEKFSYFPQMEVTQPTSNPV |
| | TPKPP |
| 388 | ETRECIYYNANWELERTNQSGLERCEGDQDKRLHC |
| | YASWRNSSGTIELVKKGCWLDDFNCYDRQECVATK |
| | ENPQVYFCCCEGNFCNEKFSYFPQMEVTQPTSNPV |
| | TPKPP |
| 389 | ETRECIYYNANWELERTNQSGLERCEGDKDKRLHC |
| | YASWRNSSGTIELVKQGCWLDDFNCYDRQECVAKK |
| | ENPQVYFCCCEGNFCNEKFSYFPQMEVTQPTSNPV |
| | TPKPP |
| 390 | ETRECIYYNANWELERTNQSGLERCEGDKDKRLHC |
| | YASWRNSSGTIEIVKQGCWLDDFNCYDRQECVAEK |
| | ENPQVYFCCCEGNFCNEKFSYFPQMEVTQPTSNPV |
| | TPKPP |
| 391 | ETRECIYYNANWELERTNQSGLERCYGDQDKRLHC |
| | YASWRNSSGSIEIVKQGCWLDDFNCYDRQECVAKK |
| | ENPQVYFCCCEGNFCNEKFSYFPQMEVTQPTSNPV |
| | TPKPP |
| 392 | ETRECIYYNANWELERTNQSGLERCEGEKDKRRHC |
| | YASWRNSSGTIEIVKKGCWLDDFNCYDRQECVATK |
| | ENPQVYFCCCEGNFCNEKFSYFPQMEVTQPTSNPV |
| | TPKPP |
| 393 | ETRECIYYNANWELERTNQSGLERCYGDQDKRRHC |
| | YASWRNSSGTIELVKKGCWLDDFNCYDRQECVATE |
| | ENPEVYFCCCEGNFCNEKFSYFPQMEVTQPTSNPV |
| | TPKPP |
| 394 | ETRECIYYNANWELERTNQSGLERCEGEQDKRRHC |
| | YASWRNSSGTIELVKKGCWLDDFNCYDRQECVATE |
| | ENPQVYFCCCEGNFCNEKFSYFPQMEVTQPTSNPV |
| | TPKPP |
| 395 | ETRECIYYNANWELERTNQSGLERCYGEQDKRLHC |
| | YASWRNSSGSIEIVKKGCWLDDFNCYDRTDCVATE |
| | ENPQVYFCCCEGNFCNEKFSYFPQMEVTQPTSNPV |
| | TPKPP |
| 396 | ETRECIYYNANWELERTNQSGLERCEGEQDKRRHC |
| | YASWRNSSGSIELVKKGCWLDDFNCYDRQECVAKE |
| | ENPQVYFCCCEGNFCNEKFSYFPQMEVTQPTSNPV |
| | TPKPP |
| 397 | ETRECIYYNANWELERTNQSGLERCEGEQDKRRHC |
| | YASWRNSSGTIEIVKKGCWLDDFNCYDRQECVAKE |
| | ENPQVYFCCCEGNFCNEKFSYFPQMEVTQPTSNPV |
| | TPKPP |
| 398 | ETRECIYYNANWELERTNQSGLERCEGEQDKRRHC |
| | YASWRNSSGSIEIVKKGCWLDDFNCYDRQECVATK |
| | ENPQVYFCCCEGNFCNEKFSYFPQMEVTQPTSNPV |
| | TPKPP |
| 399 | ETRECIYYNANWELERTNQSGLERCEGDQDKRLHC |
| | YASWRNSSGTIEIVKKGCWLDDFNCYDRQECVATK |
| | ENPQVYFCCCEGNFCNEKFSYFPQMEVTQPTSNPV |
| | TPKPP |
| 400 | ETRECIYYNANWELERTNQSGLERCEGDQDKRLHC |
| | YASWRNSSGSIELVKKGCWLDDFNCYDRQECVATK |
| | ENPQVYFCCCEGNFCNEKFSYFPQMEVTQPTSNPV |
| | TPKPP |
| 401 | ETRECIYYNANWELERTNQSGLERCEGDQDKRLHC |
| | YASWRNSSGTIELVKKGCWLDDFNCYDRQECVATK |
| | ENPQVYFCCCEGNFCNEKFSYFPQMEVTQPTSNPV |
| | TPKPP |
| 402 | ETRECIYYNANWELERTNQSGLERCEGEQDKRLHC |
| | YASWRNSSGTIEIVKKGCWLDDFNCYDRQECVATK |
| | ENPQVYFCCCEGNFCNEKFSYFPQMEVTQPTSNPV |
| | TPKPP |
| 403 | ETRECIYYNANWELERTNQSGLERCEGEQDKRLHC |
| | YASWRNSSGTIELVKKGCWLDDFNCYDRQECVATK |
| | ENPQVYFCCCEGNFCNEKFSYFPQMEVTQPTSNPV |
| | TPKPP |
| 404 | ETRECIYYNANWELERTNQSGLERCEGDQDKRLHC |
| | YASWRNSSGTIEIVKKGCWLDDFNCYDRQECVAKK |
| | ENPQVYFCCCEGNFCNEKFSYFPQMEVTQPTSNPV |
| | TPKPP |
| 405 | ETQECLFFNANWEKDRTNQTGVEPCYGDKDKRRHC |
| | FATWKNISGSIEIVKQGCWLDDINCYDRTDCVEKK |
| | DSPEVYFCCCEGNMCNERFTHLPEAGGPEVTYEPP |
| | PTAPT |
| 406 | ETRECIYYNANWELERTNQSGLERCYGDKDKRRHC |
| | YASWRNSSGTIELVKKGCWLDDINCYDRQECVATK |
| | ENPQVYFCCCEGNFCNERFTHLPEAGGPEVTYEPP |
| | PTAPT |
| 407 | SGRGEAETRECIYYNANWELERTNQSGLERCEGEQ |
| | DKRLHCYASWRNSSGTIELVKKGCWLDDFNCYDRQ |
| | ECVATEENPQVYFCCCEGNFCNERFTHLPEAGGPE |
| | VTYEPPPTAPT |
| 408 | MGAATKLAFAVFLISCSSGAILGRSETQECIYYNA |
| | NWEKDKTNRSGIEPCYGDKDKRRHCFATWKNISGS |
| | IEIVKQGCWLDDINCYDRNDCIEKKDSPEVFFCCC |
| | EGNMCNERFFYFPEMEVTQPTSNPVTPKPPLFNTL |
| | LYSLVPIMGIAVIVLFSFWMYRHHKLAYPPVLVPT |
| | QDPGPPPPSPLMGLKPLQLLEIKARGRFGCVWKAQ |
| | LLNEYVAVKIFPIQDKQSWQNEYEIYSLPGMKHDN |
| | ILQFIGAEKRGTSIDVDLWLITAFHEKGSLTDFLK |
| | ANVVSWNELCHIAQTMARGLAYLHEDIPGLKDGHK |
| | PAISHRDIKSKNVLLKNNLTACIADFGLALKFEAG |
| | KSAGDTHGQVGTRRYMAPEVLEGAINFQRDAFLRI |
| | DMYAMGLVLWELASRCTASDGPVDEYMLPFEEEIG |
| | QHPSLEDMQEVVVHKKKRPVLRECWQKHSGMAMLC |
| | ETIEECWDHDAEARLSAGCVEERIIQMQKLTNIIT |
| | TEDIVTVVTMVTNVDFPPKESSL |
| 409 | AILGRSETQECLFFNANWEKDRTNQTGVEPCYGDK |
| | DKRRHCFATWKNISGSIEIVKQGCWLDDINCYDRT |
| | DCVEKKDSPEVYFCCCEGNMCNEKFSYFPEMEVTQ |
| | PTSNPVTPKPP |

### 10. Exemplification

The disclosure above will be more readily understood by reference to the following examples, which are included merely for purposes of illustration of certain embodiments of the present invention, and are not intended to be limiting.

### Example 1: ActRIIA-Fc Fusion Proteins

A soluble ActRII fusion protein was constructed that has the extracellular domain of human ActRIIA fused to a human or mouse Fc domain with a minimal linker in between. The constructs are referred to as ActRIIA-hFc and ActRIIA-mFc, respectively.

ActRIIA-hFc is shown below as purified
from CHO cell lines (SEQ ID NO: 32):
ILGRSETQECLFFNANWEKDRTNQTGVEPCYGDKDKRRHCFATWK
NISGSIEIVKQGCWLDDINCYDRTDCVEKKDSPEVYFCCCEGNMC
NEKFSYFPEMEVTQPTSNPVTPKPPTGGGTHTCPPCPAPELLGGP
SVFLFPPKPKDTLMISRTPEVTCVVVDVSHEDPEVKFNWYVDGVE
VHNAKTKPREEQYNSTYRVVSVLTVLHQDWLNGKEYKCKVSNKAL
PVPIEKTISKAKGQPREPQVYTLPPSREEMTKNQVSLTCLVKGFY
PSDIAVEWESNGQPENNYKTTPPVLDSDGSFFLYSKLTVDKSRWQ
QGNVFSCSVMHEALHNHYTQKSLSLSPGK

The ActRIIA-hFc and ActRIIA-mFc proteins were expressed in CHO cell lines. Three different leader sequences were considered:

(i) Honey bee melittin (HBML):
(SEQ ID NO: 33)
MKFLVNVALVFMVVYISYIYA
(ii) Tissue plasminogen activator (TPA):
(SEQ ID NO: 34)
MDAMKRGLCCVLLLCGAVFVSP
(iii) Native:
(SEQ ID NO: 35)
MGAAAKLAFAVFLISCSSGA.

The selected form employs the TPA leader and has the following unprocessed amino acid sequence:

(SEQ ID NO: 36)
MDAMKRGLCCVLLLCGAVFVSPGAAILGRSETQECLFFNANWEKD
RTNQTGVEPCYGDKDKRRHCFATWKNISGSIEIVKQGCWLDDINC
YDRTDCVEKKDSPEVYFCCCEGNMCNEKFSYFPEMEVTQPTSNPV
TPKPPTGGGTHTCPPCPAPELLGGPSVFLFPPKPKDTLMISRTPE
VTCVVVDVSHEDPEVKFNWYVDGVEVHNAKTKPREEQYNSTYRVV
SVLTVLHQDWLNGKEYKCKVSNKALPVPIEKTISKAKGQPREPQV
YTLPPSREEMTKNQVSLTCLVKGFYPSDIAVEWESNGQPENNYKT
TPPVLDSDGSFFLYSKLTVDKSRWQQGNVFSCSVMHEALHNHYTQ
KSLSLSPGK

This polypeptide is encoded by the following nucleic acid sequence:

(SEQ ID NO: 37)
ATGGATGCAATGAAGAGAGGGCTCTGCTGTGTGCTGCTGCTGTGT
GGAGCAGTCTTCGTTTCGCCCGGCGCCGCTATACTTGGTAGATCA
GAAACTCAGGAGTGTCTTTTTTTAATGCTAATTGGGAAAAAGACA
GAACCAATCAAACTGGTGTTGAACCGTGTTATGGTGACAAAGATA
AACGGCGGCATTGTTTTGCTACCTGGAAGAATATTTCTGGTTCCA
TTGAATAGTGAAACAAGGTTGTTGGCTGGATGATATCAACTGCTA
TGACAGGACTGATTGTGTAGAAAAAAAAGACAGCCCTGAAGTATA
TTTCTGTTGCTGTGAGGGCAATATGTGTAATGAAAAGTTTTCTTA
TTTTCCGGAGATGGAAGTCACACAGCCCACTTCAAATCCAGTTAC
ACCTAAGCCACCCACCGGTGGTGGAACTCACACATGCCCACCGTG
CCCAGCACCTGAACTCCTGGGGGGACCGTCAGTCTTCCTCTTCCC
CCCAAAACCCAAGGACACCCTCATGATCTCCCGGACCCCTGAGGT
CACATGCGTGGTGGTGGACGTGAGCCACGAAGACCCTGAGGTCAA
GTTCAACTGGTACGTGGACGGCGTGGAGGTGCATAATGCCAAGAC
AAAGCCGCGGGAGGAGCAGTACAACAGCACGTACCGTGTGGTCAG
CGTCCTCACCGTCCTGCACCAGGACTGGCTGAATGGCAAGGAGTA
CAAGTGCAAGGTCTCCAACAAAGCCCTCCCAGTCCCCATCGAGAA
AACCATCTCCAAAGCCAAAGGGCAGCCCCGAGAACCACAGGTGTA
CACCCTGCCCCCATCCCGGGAGGAGATGACCAAGAACCAGGTCAG
CCTGACCTGCCTGGTCAAAGGCTTCTATCCCAGCGACATCGCCGT
GGAGTGGGAGAGCAATGGGCAGCCGGAGAACAACTACAAGACCAC
GCCTCCCGTGCTGGACTCCGACGGCTCCTTCTTCCTCTATAGCAA
GCTCACCGTGGACAAGAGCAGGTGGCAGCAGGGGAACGTCTTCTC
ATGCTCCGTGATGCATGAGGCTCTGCACAACCACTACACGCAGAA
GAGCCTCTCCCTGTCTCCGGGTAAATGAGAATTC

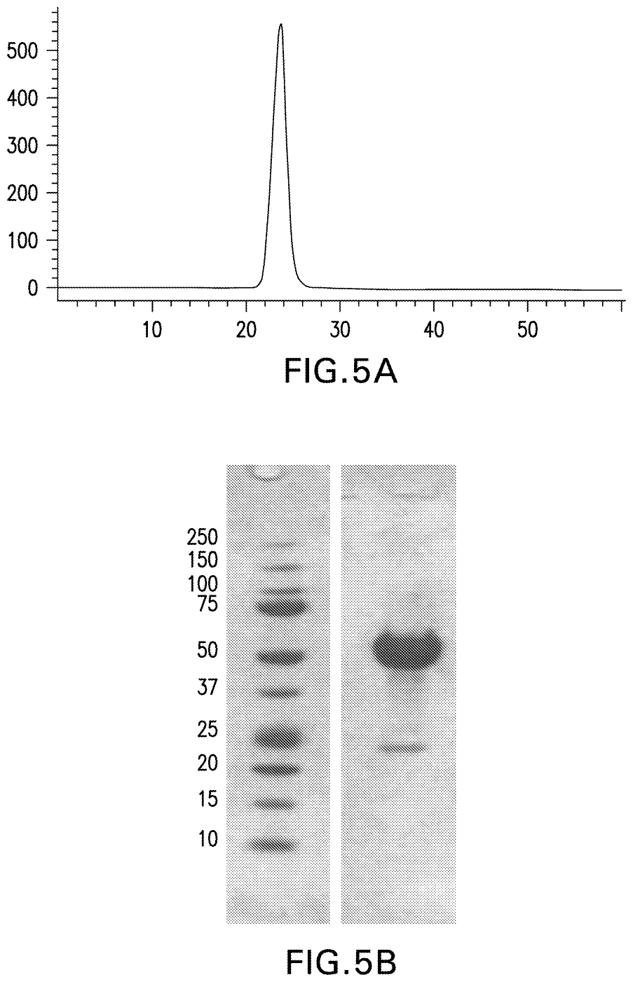

Both ActRIIA-hFc and ActRIIA-mFc were remarkably amenable to recombinant expression. As shown in FIG. 5, the protein was purified as a single, well-defined peak of protein. N-terminal sequencing revealed a single sequence of -ILGRSETQE (SEQ ID NO: 38). The ActRIIA-hFc protein was purified to a purity of >98% as determined by size exclusion chromatography and >95% as determined by SDS PAGE.

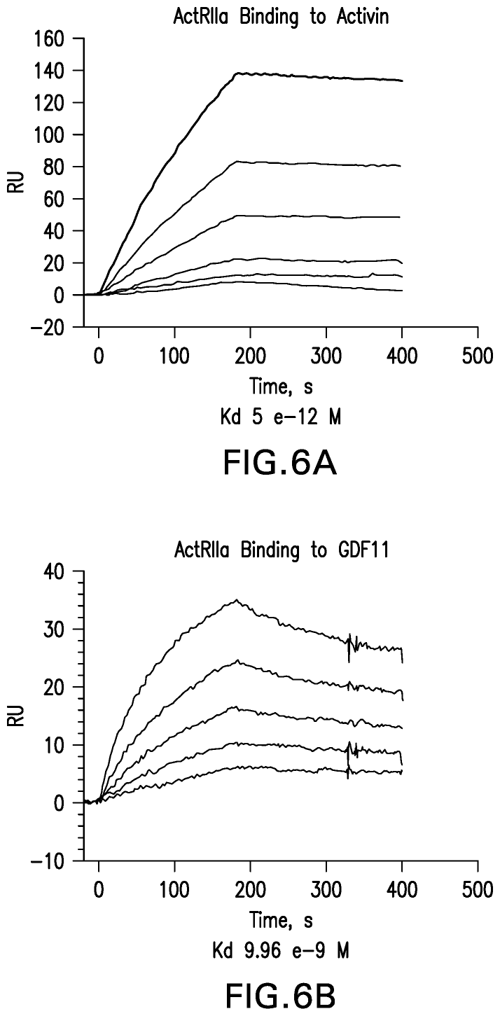

ActRIIA-hFc and ActRIIA-mFc showed a high affinity for ligands. GDF11 or activin A were immobilized on a Biacore™ CM5 chip using standard amine-coupling procedure. ActRIIA-hFc and ActRIIA-mFc proteins were loaded onto the system, and binding was measured. ActRIIA-hFc bound to activin with a dissociation constant (K_{D}) of 5×10⁻¹² and bound to GDF11 with a K_{D} of 9.96×10⁻⁹. See FIG. 6. Using a similar binding assay, ActRIIA-hFc was determined to have high to moderate affinity for other TGF-beta superfamily ligands including, for example, activin B, GDF8, BMP6, and BMP10. ActRIIA-mFc behaved similarly.

The ActRIIA-hFc was very stable in pharmacokinetic studies. Rats were dosed with 1 mg/kg, 3 mg/kg, or 10 mg/kg of ActRIIA-hFc protein, and plasma levels of the protein were measured at 24, 48, 72, 144 and 168 hours. In a separate study, rats were dosed at 1 mg/kg, 10 mg/kg, or 30 mg/kg. In rats, ActRIIA-hFc had an 11-14 day serum half-life, and circulating levels of the drug were quite high after two weeks (11 μg/ml, 110 μg/ml, or 304 μg/ml for initial administrations of 1 mg/kg, 10 mg/kg, or 30 mg/kg, respectively.) In cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 2: Characterization of an ActRIIA-hFc Protein

ActRIIA-hFc fusion protein was expressed in stably transfected CHO-DUKX B11 cells from a pAID4 vector (SV40 ori/enhancer, CMV promoter), using a tissue plasminogen leader sequence of SEQ ID NO: 34. The protein, purified as described above in Example 1, had a sequence of SEQ ID NO: 32. The Fc portion is a human IgG1 Fc sequence, as shown in SEQ ID NO: 32. Protein analysis revealed that the ActRIIA-hFc fusion protein is formed as a homodimer with disulfide bonding.

The CHO-cell-expressed material has a higher affinity for activin B ligand than that reported for an ActRIIA-hFc fusion protein expressed in human 293 cells [see, del Re et al. (2004) J Biol Chem. 279 (51): 53126-53135]. Additionally, the use of the TPA leader sequence provided greater production than other leader sequences and, unlike ActRIIA-Fc expressed with a native leader, provided a highly pure N-terminal sequence. Use of the native leader sequence resulted in two major species of ActRIIA-Fc, each having a different N-terminal sequence.

### Example 3: Alternative ActRIIA-Fc Proteins

A variety of ActRIIA variants that may be used according to the methods described herein are described in the International Patent Application published as WO2006/012627 (see e.g., pp. 55-58), incorporated herein by reference in its entirety. An alternative construct may have a deletion of the C-terminal tail (the final 15 amino acids of the extracellular

The sequence for such a construct
is presented below (Fc portion
underlined) (SEQ ID NO: 39):
ILGRSETQECLFFNANWEKDRTNQTGVEPCYGDKDKRRHCFATWK
NISGSIEIVKQGCWLDDINCYDRTDCVEKKDSPEVYFCCCEGNMC
NEKFSYFPEMTGGGTHTCPPCPAPELLGGPSVFLFPPKPKDTLMI
SRTPEVTCVVVDVSHEDPEVKENWYVDGVEVHNAKTKPREEQYNS
TYRVVSVLTVLHQDWLNGKEYKCKVSNKALPVPIEKTISKAKGQP
REPQVYTLPPSREEMTKNQVSLTCLVKGFYPSDIAVEWESNGQPE
NNYKTTPPVLDSDGSFFLYSKLTVDKSRWQQGNVFSCSVMHEALH
NHYTQKSLSLSPGK.

### Example 4: Generation of ActRIIB-Fc Fusion Proteins

Applicants constructed a soluble ActRIIB fusion protein that has the extracellular domain of human ActRIIB fused to a human or mouse Fc domain with a minimal linker in between. The constructs are referred to as ActRIIB-hFc and ActRIIB-mFc, respectively.

ActRIIB-hFc is shown below as purified from
CHO cell lines (SEQ ID NO: 40):
GRGEAETRECIYYNANWELERTNQSGLERCEGEQDKRLHCYASWR
NSSGTIELVKKGCWLDDFNCYDRQECVATEENPQVYFCCCEGNFC
NERFTHLPEAGGPEVTYEPPPTAPTGGGTHTCPPCPAPELLGGPS
VFLFPPKPKDTLMISRTPEVTCVVVDVSHEDPEVKFNWYVDGVEV
HNAKTKPREEQYNSTYRVVSVLTVLHQDWLNGKEYKCKVSNKALP
VPIEKTISKAKGQPREPQVYTLPPSREEMTKNQVSLTCLVKGFYP
SDIAVEWESNGQPENNYKTTPPVLDSDGSFFLYSKLTVDKSRWQQ
GNVFSCSVMHEALHNHYTQKSLSLSPGK.

The ActRIIB-hFc and ActRIIB-mFc proteins were expressed in CHO cell lines. Three different leader sequences were considered: (i) Honey bee melittin (HBML), ii) Tissue plasminogen activator (TPA), and (iii) Native: MGAAAKLAFAVFLISCSSGA (SEQ ID NO: 41).

The selected form employs the TPA leader and
has the following unprocessed amino
acid sequence (SEQ ID NO: 42):
MDAMKRGLCCVLLLCGAVFVSPGASGRGEAETRECIYYNANWELE
RTNQSGLERCEGEQDKRLHCYASWRNSSGTIELVKKGCWLDDENC
YDRQECVATEENPQVYFCCCEGNFCNERFTHLPEAGGPEVTYEPP
PTAPTGGGTHTCPPCPAPELLGGPSVFLFPPKPKDTLMISRTPEV
TCVVVDVSHEDPEVKFNWYVDGVEVHNAKTKPREEQYNSTYRVVS
VLTVLHQDWLNGKEYKCKVSNKALPVPIEKTISKAKGQPREPQVY
TLPPSREEMTKNQVSLTCLVKGFYPSDIAVEWESNGQPENNYKTT
PPVLDSDGSFFLYSKLTVDKSRWQQGNVFSCSVMHEALHNHYTQK
SLSLSPGK
This polypeptide is encoded by the following
nucleic acid sequence (SEQ ID NO: 43):
A TGGATGCAAT GAAGAGAGGG CTCTGCTGTG TGCTGCTGCT
GTGTGGAGCA GTCTTCGTTT CGCCCGGCGC CTCTGGGCGT
GGGGAGGCTG AGACACGGGA GTGCATCTAC TACAACGCCA
ACTGGGAGCT GGAGCGCACC AACCAGAGCG GCCTGGAGCG
CTGCGAAGGC GAGCAGGACA AGCGGCTGCA CTGCTACGCC
TCCTGGCGCA ACAGCTCTGG CACCATCGAG CTCGTGAAGA
AGGGCTGCTG GCTAGATGAC TTCAACTGCT ACGATAGGCA
GGAGTGTGTG GCCACTGAGG AGAACCCCCA GGTGTACTTC
TGCTGCTGTG AAGGCAACTT CTGCAACGAG CGCTTCACTC
ATTTGCCAGA GGCTGGGGGC CCGGAAGTCA CGTACGAGCC
ACCCCCGACA GCCCCCACCG GTGGTGGAAC TCACACATGC
CCACCGTGCC CAGCACCTGA ACTCCTGGGG GGACCGTCAG
TCTTCCTCTT CCCCCCAAAA CCCAAGGACA CCCTCATGAT
CTCCCGGACC CCTGAGGTCA CATGCGTGGT GGTGGACGTG
AGCCACGAAG ACCCTGAGGT CAAGTTCAAC TGGTACGTGG
ACGGCGTGGA GGTGCATAAT GCCAAGACAA AGCCGCGGGA
GGAGCAGTAC AACAGCACGT ACCGTGTGGT CAGCGTCCTC
ACCGTCCTGC ACCAGGACTG GCTGAATGGC AAGGAGTACA
AGTGCAAGGT CTCCAACAAA GCCCTCCCAG TCCCCATCGA
GAAAACCATC TCCAAAGCCA AAGGGCAGCC CCGAGAACCA
CAGGTGTACA CCCTGCCCCC ATCCCGGGAG GAGATGACCA
AGAACCAGGT CAGCCTGACC TGCCTGGTCA AAGGCTTCTA
TCCCAGCGAC ATCGCCGTGG AGTGGGAGAG CAATGGGCAG
CCGGAGAACA ACTACAAGAC CACGCCTCCC GTGCTGGACT
CCGACGGCTC CTTCTTCCTC TATAGCAAGC TCACCGTGGA
CAAGAGCAGG TGGCAGCAGG GGAACGTCTT CTCATGCTCC
GTGATGCATG AGGCTCTGCA CAACCACTAC ACGCAGAAGA
GCCTCTCCCT GTCTCCGGGT AAATGA.

N-terminal sequencing of the CHO-cell-produced material revealed a major sequence of -GRGEAE (SEQ ID NO: 44). Notably, other constructs reported in the literature begin with an -SGR . . . sequence.

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange.

ActRIIB-Fc fusion proteins were also expressed in HEK293 cells and COS cells. Although material from all cell lines and reasonable culture conditions provided protein with muscle-building activity in vivo, variability in potency was observed perhaps relating to cell line selection and/or culture conditions.

Applicants generated a series of mutations in the extracellular domain of ActRIIB and produced these mutant proteins as soluble fusion proteins between extracellular ActRIIB and an Fc domain. The background ActRIIB-Fc fusion has the sequence of SEQ ID NO: 40.

Various mutations, including N- and C-terminal truncations, were introduced into the background ActRIIB-Fc protein. Based on the data presented herein, it is expected that these constructs, if expressed with a TPA leader, will lack the N-terminal serine. Mutations were generated in ActRIIB extracellular domain by PCR mutagenesis. After PCR, fragments were purified through a Qiagen column, digested with SfoI and AgeI and gel purified. These fragments were ligated into expression vector pAID4 (see WO2006/012627) such that upon ligation it created fusion chimera with human IgG1. Upon transformation into E. coli DH5 alpha, colonies were picked and DNAs were isolated. For murine constructs (mFc), a murine IgG2a was substituted for the human IgG1. Sequences of all mutants were verified. All of the mutants were produced in HEK293T cells by transient transfection. In summary, in a 500 ml spinner, HEK293T cells were set up at 6×10⁵ cells/ml in Freestyle (Invitrogen) media in 250 ml volume and grown overnight. Next day, these cells were treated with DNA: PEI (1:1) complex at 0.5 μg/ml final DNA concentration. After 4 hrs, 250 ml media was added and cells were grown for 7 days. Conditioned media was harvested by spinning down the cells and concentrated.

Mutants were purified using a variety of techniques, including, for example, a protein A column, and eluted with low pH (3.0) glycine buffer. After neutralization, these were dialyzed against PBS.

Mutants were also produced in CHO cells by similar methodology. Mutants were tested in binding assays and/or bioassays described in WO 2008/097541 and WO 2006/012627 incorporated by reference herein. In some instances, assays were performed with conditioned medium rather than purified proteins. Additional variations of ActRIIB are described in U.S. Pat. No. 7,842,663.

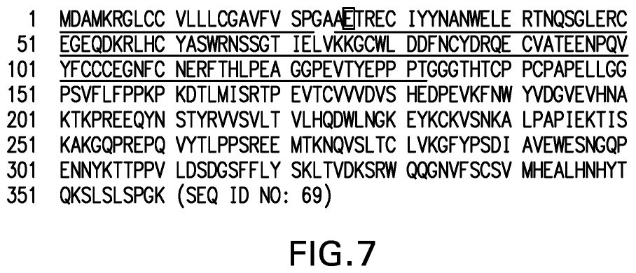

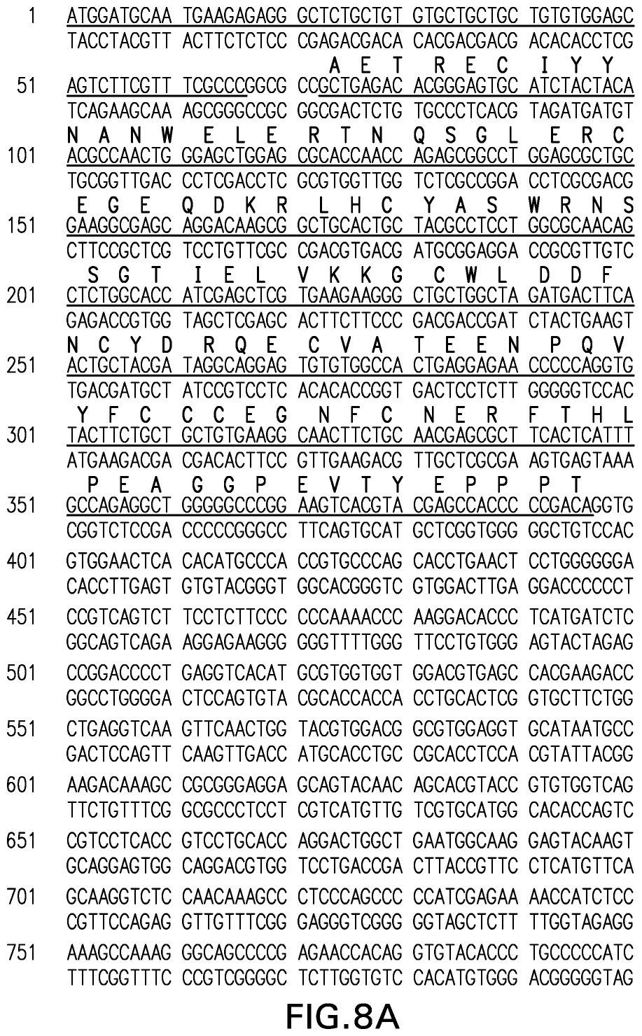

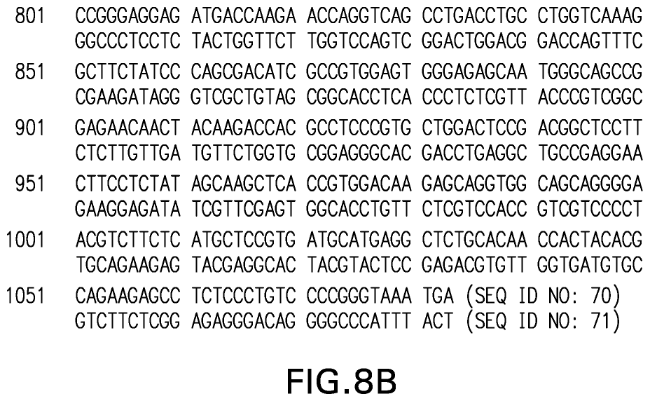

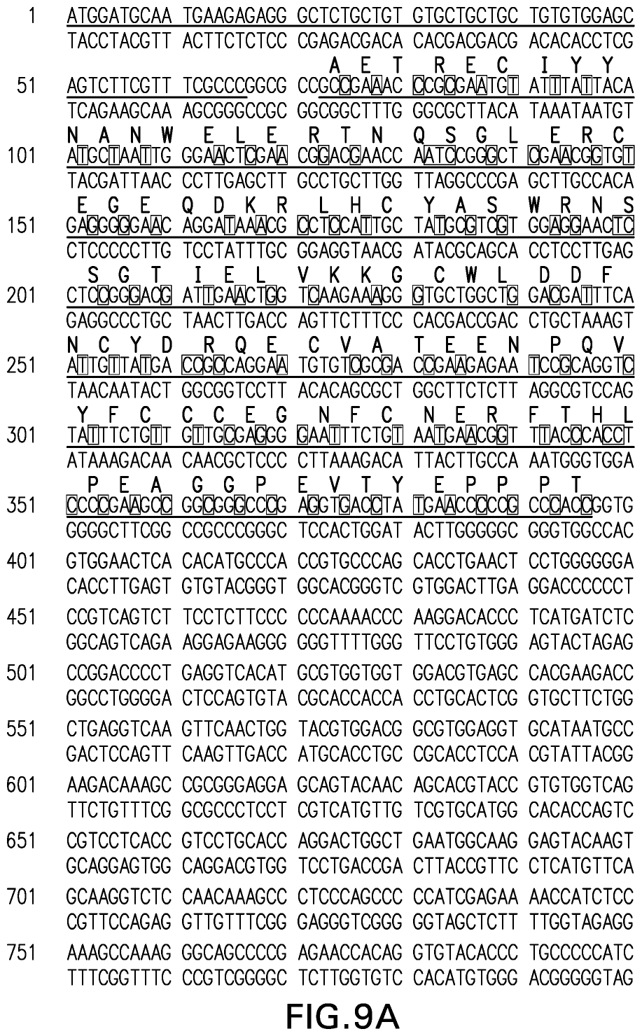

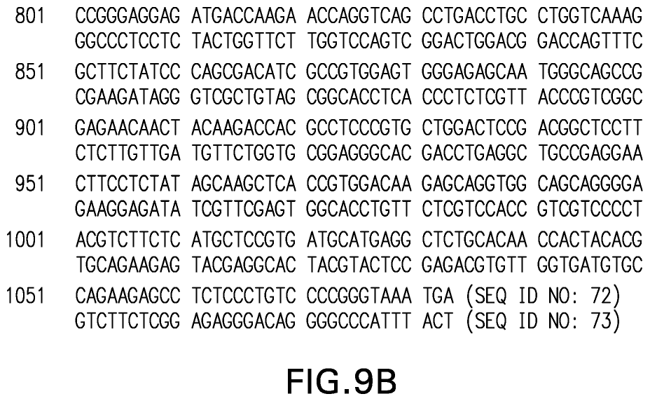

Applicant generated an ActRIIB (25-131)-hFc fusion protein, which comprises the human ActRIIB extracellular domain with N-terminal and C-terminal truncations (residues 25-131 of the native protein SEQ ID NO: 1) fused N-terminally with a TPA leader sequence substituted for the native ActRIIB leader and C-terminally with a human Fc domain via a minimal linker (three glycine residues) (FIG. 7). A nucleotide sequence encoding this fusion protein is shown in FIGS. 8A and 8B. Applicants modified the codons and found a variant nucleic acid encoding the ActRIIB (25-131)-hFc protein that provided substantial improvement in the expression levels of initial transformants (FIGS. 9A and 9B).

The mature protein has an amino acid sequence
as follows (N-terminus confirmed by
N-terminal sequencing)(SEQ ID NO: 45):
ETRECIYYNA NWELERTNQS GLERCEGEQD KRLHCYASWR
NSSGTIELVK KGCWLDDFNC YDRQECVATE ENPQVYFCCC
EGNFCNERFT HLPEAGGPEV TYEPPPTGGG THTCPPCPAP
ELLGGPSVFL FPPKPKDTLM ISRTPEVTCV VVDVSHEDPE
VKFNWYVDGV EVHNAKTKPR EEQYNSTYRV VSVLTVLHQD
WLNGKEYKCK VSNKALPAPI EKTISKAKGQ PREPQVYTLP
PSREEMTKNQ VSLTCLVKGF YPSDIAVEWE SNGQPENNYK
TTPPVLDSDG SFFLYSKLTV DKSRWQQGNV FSCSVMHEAL
HNHYTQKSLS LSPGK

The expressed molecule was purified using a series of column chromatography steps, including for example, three or more of the following, in any order: Protein A chromatography, Q sepharose chromatography, phenylsepharose chromatography, size exclusion chromatography and cation exchange chromatography. The purification could be completed with viral filtration and buffer exchange.

Affinities of several ligands for ActRIIB (25-131)-hFc and its full-length counterpart ActRIIB (20-134)-hFc were evaluated in vitro with a Biacore™ instrument, and the results are summarized in Table 9 below. Kd values were obtained by steady-state affinity fit due to very rapid association and dissociation of the complex, which prevented accurate determination of kₒₙ and k_{off}. ActRIIB (25-131)-hFc bound, for example, activin A, activin B, and GDF11 with high affinity.

TABLE 9
Ligand Affinities of ActRIIB-hFc Forms:

| | Activin A | Activin B | GDF11 |
|---|---|---|---|
| Fusion Construct | (e−11) | (e−11) | (e−11) |
| ActRIIB(20-134)-hFc | 1.6 | 1.2 | 3.6 |
| ActRIIB(25-131)-hFc | 1.8 | 1.2 | 3.1 |

A person of skill in the art would readily appreciate that the process outlined above could be used to produce a variant ActRII polypeptide (e.g., a variant ActRIIB polypeptide) Fc fusion protein based on the disclosure herein.

### Example 5: Generation of a ActRIIB Variant Fc Fusion Polypeptide

An ActRIIB variant Fc fusion polypeptide was constructed as follows. A polypeptide having a modified extracellular domain of ActRIIB (amino acids 20-134 of SEQ ID NO: 1 with an L79D substitution) with greatly reduced activin A binding relative to GDF11 and/or myostatin (as a consequence of a leucine-to-aspartate substitution at position 79 in SEQ ID NO: 1) was fused to a human or mouse Fc domain with a minimal linker in between. The constructs are referred to as ActRIIB (L79D 20-134)-hFc and ActRIIB (L79D 20-134)-mFc, respectively. Alternative forms with a glutamate rather than an aspartate at position 79 performed similarly (L79E). Alternative forms with an alanine rather than a valine at position 226 with respect to SEQ ID NO: 64, below were also generated and performed equivalently in all respects tested. The aspartate at position 79 (relative to SEQ ID NO: 1) is indicated with double underlining below. The valine at position 226 relative to SEQ ID NO: 64 is also indicated by double underlining below.

The ActRIIB variant Fc fusion polypeptide
ActRIIB(L79D 20-134)-hFc is shown below
as purified from CHO cell lines
(SEQ ID NO: 46)
GRGEAETRECIYYNANWELERTNQSGLERCEGEQDKRLHCYASWR
NSSGTIELVKKGCWDDDFNCYDRQECVATEENPQVYFCCCEGNFC
NERFTHLPEAGGPEVTYEPPPTAPTGGGTHTCPPCPAPELLGGPS
VFLFPPKPKDTLMISRTPEVTCVVVDVSHEDPEVKFNWYVDGVEV
HNAKTKPREEQYNSTYRVVSVLTVLHQDWLNGKEYKCKVSNKALP
VPIEKTISKAKGQPREPQVYTLPPSREEMTKNQVSLTCLVKGFYP
SDIAVEWESNGQPENNYKTTPPVLDSDGSFFLYSKLTVDKSRWQQ
GNVESCSVMHEALHNHYTQKSLSLSPGK.

The ActRIIB-derived portion of the ActRIIB variant Fc fusion polypeptide has an amino acid sequence set forth below (SEQ ID NO: 47), and that portion could be used as a monomer or as a non-Fc fusion protein as a monomer, dimer, or greater-order complex.

(SEQ ID NO: 47)
GRGEAETRECIYYNANWELERTNQSGLERCEGEQDKRLHCYASWR
NSSGTIELVKKGCWDDDFNCYDRQECVATEENPQVYFCCCEGNFC
NERFTHLPEAGGPEVTYEPPPTAPT

The ActRIIB variant Fc fusion polypeptide protein was expressed in CHO cell lines. Three different leader sequences were considered:
(i) Honey bee melittin (HBML), (ii) Tissue plasminogen activator (TPA), and (iii) Native.

The selected form employs the TPA leader and has the following unprocessed amino acid sequence:

(SEQ ID NO: 48)
MDAMKRGLCCVLLLCGAVFVSPGASGRGEAETRECIYYNANWELE
RTNQSGLERCEGEQDKRLHCYASWRNSSGTIELVKKGCWDDDFNC
YDRQECVATEENPQVYFCCCEGNFCNERFTHLPEAGGPEVTYEPP
PTAPTGGGTHTCPPCPAPELLGGPSVFLFPPKPKDTLMISRTPEV
TCVVVDVSHEDPEVKFNWYVDGVEVHNAKTKPREEQYNSTYRVVS
VLTVLHQDWLNGKEYKCKVSNKALPAPIEKTISKAKGQPREPQVY
TLPPSREEMTKNQVSLTCLVKGFYPSDIAVEWESNGQPENNYKTT
PPVLDSDGSFFLYSKLTVDKSRWQQGNVFSCSVMHEALHNHYTQK
SLSLSPGK
This polypeptide is encoded by the following
nucleic acid sequence (SEQ ID NO: 49):
A TGGATGCAAT GAAGAGAGGG CTCTGCTGTG TGCTGCTGCT
GTGTGGAGCA GTCTTCGTTT CGCCCGGCGC CTCTGGGCGT
GGGGAGGCTG AGACACGGGA GTGCATCTAC TACAACGCCA
ACTGGGAGCT GGAGCGCACC AACCAGAGCG GCCTGGAGCG
CTGCGAAGGC GAGCAGGACA AGCGGCTGCA CTGCTACGCC
TCCTGGCGCA ACAGCTCTGG CACCATCGAG CTCGTGAAGA
AGGGCTGCTG GGACGATGAC TTCAACTGCT ACGATAGGCA
GGAGTGTGTG GCCACTGAGG AGAACCCCCA GGTGTACTTC
TGCTGCTGTG AAGGCAACTT CTGCAACGAG CGCTTCACTC
ATTTGCCAGA GGCTGGGGGC CCGGAAGTCA CGTACGAGCC
ACCCCCGACA GCCCCCACCG GTGGTGGAAC TCACACATGC
CCACCGTGCC CAGCACCTGA ACTCCTGGGG GGACCGTCAG
TCTTCCTCTT CCCCCCAAAA CCCAAGGACA CCCTCATGAT
CTCCCGGACC CCTGAGGTCA CATGCGTGGT GGTGGACGTG
AGCCACGAAG ACCCTGAGGT CAAGTTCAAC TGGTACGTGG
ACGGCGTGGA GGTGCATAAT GCCAAGACAA AGCCGCGGGA
GGAGCAGTAC AACAGCACGT ACCGTGTGGT CAGCGTCCTC
ACCGTCCTGC ACCAGGACTG GCTGAATGGC AAGGAGTACA
AGTGCAAGGT CTCCAACAAA GCCCTCCCAG TCCCCATCGA
GAAAACCATC TCCAAAGCCA AAGGGCAGCC CCGAGAACCA
CAGGTGTACA CCCTGCCCCC ATCCCGGGAG GAGATGACCA
AGAACCAGGT CAGCCTGACC TGCCTGGTCA AAGGCTTCTA
TCCCAGCGAC ATCGCCGTGG AGTGGGAGAG CAATGGGCAG
CCGGAGAACA ACTACAAGAC CACGCCTCCC GTGCTGGACT
CCGACGGCTC CTTCTTCCTC TATAGCAAGC TCACCGTGGA
CAAGAGCAGG TGGCAGCAGG GGAACGTCTT CTCATGCTCC
GTGATGCATG AGGCTCTGCA CAACCACTAC ACGCAGAAGA
GCCTCTCCCT GTCTCCGGGT AAATGA

Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. In an example of a purification scheme, the cell culture medium is passed over a protein A column, washed in 150 mM Tris/NaCl (pH 8.0), then washed in 50 mM Tris/NaCl (pH 8.0) and eluted with 0.1 M glycine, pH 3.0. The low pH eluate is kept at room temperature for 30 minutes as a viral clearance step. The eluate is then neutralized and passed over a Q-sepharose ion-exchange column and washed in 50 mM Tris pH 8.0, 50 mM NaCl, and eluted in 50 mM Tris pH 8.0, with an NaCl concentration between 150 mM and 300 mM. The eluate is then changed into 50 mM Tris pH 8.0, 1.1 M ammonium sulfate and passed over a phenyl sepharose column, washed, and eluted in 50 mM Tris pH 8.0 with ammonium sulfate between 150 and 300 mM. The eluate is dialyzed and filtered for use.

Additional ActRIIB variant Fc fusion polypeptides (ActRIIB-Fc fusion proteins modified so as to reduce the ratio of activin A binding relative to myostatin or GDF11 binding) are described in WO 2008/097541 and WO 2006/012627, incorporated by reference herein.

### Example 6: Bioassay for GDF11- and Activin-Mediated Signaling

An A-204 reporter gene assay was used to evaluate the effects of ActRIIB-Fc proteins and ActRIIB variant Fc fusion polypeptides on signaling by GDF-11 and activin A. Cell line: human rhabdomyosarcoma (derived from muscle). Reporter vector: pGL3 (CAGA) 12 (described in Dennler et al, 1998, EMBO 17:3091-3100). The CAGA12 motif is present in TGF-beta responsive genes (e.g., PAI-1 gene), so this vector is of general use for factors signaling through SMADs.
Day 1: Split A-204 cells into 48-well plate.
Day 2: A-204 cells transfected with 10 μg pGL3 (CAGA) 12 or pGL3 (CAGA) 12 (10 μg)+pRLCMV (1 μg) and Fugene.
Day 3: Add factors (diluted into medium+0.1% BSA). Inhibitors need to be preincubated with factors for 1 hr before adding to cells. Six hrs later, cells were rinsed with PBS and lysed.

This is followed by a luciferase assay. In the absence of any inhibitors, activin A showed 10-fold stimulation of reporter gene expression and an ED50˜2 ng/ml. GDF-11:16 fold stimulation, ED50: ˜1.5 ng/ml.

ActRIIB (20-134) is a potent inhibitor of, for example, activin A, GDF-8, and GDF-11 activity in this assay. Certain ActRIIB variants were also tested in this assay and the results are reproduced below.

### Example 7: ActRIIB-Fc Variants, Cell-Based Activity

Activity of ActRIIB-Fc proteins and ActRIIB variant Fc fusion polypeptides was tested in a cell-based assay as described above. Results are summarized in Table 10 below. Some variants were tested in different C-terminal truncation constructs. As discussed above, truncations of five or fifteen amino acids caused reduction in activity. The ActRIIB variant Pc fusion polypeptides (L79D and L79E variants) showed substantial loss of activin A inhibition while retaining almost wild-type inhibition of GDF11.

TABLE 10
Soluble ActRIIB-Fc binding to GDF11 and Activin A:

| | Portion of ActRIIB | | |
|---|---|---|---|
| ActRIIB-Fc | (corresponds to amino | GDF11 Inhibition | Activin Inhibition |
| Variations | acids of SEQ ID NO: 1) | Activity | Activity |
| R64 | 20-134 | +++ | +++ |
| | | (approx. 10⁻⁸M K_{I}) | (approx. 10⁻⁸M K_{I}) |
| A64 | 20-134 | + | + |
| | | (approx. 10⁻⁶M K_{I}) | (approx. 10⁻⁶M K_{I}) |
| R64 | 20-129 | +++ | +++ |
| R64 K74A | 20-134 | ++++ | ++++ |
| R64 A24N | 20-134 | +++ | +++ |
| R64 A24N | 20-119 | ++ | ++ |
| R64 A24N K74A | 20-119 | + | + |
| R64 L79P | 20-134 | + | + |
| R64 L79P K74A | 20-134 | + | + |
| R64 L79D | 20-134 | +++ | + |
| R64 L79E | 20-134 | +++ | + |
| R64K | 20-134 | +++ | +++ |
| R64K | 20-129 | +++ | +++ |
| R64 P129S P130A | 20-134 | +++ | +++ |
| R64N | 20-134 | + | + |

+ Poor activity (roughly 1 × 10⁻⁶ K_{I})
++ Moderate activity (roughly 1 × 10⁻⁷ K_{I})
+++ Good (wild-type) activity (roughly 1 × 10⁻⁸ K_{I})
++++ Greater than wild-type activity

The A24N variant has activity in the cell-based assay (above) and that is equivalent to the wild-type molecule. The A24N variant, and any of the other variants tested above, may be combined with the ActRIIB variant Fc fusion polypeptides, such as the L79D or L79E variants.

### Example 8: GDF11 and Activin a Binding

Binding of certain ActRIIB-Fc proteins and ActRIIB variant Fc fusion polypeptides to ligands was tested in a Biacore™ assay.

The ActRIIB-Fc variants or wild-type protein were captured onto the system using an anti-hFc antibody. Ligands were injected and flowed over the captured receptor proteins. Results are summarized in the tables below.

TABLE 11
Ligand-binding specificity IIB variants.

| Protein | Kon (1/Ms) | Koff (1/s) | KD (M) |
|---|---|---|---|
| | GDF11 | | |
| ActRIIB(20-134)-hFc | 1.34e−6 | 1.13e−4 | 8.42e−11 |
| ActRIIB(A24N 20-134)-hFc | 1.21e−6 | 6.35e−5 | 5.19e−11 |
| ActRIIB(L79D 20-134)-hFc | 6.7e−5 | 4.39e−4 | 6.55e−10 |
| ActRIIB(L79E 20-134)-hFc | 3.8e−5 | 2.74e−4 | 7.16e−10 |
| ActRIIB(R64K 20-134)-hFc | 6.77e−5 | 2.41e−5 | 3.56e−11 |
| | GDF8 | | |
| ActRIIB(20-134)-hFc | 3.69e−5 | 3.45e−5 | 9.35e−11 |
| ActRIIB(A24N 20-134)-hFc | | | |
| ActRIIB(L79D 20-134)-hFc | 3.85e−5 | 8.3e−4 | 2.15e−9 |
| ActRIIB(L79E 20-134)-hFc | 3.74e−5 | 9e−4 | 2.41e−9 |
| ActRIIB(R64K 20-134)-hFc | 2.25e−5 | 4.71e−5 | 2.1e−10 |
| ActRIIB(R64K 20-129)-hFc | 9.74e−4 | 2.09e−4 | 2.15e−9 |
| ActRIIB(P129S, P130R | 1.08e−5 | 1.8e−4 | 1.67e−9 |
| 20-134)-hFc | | | |
| ActRIIB(K74A 20-134)-hFc | 2.8e−5 | 2.03e−5 | 7.18e−11 |
| | Activin A | | |
| ActRIIB(20-134)-hFc | 5.94e6 | 1.59e−4 | 2.68e−11 |
| ActRIIB(A24N 20-134)-hFc | 3.34e6 | 3.46e−4 | 1.04e−10 |
| ActRIIB(L79D 20-134)-hFc | | | Low binding |
| ActRIIB(L79E 20-134)-hFc | | | Low binding |
| ActRIIB(R64K 20-134)-hFc | 6.82e6 | 3.25e−4 | 4.76e−11 |
| ActRIIB(R64K 20-129)-hFc | 7.46e6 | 6.28e−4 | 8.41e−11 |
| ActRIIB(P129S, P130R | 5.02e6 | 4.17e−4 | 8.31e−11 |
| 20-134)-hFc | | | |

These data obtained in a cell-free assay confirm the cell-based assay data, demonstrating that the A24N variant retains ligand-binding activity that is similar to that of the ActRIIB (20-134)-hFc molecule and that the L79D or L79E molecule retains myostatin and GDF11 binding but shows markedly decreased (non-quantifiable) binding to activin A.

Other variants have been generated and tested, as reported in WO2006/012627 (incorporated herein by reference in its entirety). See, e.g., pp. 59-60, using ligands coupled to the device and flowing receptor over the coupled ligands. Notably, K74Y, K74F, K74I (and presumably other hydrophobic substitutions at K74, such as K74L), and D80I, cause a decrease in the ratio of activin A (ActA) binding to GDF11 binding, relative to the wild-type K74 molecule. Table 12 showing data with respect to these variants is reproduced below:

TABLE 12
Soluble ActRIIB-Fc variants binding to GDF11
and Activin A (Biacore ™ assay)

| ActRIIB | ActA | GDF11 |
|---|---|---|
| WT (64A) | KD = 1.8e−7M | KD = 2.6e−7M |
| | (+) | (+) |
| WT (64R) | na | KD = 8.6e−8M |
| | | (+++) |
| +15tail | KD ~2.6e−8M | KD = 1.9e−8M |
| | (+++) | (++++) |
| E37A | * | * |
| R40A | − | − |
| D54A | − | * |
| K55A | ++ | * |
| R56A | * | * |
| K74A | KD = 4.35e−9M | KD = 5.3e−9M |
| | +++++ | +++++ |
| K74Y | * | −− |
| K74F | * | −− |
| K74I | * | −− |
| W78A | * | * |
| L79A | + | * |
| D80K | * | * |
| D80R | * | * |
| D80A | * | * |
| D80F | * | * |
| D80G | * | * |
| D80M | * | * |
| D80N | * | * |
| D80I | * | −− |
| F82A | ++ | − |

* No observed binding
−− <⅕ WT binding
− ~½ WT binding
+ WT
++ <2x increased binding
+++ ~5x increased binding
++++ ~10x increased binding
+++++ ~40x increased binding

### Example 9: Generation of an ActRIIB Variant with Truncated ActRIIB Extracellular Domain

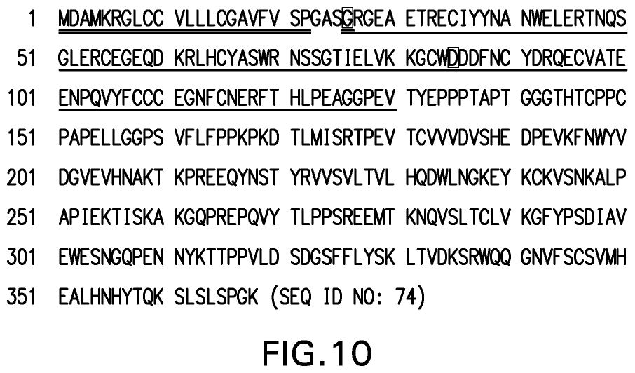

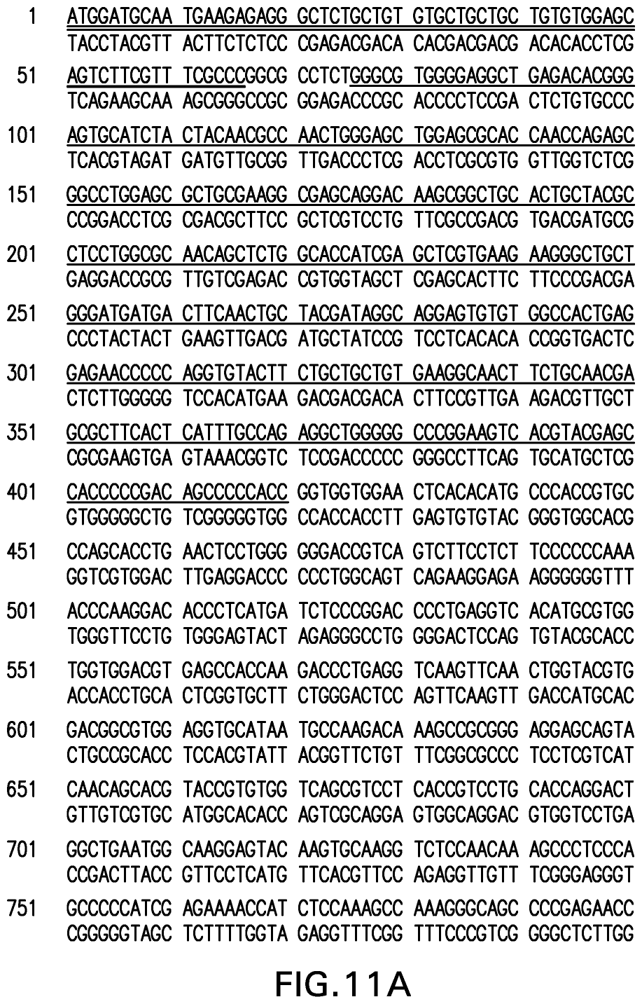

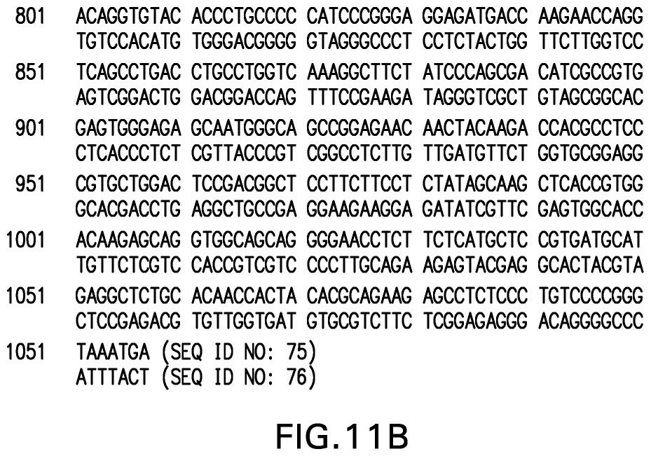

An ActRIIB variant referred to as ActRIIB (L79D 20-134)-hFc was generated by N-terminal fusion of TPA leader to the ActRIIB extracellular domain (residues 20-134 in SEQ ID NO: 1) containing a leucine-to-aspartate substitution (at residue 79 in SEQ ID NO: 1) and C-terminal fusion of human Fc domain with minimal linker (three glycine residues) (FIG. 10; SEQ ID NO: 74). A nucleotide sequence corresponding to this fusion protein is shown in FIGS. 11A and 11B (SEQ ID NO: 75, sense strand; and SEQ ID NO: 76, antisense strand).

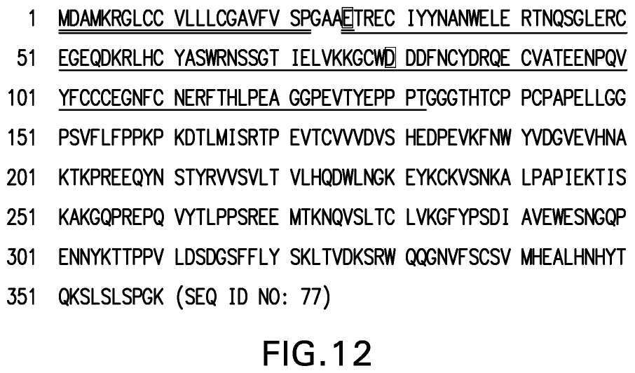

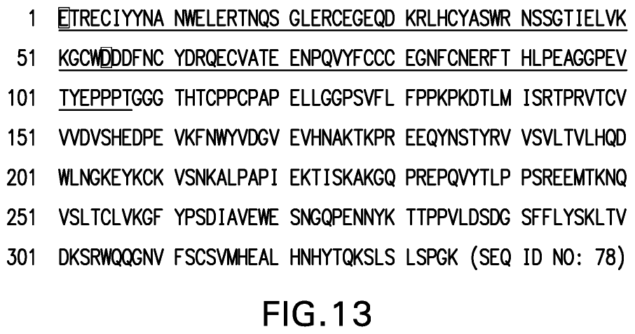

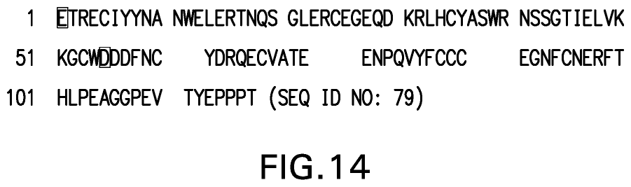

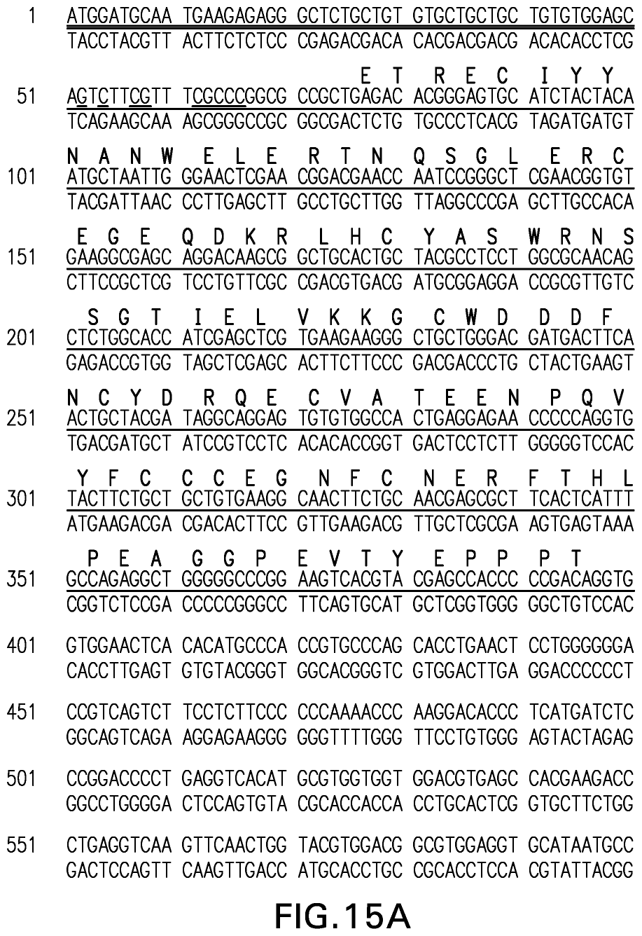

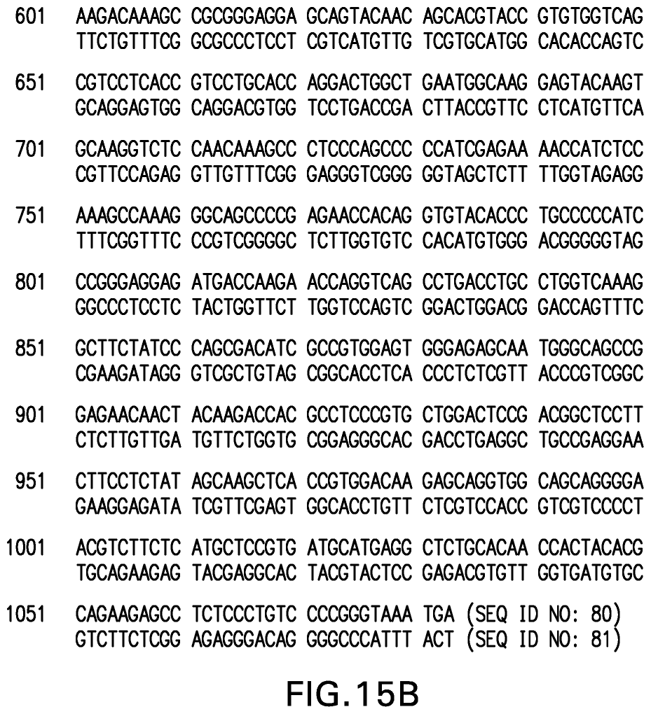

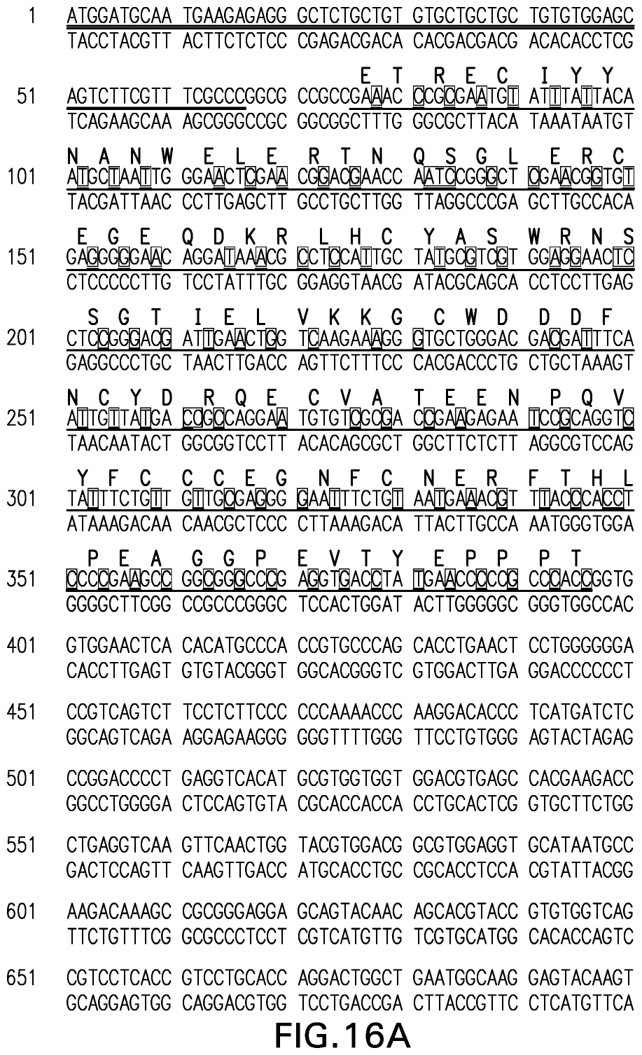

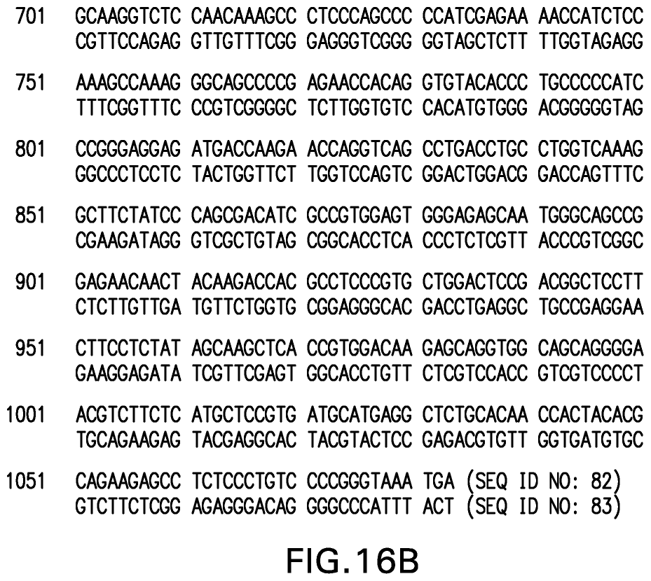

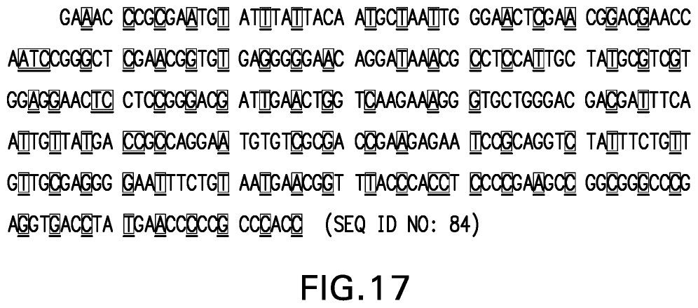

An ActRIIB variant with truncated ActRIIB extracellular domain, referred to as ActRIIB (L79D 25-131)-hFc, was generated by N-terminal fusion of TPA leader to truncated extracellular domain (residues 25-131 in SEQ ID NO:1) containing a leucine-to-aspartate substitution (at residue 79 in SEQ ID NO:1) and C-terminal fusion of human Fc domain with minimal linker (three glycine residues) (FIG. 12, SEQ ID NO: 77). The sequence of the cell purified form of ActRIIB (L79D 25-131)-hFc is presented in FIG. 13 (SEQ ID NO: 78). The sequence of the truncated ActRIIB (L79D 25-131) region without the leader, hFc domain, or linker is presented in FIG. 14 (SEQ ID NO: 79) One nucleotide sequence encoding the fusion protein is shown in FIGS. 15A and 15B (SEQ ID NO: 80) along with its complementary sequence (SEQ ID NO: 81), and an alternative nucleotide sequence encoding exactly the same fusion protein (SEQ ID NO: 82) and its complementary sequence (SEQ ID NO: 83) is shown in FIGS. 16A and 16B. An alternative nucleotide sequence (SEQ ID NO: 84) encoding only the truncated ActRIIB extracellular domain (corresponding to residues 25-131 of SEQ ID NO: 1) with the L79D substitution (SEQ ID NO: 82) is shown in FIG. 17.

### Example 10: Selective Ligand Binding by ActRIIB Variants with Double-Truncated ActRIIB Extracellular Domain

The affinity of ActRIIB variants and other ActRIIB-hFc proteins for several ligands was evaluated in vitro with a Biacore™ instrument. Results are summarized in Table 13 below. Kd values were obtained by steady-state affinity fit due to the very rapid association and dissociation of the complex, which prevented accurate determination of kₒₙ and k_{off}.

TABLE 13
Ligand Selectivity of ActRIIB-hFc Variants:

| | Activin A | Activin B | GDF11 |
|---|---|---|---|
| Fusion Construct | (Kd e−11) | (Kd e−11) | (Kd e−11) |
| ActRIIB(L79 20-134)-hFc | 1.6 | 1.2 | 3.6 |
| ActRIIB(L79D 20-134)-hFc | 1350.0 | 78.8 | 12.3 |
| ActRIIB(L79 25-131)-hFc | 1.8 | 1.2 | 3.1 |
| ActRIIB(L79D 25-131)-hFc | 2290.0 | 62.1 | 7.4 |

The ActRIIB variant with a truncated extracellular domain, ActRIIB (L79D 25-131)-hFc, equaled or surpassed the ligand selectivity displayed by the longer variant, ActRIIB (L79D 20-134)-hFc, with pronounced loss of activin A binding, partial loss of activin B binding, and nearly full retention of GDF11 binding compared to ActRIIB-hFc counterparts lacking the L79D substitution. Note that truncation alone (without L79D substitution) did not alter selectivity among the ligands displayed here (compare ActRIIB (L79 25-131)-hFc with ActRIIB (L79 20-134)-hFc). ActRIIB (L79D 25-131)-hFc also retains strong to intermediate binding to the SMAD signaling ligands GDF8, BMP6, and BMP10.

### Example 11: ActRIIB5 Variant Derived from ActRIIB5

Others have reported an alternate, soluble form of ActRIIB (designated ActRIIB5), in which exon 4, including the ActRIIB transmembrane domain, has been replaced by a different C-terminal sequence (see, e.g., WO 2007/053775).

The sequence of native human ActRIIB5 without its leader is as follows:

(SEQ ID NO: 50)
GRGEAETRECIYYNANWELERTNQSGLERCEGEQDKRLHCYASWR
NSSGTIELVKKGCWLDDFNCYDRQECVATEENPQVYFCCCEGNFC
NERFTHLPEAGGPEGPWASTTIPSGGPEATAAAGDQGSGALWLCL
EGPAHE

An leucine-to-aspartate substitution, or other acidic substitutions, may be performed at native position 79 (underlined) as described to construct the variant ActRIIB5 (L79D), which has the following sequence:

(SEQ ID NO: 51)
GRGEAETRECIYYNANWELERTNQSGLERCEGEQDKRLHCYASWR
NSSGTIELVKKGCWDDDFNCYDRQECVATEENPQVYFCCCEGNFC
NERFTHLPEAGGPEGPWASTTIPSGGPEATAAAGDQGSGALWLCL
EGPAHE

This variant may be connected to human Fc (double underline) with a TGGG linker (SEQ ID NO: 23) (single underline) to generate a human ActRIIB5 (L79D)-hFc fusion protein with the following sequence:

(SEQ ID NO: 52)
GRGEAETRECIYYNANWELERTNQSGLERCEGEQDKRLHCYASWR
NSSGTIELVKKGCWDDDFNCYDRQECVATEENPQVYFCCCEGNFC
NERFTHLPEAGGPEGPWASTTIPSGGPEATAAAGDQGSGALWLCL
EGPAHETGGGTHTCPPCPAPELLGGPSVFLFPPKPKDTLMISRTP
EVTCVVVDVSHEDPEVKENWYVDGVEVHNAKTKPREEQYNSTYRV
VSVLTVLHQDWLNGKEYKCKVSNKALPAPIEKTISKAKGQPREPQ
VYTLPPSREEMTKNQVSLTCLVKGFYPSDIAVEWESNGQPENNYK
TTPPVLDSDGSFFLYSKLTVDKSRWQQGNVESCSVMHEALHNHYT
QKSLSLSPGK.

This construct may be expressed in CHO cells.

1-143. (canceled)

144. A method of treating a renal disease or condition, comprising administering to a patient in need thereof an effective amount of an ActRIIA fusion protein, wherein the ActRIIA fusion protein comprises:
i) an ActRIIA variant polypeptide comprising an amino acid sequence that is at least 90% identical to SEQ ID NO: 196 or 207;
ii) an immunoglobulin Fc domain; and
iii) a linker domain positioned between the ActRIIA variant polypeptide and the immunoglobulin Fc domain;
wherein the ActRIIA variant polypeptide or fusion protein binds to one or more ligands selected from the group consisting of: activin A, activin B, GDF11, GDF8, and BMP6.

145. The method of claim 144, wherein the ActRIIA variant polypeptide comprises an amino acid sequence that is at least 95% identical to SEQ ID NO: 196 or 207.

146. The method of claim 144, wherein the ActRIIA variant polypeptide comprises an amino acid sequence that is at least 98% identical to SEQ ID NO: 196 or 207.

147. The method of claim 144, wherein the ActRIIA variant polypeptide comprises an amino acid sequence that is identical to SEQ ID NO: 196 or 207.

148. The method of claim 144, wherein the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24).

149. The method of claim 144, wherein the immunoglobulin Fc domain comprises an amino acid sequence that is at least 95% identical to a polypeptide selected from the group consisting of SEQ ID NOs: 14, 15, 16, 17, 18, 133, 134, 135, 136, 233, and 284.

150. The method of claim 144, wherein the immunoglobulin Fc domain comprises an amino acid sequence that is at least 98% identical to a polypeptide selected from the group consisting of SEQ ID NOs: 14, 15, 16, 17, 18, 133, 134, 135, 136, 233, and 284.

151. The method of claim 144, wherein the ActRIIA fusion protein comprises one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety.

152. The method of claim 144, wherein the renal disease or condition is selected from the group consisting of Alport syndrome, focal segmental glomerulosclerosis (FSGS), polycystic kidney disease, and chronic kidney disease.

153. The method of claim 144, wherein the method reduces severity, occurrence and/or duration of Stage A1 albuminuria, Stage A2 albuminuria, and/or Stage A3 albuminuria.

154. A method of treating a renal disease or condition, comprising administering to a patient in need thereof an effective amount of an ActRIIA fusion protein, wherein the ActRIIA fusion protein comprises:
i) an ActRIIA variant polypeptide comprising an amino acid sequence that is at least 90% identical to SEQ ID NO: 10;
ii) an immunoglobulin Fc domain; and
iii) a linker domain positioned between the ActRIIA variant polypeptide and the immunoglobulin Fc domain;
wherein the ActRIIA variant polypeptide or fusion protein binds to one or more ligands selected from the group consisting of: activin A, activin B, GDF11, GDF8, and BMP6.

155. The method of claim 154, wherein the ActRIIA variant polypeptide comprises an amino acid sequence that is at least 95% identical to SEQ ID NO: 10.

156. The method of claim 154, wherein the ActRIIA variant polypeptide comprises an amino acid sequence that is at least 98% identical to SEQ ID NO: 10.

157. The method of claim 154, wherein the ActRIIA variant polypeptide comprises an amino acid sequence that is identical to SEQ ID NO: 10.

158. The method of claim 154, wherein the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24).

159. The method of claim 154, wherein the immunoglobulin Fc domain comprises an amino acid sequence that is at least 95% identical to a polypeptide selected from the group consisting of SEQ ID NOs: 14, 15, 16, 17, 18, 133, 134, 135, 136, 233, and 284.

160. The method of claim 154, wherein the immunoglobulin Fc domain comprises an amino acid sequence that is at least 98% identical to a polypeptide selected from the group consisting of SEQ ID NOs: 14, 15, 16, 17, 18, 133, 134, 135, 136, 233, and 284.

161. The method of claim 154, wherein the ActRIIA fusion protein comprises an amino acid sequence that is at least 99% identical to SEQ ID NO: 32.

162. The method of claim 154, wherein the ActRIIA fusion protein comprises an amino acid sequence that is identical to SEQ ID NO: 32.

163. The method of claim 154, wherein the ActRIIA fusion protein comprises one or more amino acid modifications selected from the group consisting of: a glycosylated amino acid, a PEGylated amino acid, a farnesylated amino acid, an acetylated amino acid, a biotinylated amino acid, and an amino acid conjugated to a lipid moiety.

164. The method of claim 154, wherein the renal disease or condition is selected from the group consisting of Alport syndrome, focal segmental glomerulosclerosis (FSGS), polycystic kidney disease, and chronic kidney disease.

165. The method of claim 154, wherein the method reduces severity, occurrence and/or duration of Stage A1 albuminuria, Stage A2 albuminuria, and/or Stage A3 albuminuria.


SEQUENCE LISTING (416 sequences)

SEQ ID NO: 1 - protein, 512 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 2 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 3 - protein, 100 aa, synthetic construct
GRGEAETRECIYYNANWELERTNQSGLERCEGEQDKRLHCYASWRNSSGTIELVKKGCWLDDFNCYDRQECVATEENPQVYFCCCEGNFCNERFTHLPEA

SEQ ID NO: 4 - protein, 512 aa, synthetic construct
MTAPWVALALLWGSLCAGSGRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 5 - protein, 115 aa, synthetic construct
GRGEAETRECIYYNANWELERTNQSGLERCEGEQDKRLHCYASWANSSGTIELVKKGCWLDDFNCYDRQECVATEENPQVYFCCCEGNFCNERFTHLPEAGGPEVTYEPPPTAPT

SEQ ID NO: 6 - protein, 100 aa, synthetic construct
GRGEAETRECIYYNANWELERTNQSGLERCEGEQDKRLHCYASWANSSGTIELVKKGCWLDDFNCYDRQECVATEENPQVYFCCCEGNFCNERFTHLPEA

SEQ ID NO: 7 - DNA, 1536 bp, Homo sapiens
  source 1..1536: mol_type = genomic DNA
atgacggcgccctgggtggccctcgccctcctctggggatcgctgtgcgccggctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccaccctgctcacggtgctggcctactcactgctgcccatcgggggcctttccctcatcgtcctgctggccttttggatgtaccggcatcgcaagcccccctacggtcatgtggacatccatgaggaccctgggcctccaccaccatcccctctggtgggcctgaagccactgcagctgctggagatcaaggctcgggggcgctttggctgtgtctggaaggcccagctcatgaatgactttgtagctgtcaagatcttcccactccaggacaagcagtcgtggcagagtgaacgggagatcttcagcacacctggcatgaagcacgagaacctgctacagttcattgctgccgagaagcgaggctccaacctcgaagtagagctgtggctcatcacggccttccatgacaagggctccctcacggattacctcaaggggaacatcatcacatggaacgaactgtgtcatgtagcagagacgatgtcacgaggcctctcatacctgcatgaggatgtgccctggtgccgtggcgagggccacaagccgtctattgcccacagggactttaaaagtaagaatgtattgctgaagagcgacctcacagccgtgctggctgactttggcttggctgttcgatttgagccagggaaacctccaggggacacccacggacaggtaggcacgagacggtacatggctcctgaggtgctcgagggagccatcaacttccagagagatgccttcctgcgcattgacatgtatgccatggggttggtgctgtgggagcttgtgtctcgctgcaaggctgcagacggacccgtggatgagtacatgctgccctttgaggaagagattggccagcacccttcgttggaggagctgcaggaggtggtggtgcacaagaagatgaggcccaccattaaagatcactggttgaaacacccgggcctggcccagctttgtgtgaccatcgaggagtgctgggaccatgatgcagaggctcgcttgtccgcgggctgtgtggaggagcgggtgtccctgattcggaggtcggtcaacggcactacctcggactgtctcgtttccctggtgacctctgtcaccaatgtggacctgccccctaaagagtcaagcatc

SEQ ID NO: 8 - DNA, 345 bp, Homo sapiens
  source 1..345: mol_type = genomic DNA
gggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgaacgcttcactcatttgccagaggctgggggcccggaagtcacgtacgagccacccccgacagcccccacc

SEQ ID NO: 9 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 10 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 11 - protein, 100 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 12 - DNA, 1539 bp, Homo sapiens
  source 1..1539: mol_type = genomic DNA
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 13 - DNA, 345 bp, Homo sapiens
  source 1..345: mol_type = genomic DNA
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 14 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 17 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 18 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - skipped

SEQ ID NO: 20 - protein, 4 aa, synthetic construct
GGGG

SEQ ID NO: 21 - protein, 5 aa, synthetic construct
TGGGG

SEQ ID NO: 22 - protein, 5 aa, synthetic construct
SGGGG

SEQ ID NO: 23 - protein, 4 aa, synthetic construct
TGGG

SEQ ID NO: 24 - protein, 4 aa, synthetic construct
SGGG

SEQ ID NO: 25 - protein, 5 aa, synthetic construct
GGGGS

SEQ ID NO: 26-31 - skipped (6 entries)

SEQ ID NO: 32 - protein, 344 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 21 aa, Apis cerana
MKFLVNVALVFMVVYISYIYA

SEQ ID NO: 34 - protein, 22 aa, unidentified
MDAMKRGLCCVLLLCGAVFVSP

SEQ ID NO: 35 - protein, 20 aa, synthetic construct
MGAAAKLAFAVFLISCSSGA

SEQ ID NO: 36 - protein, 369 aa, synthetic construct
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 1114 bp, synthetic construct
  source 1..1114: mol_type = other DNA
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 38 - protein, 9 aa, synthetic construct
ILGRSETQE

SEQ ID NO: 39 - protein, 329 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 343 aa, synthetic construct
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 20 aa, synthetic construct
MGAAAKLAFAVFLISCSSGA

SEQ ID NO: 42 - protein, 368 aa, synthetic construct
MDAMKRGLCCVLLLCGAVFVSPGASGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 1107 bp, synthetic construct
  source 1..1107: mol_type = other DNA
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 44 - protein, 6 aa, synthetic construct
GRGEAE

SEQ ID NO: 45 - protein, 335 aa, synthetic construct
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 343 aa, synthetic construct
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 115 aa, synthetic construct
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPT

SEQ ID NO: 48 - protein, 368 aa, synthetic construct
MDAMKRGLCCVLLLCGAVFVSPGASGRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTAPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 1107 bp, synthetic construct
  source 1..1107: mol_type = other DNA
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctgggacgatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 50 - protein, 141 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEGPWASTTIPSGGPEATAAAGDQGSGALWLCLEGPAHE

SEQ ID NO: 51 - protein, 141 aa, synthetic construct
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEGPWASTTIPSGGPEATAAAGDQGSGALWLCLEGPAHE

SEQ ID NO: 52 - protein, 370 aa, synthetic construct
GRGEAETRECIYYNANWELERTNQSGLERCEGEQDKRLHCYASWRNSSGTIELVKKGCWDDDFNCYDRQECVATEENPQVYFCCCEGNFCNERFTHLPEAGGPEGPWASTTIPSGGPEATAAAGDQGSGALWLCLEGPAHETGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 150 aa, Rattus sp.
MTAPWAALALLWGSLCAGSGRGEAETRECIYYNANWELERTNQSGLERCEGEQDKRLHCYASWPNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 54 - protein, 150 aa, Sus sp.
MTAPWAALALLWGSLCVGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 55 - protein, 150 aa, Mus sp.
MTAPWAALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEPGGPEVTYEPPPTAPTLLTVLAYSLLPIGGLS

SEQ ID NO: 56 - protein, 150 aa, Homo sapiens
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLS

SEQ ID NO: 57 - protein, 150 aa, Bos taurus
MTAPWAALALLWGSLCAGSGRGEAETRECIYYNANWELERTNQSGLERCEGERDKRLHCYASWRNSSGTIELVKKGCWLDDFNCYDRQECVATEENPQVYFCCCEGNFCNERFTHLPEAGGPEVTYEPPPTAPTLLTVLAYSLLPVGGLS

SEQ ID NO: 58 - protein, 150 aa, Xenopus sp.
MGASVALTFLLLLATFRAGSGHDEVETRECIYYNANWELEKTNQSGVERLVEGKKDKRLHCYASWRNNSGFIELVKKGCWLDDFNCYDRQECIAKEENPQVFFCCCEGNYCNKKFTHLPEVETFDPKPQPSASVLNILIYSLLPIVGLSM

SEQ ID NO: 59 - protein, 150 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLI

SEQ ID NO: 60 - protein, 154 aa, synthetic construct
  VARIANT 8: note = threonine, alanine, or absent
  VARIANT 121: note = proline, alanine, valine, or methionine
MTAPWAAXLALLWGSLCAGSGRGEAETRECIYYNANWELERTNQSGLERLCEGEQDKRLHCYASWRNSSGTLELVKKGCWLDDFNCYDRQECVATEENPQVYFCCCEGNFCNERFTHLPEXGGPEVTYEPKPPTAPTLLTVLAYSLLPIGGLSM

SEQ ID NO: 61 - skipped

SEQ ID NO: 62 - protein, 115 aa, Ovis aries
ILGRSETQECIFYNANWERDRTNRTGVESCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 63 - protein, 115 aa, unidentified
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKAP

SEQ ID NO: 64 - protein, 115 aa, Mus sp.
ILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKPP

SEQ ID NO: 65 - protein, 115 aa, Gallus gallus
ILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKPP

SEQ ID NO: 66 - protein, 115 aa, Bos taurus; identical to SEQ ID NO: 62

SEQ ID NO: 67 - protein, 115 aa, Tyto alba; identical to SEQ ID NO: 65

SEQ ID NO: 68 - protein, 115 aa, Myotis davidii
ILGRSETQECIFYNANWERDKTNRTGVELCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 69 - protein, 360 aa, synthetic construct
MDAMKRGLCCVLLLCGAVFVSPGAAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - DNA, 1083 bp, synthetic construct
  source 1..1083: mol_type = other DNA
atggatgcaatgaagagagggctctgctgtgtgctgctgctgtgtggagcagtcttcgtttcgcccggcgccgctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctggctagatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacaggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 71 - DNA, 1083 bp, synthetic construct
  source 1..1083: mol_type = other DNA
tacctacgttacttctctcccgagacgacacacgacgacgacacacctcgtcagaagcaaagcgggccgcggcgactctgtgccctcacgtagatgatgttgcggttgaccctcgacctcgcgtggttggtctcgccggacctcgcgacgcttccgctcgtcctgttcgccgacgtgacgatgcggaggaccgcgttgtcgagaccgtggtagctcgagcacttcttcccgacgaccgatctactgaagttgacgatgctatccgtcctcacacaccggtgactcctcttgggggtccacatgaagacgacgacacttccgttgaagacgttgctcgcgaagtgagtaaacggtctccgacccccgggccttcagtgcatgctcggtgggggctgtccaccaccttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctcctctactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatatcgttcgagtggcacctgttctcgtccaccgtcgtccccttgcagaagagtacgaggcactacgtactccgagacgtgttggtgatgtgcgtcttctcggagagggacaggggcccatttact

SEQ ID NO: 72 - DNA, 1083 bp, synthetic construct
  source 1..1083: mol_type = other DNA
atggatgcaatgaagagagggctctgctgtgtgctgctgctgtgtggagcagtcttcgtttcgcccggcgccgccgaaacccgcgaatgtatttattacaatgctaattgggaactcgaacggacgaaccaatccgggctcgaacggtgtgagggggaacaggataaacgcctccattgctatgcgtcgtggaggaactcctccgggacgattgaactggtcaagaaagggtgctggctggacgatttcaattgttatgaccgccaggaatgtgtcgcgaccgaagagaatccgcaggtctatttctgttgttgcgaggggaatttctgtaatgaacggtttacccacctccccgaagccggcgggcccgaggtgacctatgaacccccgcccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 73 - DNA, 1083 bp, synthetic construct
  source 1..1083: mol_type = other DNA
tacctacgttacttctctcccgagacgacacacgacgacgacacacctcgtcagaagcaaagcgggccgcggcggctttgggcgcttacataaataatgttacgattaacccttgagcttgcctgcttggttaggcccgagcttgccacactcccccttgtcctatttgcggaggtaacgatacgcagcacctccttgaggaggccctgctaacttgaccagttctttcccacgaccgacctgctaaagttaacaatactggcggtccttacacagcgctggcttctcttaggcgtccagataaagacaacaacgctccccttaaagacattacttgccaaatgggtggaggggcttcggccgcccgggctccactggatacttgggggcgggtggccaccaccttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctcctctactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatatcgttcgagtggcacctgttctcgtccaccgtcgtccccttgcagaagagtacgaggcactacgtactccgagacgtgttggtgatgtgcgtcttctcggagagggacaggggcccatttact

SEQ ID NO: 74 - protein, 368 aa, synthetic construct; identical to SEQ ID NO: 48

SEQ ID NO: 75 - DNA, 1107 bp, synthetic construct
  source 1..1107: mol_type = other DNA
atggatgcaatgaagagagggctctgctgtgtgctgctgctgtgtggagcagtcttcgtttcgcccggcgcctctgggcgtggggaggctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctgggatgatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacagcccccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccaccaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 76 - DNA, 1107 bp, synthetic construct
  source 1..1107: mol_type = other DNA
tacctacgttacttctctcccgagacgacacacgacgacgacacacctcgtcagaagcaaagcgggccgcggagacccgcacccctccgactctgtgccctcacgtagatgatgttgcggttgaccctcgacctcgcgtggttggtctcgccggacctcgcgacgcttccgctcgtcctgttcgccgacgtgacgatgcggaggaccgcgttgtcgagaccgtggtagctcgagcacttcttcccgacgaccctactactgaagttgacgatgctatccgtcctcacacaccggtgactcctcttgggggtccacatgaagacgacgacacttccgttgaagacgttgctcgcgaagtgagtaaacggtctccgacccccgggccttcagtgcatgctcggtgggggctgtcgggggtggccaccaccttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctcctctactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatatcgttcgagtggcacctgttctcgtccaccgtcgtccccttgcagaagagtacgaggcactacgtactccgagacgtgttggtgatgtgcgtcttctcggagagggacaggggcccatttact

SEQ ID NO: 77 - protein, 360 aa, synthetic construct
MDAMKRGLCCVLLLCGAVFVSPGAAETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 335 aa, synthetic construct
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPTGGGTHTCPPCPAPELLGGPSVFLFPPKPKDTLMISRTPRVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 107 aa, synthetic construct
ETRECIYYNANWELERTNQSGLERCEGEQDKRLHCYASWRNSSGTIELVKKGCWDDDFNCYDRQECVATEENPQVYFCCCEGNFCNERFTHLPEAGGPEVTYEPPPT

SEQ ID NO: 80 - DNA, 1083 bp, synthetic construct
  source 1..1083: mol_type = other DNA
atggatgcaatgaagagagggctctgctgtgtgctgctgctgtgtggagcagtcttcgtttcgcccggcgccgctgagacacgggagtgcatctactacaacgccaactgggagctggagcgcaccaaccagagcggcctggagcgctgcgaaggcgagcaggacaagcggctgcactgctacgcctcctggcgcaacagctctggcaccatcgagctcgtgaagaagggctgctgggacgatgacttcaactgctacgataggcaggagtgtgtggccactgaggagaacccccaggtgtacttctgctgctgtgaaggcaacttctgcaacgagcgcttcactcatttgccagaggctgggggcccggaagtcacgtacgagccacccccgacaggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 81 - DNA, 1083 bp, synthetic construct
  source 1..1083: mol_type = other DNA
tacctacgttacttctctcccgagacgacacacgacgacgacacacctcgtcagaagcaaagcgggccgcggcgactctgtgccctcacgtagatgatgttgcggttgaccctcgacctcgcgtggttggtctcgccggacctcgcgacgcttccgctcgtcctgttcgccgacgtgacgatgcggaggaccgcgttgtcgagaccgtggtagctcgagcacttcttcccgacgaccctgctactgaagttgacgatgctatccgtcctcacacaccggtgactcctcttgggggtccacatgaagacgacgacacttccgttgaagacgttgctcgcgaagtgagtaaacggtctccgacccccgggccttcagtgcatgctcggtgggggctgtccaccaccttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccctgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctcctctactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatatcgttcgagtggcacctgttctcgtccaccgtcgtccccttgcagaagagtacgaggcactacgtactccgagacgtgttggtgatgtgcgtcttctcggagagggacaggggcccatttact

SEQ ID NO: 82 - DNA, 1083 bp, synthetic construct
  source 1..1083: mol_type = other DNA
atggatgcaatgaagagagggctctgctgtgtgctgctgctgtgtggagcagtcttcgtttcgcccggcgccgccgaaacccgcgaatgtatttattacaatgctaattgggaactcgaacggacgaaccaatccgggctcgaacggtgtgagggggaacaggataaacgcctccattgctatgcgtcgtggaggaactcctccgggacgattgaactggtcaagaaagggtgctgggacgacgatttcaattgttatgaccgccaggaatgtgtcgcgaccgaagagaatccgcaggtctatttctgttgttgcgaggggaatttctgtaatgaacggtttacccacctccccgaagccggcgggcccgaggtgacctatgaacccccgcccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 83 - DNA, 1083 bp, synthetic construct
  source 1..1083: mol_type = other DNA
tacctacgttacttctctcccgagacgacacacgacgacgacacacctcgtcagaagcaaagcgggccgcggcggctttgggcgcttacataaataatgttacgattaacccttgagcttgcctgcttggttaggcccgagcttgccacactcccccttgtcctatttgcggaggtaacgatacgcagcacctccttgaggaggccctgctaacttgaccagttctttcccacgaccctgctgctaaagttaacaatactggcggtccttacacagcgctggcttctcttaggcgtccagataaagacaacaacgctccccttaaagacattacttgccaaatgggtggaggggcttcggccgcccgggctccactggatacttgggggcgggtggccaccaccttgagtgtgtacgggtggcacgggtcgtggacttgaggacccccctggcagtcagaaggagaaggggggttttgggttcctgtgggagtactagagggcctggggactccagtgtacgcaccaccacctgcactcggtgcttctgggactccagttcaagttgaccatgcacctgccgcacctccacgtattacggttctgtttcggcgccctcctcgtcatgttgtcgtgcatggcacaccagtcgcaggagtggcaggacgtggtcctgaccgacttaccgttcctcatgttcacgttccagaggttgtttcgggagggtcgggggtagctcttttggtagaggtttcggtttcccgtcggggctcttggtgtccacatgtgggacgggggtagggccctcctctactggttcttggtccagtcggactggacggaccagtttccgaagatagggtcgctgtagcggcacctcaccctctcgttacccgtcggcctcttgttgatgttctggtgcggagggcacgacctgaggctgccgaggaagaaggagatatcgttcgagtggcacctgttctcgtccaccgtcgtccccttgcagaagagtacgaggcactacgtactccgagacgtgttggtgatgtgcgtcttctcggagagggacaggggcccatttact

SEQ ID NO: 84 - DNA, 321 bp, synthetic construct
  source 1..321: mol_type = other DNA
gaaacccgcgaatgtatttattacaatgctaattgggaactcgaacggacgaaccaatccgggctcgaacggtgtgagggggaacaggataaacgcctccattgctatgcgtcgtggaggaactcctccgggacgattgaactggtcaagaaagggtgctgggacgacgatttcaattgttatgaccgccaggaatgtgtcgcgaccgaagagaatccgcaggtctatttctgttgttgcgaggggaatttctgtaatgaacggtttacccacctccccgaagccggcgggcccgaggtgacctatgaacccccgcccacc

SEQ ID NO: 85 - protein, 10 aa, synthetic construct
GGGGSGGGGS

SEQ ID NO: 86 - protein, 11 aa, synthetic construct
TGGGGSGGGGS

SEQ ID NO: 87 - protein, 16 aa, synthetic construct
TGGGGSGGGGSGGGGS

SEQ ID NO: 88 - protein, 21 aa, synthetic construct
TGGGGSGGGGSGGGGSGGGGS

SEQ ID NO: 89 - protein, 26 aa, synthetic construct
TGGGGSGGGGSGGGGSGGGGSGGGGS

SEQ ID NO: 90 - protein, 31 aa, synthetic construct
TGGGGSGGGGSGGGGSGGGGSGGGGSGGGGS

SEQ ID NO: 91 - protein, 36 aa, synthetic construct
TGGGGSGGGGSGGGGSGGGGSGGGGSGGGGSGGGGS

SEQ ID NO: 92 - protein, 10 aa, synthetic construct
TGGGPKSCDK

SEQ ID NO: 93-132 - skipped (40 entries)

SEQ ID NO: 133 - protein, 225 aa, Homo sapiens; identical to SEQ ID NO: 14

SEQ ID NO: 134 - protein, 229 aa, Homo sapiens; identical to SEQ ID NO: 18

SEQ ID NO: 135 - protein, 223 aa, Homo sapiens; identical to SEQ ID NO: 15

SEQ ID NO: 136 - protein, 232 aa, Homo sapiens; identical to SEQ ID NO: 16

SEQ ID NO: 137 - protein, 6 aa, synthetic construct
HHHHHH

SEQ ID NO: 138 - protein, 108 aa, synthetic construct
AETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPT

SEQ ID NO: 139 - protein, 109 aa, synthetic construct
  VARIANT 14: note = phenylalanine, alanine, valine, isoleucine, leucine, methionine, phenylalanine, tyrosine or tryptophan
  VARIANT 15: note = phenylalanine, alanine, valine, isoleucine, leucine, methionine, phenylalanine, tyrosine or tryptophan
  VARIANT 20: note = aspartic acid, glutamic acid or alanine
  VARIANT 21: note = lysine or leucine
  VARIANT 22: note = aspartic acid, glutamic acid, or alanine
  VARIANT 23: note = arginine, histidine, lysine, or alanine
  VARIANT 31: note = proline or arginine
  VARIANT 33: note = tyrosine or glutamic acid
  VARIANT 35: note = aspartic acid, glutamic acid, or alanine
  VARIANT 36: note = lysine or glutamine
  VARIANT 37: note = aspartic acid, glutamic acid, or alanine
  VARIANT 38: note = lysine, arginine, histidine, or alanine
  VARIANT 39: note = arginine, histidine, lysine, or alanine
  VARIANT 40: note = arginine or leucine
  VARIANT 43: note = phenylalanine, alanine, valine, isoleucine, leucine, methionine, phenylalanine, tyrosine or tryptophan
  VARIANT 47: note = lysine, arginine, histidine, or alanine
  VARIANT 57: note = lysine, alanine, tyrosine, phenylalanine, isoleucine, valine, leucine, methionine, or tryptophan
  VARIANT 58: note = glutamine or lysine
  VARIANT 61: note = tryptophan or alanine
  VARIANT 62: note = leucine or alanine
  VARIANT 63: note = aspartic acid, lysine, arginine, histidine, alanine, phenylalanine, glycine, methionine, asparagine, or isoleucine
  VARIANT 65: note = isoleucine, phenylalanine, alanine, valine, leucine, methionine, tyrosine or tryptophan
  VARIANT 76: note = lysine or threonine
  VARIANT 77: note = lysine, arginine, histidine, aspartic acid, or glutamic acid
  VARIANT 78: note = aspartic acid, glutamic acid, or alanine
  VARIANT 79: note = serine, asparagine, threonine, or glutamine
  VARIANT 81: note = glutamic acid or glutamine
GAILGRSETQECLXXNANWXXXXTNQTGVEXCXGXXXXXXHCXATWXNISGSIEIVXXGCXXXDXNCYDRTDCVEXXXXPXVYFCCCEGNMCNEKFSYFPEMEVTQPTS

SEQ ID NO: 140 - protein, 109 aa, synthetic construct
  VARIANT 15: note = phenylalanine, alanine, valine, isoleucine, leucine, methionine, phenylalanine, tyrosine or tryptophan
  VARIANT 20: note = aspartic acid, glutamic acid or alanine
  VARIANT 21: note = lysine or leucine
  VARIANT 22: note = aspartic acid, glutamic acid, or alanine
  VARIANT 23: note = arginine, histidine, lysine, or alanine
  VARIANT 31: note = proline or arginine
  VARIANT 33: note = tyrosine or glutamic acid
  VARIANT 35: note = aspartic acid, glutamic acid, or alanine
  VARIANT 37: note = aspartic acid, glutamic acid, or alanine
  VARIANT 38: note = lysine, arginine, histidine, or alanine
  VARIANT 39: note = arginine, histidine, lysine, or alanine
  VARIANT 40: note = arginine or leucine
  VARIANT 43: note = phenylalanine, alanine, valine, isoleucine, leucine, methionine, phenylalanine, tyrosine or tryptophan
  VARIANT 47: note = lysine, arginine, histidine, or alanine
  VARIANT 57: note = lysine, alanine, tyrosine, phenylalanine, isoleucine, valine, leucine, methionine, or tryptophan
  VARIANT 58: note = glutamine or lysine
  VARIANT 61: note = tryptophan or alanine
  VARIANT 62: note = leucine or alanine
  VARIANT 63: note = aspartic acid, lysine, arginine, histidine, alanine, phenylalanine, glycine, methionine, asparagine, or isoleucine
  VARIANT 65: note = isoleucine, phenylalanine, alanine, valine, leucine, methionine, tyrosine or tryptophan
  VARIANT 76: note = lysine or threonine
  VARIANT 77: note = lysine, arginine, histidine, aspartic acid, or glutamic acid
  VARIANT 78: note = aspartic acid, glutamic acid, or alanine
  VARIANT 79: note = serine, asparagine, threonine, or glutamine
  VARIANT 81: note = glutamic acid or glutamine
GAILGRSETQECLFXNANWXXXXTNQTGVEXCXGXKXXXXHCXATWXNISGSIEIVXXGCXXXDXNCYDRTDCVEXXXXPXVYFCCCEGNMCNEKFSYFPEMEVTQPTS

SEQ ID NO: 141 - protein, 109 aa, synthetic construct
  VARIANT 15: note = phenylalanine, alanine, valine, isoleucine, leucine, methionine, phenylalanine, tyrosine or tryptophan
  VARIANT 21: note = lysine or leucine
  VARIANT 22: note = aspartic acid, glutamic acid, or alanine
  VARIANT 31: note = proline or arginine
  VARIANT 33: note = tyrosine or glutamic acid
  VARIANT 35: note = aspartic acid, glutamic acid, or alanine
  VARIANT 40: note = arginine or leucine
  VARIANT 43: note = phenylalanine, alanine, valine, isoleucine, leucine, methionine, phenylalanine, tyrosine or tryptophan
  VARIANT 47: note = lysine, arginine, histidine, or alanine
  VARIANT 58: note = glutamine or lysine
  VARIANT 65: note = isoleucine, phenylalanine, alanine, valine, leucine, methionine, tyrosine or tryptophan
  VARIANT 76: note = lysine or threonine
  VARIANT 77: note = lysine, arginine, histidine, aspartic acid, or glutamic acid
  VARIANT 78: note = aspartic acid, glutamic acid, or alanine
  VARIANT 79: note = serine, asparagine, threonine, or glutamine
  VARIANT 81: note = glutamic acid or glutamine
GAILGRSETQECLFXNANWEXXRTNQTGVEXCXGXKDKRXHCXATWXNISGSIEIVKXGCWLDDXNCYDRTDCVEXXXXPXVYFCCCEGNMCNEKFSYFPEMEVTQPTS

SEQ ID NO: 142 - protein, 109 aa, synthetic construct
  VARIANT 15: note = phenylalanine, alanine, valine, isoleucine, leucine, methionine, phenylalanine, tyrosine or tryptophan
  VARIANT 21: note = lysine or leucine
  VARIANT 31: note = proline or arginine
  VARIANT 33: note = tyrosine or glutamic acid
  VARIANT 35: note = aspartic acid, glutamic acid, or alanine
  VARIANT 40: note = arginine or leucine
  VARIANT 43: note = phenylalanine, alanine, valine, isoleucine, leucine, methionine, phenylalanine, tyrosine or tryptophan
  VARIANT 47: note = lysine, arginine, histidine, or alanine
  VARIANT 58: note = glutamine or lysine
  VARIANT 65: note = isoleucine, phenylalanine, alanine, valine, leucine, methionine, tyrosine or tryptophan
  VARIANT 76: note = lysine or threonine
  VARIANT 78: note = aspartic acid, glutamic acid, or alanine
  VARIANT 79: note = serine, asparagine, threonine, or glutamine
  VARIANT 81: note = glutamic acid or glutamine
GAILGRSETQECLFXNANWEXDRTNQTGVEXCXGXKDKRXHCXATWXNISGSIEIVKXGCWLDDXNCYDRTDCVEXKXXPXVYFCCCEGNMCNEKFSYFPEMEVTQPTS

SEQ ID NO: 143 - protein, 109 aa, synthetic construct
  VARIANT 15: note = phenylalanine, alanine, valine, isoleucine, leucine, methionine, phenylalanine, tyrosine or tryptophan
  VARIANT 21: note = lysine or leucine
  VARIANT 33: note = tyrosine or glutamic acid
  VARIANT 35: note = aspartic acid, glutamic acid, or alanine
  VARIANT 40: note = arginine or leucine
  VARIANT 58: note = glutamine or lysine
  VARIANT 76: note = lysine or threonine
  VARIANT 78: note = aspartic acid, glutamic acid, or alanine
  VARIANT 79: note = serine, asparagine, threonine, or glutamine
  VARIANT 81: note = glutamic acid or glutamine
GAILGRSETQECLFXNANWEXDRTNQTGVEPCXGXKDKRXHCFATWKNISGSIEIVKXGCWLDDINCYDRTDCVEXKXXPXVYFCCCEGNMCNEKFSYFPEMEVTQPTS

SEQ ID NO: 144 - protein, 109 aa, synthetic construct
GAILGRSETQECLFYNANWELDRTNQTGVEPCEGEKDKRLHCFATWKNISGSIEIVKKGCWLDDINCYDRTDCVETKENPQVYFCCCEGNMCNEKFSYFPEMEVTQPTS

SEQ ID NO: 145 - protein, 109 aa, synthetic construct
GAILGRSETQECLFYNANWELERTNQTGVEPCEGEKDKRLHCFATWKNISGSIEIVKKGCWLDDINCYDRTDCVETKENPQVYFCCCEGNMCNEKFSYFPEMEVTQPTS

SEQ ID NO: 146 - protein, 109 aa, synthetic construct
GAILGRSETQECLFYNANWELDRTNQTGVERCEGEKDKRLHCFATWKNISGSIEIVKKGCWLDDINCYDRTDCVETKENPQVYFCCCEGNMCNEKFSYFPEMEVTQPTS

SEQ ID NO: 147 - protein, 109 aa, synthetic construct
GAILGRSETQECLFYNANWELDRTNQTGVEPCEGEKDKRLHCYATWKNISGSIEIVKKGCWLDDINCYDRTDCVETKENPQVYFCCCEGNMCNEKFSYFPEMEVTQPTS

SEQ ID NO: 148 - protein, 109 aa, synthetic construct
GAILGRSETQECLFYNANWELDRTNQTGVEPCEGEKDKRLHCFATWRNISGSIEIVKKGCWLDDINCYDRTDCVETKENPQVYFCCCEGNMCNEKFSYFPEMEVTQPTS

SEQ ID NO: 149 - protein, 109 aa, synthetic construct
GAILGRSETQECLFYNANWELDRTNQTGVEPCEGEKDKRLHCFATWKNISGSIEIVKKGCWLDDFNCYDRTDCVETKENPQVYFCCCEGNMCNEKFSYFPEMEVTQPTS

SEQ ID NO: 150 - protein, 109 aa, synthetic construct
GAILGRSETQECLFYNANWELDRTNQTGVEPCEGEKDKRLHCFATWKNISGSIEIVKKGCWLDDINCYDRTDCVETEENPQVYFCCCEGNMCNEKFSYFPEMEVTQPTS

SEQ ID NO: 151 - protein, 109 aa, synthetic construct
GAILGRSETQECLFYNANWELERTNQTGVERCEGEKDKRLHCFATWKNISGSIEIVKKGCWLDDINCYDRTDCVETKENPQVYFCCCEGNMCNEKFSYFPEMEVTQPTS

SEQ ID NO: 152 - protein, 109 aa, synthetic construct
GAILGRSETQECLFYNANWELERTNQTGVEPCEGEKDKRLHCYATWKNISGSIEIVKKGCWLDDINCYDRTDCVETKENPQVYFCCCEGNMCNEKFSYFPEMEVTQPTS

SEQ ID NO: 153 - protein, 109 aa, synthetic construct
GAILGRSETQECLFYNANWELERTNQTGVEPCEGEKDKRLHCFATWRNISGSIEIVKKGCWLDDINCYDRTDCVETKENPQVYFCCCEGNMCNEKFSYFPEMEVTQPTS

SEQ ID NO: 154 - protein, 109 aa, synthetic construct
GAILGRSETQECLFYNANWELERTNQTGVEPCEGEKDKRLHCFATWKNISGSIEIVKKGCWLDDFNCYDRTDCVETKENPQVYFCCCEGNMCNEKFSYFPEMEVTQPTS

SEQ ID NO: 155 - protein, 109 aa, synthetic construct
GAILGRSETQECLFYNANWELERTNQTGVEPCEGEKDKRLHCFATWKNISGSIEIVKKGCWLDDINCYDRTDCVETEENPQVYFCCCEGNMCNEKFSYFPEMEVTQPTS

SEQ ID NO: 156 - protein, 109 aa, synthetic construct
GAILGRSETQECLFYNANWELDRTNQTGVERCEGEKDKRLHCYATWKNISGSIEIVKKGCWLDDINCYDRTDCVETKENPQVYFCCCEGNMCNEKFSYFPEMEVTQPTS

SEQ ID NO: 157 - protein, 109 aa, synthetic construct
GAILGRSETQECLFYNANWELDRTNQTGVERCEGEKDKRLHCFATWRNISGSIEIVKKGCWLDDINCYDRTDCVETKENPQVYFCCCEGNMCNEKFSYFPEMEVTQPTS

SEQ ID NO: 158 - protein, 109 aa, synthetic construct
GAILGRSETQECLFYNANWELDRTNQTGVERCEGEKDKRLHCFATWKNISGSIEIVKKGCWLDDFNCYDRTDCVETKENPQVYFCCCEGNMCNEKFSYFPEMEVTQPTS

SEQ ID NO: 159 - protein, 109 aa, synthetic construct
GAILGRSETQECLFYNANWELDRTNQTGVERCEGEKDKRLHCFATWKNISGSIEIVKKGCWLDDINCYDRTDCVETEENPQVYFCCCEGNMCNEKFSYFPEMEVTQPTS

SEQ ID NO: 160 - protein, 109 aa, synthetic construct
GAILGRSETQECLFYNANWELDRTNQTGVEPCEGEKDKRLHCYATWRNISGSIEIVKKGCWLDDINCYDRTDCVETKENPQVYFCCCEGNMCNEKFSYFPEMEVTQPTS

SEQ ID NO: 161 - protein, 109 aa, synthetic construct
GAILGRSETQECLFYNANWELDRTNQTGVEPCEGEKDKRLHCYATWKNISGSIEIVKKGCWLDDFNCYDRTDCVETKENPQVYFCCCEGNMCNEKFSYFPEMEVTQPTS

SEQ ID NO: 162 - protein, 109 aa, synthetic construct
GAILGRSETQECLFYNANWELDRTNQTGVEPCEGEKDKRLHCYATWKNISGSIEIVKKGCWLDDINCYDRTDCVETEENPQVYFCCCEGNMCNEKFSYFPEMEVTQPTS

SEQ ID NO: 163 - protein, 109 aa, synthetic construct
GAILGRSETQECLFYNANWELDRTNQTGVEPCEGEKDKRLHCFATWRNISGSIEIVKKGCWLDDFNCYDRTDCVETKENPQVYFCCCEGNMCNEKFSYFPEMEVTQPTS

SEQ ID NO: 164 - protein, 109 aa, synthetic construct
GAILGRSETQECLFYNANWELDRTNQTGVEPCEGEKDKRLHCFATWRNISGSIEIVKKGCWLDDINCYDRTDCVETEENPQVYFCCCEGNMCNEKFSYFPEMEVTQPTS

SEQ ID NO: 165 - protein, 109 aa, synthetic construct
GAILGRSETQECLFYNANWELDRTNQTGVEPCEGEKDKRLHCFATWKNISGSIEIVKKGCWLDDFNCYDRTDCVETEENPQVYFCCCEGNMCNEKFSYFPEMEVTQPTS

SEQ ID NO: 166 - protein, 109 aa, synthetic construct
GAILGRSETQECLFYNANWELERTNQTGVERCEGEKDKRLHCYATWKNISGSIEIVKKGCWLDDINCYDRTDCVETKENPQVYFCCCEGNMCNEKFSYFPEMEVTQPTS

SEQ ID NO: 167 - protein, 109 aa, synthetic construct
GAILGRSETQECLFYNANWELERTNQTGVERCEGEKDKRLHCFATWRNISGSIEIVKKGCWLDDINCYDRTDCVETKENPQVYFCCCEGNMCNEKFSYFPEMEVTQPTS

SEQ ID NO: 168 - protein, 109 aa, synthetic construct
GAILGRSETQECLFYNANWELERTNQTGVERCEGEKDKRLHCFATWKNISGSIEIVKKGCWLDDFNCYDRTDCVETKENPQVYFCCCEGNMCNEKFSYFPEMEVTQPTS

SEQ ID NO: 169 - protein, 109 aa, synthetic construct
GAILGRSETQECLFYNANWELERTNQTGVERCEGEKDKRLHCFATWKNISGSIEIVKKGCWLDDINCYDRTDCVETEENPQVYFCCCEGNMCNEKFSYFPEMEVTQPTS

SEQ ID NO: 170 - protein, 109 aa, synthetic construct
GAILGRSETQECLFYNANWELERTNQTGVEPCEGEKDKRLHCYATWRNISGSIEIVKKGCWLDDINCYDRTDCVETKENPQVYFCCCEGNMCNEKFSYFPEMEVTQPTS

SEQ ID NO: 171 - protein, 109 aa, synthetic construct
GAILGRSETQECLFYNANWELERTNQTGVEPCEGEKDKRLHCYATWKNISGSIEIVKKGCWLDDFNCYDRTDCVETKENPQVYFCCCEGNMCNEKFSYFPEMEVTQPTS

SEQ ID NO: 172 - protein, 109 aa, synthetic construct
GAILGRSETQECLFYNANWELERTNQTGVEPCEGEKDKRLHCYATWKNISGSIEIVKKGCWLDDINCYDRTDCVETEENPQVYFCCCEGNMCNEKFSYFPEMEVTQPTS

SEQ ID NO: 173 - protein, 109 aa, synthetic construct
GAILGRSETQECLFYNANWELERTNQTGVEPCEGEKDKRLHCFATWRNISGSIEIVKKGCWLDDFNCYDRTDCVETKENPQVYFCCCEGNMCNEKFSYFPEMEVTQPTS

SEQ ID NO: 174 - protein, 109 aa, synthetic construct
GAILGRSETQECLFYNANWELERTNQTGVEPCEGEKDKRLHCFATWRNISGSIEIVKKGCWLDDINCYDRTDCVETEENPQVYFCCCEGNMCNEKFSYFPEMEVTQPTS

SEQ ID NO: 175 - protein, 109 aa, synthetic construct
GAILGRSETQECLFYNANWELERTNQTGVEPCEGEKDKRLHCFATWKNISGSIEIVKKGCWLDDFNCYDRTDCVETEENPQVYFCCCEGNMCNEKFSYFPEMEVTQPTS

SEQ ID NO: 176 - protein, 109 aa, synthetic construct
GAILGRSETQECLFYNANWELDRTNQTGVERCEGEKDKRLHCYATWRNISGSIEIVKKGCWLDDINCYDRTDCVETKENPQVYFCCCEGNMCNEKFSYFPEMEVTQPTS

SEQ ID NO: 177 - protein, 109 aa, synthetic construct
GAILGRSETQECLFYNANWELDRTNQTGVERCEGEKDKRLHCYATWKNISGSIEIVKKGCWLDDFNCYDRTDCVETKENPQVYFCCCEGNMCNEKFSYFPEMEVTQPTS

SEQ ID NO: 178 - protein, 109 aa, synthetic construct
GAILGRSETQECLFYNANWELDRTNQTGVERCEGEKDKRLHCYATWKNISGSIEIVKKGCWLDDINCYDRTDCVETEENPQVYFCCCEGNMCNEKFSYFPEMEVTQPTS

SEQ ID NO: 179 - protein, 109 aa, synthetic construct
GAILGRSETQECLFYNANWELDRTNQTGVERCEGEKDKRLHCFATWRNISGSIEIVKKGCWLDDFNCYDRTDCVETKENPQVYFCCCEGNMCNEKFSYFPEMEVTQPTS

SEQ ID NO: 180 - protein, 109 aa, synthetic construct
GAILGRSETQECLFYNANWELDRTNQTGVERCEGEKDKRLHCFATWRNISGSIEIVKKGCWLDDINCYDRTDCVETEENPQVYFCCCEGNMCNEKFSYFPEMEVTQPTS

SEQ ID NO: 181 - protein, 109 aa, synthetic construct
GAILGRSETQECLFYNANWELDRTNQTGVERCEGEKDKRLHCFATWKNISGSIEIVKKGCWLDDFNCYDRTDCVETEENPQVYFCCCEGNMCNEKFSYFPEMEVTQPTS

SEQ ID NO: 182 - protein, 109 aa, synthetic construct
GAILGRSETQECLFYNANWELDRTNQTGVEPCEGEKDKRLHCYATWRNISGSIEIVKKGCWLDDFNCYDRTDCVETKENPQVYFCCCEGNMCNEKFSYFPEMEVTQPTS

SEQ ID NO: 183 - protein, 109 aa, synthetic construct
GAILGRSETQECLFYNANWELDRTNQTGVEPCEGEKDKRLHCYATWRNISGSIEIVKKGCWLDDINCYDRTDCVETEENPQVYFCCCEGNMCNEKFSYFPEMEVTQPTS

SEQ ID NO: 184 - protein, 109 aa, synthetic construct
GAILGRSETQECLFYNANWELDRTNQTGVEPCEGEKDKRLHCYATWKNISGSIEIVKKGCWLDDFNCYDRTDCVETEENPQVYFCCCEGNMCNEKFSYFPEMEVTQPTS

SEQ ID NO: 185 - protein, 109 aa, synthetic construct
GAILGRSETQECLFYNANWELDRTNQTGVEPCEGEKDKRLHCFATWRNISGSIEIVKKGCWLDDFNCYDRTDCVETEENPQVYFCCCEGNMCNEKFSYFPEMEVTQPTS

SEQ ID NO: 186 - protein, 109 aa, synthetic construct
GAILGRSETQECLFYNANWELERTNQTGVERCEGEKDKRLHCYATWRNISGSIEIVKKGCWLDDINCYDRTDCVETKENPQVYFCCCEGNMCNEKFSYFPEMEVTQPTS

SEQ ID NO: 187 - protein, 109 aa, synthetic construct
GAILGRSETQECLFYNANWELERTNQTGVERCEGEKDKRLHCYATWKNISGSIEIVKKGCWLDDFNCYDRTDCVETKENPQVYFCCCEGNMCNEKFSYFPEMEVTQPTS

SEQ ID NO: 188 - protein, 109 aa, synthetic construct
GAILGRSETQECLFYNANWELERTNQTGVERCEGEKDKRLHCYATWKNISGSIEIVKKGCWLDDINCYDRTDCVETEENPQVYFCCCEGNMCNEKFSYFPEMEVTQPTS

SEQ ID NO: 189 - protein, 109 aa, synthetic construct
GAILGRSETQECLFYNANWELERTNQTGVERCEGEKDKRLHCFATWRNISGSIEIVKKGCWLDDFNCYDRTDCVETKENPQVYFCCCEGNMCNEKFSYFPEMEVTQPTS

SEQ ID NO: 190 - protein, 109 aa, synthetic construct
GAILGRSETQECLFYNANWELERTNQTGVERCEGEKDKRLHCFATWRNISGSIEIVKKGCWLDDINCYDRTDCVETEENPQVYFCCCEGNMCNEKFSYFPEMEVTQPTS

SEQ ID NO: 191 - protein, 109 aa, synthetic construct
GAILGRSETQECLFYNANWELERTNQTGVERCEGEKDKRLHCFATWKNISGSIEIVKKGCWLDDFNCYDRTDCVETEENPQVYFCCCEGNMCNEKFSYFPEMEVTQPTS

SEQ ID NO: 192 - protein, 109 aa, synthetic construct
GAILGRSETQECLFYNANWELERTNQTGVEPCEGEKDKRLHCYATWRNISGSIEIVKKGCWLDDFNCYDRTDCVETKENPQVYFCCCEGNMCNEKFSYFPEMEVTQPTS

SEQ ID NO: 193 - protein, 109 aa, synthetic construct
GAILGRSETQECLFYNANWELERTNQTGVEPCEGEKDKRLHCYATWRNISGSIEIVKKGCWLDDINCYDRTDCVETEENPQVYFCCCEGNMCNEKFSYFPEMEVTQPTS

SEQ ID NO: 194 - protein, 109 aa, synthetic construct
GAILGRSETQECLFYNANWELERTNQTGVEPCEGEKDKRLHCYATWKNISGSIEIVKKGCWLDDFNCYDRTDCVETEENPQVYFCCCEGNMCNEKFSYFPEMEVTQPTS

SEQ ID NO: 195 - protein, 109 aa, synthetic construct
GAILGRSETQECLFYNANWELERTNQTGVEPCEGEKDKRLHCFATWRNISGSIEIVKKGCWLDDFNCYDRTDCVETEENPQVYFCCCEGNMCNEKFSYFPEMEVTQPTS

SEQ ID NO: 196 - protein, 109 aa, synthetic construct
GAILGRSETQECLFYNANWELDRTNQTGVERCEGEKDKRLHCYATWRNISGSIEIVKKGCWLDDFNCYDRTDCVETKENPQVYFCCCEGNMCNEKFSYFPEMEVTQPTS

SEQ ID NO: 197 - protein, 109 aa, synthetic construct
GAILGRSETQECLFYNANWELDRTNQTGVERCEGEKDKRLHCYATWRNISGSIEIVKKGCWLDDINCYDRTDCVETEENPQVYFCCCEGNMCNEKFSYFPEMEVTQPTS

SEQ ID NO: 198 - protein, 109 aa, synthetic construct
GAILGRSETQECLFYNANWELDRTNQTGVERCEGEKDKRLHCYATWKNISGSIEIVKKGCWLDDFNCYDRTDCVETEENPQVYFCCCEGNMCNEKFSYFPEMEVTQPTS

SEQ ID NO: 199 - protein, 109 aa, synthetic construct
GAILGRSETQECLFYNANWELDRTNQTGVERCEGEKDKRLHCFATWRNISGSIEIVKKGCWLDDFNCYDRTDCVETEENPQVYFCCCEGNMCNEKFSYFPEMEVTQPTS

SEQ ID NO: 200 - protein, 109 aa, synthetic construct
GAILGRSETQECLFYNANWELDRTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 201 - protein, 109 aa, synthetic construct
GAILGRSETQECLFYNANWELERTNQTGVERCEGEKDKRLHCYATWRNISGSIEIVKKGCWLDDFNCYDRTDCVETKENPQVYFCCCEGNMCNEKFSYFPEMEVTQPTS

SEQ ID NO: 202 - protein, 109 aa, synthetic construct
GAILGRSETQECLFYNANWELERTNQTGVERCEGEKDKRLHCYATWRNISGSIEIVKKGCWLDDINCYDRTDCVETEENPQVYFCCCEGNMCNEKFSYFPEMEVTQPTS

SEQ ID NO: 203 - protein, 109 aa, synthetic construct
GAILGRSETQECLFYNANWELERTNQTGVERCEGEKDKRLHCYATWKNISGSIEIVKKGCWLDDFNCYDRTDCVETEENPQVYFCCCEGNMCNEKFSYFPEMEVTQPTS

SEQ ID NO: 204 - protein, 109 aa, synthetic construct
GAILGRSETQECLFYNANWELERTNQTGVERCEGEKDKRLHCFATWRNISGSIEIVKKGCWLDDFNCYDRTDCVETEENPQVYFCCCEGNMCNEKFSYFPEMEVTQPTS

SEQ ID NO: 205 - protein, 109 aa, synthetic construct
GAILGRSETQECLFYNANWELDRTNQTGVERCEGEKDKRLHCYATWRNISGSIEIVKKGCWLDDFNCYDRTDCVETEENPQVYFCCCEGNMCNEKFSYFPEMEVTQPTS

SEQ ID NO: 206 - protein, 109 aa, synthetic construct
GAILGRSETQECLFYNANWELERTNQTGVEPCEGEKDKRLHCYATWRNISGSIEIVKKGCWLDDFNCYDRTDCVETEENPQVYFCCCEGNMCNEKFSYFPEMEVTQPTS

SEQ ID NO: 207 - protein, 109 aa, synthetic construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTS

SEQ ID NO: 208 - protein, 109 aa, synthetic construct
GAILGRSETQECLYYNANWELERTNQTGVERCEGEQDKRLHCYATWRNISGSIEIVKKGCWLDDFNCYDRTDCVETEENPQVYFCCCEGNMCNEKFSYFPEMEVTQPTS

SEQ ID NO: 209 - protein, 111 aa, synthetic construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSNP

SEQ ID NO: 210 - protein, 115 aa, synthetic construct
GAILGRSETQECLFYNANWELERTNQTGVERCEGEKDKRLHCYATWRNISGSIEIVKKGCWLDDFNCYDRTDCVETEENPQVYFCCCEGNMCNEKFSYFPEMEVTQPTSNPVTPK

SEQ ID NO: 211 - protein, 109 aa, synthetic construct
GAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 212 - protein, 109 aa, synthetic construct
GAILGRSETQECLFFNANWEKDRTNQTGVEPCYGDKDKRRHCFATWANISGSIEIVKQGCWLDDINCYDRTDCVEKKDSPEVYFCCCEGNMCNEKFSYFPEMEVTQPTS

SEQ ID NO: 213 - protein, 109 aa, synthetic construct
GAILGRSETQECLFFNANWAKDRTNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 214 - protein, 109 aa, synthetic construct
GAILGRSETQECLFFNANWEKDATNQTGVEPCYGDKDKRRHCFATWKNISGSIEIVKQGCWLDDINCYDRTDCVEKKDSPEVYFCCCEGNMCNEKFSYFPEMEVTQPTS

SEQ ID NO: 215 - protein, 109 aa, synthetic construct
GAILGRSETQECLFFNANWEKDRTNQTGVEPCYGDKAKRRHCFATWKNISGSIEIVKQGCWLDDINCYDRTDCVEKKDSPEVYFCCCEGNMCNEKFSYFPEMEVTQPTS

SEQ ID NO: 216 - protein, 109 aa, synthetic construct
GAILGRSETQECLFFNANWEKDRTNQTGVEPCYGDKDARRHCFATWKNISGSIEIVKQGCWLDDINCYDRTDCVEKKDSPEVYFCCCEGNMCNEKFSYFPEMEVTQPTS

SEQ ID NO: 217 - protein, 109 aa, synthetic construct
GAILGRSETQECLFFNANWEKDRTNQTGVEPCYGDKDKARHCFATWKNISGSIEIVKQGCWLDDINCYDRTDCVEKKDSPEVYFCCCEGNMCNEKFSYFPEMEVTQPTS

SEQ ID NO: 218 - protein, 109 aa, synthetic construct
GAILGRSETQECLFFNANWEKDRTNQTGVEPCYGDKDKRRHCFATWKNISGSIEIVAQGCWLDDINCYDRTDCVEKKDSPEVYFCCCEGNMCNEKFSYFPEMEVTQPTS

SEQ ID NO: 219 - protein, 109 aa, synthetic construct
GAILGRSETQECLFFNANWEKDRTNQTGVEPCYGDKDKRRHCFATWKNISGSIEIVYQGCWLDDINCYDRTDCVEKKDSPEVYFCCCEGNMCNEKFSYFPEMEVTQPTS

SEQ ID NO: 220 - protein, 109 aa, synthetic construct
GAILGRSETQECLFFNANWEKDRTNQTGVEPCYGDKDKRRHCFATWKNISGSIEIVFQGCWLDDINCYDRTDCVEKKDSPEVYFCCCEGNMCNEKFSYFPEMEVTQPTS

SEQ ID NO: 221 - protein, 109 aa, synthetic construct
GAILGRSETQECLFFNANWEKDRTNQTGVEPCYGDKDKRRHCFATWKNISGSIEIVIQGCWLDDINCYDRTDCVEKKDSPEVYFCCCEGNMCNEKFSYFPEMEVTQPTS

SEQ ID NO: 222 - protein, 109 aa, synthetic construct
GAILGRSETQECLFFNANWEKDRTNQTGVEPCYGDKDKRRHCFATWKNISGSIEIVKQGCALDDINCYDRTDCVEKKDSPEVYFCCCEGNMCNEKFSYFPEMEVTQPTS

SEQ ID NO: 223 - protein, 109 aa, synthetic construct
GAILGRSETQECLFFNANWEKDRTNQTGVEPCYGDKDKRRHCFATWKNISGSIEIVKQGCWADDINCYDRTDCVEKKDSPEVYFCCCEGNMCNEKFSYFPEMEVTQPTS

SEQ ID NO: 224 - protein, 109 aa, synthetic construct
GAILGRSETQECLFFNANWEKDRTNQTGVEPCYGDKDKRRHCFATWKNISGSIEIVKQGCWLKDINCYDRTDCVEKKDSPEVYFCCCEGNMCNEKFSYFPEMEVTQPTS

SEQ ID NO: 225 - protein, 109 aa, synthetic construct
GAILGRSETQECLFFNANWEKDRTNQTGVEPCYGDKDKRRHCFATWKNISGSIEIVKQGCWLRDINCYDRTDCVEKKDSPEVYFCCCEGNMCNEKFSYFPEMEVTQPTS

SEQ ID NO: 226 - protein, 109 aa, synthetic construct
GAILGRSETQECLFFNANWEKDRTNQTGVEPCYGDKDKRRHCFATWKNISGSIEIVKQGCWLADINCYDRTDCVEKKDSPEVYFCCCEGNMCNEKFSYFPEMEVTQPTS

SEQ ID NO: 227 - protein, 109 aa, synthetic construct
GAILGRSETQECLFFNANWEKDRTNQTGVEPCYGDKDKRRHCFATWKNISGSIEIVKQGCWLFDINCYDRTDCVEKKDSPEVYFCCCEGNMCNEKFSYFPEMEVTQPTS

SEQ ID NO: 228 - protein, 109 aa, synthetic construct
GAILGRSETQECLFFNANWEKDRTNQTGVEPCYGDKDKRRHCFATWKNISGSIEIVKQGCWLGDINCYDRTDCVEKKDSPEVYFCCCEGNMCNEKFSYFPEMEVTQPTS

SEQ ID NO: 229 - protein, 109 aa, synthetic construct
GAILGRSETQECLFFNANWEKDRTNQTGVEPCYGDKDKRRHCFATWKNISGSIEIVKQGCWLMDINCYDRTDCVEKKDSPEVYFCCCEGNMCNEKFSYFPEMEVTQPTS

SEQ ID NO: 230 - protein, 109 aa, synthetic construct
GAILGRSETQECLFFNANWEKDRTNQTGVEPCYGDKDKRRHCFATWKNISGSIEIVKQGCWLNDINCYDRTDCVEKKDSPEVYFCCCEGNMCNEKFSYFPEMEVTQPTS

SEQ ID NO: 231 - protein, 109 aa, synthetic construct
GAILGRSETQECLFFNANWEKDRTNQTGVEPCYGDKDKRRHCFATWKNISGSIEIVKQGCWLIDINCYDRTDCVEKKDSPEVYFCCCEGNMCNEKFSYFPEMEVTQPTS

SEQ ID NO: 232 - protein, 109 aa, synthetic construct
GAILGRSETQECLFFNANWEKDRTNQTGVEPCYGDKDKRRHCFATWKNISGSIEIVKQGCWLDDANCYDRTDCVEKKDSPEVYFCCCEGNMCNEKFSYFPEMEVTQPTS

SEQ ID NO: 233 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGPFFLYSKLTVDKSRWQQGNVFSCSVMHEALHNHYTQKSLSLSPGK

SEQ ID NO: 234 - protein, 4 aa, synthetic construct
GGGA

SEQ ID NO: 235 - protein, 4 aa, synthetic construct
GGGS

SEQ ID NO: 236 - protein, 5 aa, synthetic construct
GGGGA

SEQ ID NO: 237 - protein, 5 aa, synthetic construct
GGGGG

SEQ ID NO: 238 - protein, 4 aa, synthetic construct
GGAG

SEQ ID NO: 239 - protein, 4 aa, synthetic construct
GGSG

SEQ ID NO: 240 - protein, 4 aa, synthetic construct
AGGG

SEQ ID NO: 241 - protein, 4 aa, synthetic construct
GAGA

SEQ ID NO: 242 - protein, 4 aa, synthetic construct
GSGS

SEQ ID NO: 243 - protein, 6 aa, synthetic construct
GAGAGA

SEQ ID NO: 244 - protein, 6 aa, synthetic construct
GSGSGS

SEQ ID NO: 245 - protein, 8 aa, synthetic construct
GAGAGAGA

SEQ ID NO: 246 - protein, 8 aa, synthetic construct
GSGSGSGS

SEQ ID NO: 247 - protein, 10 aa, synthetic construct
GAGAGAGAGA

SEQ ID NO: 248 - protein, 10 aa, synthetic construct
GSGSGSGSGS

SEQ ID NO: 249 - protein, 12 aa, synthetic construct
GAGAGAGAGAGA

SEQ ID NO: 250 - protein, 12 aa, synthetic construct
GSGSGSGSGSGS

SEQ ID NO: 251 - protein, 6 aa, synthetic construct
GGAGGA

SEQ ID NO: 252 - protein, 6 aa, synthetic construct
GGSGGS

SEQ ID NO: 253 - protein, 9 aa, synthetic construct
GGAGGAGGA

SEQ ID NO: 254 - protein, 9 aa, synthetic construct
GGSGGSGGS

SEQ ID NO: 255 - protein, 12 aa, synthetic construct
GGAGGAGGAGGA

SEQ ID NO: 256 - protein, 12 aa, synthetic construct
GGSGGSGGSGGS

SEQ ID NO: 257 - protein, 8 aa, synthetic construct
GGAGGGAG

SEQ ID NO: 258 - protein, 8 aa, synthetic construct
GGSGGGSG

SEQ ID NO: 259 - protein, 12 aa, synthetic construct
GGAGGGAGGGAG

SEQ ID NO: 260 - protein, 12 aa, synthetic construct
GGSGGGSGGGSG

SEQ ID NO: 261 - protein, 15 aa, synthetic construct
GGGGAGGGGAGGGGA

SEQ ID NO: 262 - protein, 15 aa, synthetic construct
GGGGSGGGGSGGGGS

SEQ ID NO: 263 - protein, 5 aa, synthetic construct
GGGAG

SEQ ID NO: 264 - protein, 6 aa, synthetic construct
GGGAGG

SEQ ID NO: 265 - protein, 7 aa, synthetic construct
GGGAGGG

SEQ ID NO: 266 - protein, 4 aa, synthetic construct
AAAL

SEQ ID NO: 267 - protein, 4 aa, synthetic construct
AAAK

SEQ ID NO: 268 - protein, 4 aa, synthetic construct
AAAR

SEQ ID NO: 269 - protein, 14 aa, synthetic construct
EGKSSGSGSESKST

SEQ ID NO: 270 - protein, 12 aa, synthetic construct
GSAGSAAGSGEF

SEQ ID NO: 271 - protein, 12 aa, synthetic construct
AEAAAKEAAAKA

SEQ ID NO: 272 - protein, 18 aa, synthetic construct
KESGSVSSEQLAQFRSLD

SEQ ID NO: 273 - protein, 10 aa, synthetic construct
GENLYFQSGG

SEQ ID NO: 274 - protein, 8 aa, synthetic construct
SACYCELS

SEQ ID NO: 275 - protein, 5 aa, synthetic construct
RSIAT

SEQ ID NO: 276 - protein, 17 aa, synthetic construct
RPACKIPNDLKQKVMNH

SEQ ID NO: 277 - protein, 36 aa, synthetic construct
GGSAGGSGSGSSGGSSGASGTGTAGGTGSGSGTGSG

SEQ ID NO: 278 - protein, 17 aa, synthetic construct
AAANSSIDLISVPVDSR

SEQ ID NO: 279 - protein, 36 aa, synthetic construct
GGSGGGSEGGGSEGGGSEGGGSEGGGSEGGGSGGGS

SEQ ID NO: 280 - protein, 5 aa, synthetic construct
EAAAK

SEQ ID NO: 281 - protein, 5 aa, synthetic construct
PAPAP

SEQ ID NO: 282 - protein, 115 aa, synthetic construct
GRGEAETRECIFYNANWEKDRTNQSGLEPCYGDQDKRRHCFASWKNSSGTIELVKQGCWLDDINCYDRQECVAKKDSPEVYFCCCEGNFCNERFTHLPEAGGPEVTYEPPPTAPT

SEQ ID NO: 283 - protein, 109 aa, synthetic construct
GAILGRSETQECLFYNANWELERTNQTGVERCEGEKDKRLHCYATWRNISGSIEIVAKGCWLDDFNCYDRTDCVETEENPQVYFCCCEGNMCNEKFSYFPEMEVTQPTS

SEQ ID NO: 284 - protein, 246 aa, synthetic construct
MEWSWVFLFFLSVTTGVHSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 285 - protein, 11 aa, unidentified
DICLPRWGCLW

SEQ ID NO: 286 - protein, 93 aa, synthetic construct
GPVEVFITETPSQPNSHPIQWNAPQPSHISKYILRWRPKNSVGRWKEATIPGHLNSYTIKGLKPGVVYEGQLISIQQYGHQEVTRFDFTTTST

SEQ ID NO: 287 - protein, 609 aa, Homo sapiens [sequence omitted]

SEQ ID NO: 288 - protein, 6 aa, synthetic construct
NPVTPK

SEQ ID NO: 289 - protein, 115 aa, synthetic construct
  VARIANT 11: note = isoleucine or leucine
  VARIANT 12: note = phenylalanine, tyrosine, alanine, valine, isoleucine, leucine, methionine, or tryptophan
  VARIANT 19: note = leucine or lysine
  VARIANT 20: note = aspartic acid, glutamic acid, or alanine
  VARIANT 25: note = threonine, serine, asparagine, or glutamine
  VARIANT 27: note = leucine, valine, alanine, isoleucine, methionine, phenylalanine, tyrosine, or tryptophan
  VARIANT 29: note = proline or arginine
  VARIANT 31: note = tyrosine or glutamic acid
  VARIANT 33: note = aspartic acid, glutamic acid, or alanine
  VARIANT 34: note = lysine or glutamine
  VARIANT 38: note = arginine or leucine
  VARIANT 41: note = alanine, valine, isoleucine, leucine, methionine, tyrosine, tryptophan or phenylalanine
  VARIANT 45: note = arginine, histidine, or lysine
  VARIANT 47: note = serine or isoleucine
  VARIANT 48: note = serine, asparagine, glutamine, or threonine
  VARIANT 50: note = serine, asparagine, glutamine, or threonine
  VARIANT 51: note = leucine, valine, alanine, isoleucine, methionine, phenylalanine, tyrosine, or tryptophan
  VARIANT 53: note = leucine, valine, alanine, isoleucine, methionine, phenylalanine, tyrosine, or tryptophan
  VARIANT 56: note = lysine or glutamine
  VARIANT 63: note = leucine, valine, alanine, isoleucine, methionine, phenylalanine, tyrosine, or tryptophan
  VARIANT 69: note = glutamine, threonine, serine, asparagine or aspartic acid
  VARIANT 70: note = glutamic acid, aspartic acid, alanine, or threonine
  VARIANT 74: note = lysine or threonine
  VARIANT 75: note = lysine or glutamic acid
  VARIANT 76: note = aspartic acid, glutamic acid, or alanine
  VARIANT 77: note = threonine, serine, asparagine, or glutamine
  VARIANT 79: note = glutamic acid or glutamine
  VARIANT 89: note = leucine, valine, alanine, isoleucine, methionine, phenylalanine, tyrosine, or tryptophan
GRGEAETRECXXYNANWEXXRTNQXGXEXCXGXXDKRXHCXASWXNXXGXXEXVKXGCWLDDXNCYDRXXCVAXXXXPXVYFCCCEGNXCNERFTHLPEAGGPEVTYEPPPTAPT

SEQ ID NO: 290 - protein, 115 aa, synthetic construct
GRGEAETRECIYYNANWELDRTNQSGLERCEGEQDKRLHCYASWRNSSGTIELVKKGCWLDDINCYDRQECVATKENPQVYFCCCEGNFCNERFTHLPEAGGPEVTYEPPPTAPT

SEQ ID NO: 291 - protein, 115 aa, synthetic construct
GRGEAETRECIYYNANWELERTNQSGLERCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 292 - protein, 115 aa, synthetic construct
GRGEAETRECIYYNANWELERTNQTGLERCEGEQDKRLHCYASWRNISGTIELVKKGCWLDDFNCYDRQECVAKKDSPEVYFCCCEGNFCNERFTHLPEAGGPEVTYEPPPTAPT

SEQ ID NO: 293 - protein, 115 aa, synthetic construct
GRGEAETRECIYYNANWELERTNQTGLERCEGEQDKRLHCYASWRNITGTIELVKKGCWLDDFNCYDRQECVAKKDSPEVYFCCCEGNFCNERFTHLPEAGGPEVTYEPPPTAPT

SEQ ID NO: 294 - protein, 115 aa, synthetic construct
GRGEAETRECIYYNANWELERTNQSGLEPCEGEQDKRLHCYASWRNSSGTIELVKKGCWLDDFNCYDRQECVAKKDSPEVYFCCCEGNFCNERFTHLPEAGGPEVTYEPPPTAPT

SEQ ID NO: 295 - protein, 115 aa, synthetic construct
GRGEAETRECIYYNANWELERTNQSGLERCYGDKDKRLHCFASWKNSSGTIELVKQGCWLDDFNCYDRQECVAKKDSPEVYFCCCEGNFCNERFTHLPEAGGPEVTYEPPPTAPT

SEQ ID NO: 296 - protein, 115 aa, synthetic construct; identical to SEQ ID NO: 291

SEQ ID NO: 297 - protein, 115 aa, synthetic construct
GRGEAETRECIFYNANWEKDRTNQSGLERCYGDQDKRRHCYASWRNSSGTIELVKKGCWLDDFNCYDRQECVAKKDSPEVYFCCCEGNFCNERFTHLPEAGGPEVTYEPPPTAPT

SEQ ID NO: 298 - protein, 115 aa, synthetic construct; identical to SEQ ID NO: 291

SEQ ID NO: 299 - protein, 115 aa, synthetic construct
GRGEAETRECLYYNANWELERTNQSGVERCEGEKDKRLHCYASWRNSSGSLEIVKKGCWLDDFNCYDRTDCVATEENPQVYFCCCEGNMCNERFTHLPEAGGPEVTYEPPPTAPT

SEQ ID NO: 300 - protein, 115 aa, synthetic construct
GRGEAETRECLYYNANWELERTNQSGVERCEGEKDKRLHCYASWRNSSGSLEIVKKGCWLDDFNCYDRDTCVATEENPQVYFCCCEGNMCNERFTHLPEAGGPEVTYEPPPTAPT

SEQ ID NO: 301 - protein, 115 aa, synthetic construct
GRGEAETRECLYYNANWELERTNQSGVERCEGEKDKRLHCYASWRNSSGSLEIVKKGCWLDDFNCYDRTDCVATKENPQVYFCCCEGNMCNERFTHLPEAGGPEVTYEPPPTAPT

SEQ ID NO: 302 - protein, 115 aa, synthetic construct
GRGEAETRECLYYNANWELERTNQSGVERCEGEKDKRLHCYASWRNSSGSLEIVKKGCWLDDFNCYDRDTCVATKENPQVYFCCCEGNMCNERFTHLPEAGGPEVTYEPPPTAPT

SEQ ID NO: 303 - protein, 110 aa, synthetic construct
ETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 304 - protein, 110 aa, synthetic construct
ETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 305 - protein, 110 aa, synthetic construct
ETRECIYYNANWELERTNQSGLERCYGDKDKRRHCYASWRNSSGTIELVKKGCWLDDINCYDRTDCVEKKDSPEVYFCCCEGNFCNERFTHLPEAGGPEVTYEPPPTAPT

SEQ ID NO: 306 - protein, 110 aa, synthetic construct
ETRECIYYNANWELERTNQSGLERCYGDKDKRRHCYASWRNSSGTIELVKKGCWLDDFNCYDRTDCVEKKDSPEVYFCCCEGNFCNERFTHLPEAGGPEVTYEPPPTAPT

SEQ ID NO: 307 - protein, 110 aa, synthetic construct
ETRECIYYNANWELERTNQSGLERCEGEQDKRLHCYASWRNSSGTIELVKKGCWLDDINCYDRTDCVEKKDSPEVYFCCCEGNFCNERFTHLPEAGGPEVTYEPPPTAPT

SEQ ID NO: 308 - protein, 110 aa, synthetic construct
ETRECIYYNANWELERTNQSGLERCEGEQDKRLHCYASWRNSSGTIELVKKGCWLDDINCYDRTDCVEKKDSPQVYFCCCEGNFCNERFTHLPEAGGPEVTYEPPPTAPT

SEQ ID NO: 309 - protein, 110 aa, synthetic construct
ETRECIYYNANWELERTNQSGLERCEGEQDKRLHCYASWRNSSGTIELVKKGCWLDDFNCYDRTDCVEKKDSPQVYFCCCEGNFCNERFTHLPEAGGPEVTYEPPPTAPT

SEQ ID NO: 310 - protein, 110 aa, synthetic construct
ETRECIYYNANWELERTNQSGLERCEGEQDKRLHCYASWRNSSGTIELVKKGCWLDDFNCYDRTDCVEKKDSPEVYFCCCEGNFCNERFTHLPEAGGPEVTYEPPPTAPT

SEQ ID NO: 311 - protein, 110 aa, synthetic construct
ETRECIYYNANWELERTNQSGLERCEGEQDKRLHCYASWRNSSGTIELVKKGCWLDDFNCYDRQECVEKKDSPEVYFCCCEGNFCNERFTHLPEAGGPEVTYEPPPTAPT

SEQ ID NO: 312 - protein, 110 aa, synthetic construct
ETRECIYYNANWELERTNQSGLERCEGEQDKRLHCYASWRNSSGTIELVKKGCWLDDFNCYDRQECVEKKDSPQVYFCCCEGNFCNERFTHLPEAGGPEVTYEPPPTAPT

SEQ ID NO: 313 - protein, 110 aa, synthetic construct
ETRECIYYNANWELERTNQSGLERCEGEQDKRLHCYASWRNSSGTIELVKKGCWLDDINCYDRQECVEKKDSPEVYFCCCEGNFCNERFTHLPEAGGPEVTYEPPPTAPT

SEQ ID NO: 314 - protein, 110 aa, synthetic construct
ETRECIYYNANWELERTNQSGLERCEGEQDKRLHCYASWRNSSGTIELVKKGCWLDDFNCYDRTDCVEKKDSPEVYFCCCEGNMCNERFTHLPEAGGPEVTYEPPPTAPT

SEQ ID NO: 315 - protein, 110 aa, synthetic construct
ETRECIYYNANWELERTNQSGLERCEGDQDKRLHCYASWRNSSGTIELVKKGCWLDDFNCYDRQECVEKKDSPEVYFCCCEGNMCNERFTHLPEAGGPEVTYEPPPTAPT

SEQ ID NO: 316 - protein, 110 aa, synthetic construct
ETQECIYYNANWEKDRTNQTGVEPCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 317 - protein, 110 aa, synthetic construct
ETQECIYYNANWEKDRTNQTGVEPCEGDQDKRLHCYASWRNSSGTIELVKKGCWLDDFNCYDRQECVATEENPQVYFCCCEGNFCNERFTHLPEAGGPEVTYEPPPTAPT

SEQ ID NO: 318 - protein, 110 aa, synthetic construct
ETRECIYYNANWELERTNQSGLERCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 319 - protein, 110 aa, synthetic construct
ETRECIYYNANWEKDRTNQTGVEPCEGDQDKRLHCYASWRNSSGTIELVKKGCWLDDFNCYDRQECVATEENPQVYFCCCEGNFCNERFTHLPEAGGPEVTYEPPPTAPT

SEQ ID NO: 320 - protein, 110 aa, synthetic construct; identical to SEQ ID NO: 317

SEQ ID NO: 321 - protein, 110 aa, synthetic construct
ETQECIYYNANWEKDRTNQTGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 322 - protein, 110 aa, synthetic construct
ETQECIYYNANWEKDRTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 323 - protein, 110 aa, synthetic construct
ETRECIYYNANWEKDRTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 324 - protein, 110 aa, synthetic construct
ETQECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 325 - protein, 110 aa, synthetic construct
ETRECIYYNANWELERTNQSGLERCEGEQDKRLHCFATWRNSSGTIELVKQGCWLDDFNCYDRQECVATEENPQVYFCCCEGNFCNERFTHLPEAGGPEVTYEPPPTAPT

SEQ ID NO: 326 - protein, 110 aa, synthetic construct
ETRECIYYNANWELERTNQSGLERCYGDKDKRRHCYASWRNSSGTIELVKKGCWLDDINCYDRQECVATEENPQVYFCCCEGNFCNERFTHLPEAGGPEVTYEPPPTAPT

SEQ ID NO: 327 - protein, 110 aa, synthetic construct
ETRECIYYNANWELERTNQSGLERCEGEQDKRLHCYASWRNSSGTIELVKKGCWLDDFNCYDRQECVATKENPQVYFCCCEGNFCNERFTHLPEAGGPEVTYEPPPTAPT

SEQ ID NO: 328 - protein, 110 aa, synthetic construct
ETRECIYYNANWELERTNQSGLERCEGEQDKRLHCYASWRNSSGTIELVKKGCWLDDINCYDRQECVATEENPQVYFCCCEGNFCNERFTHLPEAGGPEVTYEPPPTAPT

SEQ ID NO: 329 - protein, 110 aa, synthetic construct
ETRECIYYNANWELERTNQSGLERCEGEQDKRLHCYASWRNSSGTIELVKKGCWLDDINCYDRQECVATKENPQVYFCCCEGNFCNERFTHLPEAGGPEVTYEPPPTAPT

SEQ ID NO: 330 - protein, 110 aa, synthetic construct
ETRECIYYNANWELERTNQSGLERCEGDKDKRLHCYASWRNSSGTIELVKKGCWLDDINCYDRQECVATKENPQVYFCCCEGNFCNERFTHLPEAGGPEVTYEPPPTAPT

SEQ ID NO: 331 - protein, 110 aa, synthetic construct
ETRECIYYNANWELERTNQSGLERCEGDQDKRLHCYASWRNSSGTIELVKKGCWLDDINCYDRQECVATKENPQVYFCCCEGNFCNERFTHLPEAGGPEVTYEPPPTAPT

SEQ ID NO: 332 - protein, 110 aa, synthetic construct
ETRECIYYNANWELERTNQSGLERCEGDQDKRLHCYASWRNSSGTIELVKKGCWLDDFNCYDRQECVATKENPQVYFCCCEGNFCNERFTHLPEAGGPEVTYEPPPTAPT

SEQ ID NO: 333 - protein, 110 aa, synthetic construct
ETRECIYYNANWELERTNQSGLERCEGDQDKRLHCYASWRNSSGTIELVKKGCWLDDFNCYDRQECVATEENPQVYFCCCEGNFCNERFTHLPEAGGPEVTYEPPPTAPT

SEQ ID NO: 334 - protein, 110 aa, synthetic construct
ETRECIYYNANWELERTNQSGLERCYGDKDKRRHCYASWRNSSGTIELVKKGCWLDDINCYDRQECVEKKDSPEVYFCCCEGNFCNERFTHLPEAGGPEVTYEPPPTAPT

SEQ ID NO: 335 - protein, 110 aa, synthetic construct
ETRECIFFNANWEKDRTNQTGVEPCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 336 - protein, 110 aa, synthetic construct
ETRECIYYNANWELERTNQSGLERCEGEQDKRLHCFATWKNISGSIELVKQGCWLDDFNCYDRQECVATEENPQVYFCCCEGNFCNERFTHLPEAGGPEVTYEPPPTAPT

SEQ ID NO: 337 - protein, 110 aa, synthetic construct
ETRECIYYNANWELERTNQSGLERCEGEQDKRLHCYASWRNSSGTIELVKKGCWLDDFNCYDRTDCVATEENPQVYFCCCEGNFCNERFTHLPEAGGPEVTYEPPPTAPT

SEQ ID NO: 338 - protein, 110 aa, synthetic construct
ETRECIYYNANWELERTNQSGLERCEGEQDKRLHCYASWRNSSGTIELVKKGCWLDDFNCYDRQECVATEENPQVYFCCCEGNMCNERFTHLPEAGGPEVTYEPPPTAPT

SEQ ID NO: 339 - protein, 110 aa, synthetic construct
ETRECIYYNANWELERTNQSGLERCEGDQDKRLHCYASWRNSSGTIELVKKGCWLDDINCYDRQECVATEENPQVYFCCCEGNFCNERFTHLPEAGGPEVTYEPPPTAPT

SEQ ID NO: 340 - protein, 110 aa, synthetic construct
ETQECLFFNANWEKDRTNQSGVEPCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 341 - protein, 110 aa, synthetic construct
ETQECLFFNANWEKDRTNQSGVEPCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 342 - protein, 110 aa, synthetic construct
ETRECLFFNANWEKDRTNQSGVEPCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 343 - protein, 110 aa, synthetic construct
ETQECLFFNANWEKDRTNQSGVEPCYGEQDKRLHCYASWRNSSGTIELVKKGCWLDDFNCYDRQECVATEENPQVYFCCCEGNFCNERFTHLPEAGGPEVTYEPPPTAPT

SEQ ID NO: 344 - protein, 110 aa, synthetic construct
ETRECLFFNANWEKDRTNQSGVEPCYGDKDKRRHCYASWRNSSGTIELVKKGCWLDDFNCYDRQECVATEENPQVYFCCCEGNFCNERFTHLPEAGGPEVTYEPPPTAPT

SEQ ID NO: 345 - protein, 110 aa, synthetic construct
ETRECLFFNANWEKDRTNQTGVEPCYGDKDKRRHCFATWKNISGSIEIVKQGCWLDDFNCYDRQECVATEENPQVYFCCCEGNFCNERFTHLPEAGGPEVTYEPPPTAPT

SEQ ID NO: 346 - protein, 110 aa, synthetic construct
ETRECLFFNANWEKDRTNQTGVEPCYGDKDKRRHCFATWKNISGSIEIVKQGCWLDDINCYDRQECVATEENPQVYFCCCEGNFCNERFTHLPEAGGPEVTYEPPPTAPT

SEQ ID NO: 347 - protein, 110 aa, synthetic construct
ETRECLFFNANWEKDRTNQTGVEPCYGDKDKRRHCFATWKNISGSIEIVKQGCWLDDINCYDRTDCVEKKDSPEVYFCCCEGNFCNERFTHLPEAGGPEVTYEPPPTAPT

SEQ ID NO: 348 - protein, 110 aa, synthetic construct
ETQECIYYNANWELERTNQSGLERCYGDKDKRRHCFATWKNISGSIEIVKQGCWLDDINCYDRQECVATEENPQVYFCCCEGNFCNERFTHLPEAGGPEVTYEPPPTAPT

SEQ ID NO: 349 - protein, 110 aa, synthetic construct
ETRECIYYNANWELERTNQSGLERCYGDKDKRRHCFATWKNISGSIEIVKQGCWLDDINCYDRTDCVEKKDSPEVYFCCCEGNFCNERFTHLPEAGGPEVTYEPPPTAPT

SEQ ID NO: 350 - protein, 110 aa, synthetic construct
ETRECIYYNANWELERTNQSGLERCYGDKDKRRHCFATWKNISGSIEIVKQGCWLDDINCYDRTDCVEKKDSPEVYFCCCEGNMCNERFTHLPEAGGPEVTYEPPPTAPT

SEQ ID NO: 351 - protein, 110 aa, synthetic construct
ETRECIYYNANWELERTNQSGLERCEGEQDKRLHCFATWKNISGSIEIVKQGCWLDDINCYDRTDCVEKKDSPEVYFCCCEGNFCNERFTHLPEAGGPEVTYEPPPTAPT

SEQ ID NO: 352 - protein, 110 aa, synthetic construct
ETRECIYYNANWELERTNQSGLERCEGEQDKRLHCFATWKNISGSIEIVKQGCWLDDINCYDRTDCVATEENPQVYFCCCEGNFCNERFTHLPEAGGPEVTYEPPPTAPT

SEQ ID NO: 353 - protein, 110 aa, synthetic construct
ETRECIYYNANWELERTNQSGLERCEGEQDKRLHCFATWKNISGSIEIVKQGCWLDDFNCYDRTDCVATEENPQVYFCCCEGNFCNERFTHLPEAGGPEVTYEPPPTAPT

SEQ ID NO: 354 - protein, 110 aa, synthetic construct
ETRECIYYNANWELERTNQSGLERCEGEQDKRLHCFATWKNISGSIEIVKQGCWLDDFNCYDRQECVATEENPQVYFCCCEGNFCNERFTHLPEAGGPEVTYEPPPTAPT

SEQ ID NO: 355 - protein, 110 aa, synthetic construct
ETQECIYYNANWELERTNQSGLERCYGDKDKRRHCYASWRNSSGTIELVKKGCWLDDINCYDRTDCVEKKDSPEVYFCCCEGNFCNERFTHLPEAGGPEVTYEPPPTAPT

SEQ ID NO: 356 - protein, 110 aa, synthetic construct
ETQECIYYNANWELERTNQSGLERCYGDKDKRRHCYASWRNSSGTIELVKKGCWLDDINCYDRTDCVEKKDSPEVYFCCCEGNMCNERFTHLPEAGGPEVTYEPPPTAPT

SEQ ID NO: 357 - protein, 110 aa, synthetic construct
ETQECIYYNANWELERTNQSGLERCYGDKDKRRHCFATWKNISGSIEIVKQGCWLDDINCYDRTDCVEKKDSPEVYFCCCEGNMCNERFTHLPEAGGPEVTYEPPPTAPT

SEQ ID NO: 358 - protein, 110 aa, synthetic construct
ETQECIYYNANWELERTNQSGLERCYGDKDKRRHCFATWKNISGSIEIVKQGCWLDDINCYDRTDCVEKKDSPEVYFCCCEGNFCNERFTHLPEAGGPEVTYEPPPTAPT

SEQ ID NO: 359 - protein, 110 aa, synthetic construct
ETRECLFFNANWEKDRTNQTGVEPCEGEQDKRLHCFATWKNISGSIEIVKQGCWLDDINCYDRTDCVEKKDSPEVYFCCCEGNFCNERFTHLPEAGGPEVTYEPPPTAPT

SEQ ID NO: 360 - protein, 110 aa, synthetic construct
ETRECLFFNANWEKDRTNQSGVEPCEGEQDKRLHCYASWRNSSGTIELVKKGCWLDDINCYDRTDCVEKKDSPEVYFCCCEGNFCNERFTHLPEAGGPEVTYEPPPTAPT

SEQ ID NO: 361 - protein, 110 aa, synthetic construct
ETRECLFFNANWEKDRTNQSGVEPCYGDKDKRRHCYASWRNSSGTIELVKKGCWLDDINCYDRTDCVEKKDSPEVYFCCCEGNFCNERFTHLPEAGGPEVTYEPPPTAPT

SEQ ID NO: 362 - protein, 110 aa, synthetic construct
ETRECIYYNANWELERTNQSGLERCYGDKDKRRHCYASWRNSSGTIELVKKGCWLDDFNCYDRTDCVATEENPQVYFCCCEGNFCNERFTHLPEAGGPEVTYEPPPTAPT

SEQ ID NO: 363 - protein, 110 aa, synthetic construct
ETRECIYYNANWELERTNQSGLERCYGDKDKRRHCYASWRNSSGTIELVKQGCWLDDFNCYDRTDCVATEENPQVYFCCCEGNFCNERFTHLPEAGGPEVTYEPPPTAPT

SEQ ID NO: 364 - protein, 110 aa, synthetic construct
ETRECIYYNANWELERTNQSGLERCYGDKDKRRHCYASWRNSSGTIEIVKKGCWLDDFNCYDRTDCVATEENPQVYFCCCEGNFCNERFTHLPEAGGPEVTYEPPPTAPT

SEQ ID NO: 365 - protein, 110 aa, synthetic construct
ETRECIYYNANWELERTNQSGLERCYGDKDKRRHCYASWRNSSGSIELVKKGCWLDDFNCYDRTDCVATEENPQVYFCCCEGNFCNERFTHLPEAGGPEVTYEPPPTAPT

SEQ ID NO: 366 - protein, 110 aa, synthetic construct
ETRECIYYNANWELERTNQSGLERCYGDKDKRRHCYASWRNSSGSIEIVKKGCWLDDFNCYDRTDCVATEENPQVYFCCCEGNFCNERFTHLPEAGGPEVTYEPPPTAPT

SEQ ID NO: 367 - protein, 110 aa, synthetic construct
ETRECIYYNANWELERTNQSGLERCYGDKDKRRHCYASWRNSSGSIEIVKQGCWLDDFNCYDRTDCVATEENPQVYFCCCEGNFCNERFTHLPEAGGPEVTYEPPPTAPT

SEQ ID NO: 368 - protein, 110 aa, synthetic construct; identical to SEQ ID NO: 337

SEQ ID NO: 369 - protein, 110 aa, synthetic construct
ETRECIYYNANWELERTNQSGLERCEGEQDKRLHCYASWRNSSGTIELVKKGCWLDDFNCYDRQECVATEENPQVYFCCCEGNFCNEKFSYFPEMEVTQPTSNPVTPKPP

SEQ ID NO: 370 - protein, 110 aa, synthetic construct
ETRECIYYNANWELERTNQSGLERCEGEQDKRLHCYASWRNSSGTIELVKKGCWLDDFNCYDRQECVATEENPQVYFCCCEGNFCNEKFSYFPQMEVTQPTSNPVTPKPP

SEQ ID NO: 371 - protein, 110 aa, synthetic construct
ETRECIYYNANWELERTNQSGLERCYGDKDKRRHCYASWRNSSGTIELVKKGCWLDDINCYDRTDCVEKKDSPEVYFCCCEGNFCNEKFSYFPEMEVTQPTSNPVTPKPP

SEQ ID NO: 372 - protein, 110 aa, synthetic construct
ETRECIYYNANWELERTNQSGLERCYGDKDKRRHCYASWRNSSGTIELVKKGCWLDDINCYDRTDCVEKKDSPEVYFCCCEGNFCNEKFSYFPQMEVTQPTSNPVTPKPP

SEQ ID NO: 373 - protein, 110 aa, synthetic construct
ETRECIYYNANWELERTNQSGLERCYGDKDKRRHCYASWRNSSGTIELVKKGCWLDDFNCYDRQECVATEENPQVYFCCCEGNFCNEKFSYFPQMEVTQPTSNPVTPKPP

SEQ ID NO: 374 - protein, 110 aa, synthetic construct
ETRECIYYNANWELERTNQSGLERCYGDKDKRRHCYASWRNSSGTIELVKQGCWLDDFNCYDRQECVATEENPQVYFCCCEGNFCNEKFSYFPQMEVTQPTSNPVTPKPP

SEQ ID NO: 375 - protein, 110 aa, synthetic construct
ETRECIYYNANWELERTNQSGLERCYGDKDKRRHCYASWRNSSGTIEIVKKGCWLDDFNCYDRQECVATKENPQVYFCCCEGNFCNEKFSYFPQMEVTQPTSNPVTPKPP

SEQ ID NO: 376 - protein, 110 aa, synthetic construct
ETRECIYYNANWELERTNQSGLERCYGDKDKRRHCYASWRNSSGSIELVKKGCWLDDFNCYDRQECVATKENPQVYFCCCEGNFCNEKFSYFPQMEVTQPTSNPVTPKPP

SEQ ID NO: 377 - protein, 110 aa, synthetic construct
ETRECIYYNANWELERTNQSGLERCEGEQDKRLHCYASWRNSSGSIELVKKGCWLDDFNCYDRQECVATEENPQVYFCCCEGNFCNEKFSYFPQMEVTQPTSNPVTPKPP

SEQ ID NO: 378 - protein, 110 aa, synthetic construct
ETRECIYYNANWELERTNQSGLERCEGEQDKRLHCYASWRNSSGTIEIVKKGCWLDDFNCYDRQECVATEENPQVYFCCCEGNFCNEKFSYFPQMEVTQPTSNPVTPKPP

SEQ ID NO: 379 - protein, 110 aa, synthetic construct
ETRECIYYNANWELERTNQSGLERCEGEQDKRLHCYASWRNSSGTIELVKQGCWLDDFNCYDRQECVATEENPQVYFCCCEGNFCNEKFSYFPQMEVTQPTSNPVTPKPP

SEQ ID NO: 380 - protein, 110 aa, synthetic construct
ETRECIYYNANWELERTNQSGLERCEGEQDKRLHCYASWRNSSGTIELVKKGCWLDDFNCYDRQECVETEENPQVYFCCCEGNFCNEKFSYFPQMEVTQPTSNPVTPKPP

SEQ ID NO: 381 - protein, 110 aa, synthetic construct
ETRECIYYNANWELERTNQSGLERCEGEQDKRLHCYASWRNSSGTIELVKKGCWLDDFNCYDRQECVAKEENPQVYFCCCEGNFCNEKFSYFPQMEVTQPTSNPVTPKPP

SEQ ID NO: 382 - protein, 110 aa, synthetic construct
ETRECIYYNANWELERTNQSGLERCEGEQDKRLHCYASWRNSSGTIELVKKGCWLDDFNCYDRQECVATKENPQVYFCCCEGNFCNEKFSYFPQMEVTQPTSNPVTPKPP

SEQ ID NO: 383 - protein, 110 aa, synthetic construct
ETRECIYYNANWELERTNQSGLERCEGEQDKRLHCYASWRNSSGTIELVKKGCWLDDFNCYDRQECVATEDNPQVYFCCCEGNFCNEKFSYFPQMEVTQPTSNPVTPKPP

SEQ ID NO: 384 - protein, 110 aa, synthetic construct
ETRECIYYNANWELERTNQSGLERCEGEQDKRLHCYASWRNSSGTIELVKKGCWLDDFNCYDRQECVATEESPQVYFCCCEGNFCNEKFSYFPQMEVTQPTSNPVTPKPP

SEQ ID NO: 385 - protein, 110 aa, synthetic construct
ETRECIYYNANWELERTNQSGLERCEGEQDKRLHCYASWRNSSGTIELVKKGCWLDDFNCYDRQECVATEENPEVYFCCCEGNFCNEKFSYFPQMEVTQPTSNPVTPKPP

SEQ ID NO: 386 - protein, 110 aa, synthetic construct
ETRECIYYNANWELERTNQSGLERCEGDKDKRLHCYASWRNSSGTIELVKKGCWLDDFNCYDRQECVETEENPQVYFCCCEGNFCNEKFSYFPQMEVTQPTSNPVTPKPP

SEQ ID NO: 387 - protein, 110 aa, synthetic construct
ETRECIYYNANWELERTNQSGLERCEGEKDKRLHCYASWRNSSGTIELVKKGCWLDDFNCYDRQECVAKEENPQVYFCCCEGNFCNEKFSYFPQMEVTQPTSNPVTPKPP

SEQ ID NO: 388 - protein, 110 aa, synthetic construct
ETRECIYYNANWELERTNQSGLERCEGDQDKRLHCYASWRNSSGTIELVKKGCWLDDFNCYDRQECVATKENPQVYFCCCEGNFCNEKFSYFPQMEVTQPTSNPVTPKPP

SEQ ID NO: 389 - protein, 110 aa, synthetic construct
ETRECIYYNANWELERTNQSGLERCEGDKDKRLHCYASWRNSSGTIELVKQGCWLDDFNCYDRQECVAKKENPQVYFCCCEGNFCNEKFSYFPQMEVTQPTSNPVTPKPP

SEQ ID NO: 390 - protein, 110 aa, synthetic construct
ETRECIYYNANWELERTNQSGLERCEGDKDKRLHCYASWRNSSGTIEIVKQGCWLDDFNCYDRQECVAEKENPQVYFCCCEGNFCNEKFSYFPQMEVTQPTSNPVTPKPP

SEQ ID NO: 391 - protein, 110 aa, synthetic construct
ETRECIYYNANWELERTNQSGLERCYGDQDKRLHCYASWRNSSGSIEIVKQGCWLDDFNCYDRQECVAKKENPQVYFCCCEGNFCNEKFSYFPQMEVTQPTSNPVTPKPP

SEQ ID NO: 392 - protein, 110 aa, synthetic construct
ETRECIYYNANWELERTNQSGLERCEGEKDKRRHCYASWRNSSGTIEIVKKGCWLDDFNCYDRQECVATKENPQVYFCCCEGNFCNEKFSYFPQMEVTQPTSNPVTPKPP

SEQ ID NO: 393 - protein, 110 aa, synthetic construct
ETRECIYYNANWELERTNQSGLERCYGDQDKRRHCYASWRNSSGTIELVKKGCWLDDFNCYDRQECVATEENPEVYFCCCEGNFCNEKFSYFPQMEVTQPTSNPVTPKPP

SEQ ID NO: 394 - protein, 110 aa, synthetic construct
ETRECIYYNANWELERTNQSGLERCEGEQDKRRHCYASWRNSSGTIELVKKGCWLDDFNCYDRQECVATEENPQVYFCCCEGNFCNEKFSYFPQMEVTQPTSNPVTPKPP

SEQ ID NO: 395 - protein, 110 aa, synthetic construct
ETRECIYYNANWELERTNQSGLERCYGEQDKRLHCYASWRNSSGSIEIVKKGCWLDDFNCYDRTDCVATEENPQVYFCCCEGNFCNEKFSYFPQMEVTQPTSNPVTPKPP

SEQ ID NO: 396 - protein, 110 aa, synthetic construct
ETRECIYYNANWELERTNQSGLERCEGEQDKRRHCYASWRNSSGSIELVKKGCWLDDFNCYDRQECVAKEENPQVYFCCCEGNFCNEKFSYFPQMEVTQPTSNPVTPKPP

SEQ ID NO: 397 - protein, 110 aa, synthetic construct
ETRECIYYNANWELERTNQSGLERCEGEQDKRRHCYASWRNSSGTIEIVKKGCWLDDFNCYDRQECVAKEENPQVYFCCCEGNFCNEKFSYFPQMEVTQPTSNPVTPKPP

SEQ ID NO: 398 - protein, 110 aa, synthetic construct
ETRECIYYNANWELERTNQSGLERCEGEQDKRRHCYASWRNSSGSIEIVKKGCWLDDFNCYDRQECVATKENPQVYFCCCEGNFCNEKFSYFPQMEVTQPTSNPVTPKPP

SEQ ID NO: 399 - protein, 110 aa, synthetic construct
ETRECIYYNANWELERTNQSGLERCEGDQDKRLHCYASWRNSSGTIEIVKKGCWLDDFNCYDRQECVATKENPQVYFCCCEGNFCNEKFSYFPQMEVTQPTSNPVTPKPP

SEQ ID NO: 400 - protein, 110 aa, synthetic construct
ETRECIYYNANWELERTNQSGLERCEGDQDKRLHCYASWRNSSGSIELVKKGCWLDDFNCYDRQECVATKENPQVYFCCCEGNFCNEKFSYFPQMEVTQPTSNPVTPKPP

SEQ ID NO: 401 - protein, 110 aa, synthetic construct; identical to SEQ ID NO: 388

SEQ ID NO: 402 - protein, 110 aa, synthetic construct
ETRECIYYNANWELERTNQSGLERCEGEQDKRLHCYASWRNSSGTIEIVKKGCWLDDFNCYDRQECVATKENPQVYFCCCEGNFCNEKFSYFPQMEVTQPTSNPVTPKPP

SEQ ID NO: 403 - protein, 110 aa, synthetic construct; identical to SEQ ID NO: 382

SEQ ID NO: 404 - protein, 110 aa, synthetic construct
ETRECIYYNANWELERTNQSGLERCEGDQDKRLHCYASWRNSSGTIEIVKKGCWLDDFNCYDRQECVAKKENPQVYFCCCEGNFCNEKFSYFPQMEVTQPTSNPVTPKPP

SEQ ID NO: 405 - protein, 110 aa, synthetic construct
ETQECLFFNANWEKDRTNQTGVEPCYGDKDKRRHCFATWKNISGSIEIVKQGCWLDDINCYDRTDCVEKKDSPEVYFCCCEGNMCNERFTHLPEAGGPEVTYEPPPTAPT

SEQ ID NO: 406 - protein, 110 aa, synthetic construct
ETRECIYYNANWELERTNQSGLERCYGDKDKRRHCYASWRNSSGTIELVKKGCWLDDINCYDRQECVATKENPQVYFCCCEGNFCNERFTHLPEAGGPEVTYEPPPTAPT

SEQ ID NO: 407 - protein, 116 aa, synthetic construct
SGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 408 - protein, 513 aa, synthetic construct
MGAATKLAFAVFLISCSSGAILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKPPLFNTLLYSLVPIMGIAVIVLFSFWMYRHHKLAYPPVLVPTQDPGPPPPSPLMGLKPLQLLEIKARGRFGCVWKAQLLNEYVAVKIFPIQDKQSWQNEYEIYSLPGMKHDNILQFIGAEKRGTSIDVDLWLITAFHEKGSLTDFLKANVVSWNELCHIAQTMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTASDGPVDEYMLPFEEEIGQHPSLEDMQEVVVHKKKRPVLRECWQKHSGMAMLCETIEECWDHDAEARLSAGCVEERIIQMQKLTNIITTEDIVTVVTMVTNVDFPPKESSL

SEQ ID NO: 409 - protein, 116 aa, synthetic construct
AILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 410 - protein, 6 aa, synthetic construct
NPVTPK

SEQ ID NO: 411 - protein, 4 aa, synthetic construct
TEEN

SEQ ID NO: 412 - protein, 4 aa, synthetic construct
TKEN

SEQ ID NO: 413 - protein, 10 aa, synthetic construct
  VARIANT 1..10: note = This sequence may encompass 1-2 GGGGS repeating units
GGGGSGGGGS

SEQ ID NO: 414 - protein, 20 aa, synthetic construct
  VARIANT 1..20: note = This sequence may encompass 1-4 GGGGS repeating units
GGGGSGGGGSGGGGSGGGGS

SEQ ID NO: 415 - protein, 5 aa, synthetic construct
GGGGS

SEQ ID NO: 416 - protein, 10 aa, synthetic construct
  VARIANT 1: note = Any amino acid
  VARIANT 3: note = Any amino acid
  VARIANT 5: note = Any amino acid
  VARIANT 7: note = Any amino acid
  VARIANT 9: note = any amino acid
  VARIANT 1..10: note = This sequence may encompass 1-5 XP repeating units
XPXPXPXPXP